(19) United States
(12) Patent Application Publication
(10) Pub. No.: US 2025/0368646 A1
(43) Pub. Date: Dec. 4, 2025

(54) TRICYCLIC FUSED WRN INHIBITORS

(71) Applicant: Nimbus Wadjet, Inc., Boston, MA (US)

(72) Inventors: Derun Li, Boston, MA (US); Angela V. West, Franklin, MA (US); Justin Caravella, Cambridge, MA (US); Nathan E. Genung, Boston, MA (US); Florian Bartels, Berlin (DE); Robert Lee Dow, Groton, CT (US); Silvana Marcel Leit de Moradei, Burlington, MA (US); Nikolay Sitnikov, Berlin (DE)

(21) Appl. No.: 19/220,710

(22) Filed: May 28, 2025

Related U.S. Application Data
(60) Provisional application No. 63/652,476, filed on May 28, 2024.
(60) Provisional application No. 63/663,020, filed on Jun. 21, 2024.
(60) Provisional application No. 63/693,030, filed on Sep. 10, 2024.
(60) Provisional application No. 63/799,045, filed on May 2, 2025.

(51) Int. Cl. C07D 471/14; A61K 31/506; A61K 31/519; C07D 487/14

(52) U.S. Cl. CPC C07D 471/14; A61K 31/506; A61K 31/519; C07D 487/14

(57) ABSTRACT

The present disclosure is directed to compounds of Formula I:

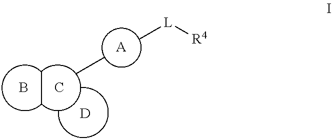

and pharmaceutically acceptable salts thereof, and compositions thereof, as well as methods of treatment of cancers such as those involving WRN protein.

### CROSS REFERENCE TO RELATED APPLICATIONS

This application claims the benefit of Provisional Application No. 63/652,476, filed May 28, 2024; Provisional Application No. 63/663,020, filed Jun. 21, 2024; Provisional Application No. 63/693,030, filed Sep. 10, 2024; and Provisional Application No. 63/799,045, filed May 2, 2025; the entirety of each of which are incorporated herein by reference.

### FIELD OF INVENTION

The invention provides tricyclic compounds and compositions, the use thereof and methods using the compounds, for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric and endometrial cancer. The invention also provides the use of said compounds as research chemicals, intermediate compounds, combinations, processes and formulations.

### SEQUENCE LISTING

This application contains a Sequence Listing which has been submitted in .xml format via EFS and is hereby incorporated by reference. The ST.26 copy, created on May 15, 2025, is named 407274-96WR_SL.xml and is 8,108 bytes in size.

### BACKGROUND

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that are deficient in mismatch repair (dMMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer (CRC) which resulted in the recent approval of pembrolizumab as first-line treatment of these cancers, there is still a significant unmet medical need in CRC and other MSI-H indications (Andre T., et al. Pembrolizumab in Microsatellite-Instability-High Advanced Colorectal Cancer. N Engl J Med 383(23):22072218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170(3):577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRN is synthetically lethal with MSI cancers. Depletion of WRN leads to anti-proliferative effects and results in activation of multiple DNA damage signaling markers, induction of cell cycle arrest and apoptosis in MSI-H cancer models but not cancer cells with an intact MMR pathway (otherwise known as microsatellite stable or MSS). The anti-proliferative effects of WRN depletion could not be rescued with a helicase deficient WRN construct, demonstrating that helicase activity of WRN is required for MSI-H viability. These findings indicate that WRN helicase provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of MSI-H cancers.

### SUMMARY

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention also provides compounds, pharmaceutically acceptable salts thereof, pharmaceutical compositions thereof and combinations thereof, said compounds being useful for the treatment of cancer, in particular cancers characterized as microsatellite instability-high (MSI-H) or mismatch repair deficient (dMMR). Also provided are compounds that bind to, and/or inhibit WRN, and are therefore useful as research chemicals, e.g., as a chemical probe, and as tool compounds. Various embodiments of the invention are described herein.

In one aspect, the disclosure provides a compound of Formula I, or a pharmaceutically acceptable salt thereof:

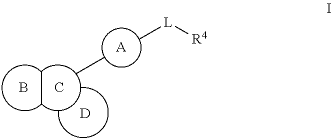

wherein tricyclic Ring BCD, linker L, R⁴, and Ring A are as described and defined herein.

In another aspect, the invention provides a pharmaceutical composition comprising a compound of Formula I of the present invention and one or more pharmaceutically acceptable carriers.

In another aspect, the invention provides a combination, in particular a pharmaceutical combination, comprising a compound of Formula I of the present invention and one or more therapeutically active agents.

In another aspect, the invention provides a compound of Formula I of the present invention for use as a medicament, in particular for the treatment of a disorder or disease which can be treated by WRN inhibition.

In another aspect, the invention provides a compound of Formula I of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of Formula I of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of Formula I of the present invention.

In another aspect, the invention provides the use of a compound of Formula I of the present invention in the manufacture of a medicament for the treatment of a disorder or disease which can be treated by WRN inhibition.

In another aspect, the invention provides a compound of Formula I of the present invention for use as a research chemical, for example as a chemical probe or as a tool compound.

In another aspect, the invention provides a solid form, process or intermediate as described herein.

### DETAILED DESCRIPTION

### 1. General Description of Certain Embodiments of the Invention

In one aspect, the disclosure provides a compound of Formula I, or a pharmaceutically acceptable salt thereof:

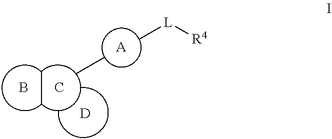

wherein tricyclic Ring BCD is selected from one of the following:

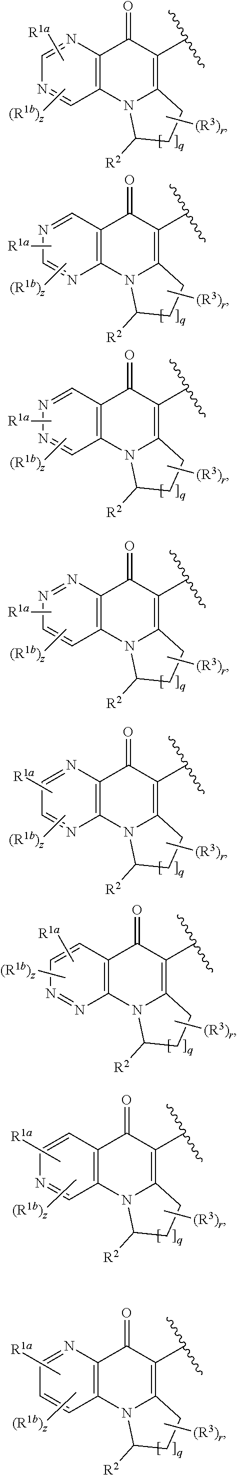

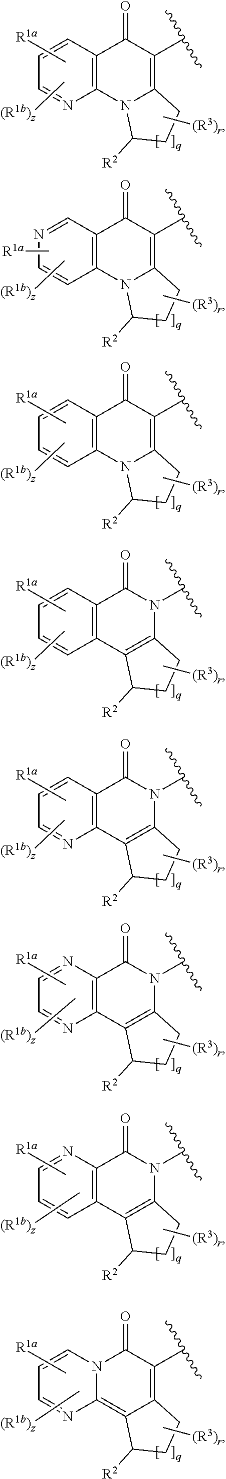

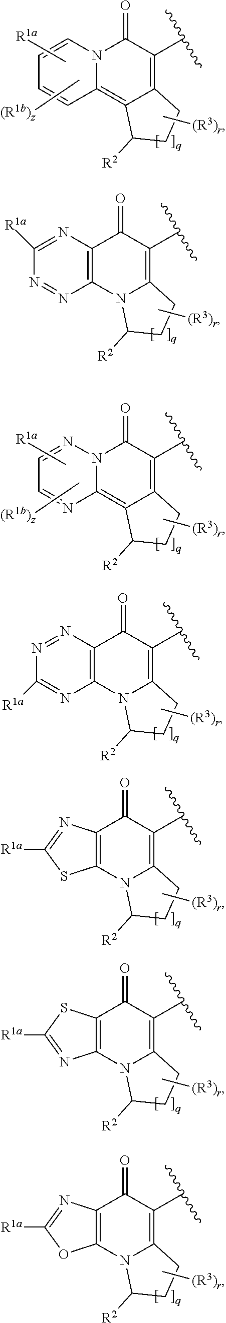

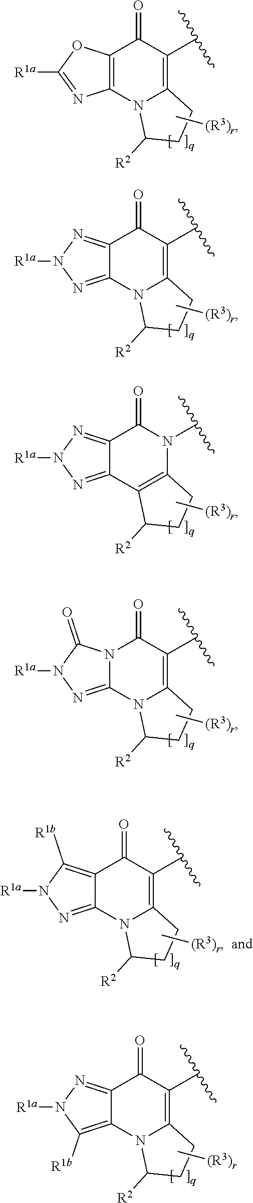

wherein [symbol] denotes the point of attachment to Ring A;
each R^{1b} group is independently selected from H, halogen, CN, OH, C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy, wherein said C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy are each independently and optionally substituted with 1-5 halogen, OH, CN, C₁-C₆alkyl, or C₃-C₆cycloalkyl groups; wherein z is 0, 1, or 2;
Ring A is:
   a) a 4-7 membered saturated or partially unsaturated bivalent monocyclic carbocyclyl or 4-7 membered saturated or partially unsaturated bivalent heterocyclyl ring (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclyl or heterocyclyl (having 1-4 heteroatoms independently selected from nitrogen, oxygen, and sulfur);
wherein Ring A is substituted with 0-4 independently selected R^{B} substituents;
-L- is a linker selected from —C(O)—, —S(O)—, —S(O)₂—, and

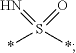

R^{1a} is selected from:
   a) a 5-6 membered heteroaryl (having 1-4 heteroatoms independently selected from nitrogen, oxygen, and sulfur) optionally substituted with 1-3 groups independently selected from halogen, C₁-C₆aliphatic, C₃-C₆cycloalkyl, haloC₁-C₆alkyl, C₁-C₆alkoxy, and C₃-C₆cycloalkoxy, wherein said 5-6 membered heteroaryl is further substituted with 0-3 independently selected R^{B};
   b) a 4-7 membered saturated or partially unsaturated heterocyclyl (having 1-2 heteroatoms independently selected from nitrogen, oxygen, and sulfur), optionally substituted with 1 or 2 groups independently selected from C₁-C₆aliphatic, C₃-C₆cycloalkyl, C₁-C₆alkoxy, C₃-C₆cycloalkoxy, and —OR, wherein said 4-7 membered saturated or partially unsaturated heterocyclyl is further substituted with 0-3 independently selected R^{B}, and two R^{B} along with their intervening atoms optionally join to form a 3-5 membered carbocyclyl;
   c) a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), wherein said carbocyclyl or heterocyclyl is substituted with 0-3 independently selected R^{B}; and
   d) H, halogen, C₁-C₆aliphatic, C₃-C₇cycloalkyl, C₁-C₆alkylene-O—C₁-C₆alkyl, CN, —OR, —OR¹⁰, —NR¹⁰R¹¹, —C(O)NR¹⁰R¹¹, —CH₂NR¹⁰R¹¹, or —SO₂R¹², wherein said C₁-C₆aliphatic, C₃-C₇cycloalkyl, or C₁-C₆alkylene-O—C₁-C₆alkyl is substituted with 0-5 independently selected R^{B};
or R^{1a} and one R^{1b} on adjacent atoms of Ring B, taken together with the adjacent Ring B atoms to which they are attached, form a cyclic group fused to Ring B selected from phenyl, a 5-6 membered heteroaryl (having 1-3 heteroatoms independently selected from nitrogen, oxygen and sulfur), a 4-7 membered saturated or partially unsaturated carbocyclyl, or a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B};
R² is selected from C(O)N(R)R^{2A};
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅, and wherein two substituents on adjacent atoms of the phenyl or pyridyl, together with said adjacent atoms, optionally form a 4-7 membered carbocyclyl fused to the phenyl or pyridyl, and wherein two substituents on adjacent atoms of the phenyl or pyridyl together with said adjacent atoms optionally form a 4-7 membered heterocyclyl (having 1-4 heteroatoms independently selected from nitrogen, oxygen, and sulfur) fused to the phenyl or pyridyl, wherein said fused 4-7 membered carbocyclyl or fused 4-7 membered heterocyclyl is substituted with 0-5 independently selected halogen or methyl; or
R^{2A} is 2-benzimidazolyl, 2-naphthyl, or 3-quinolinyl, each of which is optionally substituted with 1, 2, or 3 substituents independently selected from halogen, C₁₋₄alkyl, and —OH;
each R³ is independently selected from:
   hydrogen, halo, and OH;
   C₁-C₄aliphatic unsubstituted or substituted by 1, 2 or 3 substituents independently selected from halo and OH; and
   C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, OR, —C(O)NR¹⁰R¹¹, or N(R)C(O)R; wherein each R^{3A} is independently selected from C₁-C₄alkyl;
or two R³ substituents on the same ring carbon atom may join, together with the carbon atom to which they are attached, to form a cyclopropyl ring;
R⁴ is phenyl or a first 5-6 membered heteroaryl (having 1-4 heteroatoms independently selected from nitrogen, oxygen, and sulfur) wherein said phenyl or first 5-6 membered heteroaryl is substituted with 0-5 R^{B}; and optionally two adjacent atoms of said phenyl or first 5-6 membered heteroaryl have two substituents that together with said adjacent atoms form a cyclic group fused to the phenyl or first 5-6 membered heteroaryl selected from a 4-7 membered carbocyclyl, a 4-7 membered heterocyclyl (having 1-4 heteroatoms independently selected from nitrogen, oxygen, and sulfur), or a second 5-6 membered heteroaryl (having 1-4 heteroatoms independently selected from nitrogen, oxygen, and sulfur); wherein said fused cyclic group is substituted with 0-3 independently selected R^{B}; or
R⁴ is a C₁-C₄aliphatic, C₁-C₄alkoxy, or C₃-C₆cycloalkyl, each of which is substituted with 0-3 groups independently selected from halogen, —CN, —OH, C₁-C₄alkyl, C₁-C₄alkoxy, and optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur;
R¹⁰ is H, C₁-C₆aliphatic, haloC₁-C₆alkyl, C₃-C₆cycloalkyl, haloC₃-C₆cycloalkyl, —C(O)C₁-C₆alkyl, or a 5-6 membered heteroaryl (having 1-4 heteroatoms independently selected from nitrogen, oxygen, and sulfur); each R¹⁰ except H is optionally substituted with 1 or 2 independently selected R^{B};
R¹¹ is H, C₁-C₆aliphatic, or C₃-C₆cycloalkyl, or R¹⁰ and R¹¹ are taken together with the nitrogen atom to which they are attached to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur); each R¹² is optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{B} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, haloC₁-C₆alkyl, C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, NR₂, S(O)₂R, S(O)₂NR₂, S(O)R, S(O)NR₂, C(O)R, C(O)OR, —C(O)NR₂, C(O)N(R)OR, OC(O)R, OC(O)NR₂, —N(R)C(O)OR, N(R)C(O)R, N(R)C(O)NR₂, N(R)C(NR)NR₂, N(R)S(O)₂NR₂, and —N(R)S(O)₂R;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form a cyclic group selected from an optionally substituted 4-7 membered saturated ring, a 4-7 membered partially unsaturated ring, or a 5-6 membered heteroaryl ring (having 1-4 heteroatoms independently selected from nitrogen, oxygen, and sulfur); wherein said cyclic group has 0-3 heteroatoms independently selected from nitrogen, oxygen, and sulfur);
q is 1 or 2;
r is 0, 1, 2 or 3.

In another aspect, the invention provides a method of treating a disorder or disease which can be treated by WRN inhibition in a subject, comprising administering to the subject a therapeutically effective amount of a compound of Formula I of the present invention, or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001.

Compound structures shown throughout the present specification and in the examples or claims contain designations at certain stereocenters which indicate the following: “or1” and is intended to cover stereochemically pure compounds wherein the stereochemistry at the stereocenter marked with “or1” is either the stereochemistry shown in the diagram or wherein the marked stereocenter has a configuration opposite to what is shown in the diagram. In structures with stereocenters with the same label such as “or1” the relative stereochemistry between two stereocenters with said label is as drawn.

A stereocenter marked with “abs” describes a compound wherein the marked stereocenter is of the absolute stereochemistry shown in the chemical structure. Stereocenters marked with “&1” or “and1” indicate that the compound has a mixture of R and S-configured stereoisomers with respect to the marked stereocenter and is in the same relative configuration to each other if they share the same label such as “and1” or “&1.”

Compound structures shown throughout the present specification and in the examples or claims which contain designations at certain stereocenters which indicate “or1” and contain other designations at certain stereocenters which are absolute and indicate “S” is intended to cover mixtures of stereochemically pure compounds wherein the stereochemistry at the stereocenter marked with “or1” is either the stereochemistry shown in the diagram or wherein the marked “or1” stereocenter has a configuration opposite to what is shown in the diagram and the stereocenters marked “S” are absolute.

Compound structures with stereocenters marked with “or1” and “or2” are intended to cover stereochemically pure compounds wherein the exact stereochemistry at the stereocenter marked with “or1” is independently either the stereochemistry shown in the diagram or wherein the marked stereocenter has a configuration opposite to what is shown in the diagram but has not yet been confirmed and the exact stereochemistry at the stereocenter marked with “or2” is independently either the stereochemistry shown in the diagram or wherein the marked stereocenter has a configuration opposite to what is shown in the diagram but has not yet been confirmed as in I-1, I-2, I-3 and I-4. In structures with stereocenters with different labels such as “or1” and “or2” the relative stereochemistry between the said stereocenters is either as drawn in the diagram or opposite to what is drawn in the diagram as in I-1, I-2, I-3 and I-4. In structures with stereocenters with the same label such as “or1” the relative stereochemistry between two stereocenters with said label is as drawn in the diagram.

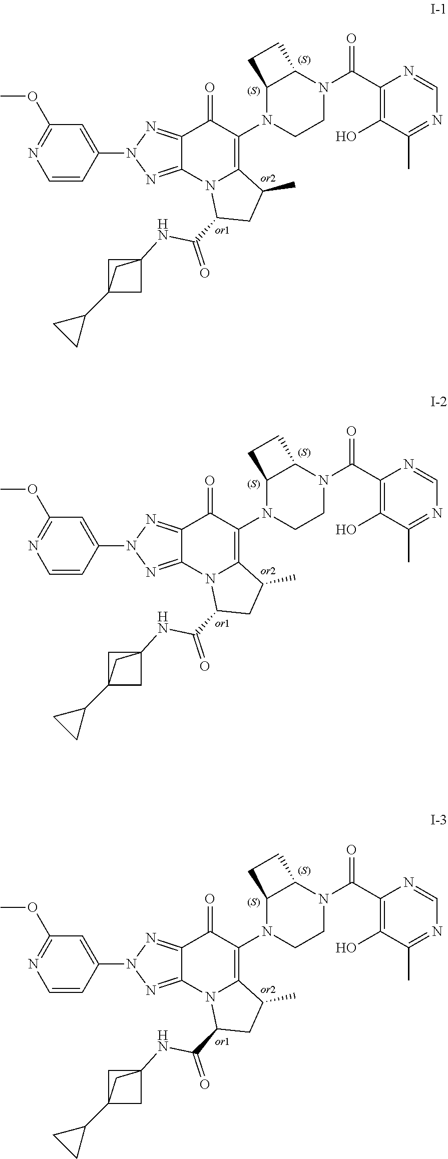

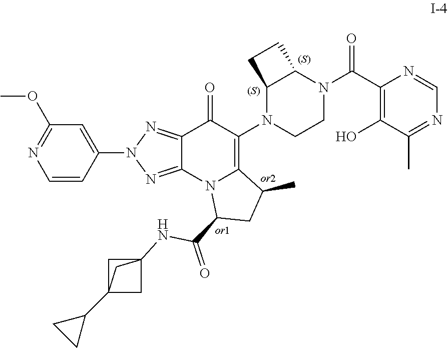

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 5-12 ring members and 0-4 heteroatoms independently selected from nitrogen, oxygen, or sulfur unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl (—CH₃), ethyl (—CH₂CH₃), propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₂₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CHCH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₂₋₆ straight or branched partially unsaturated aliphatic group comprising at least one unsaturated carbon carbon triple bond. The term “lower alkynyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

The term “cubanyl” refers to a substituent of cubane as shown below.

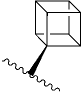

The substituent -Me, as used herein refers to a methyl group, —CH₃.

As used herein, the term “bivalent C₁₋₈ (or C₁₋₆ i.e., C₁-C₆) saturated or unsaturated, straight or branched, hydrocarbon chain,” refers to bivalent alkylene, alkenylene, and alkynylene chains that are straight or branched as defined herein.

As used herein, the term “bivalent,” to describe a cyclic (and noncyclic) group refers to, for example, bivalent carbocyclylene, phenylene, heterocyclylene, and heteroarylene that are bivalent moieties of carbocycles, phenyls, heterocycles, and heteroaryls described herein. Non-limiting examples include

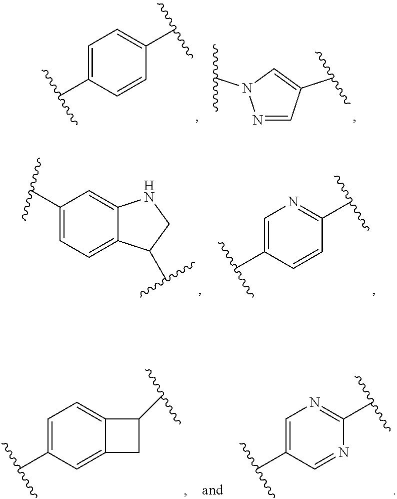 [SMILES: *c1ccc(*)cc1 ; *c1cnn(*)c1 ; *c1ccc2c(c1)NCC2* ; *c1ccc(*)nc1 ; *c1ccc2c(c1)CC2* ; *c1cnc(*)nc1]

“Carbocyclylene” as used herein refers to a carbocyclic or cycloalkyl moiety that is bivalent as described above (i.e., attached at two different points to the rest of the compound). Non-limiting examples include cyclopropylene, cyclobutylene, cyclopentylene, or cyclohexylene as shown below.

| | | Different examples | Different examples |
|---|---|---|---|
| cyclopropylene | cyclobutylene | of cyclopentylene | of cyclohexylene |
| 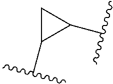 [SMILES: *C1CC1*] | 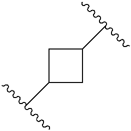 [SMILES: *C1CC(*)C1] | 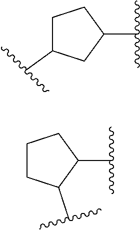 [SMILES: *C1CCC(*)C1 ; *C1CCCC1*] | 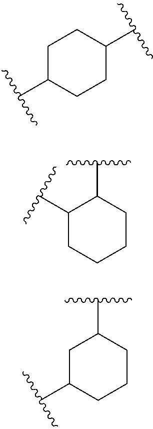 |

A carbocyclylene may be saturated as in the examples shown above or partially unsaturated as in the examples shown below.

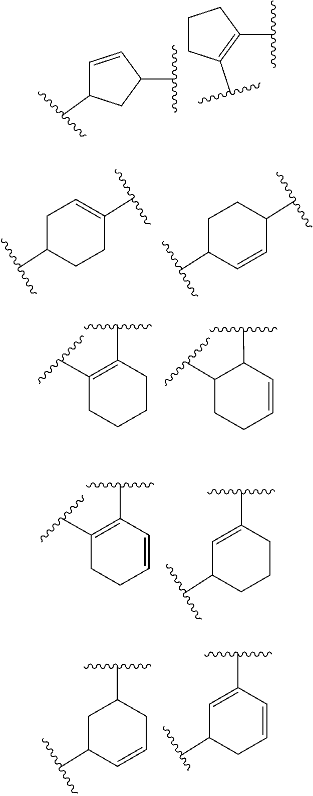

A carbocyclylene may be multi-cyclic, for example, bicyclic or tricyclic. Such multi-cyclic carbocyclylene systems may be saturated or partially unsaturated (while one ring of the bicyclic system may be aromatic it is to be understood that multi-cyclic ring systems that are not in their entirety aromatic may also fall under the definition of carbocyclylene). The rings may form bridged, fused, or spiro systems. Non-limiting examples are shown below.

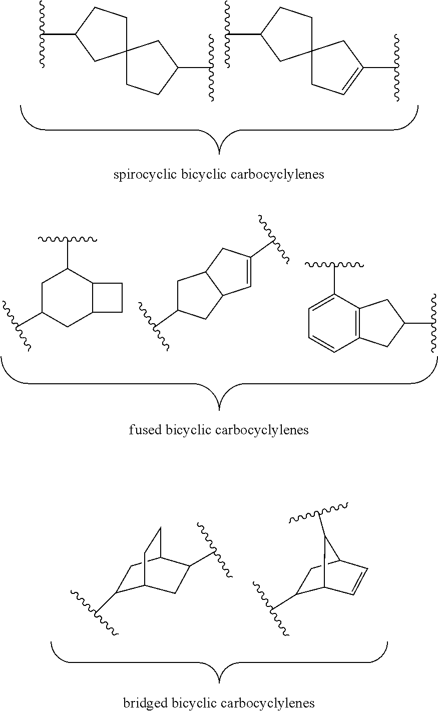

“Heterocyclylene” as used herein refers to a heterocyclic or heterocyclyl moiety that is bivalent as described above (i.e., attached at two different points to the rest of the compound) and may also be saturated or partially unsaturated. Non-limiting examples include those shown below. Heterocyclylene is understood to include bicyclic heterocyclylene systems. Non-limiting examples of bicyclic heterocyclylene moieties are also shown below and said bicyclic systems may be spirocyclic, fused, or bridged and may be saturated or partially unsaturated.

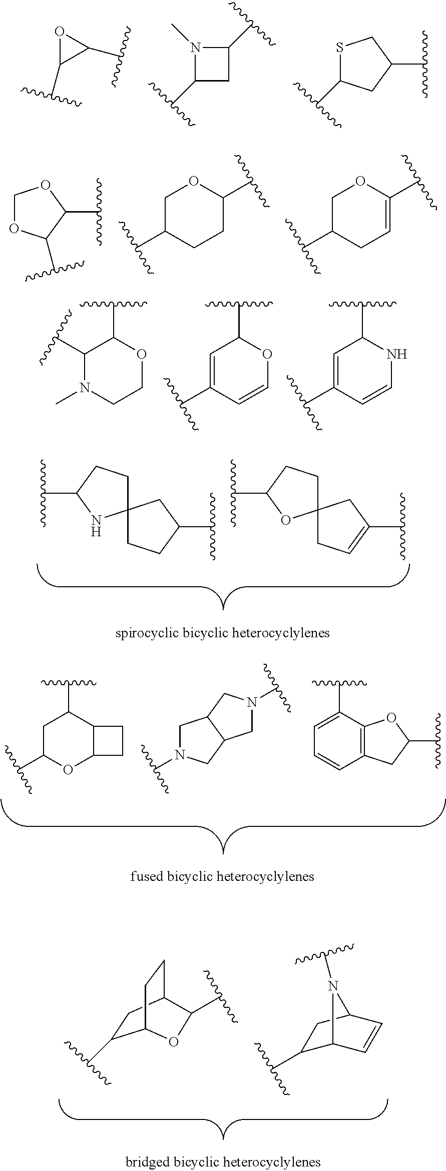

“Phenylene” as used herein refers to a phenyl moiety that is bivalent as described above (i.e., attached at two different points to the rest of the compound). Examples are shown below.

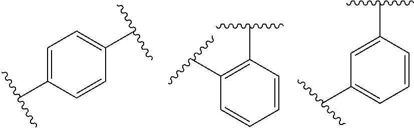

“Arylene” as used herein refers to a mono or multi-cyclic aryl (i.e., phenyl or a multi-cyclic aryl) moiety that is bivalent as described above (i.e., attached at two different points to the rest of the compound), wherein the arylene group contains no heteroatoms. Examples are shown below.

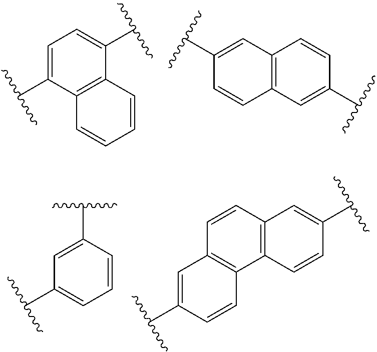 [SMILES: *c1ccc(*)c2ccccc12 ; *c1ccc2cc(*)ccc2c1 ; *c1cccc(*)c1 ; *c1ccc2c(ccc3cc(*)ccc32)c1]

“Heteroarylene,” as used herein refers to a mono or multi-cyclic aryl ring system that contains at least one heteroatom wherein the ring system is bivalent as described above (i.e., attached at two different points to the rest of the compound). Examples are shown below.

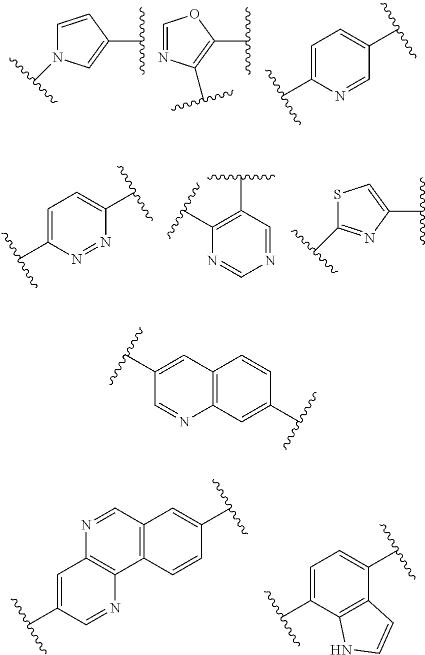

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

“Carbocyclyl (or heterocyclyl, aryl, phenyl, or heteroaryl) fused to” another phenyl, aryl, heteroaryl, carbocyclyl, or heterocyclyl, for example, a “phenyl or pyridyl” as used herein, may be referred to as “partially unsaturated” without said “carbocyclyl (or heterocyclyl, aryl, phenyl, or heteroaryl) fused to” the other ring requiring further unsaturation besides the carbon-carbon bond which it shares with the ring to which it is fused (i.e., the “phenyl or pyridyl”). This is illustrated below.

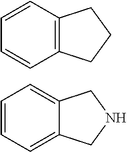

partially unsaturated cyclopentyl fused to phenyl, i.e., “cyclopentyl fused to phenyl” 5-membered partially unsaturated heterocyclyl fused to the phenyl, i.e. “heterocyclyl fused to phenyl”

A further example below shows a carbocyclyl moiety fused to a Ring E as defined in the embodiments herein. Said carbocyclyl does not explicitly require a descriptor of “partially unsaturated” to describe said carbocyclyl because it shares two carbons with the aromatic pyridine to which it is fused. Such language is used herein to describe such systems, for example, “R^{4A} and R^{4B}, along with their intervening atoms, join to form 4-7 membered carbocyclyl that is fused to Ring E” as shown in the image below. As such, “Ring E” may refer to a monocyclic ring (i.e., the pyridine shown below and its substituents which do not form a fused ring), without any further fused rings created by its substituents (i.e., R^{4A} and R^{4B}). Any further fused ring created by the substituents of Ring E is described as being “fused to Ring E.” Likewise, R^{4A} and R^{4B}, along with their intervening atoms, join to form 4-7 membered carbocyclyl or heterocyclyl (having 1-4 heteroatoms independently selected from nitrogen, oxygen, and sulfur) that is fused to Ring E (not pictured), is subject to the same interpretation.

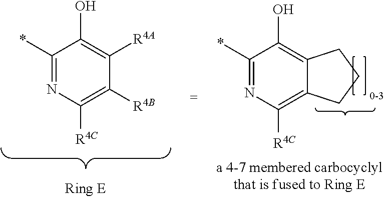

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3triazolyl), 1,2,4triazolyl, 1,2,5triazolyl, 1,3,4triazolyl, tetrazolyl, oxazolyl, isoxazolyl, oxadiazolyl, 1,2,3oxadiazolyl, 1,2,4oxadiazolyl, 1,2,5-oxadiazolyl, 1,3,4oxadiazolyl, thiazolyl, isothiazolyl, thiadiazolyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-onyl, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. Said 7-10-membered bicyclic heterocyclic moiety that is partially unsaturated may include an aryl or heteroaryl ring fused to a non-aromatic ring. For example, said 7-10-membered bicyclic heterocyclic moiety may include a bicyclic heterocyclyl as shown below:

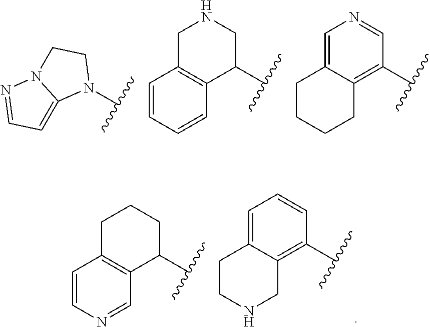

When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

“Arylene” or “heteroarylene,” as used herein (i.e., phenylene), refers to any bivalent aryl or heterocyclyl described herein, that is a bisradical substituted at each of two substitutable positions of the ring system as described in detail supra.

“Heterocyclyloxy,” as used herein, refers to an —OR group wherein the R is a heterocyclyl. Nonlimiting examples are shown below.

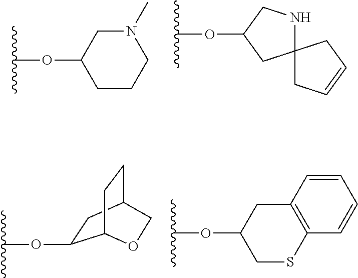

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄B(OR^{∘})₂; —(CH₂)₀₋₄R^{∘}; —(CH₂)₀₋₄OR^{∘}; —O(CH₂)₀₋₄R; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; (CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘2}; —C(S)NR^{∘}₂; —C(S)SR^{∘}; —SC(S)SR^{∘}; —(CH₂)₀₋₄OC(O)NR^{∘2}; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄OP(O)R^{∘}₂; —(CH₂)₀₋₄OP(O)(OR^{∘})₂; —SiR^{∘3}; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —SO₂—C₁₋₄ aliphatic (i.e., —SO₂CH₃)—CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group, which includes instances of R^{∘} (or the ring formed by taking two independent occurrences of R^{∘} together with their intervening atoms), include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, (haloR^{●}), OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, Ring A of a provided compound may be substituted with one or more deuterium atoms.

The structures as drawn represent relative configurations, unless labeled as absolute configurations. The invention contemplates individual enantiomers and racemic mixtures.

### 3. Description of Exemplary Embodiments

In one aspect, the disclosure provides a compound of Formula I, or a pharmaceutically acceptable salt thereof:

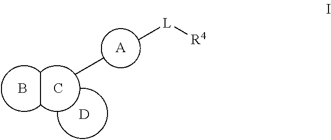

wherein tricyclic Ring BCD is selected from one of the following:

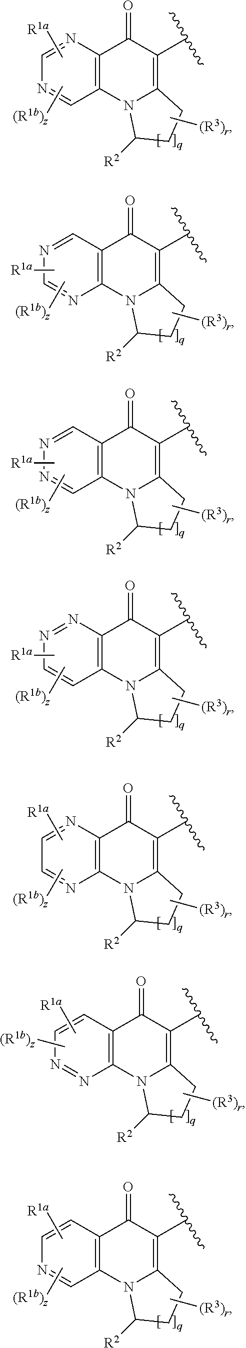

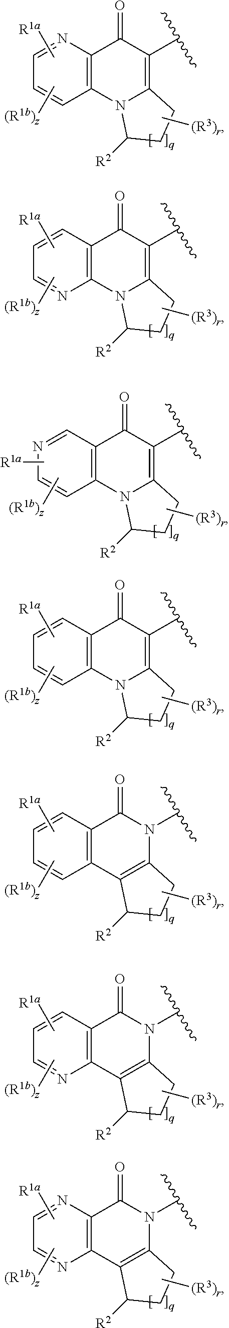

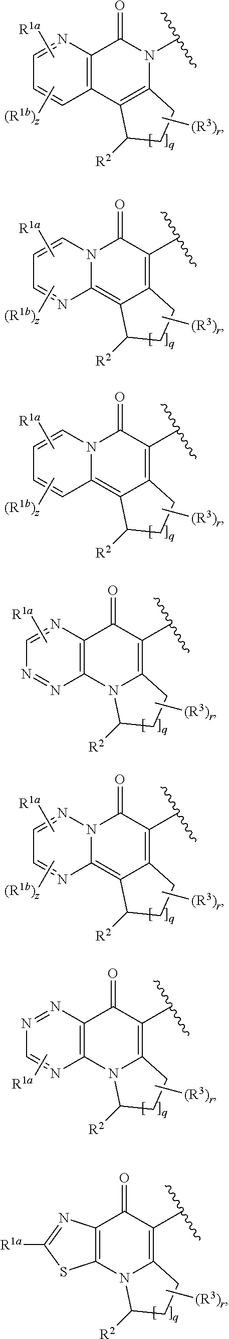

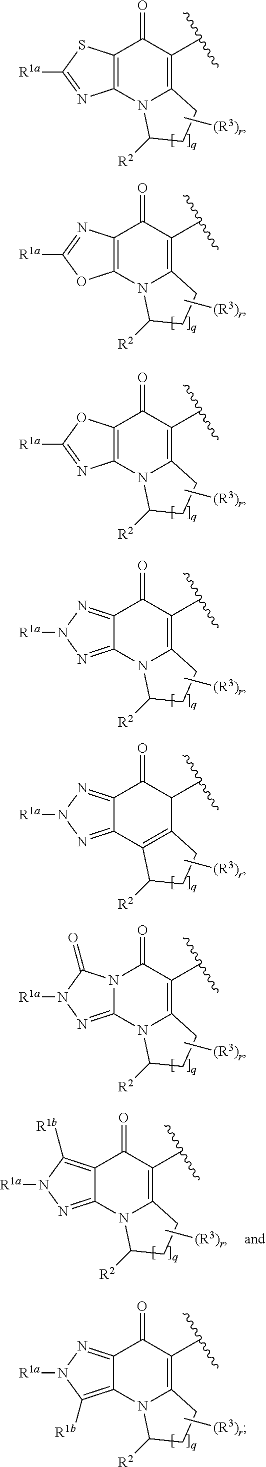

wherein [symbol] denotes the point of attachment to Ring A;
each R^{1b} group is independently selected from H, halogen, CN, OH, C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy, wherein said C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy are each independently and optionally substituted with 1-5 halogen, OH, CN, C₁-C₆alkyl, or C₃-C₆cycloalkyl groups; wherein z is 0, 1, or 2;
Ring A is:
   a) a 4-7 membered saturated or partially unsaturated bivalent monocyclic carbocyclyl or 4-7 membered saturated or partially unsaturated bivalent heterocyclyl ring (having 1-4 heteroatoms independently selected from nitrogen, oxygen, and sulfur); or
   b) a 4-12 membered saturated or partially unsaturated bivalent bicyclic ring system that is fused, bridged, or spirocyclic selected from carbocyclyl or heterocyclyl (having 1-4 heteroatoms independently selected from nitrogen, oxygen, and sulfur);
wherein Ring A is substituted with 0-4 independently selected R^{B} substituents;
-L- is a linker selected from —C(O)—, —S(O)—, —S(O)₂—, and

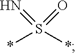

R^{1a} is selected from:
   a) a 5-6 membered heteroaryl (having 1-4 heteroatoms independently selected from nitrogen, oxygen, and sulfur) optionally substituted with 1-3 groups independently selected from halogen, C₁-C₆aliphatic, C₃-C₆cycloalkyl, haloC₁-C₆alkyl, C₁-C₆alkoxy, and C₃-C₆cycloalkoxy, wherein said 5-6 membered heteroaryl is further substituted with 0-3 independently selected R^{B};
   b) a 4-7 membered saturated or partially unsaturated heterocyclyl (having 1-2 heteroatoms independently selected from nitrogen, oxygen, and sulfur), optionally substituted with 1 or 2 groups independently selected from C₁-C₆aliphatic, C₃-C₆cycloalkyl, C₁-C₆alkoxy, C₃-C₆cycloalkoxy, and —OR, wherein said 4-7 membered saturated or partially unsaturated heterocyclyl is further substituted with 0-3 independently selected R^{B}, and two R^{B} along with their intervening atoms optionally join to form a 3-5 membered carbocyclyl;
   c) a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), wherein said carbocyclyl or heterocyclyl is substituted with 0-3 independently selected R^{B}; and
   d) H, halogen, C₁-C₆aliphatic, C₃-C₇cycloalkyl, C₁-C₆alkylene-O—C₁-C₆alkyl, CN, —OR, —OR¹⁰, —NR¹⁰R¹¹, —C(O)NR¹⁰R¹¹, —CH₂NR¹⁰R¹¹, or —SO₂R¹², wherein said C₁-C₆aliphatic, C₃-C₇cycloalkyl, or C₁-C₆alkylene-O—C₁-C₆alkyl is substituted with 0-5 independently selected R^{B};
or R^{1a} and one R^{1b} on adjacent atoms of Ring B, taken together with the adjacent Ring B atoms to which they are attached, form a cyclic group fused to Ring B selected from phenyl, a 5-6 membered heteroaryl (having 1-3 heteroatoms independently selected from nitrogen, oxygen and sulfur), a 4-7 membered saturated or partially unsaturated carbocyclyl, or a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B};
R² is selected from C(O)N(R)R^{2A};
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅, and wherein two substituents on adjacent atoms of the phenyl or pyridyl, together with said adjacent atoms, optionally form a 4-7 membered carbocyclyl fused to the phenyl or pyridyl, and wherein two substituents on adjacent atoms of the phenyl or pyridyl together with said adjacent atoms optionally form a 4-7 membered heterocyclyl (having 1-4 heteroatoms independently selected from nitrogen, oxygen, and sulfur) fused to the phenyl or pyridyl, wherein said fused 4-7 membered carbocyclyl or fused 4-7 membered heterocyclyl is substituted with 0-5 independently selected halogen or methyl; or
R^{2A} is 2-benzimidazolyl, 2-naphthyl, or 3-quinolinyl, each of which is optionally substituted with 1, 2, or 3 substituents independently selected from halogen, C₁₋₄alkyl, and —OH;
each R³ is independently selected from:
   hydrogen, halo, and OH;
   C₁-C₄aliphatic unsubstituted or substituted by 1, 2 or 3 substituents independently selected from halo and OH; and
   C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, OR, —C(O)NR¹⁰R¹¹, or N(R)C(O)R; wherein each R^{3A} is independently selected from C₁-C₄alkyl;
or two R³ substituents on the same ring carbon atom may join, together with the carbon atom to which they are attached, to form a cyclopropyl ring;
R⁴ is phenyl or a first 5-6 membered heteroaryl (having 1-4 heteroatoms independently selected from nitrogen, oxygen, and sulfur) wherein said phenyl or first 5-6 membered heteroaryl is substituted with 0-5 R^{B}; and optionally two adjacent atoms of said phenyl or first 5-6 membered heteroaryl have two substituents that together with said adjacent atoms form a cyclic group fused to the phenyl or first 5-6 membered heteroaryl selected from a 4-7 membered carbocyclyl, a 4-7 membered heterocyclyl (having 1-4 heteroatoms independently selected from nitrogen, oxygen, and sulfur), or a second 5-6 membered heteroaryl (having 1-4 heteroatoms independently selected from nitrogen, oxygen, and sulfur); wherein said fused cyclic group is substituted with 0-3 independently selected R^{B}; or
R⁴ is a C₁-C₄aliphatic, C₁-C₄alkoxy, or C₃-C₆cycloalkyl, each of which is substituted with 0-3 groups independently selected from halogen, —CN, —OH, C₁-C₄alkyl, C₁-C₄alkoxy, and optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur;
R¹⁰ is H, C₁-C₆aliphatic, haloC₁-C₆alkyl, C₃-C₆cycloalkyl, haloC₃-C₆cycloalkyl, —C(O)C₁-C₆alkyl, or a 5-6 membered heteroaryl (having 1-4 heteroatoms independently selected from nitrogen, oxygen, and sulfur); each R¹⁰ except H is optionally substituted with 1 or 2 independently selected R^{B};
R¹¹ is H, C₁-C₆aliphatic, or C₃-C₆cycloalkyl, or R¹⁰ and R¹¹ are taken together with the nitrogen atom to which they are attached to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur); each R¹² is optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{B} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, haloC₁-C₆alkyl, C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, NR₂, S(O)₂R, S(O)₂NR₂, S(O)R, S(O)NR₂, C(O)R, C(O)OR, —C(O)NR₂, C(O)N(R)OR, OC(O)R, OC(O)NR₂, —N(R)C(O)OR, N(R)C(O)R, N(R)C(O)NR₂, N(R)C(NR)NR₂, N(R)S(O)₂NR₂, and —N(R)S(O)₂R;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form a cyclic group selected from an optionally substituted 4-7 membered saturated ring, a 4-7 membered partially unsaturated ring, or a 5-6 membered heteroaryl ring (having 1-4 heteroatoms independently selected from nitrogen, oxygen, and sulfur); wherein said cyclic group has 0-3 heteroatoms independently selected from nitrogen, oxygen, and sulfur);
q is 1 or 2;
r is 0, 1, 2 or 3.

As described generally above, Ring A is:
a) a 4-7 membered saturated or partially unsaturated bivalent monocyclic carbocyclyl or 4-7 membered saturated or partially unsaturated bivalent heterocyclyl ring (having 1-4 heteroatoms independently selected from nitrogen, oxygen, and sulfur); or
b) a 4-12 membered saturated or partially unsaturated bivalent bicyclic ring system that is fused, bridged, or spirocyclic selected from carbocyclyl or heterocyclyl (having 1-4 heteroatoms independently selected from nitrogen, oxygen, and sulfur);
wherein Ring A is substituted with 0-4 independently selected R^{B} substituents.

In some embodiments, Ring A is a 4-7 membered saturated or partially unsaturated bivalent monocyclic carbocyclyl or 4-7 membered saturated or partially unsaturated bivalent heterocyclyl ring (having 1-4 heteroatoms independently selected from nitrogen, oxygen, and sulfur). In some embodiments, Ring A is a 4-7 membered saturated or partially unsaturated bivalent monocyclic carbocyclyl, wherein Ring A is substituted with 0-4 independently selected R^{B} substituents. In some embodiments, Ring A is a 4-7 membered saturated or partially unsaturated bivalent monocyclic heterocyclyl (having 1-4 heteroatoms independently selected from nitrogen, oxygen, and sulfur), wherein Ring A is substituted with 0-4 independently selected R^{B} substituents.

In some embodiments, Ring A is a 4-12 membered saturated or partially unsaturated bivalent bicyclic ring system that is fused, bridged, or spirocyclic selected from carbocyclyl or heterocyclyl (having 1-4 heteroatoms independently selected from nitrogen, oxygen, and sulfur). In some embodiments, Ring A is a 4-12 membered saturated or partially unsaturated bivalent bicyclic ring system that is fused, bridged, or spirocyclic and is a carbocyclyl, wherein Ring A is substituted with 0-4 independently selected R^{B} substituents. In some embodiments, Ring A is a 4-12 membered saturated or partially unsaturated bivalent bicyclic ring system that is fused, bridged, or spirocyclic and is a heterocyclyl (having 1-4 heteroatoms independently selected from nitrogen, oxygen, and sulfur), wherein Ring A is substituted with 0-4 independently selected R^{B} substituents.

In some embodiments, Ring A is a 4-12 membered saturated or partially unsaturated bivalent bicyclic ring system comprising 2 fused rings. In some embodiments, Ring A is a 4-12 membered saturated or partially unsaturated bivalent bicyclic ring system comprising a spirocyclic ring system. In some embodiments, Ring A is a 4-12 membered saturated or partially unsaturated bivalent bicyclic ring system comprising a bridged ring system.

In some embodiments, Ring A is

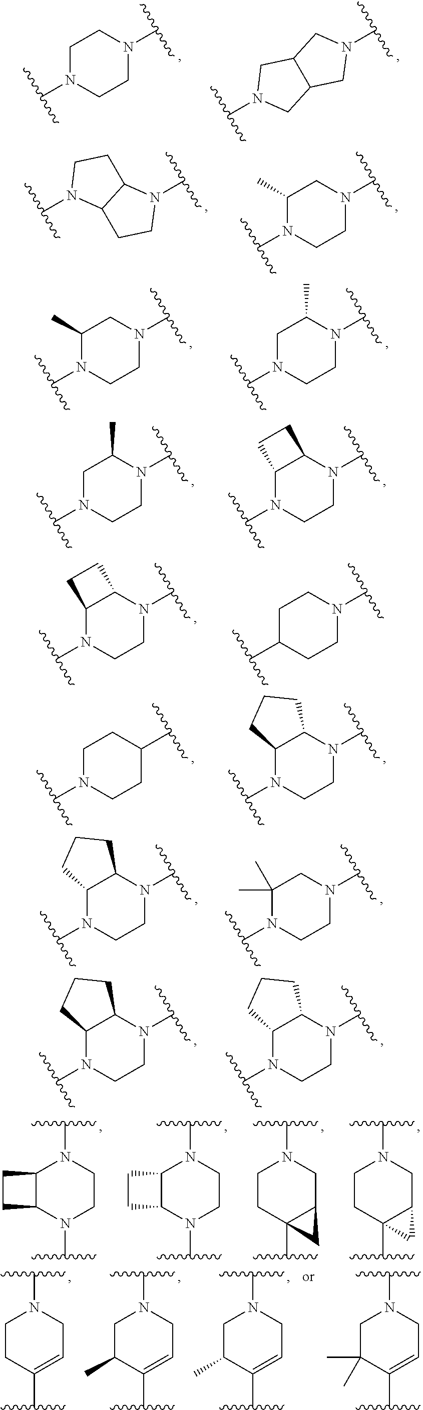

wherein Ring A is substituted with 0-4 independently selected R^{B} substituents.

In some embodiments, Ring A together with its R^{B} substituents is

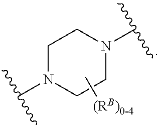

In some embodiments, Ring A together with its R^{B} substituents is

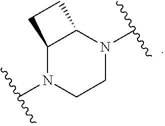 [SMILES: *N1CCN(*)[C@H]2CC[C@@H]21]

In some embodiments, Ring A together with its R^{B} substituents is

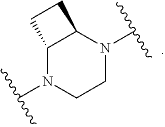 [SMILES: *N1CCN(*)[C@@H]2CC[C@H]21]

In some embodiments, Ring A is as selected from one of the substituents of Table 1 or Table 1a.

As described generally above, L is a linker selected from a bond, —C(O)—, —S(O)—, —S(O)₂—, and

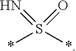

In some embodiments, linker L is —C(O)—.

In some embodiments, linker L is —S(O)—.

In some embodiments, linker L is —S(O)₂—.

In some embodiments, linker L is

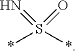

In some embodiments, linker L is as selected from one of the substituents of Table 1 or Table 1a.

As described generally above, R^{1a} is selected from:
a) a 5-6 membered heteroaryl (having 1-4 heteroatoms independently selected from nitrogen, oxygen, and sulfur) optionally substituted with 1-3 groups independently selected from halogen, C₁-C₆aliphatic, C₃-C₆cycloalkyl, haloC₁-C₆alkyl, C₁-C₆alkoxy, and C₃-C₆cycloalkoxy, wherein said 5-6 membered heteroaryl is further substituted with 0-3 independently selected R^{B};
b) a 4-7 membered saturated or partially unsaturated heterocyclyl (having 1-2 heteroatoms independently selected from nitrogen, oxygen, and sulfur), optionally substituted with 1 or 2 groups independently selected from C₁-C₆aliphatic, C₃-C₆cycloalkyl, C₁-C₆alkoxy, C₃-C₆cycloalkoxy, and —OR, wherein said 4-7 membered saturated or partially unsaturated heterocyclyl is further substituted with 0-3 independently selected R^{B}, and two R^{B} along with their intervening atoms optionally join to form a 3-5 membered carbocyclyl;
c) a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), wherein said carbocyclyl or heterocyclyl is substituted with 0-3 independently selected R^{B}; and
d) H, halogen, C₁-C₆aliphatic, C₃-C₇cycloalkyl, C₁-C₆alkylene-O—C₁-C₆alkyl, CN, —OR, —OR¹⁰, —NR¹⁰R¹¹, —C(O)NR¹⁰R¹¹, —CH₂NR¹⁰R¹¹, —SO₂R¹², wherein said C₁-C₆aliphatic, C₃-C₇cycloalkyl, or C₁-C₆alkylene-O—C₁-C₆alkyl is substituted with 0-5 independently selected R^{B};
   or R^{1a} and one R^{1b} on adjacent atoms of Ring B, taken together with the adjacent Ring B atoms to which they are attached, form a cyclic group fused to Ring B selected from phenyl, a 5-6 membered heteroaryl (having 1-3 heteroatoms independently selected from nitrogen, oxygen and sulfur), a 4-7 membered saturated or partially unsaturated carbocyclyl, or a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B}.

In some embodiments, R^{1a} is a 5-6 membered heteroaryl (having 1-4 heteroatoms independently selected from nitrogen, oxygen, and sulfur) optionally substituted with 1 or 2 groups independently selected from C₁-C₆alkyl, C₁-C₆alkoxy, C₃-C₆cycloalkyl, and C₃-C₆cycloalkoxy, wherein said 5-6 membered heteroaryl is further substituted with 0-3 independently selected R^{B}. In some embodiments, R^{1a} is a 4-6 membered saturated or partially unsaturated heterocyclyl (having 1-2 heteroatoms independently selected from nitrogen, oxygen, and sulfur), said heterocyclyl substituted with 0-2 R^{B} groups independently selected from halogen, oxo, —NR₂, optionally substituted C₁₋₄aliphatic, —OR, azetidinyl optionally substituted with 1 or 2 independently selected halogen, and pyrrolidinyl optionally substituted with 1 or 2 independently selected halogen. In some embodiments, R^{1a} is a 6-8 membered saturated or partially unsaturated bridged bicyclic heterocyclyl (having 1-4 heteroatoms independently selected from nitrogen, oxygen, and sulfur), said heterocyclyl substituted with 0-2 R^{B} groups independently selected from halogen, oxo, —NR₂, optionally substituted C₁₋₄aliphatic, —OR, azetidinyl optionally substituted with 1 or 2 independently selected halogen, and pyrrolidinyl optionally substituted with 1 or 2 independently selected halogen. In some embodiments, R^{1a} is a 3-7 membered optionally substituted carbocyclyl. In some embodiments, R^{1a} is an optionally substituted C₂-C₄alkenyl. In some embodiments, R^{1a} is cyclopropyl substituted C₂-C₄alkenyl. In some embodiments, R^{1a} is methyl substituted C₂-C₄alkenyl.

In some embodiments, R^{1a} is a 6-membered partially unsaturated heterocyclyl (having 1 oxygen atom). In some embodiments, R^{1a} is a 4-membered saturated heterocyclyl (having 1 oxygen atom). In some embodiments, R^{1a} is a 6-membered heteroaryl (having 1 nitrogen atom), said heteroaryl may be optionally substituted with 1 or 2 groups independently selected from C₁-C₆alkyl, C₁-C₆alkoxy, C₃-C₆cycloalkyl, and C₃-C₆cycloalkoxy, wherein said heteroaryl is further substituted with 0-1 R^{B}, wherein R^{B} is an optionally substituted C₁₋₆aliphatic group. In some embodiments, R^{1a} is a 6-membered heteroaryl (having 2 nitrogen atoms), said heteroaryl may be optionally substituted with 1 or 2 groups independently selected from C₁-C₆alkyl, C₁-C₆alkoxy, C₃-C₆cycloalkyl, and C₃-C₆cycloalkoxy, wherein said heteroaryl is further substituted with 0-1 R^{B}, wherein R^{B} is an optionally substituted C₁₋₆aliphatic group. In some embodiments, R^{1a} is —NR¹⁰R¹¹ wherein R¹⁰ is a 5-6 membered heteroaryl (having 1 or 2 nitrogen atoms) optionally substituted with 1 or 2 groups independently selected from halogen, CH₃, OCH₃, C₃-C₆cycloalkyl, and C₃-C₆cycloalkoxy and wherein R¹¹ is H or CH₃. In some embodiments, R^{1a} is —CH₂NR¹⁰R¹¹ wherein R¹⁰ is a 5-6 membered heteroaryl (having 1 or 2 nitrogen atoms) optionally substituted with 1 or 2 groups independently selected from halogen, CH₃, OCH₃, C₃-C₆cycloalkyl, and C₃-C₆cycloalkoxy and wherein R¹¹ is H or CH₃. In some embodiments, R^{1a} is C₂-C₄alkene wherein said alkene is optionally substituted with OCH₃ or 1, 2, or 3 fluorine. In some embodiments, R^{1a} is C₂-C₄alkyne wherein said alkyne is optionally substituted with OCH₃ or 1, 2, or 3 fluorine. In some embodiments, R^{1a} is —SO₂R¹² wherein R¹² is selected from CH₃ or a 5-6 membered heteroaryl having 1-2 nitrogen heteroatoms optionally substituted with 1 or 2 groups independently selected from halogen and CH₃. In some embodiments, R^{1a} is cyclopropyl optionally substituted with 1-2 fluorine. In some embodiments, R^{1a} is C₁-C₆alkyl optionally substituted with OH or 1-2 fluorine. In some embodiments, R^{1a} is —C(O)NR¹⁰R¹¹ wherein R¹⁰ is H or CH₃ and wherein R¹¹ is H or CH₃.

In some embodiments, R^{1a} is a 5-membered heteroaryl (having 1-3 heteroatoms independently selected from nitrogen, oxygen, and sulfur) optionally substituted with 1 or 2 groups independently selected from C₁-C₆alkyl, C₁-C₆alkoxy, C₃-C₆cycloalkyl, and C₃-C₆cycloalkoxy, wherein said 5-membered heteroaryl is optionally further substituted with 0-3 independently selected R^{B}. In some embodiments, R^{1a} is a 5-membered heteroaryl (having 1-3 heteroatoms independently selected from nitrogen, oxygen, and sulfur) optionally substituted with 1 or 2 groups independently selected from C₁-C₆alkyl, C₁-C₆alkoxy, C₃-C₆cycloalkyl, and C₃-C₆cycloalkoxy. In some embodiments, R^{1a} is a 5-membered heteroaryl (having 2 nitrogen atoms) optionally substituted with 1 or 2 groups independently selected from C₁-C₆alkyl, C₁-C₆alkoxy, C₃-C₆cycloalkyl, and C₃-C₆cycloalkoxy, wherein said 5-membered heteroaryl is optionally further substituted with 0-1 R^{B}, wherein R^{B} is hydroxyl substituted C₁-C₄alkyl.

In some embodiments, R^{1a} is a 5-6 membered heteroaryl (having 1-4 heteroatoms independently selected from nitrogen, oxygen, and sulfur) optionally substituted with one group of C₁-C₆alkoxy or C₃-C₆cycloalkyl, wherein said 5-6 membered heteroaryl is optionally further substituted with 0-3 independently selected R^{B}.

In some embodiments, R^{1a} is pyridyl substituted with C₁-C₄alkoxy and further substituted with 0-2 R^{B}.

In some embodiments, R^{1a} is 5-membered heteroaryl (having 1 heteroatom independently selected from nitrogen, oxygen, and sulfur, and 0 or 1 additional ring nitrogen atoms), wherein said 5-membered heteroaryl is optionally substituted with C₁-C₆alkyl, or C₃-C₅cycloalkyl and further substituted with 0-2 R^{B}.

In some embodiments, R^{1a} is selected from groups a-d:
a) a 5-6 membered heteroaryl (having 1-4 heteroatoms independently selected from nitrogen, oxygen, and sulfur) optionally substituted with 1 or 2 groups independently selected from C₁-C₆alkyl, C₃-C₆cycloalkyl, C₁-C₆alkoxy, and C₃-C₆cycloalkoxy, wherein said 5-6 membered heteroaryl is further substituted with 0-3 independently selected R^{B};
b) a 4-7 membered saturated or partially unsaturated heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, C₃-C₆cycloalkoxy, and —OR, wherein said 4-7 membered saturated or partially unsaturated heterocyclyl is further substituted with 0-3 independently selected R^{B}, and two R^{B} along with their intervening atoms optionally join to form a 3-5 membered carbocyclyl;
c) a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), said carbocyclyl or heterocyclyl is substituted with 0-3 independently selected R^{B}; and
d) H, halogen, C₁-C₆alkyl, C₂-C₄ alkenyl, C₂-C₄ alkynyl, C₃-C₇cycloalkyl, C₁-C₆alkyl-O—C₁-C₆alkyl, CN, —OR, —NR¹⁰R¹¹, —C(O)NR¹⁰R¹¹, —CH₂NR¹⁰R¹¹, —SO₂R¹², wherein C₁-C₆alkyl, C₂-C₄ alkenyl, C₂-C₄alkynyl, C₃-C₇cycloalkyl, or C₁-C₆alkylene-O—C₁-C₆alkyl may be substituted with 0-5 independently selected R^{B}.

In some embodiments, R^{1a} is a 4-7 membered saturated or partially unsaturated heterocyclyl (having 1-2 heteroatoms independently selected from nitrogen, oxygen, and sulfur), optionally substituted with 1 or 2 groups independently selected from C₁-C₆alkyl, C₃-C₆cycloalkyl, C₁-C₆alkoxy, and C₃-C₆cycloalkoxy, and —OR, wherein said 4-7 membered saturated or partially unsaturated heterocyclyl is further substituted with 0-3 independently selected R^{B}, and two R^{B}along with their intervening atoms optionally join to form a 3-5 membered carbocyclyl.

In some embodiments, R^{1a} is a 5-6 membered heteroaryl (having 1-4 heteroatoms independently selected from nitrogen, oxygen, and sulfur) optionally substituted with 1 or 2 groups independently selected from C₁-C₆alkyl, C₃-C₆cycloalkyl, C₁-C₆alkoxy, and C₃-C₆cycloalkoxy, wherein said 5-6 membered heteroaryl is further substituted with 0-3 independently selected R^{B}.

In some embodiments, R^{1a} is selected from the group consisting of:

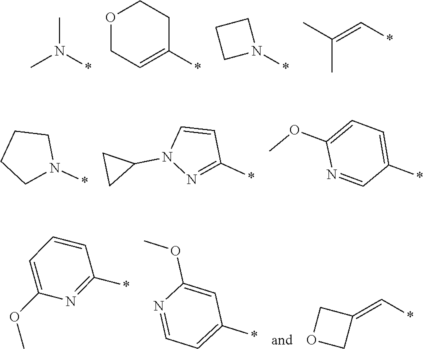

wherein * is the point of attachment to Ring B.

In some embodiments, R^{1a} is

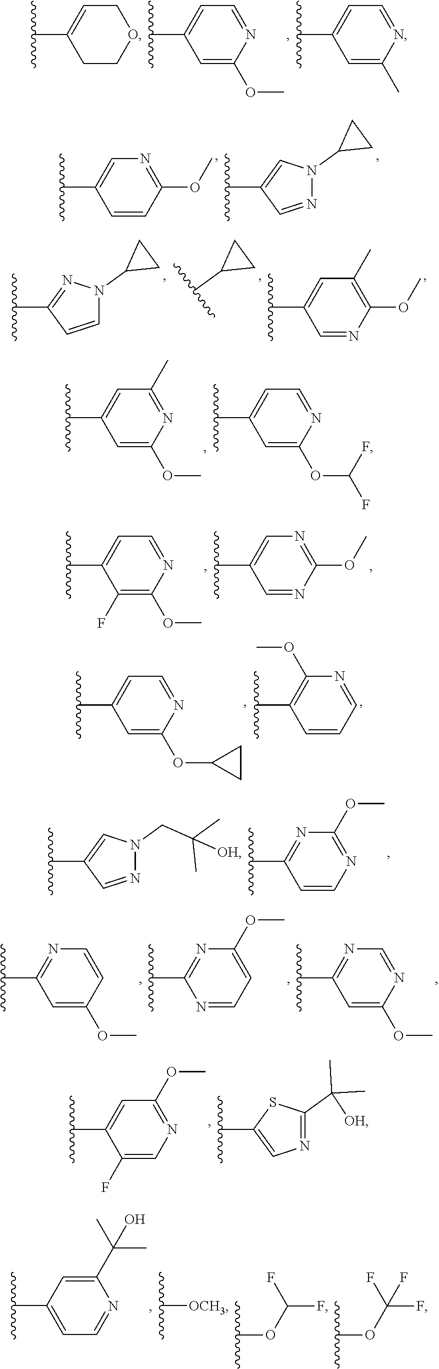

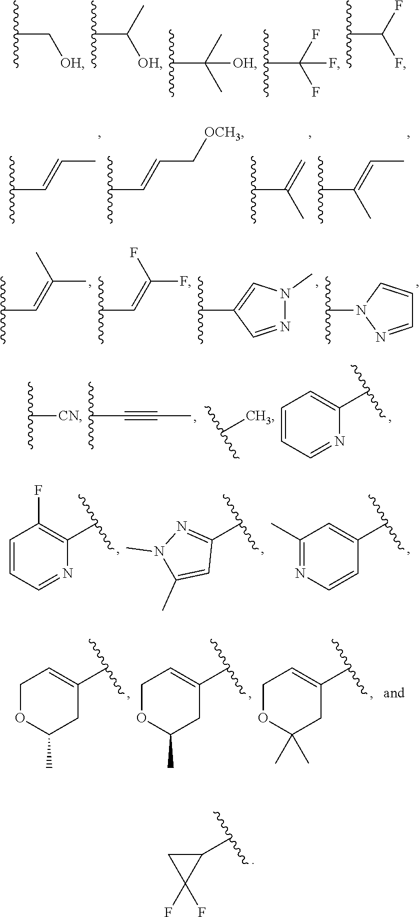

In some embodiments, R^{1a} is

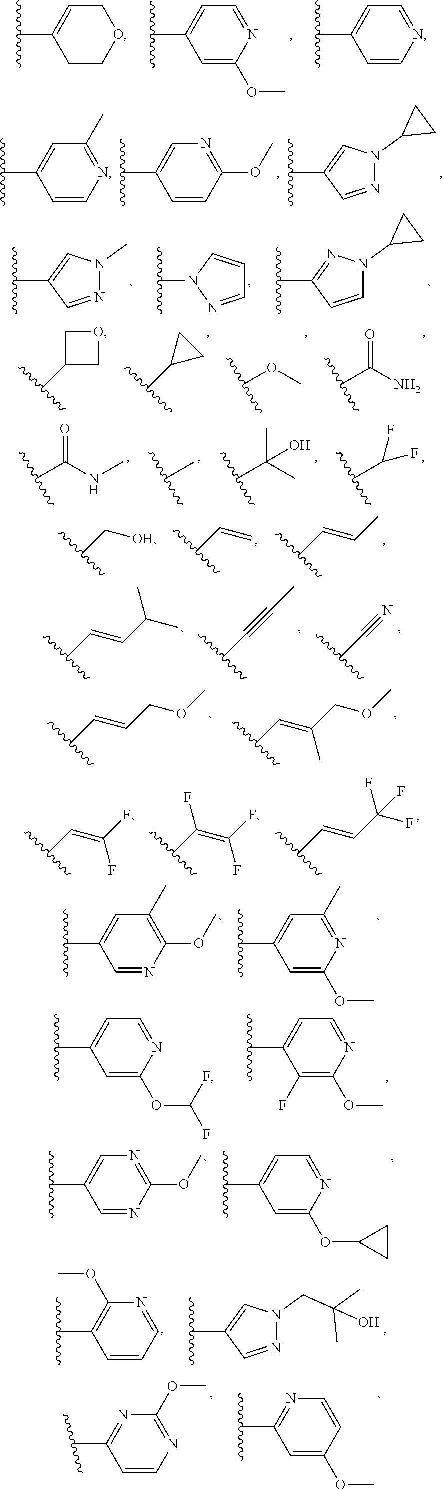

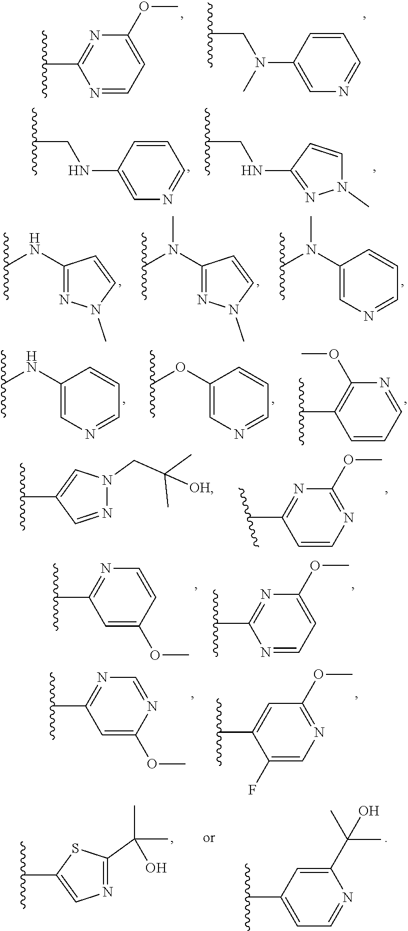

In some embodiments, R^{1a} is

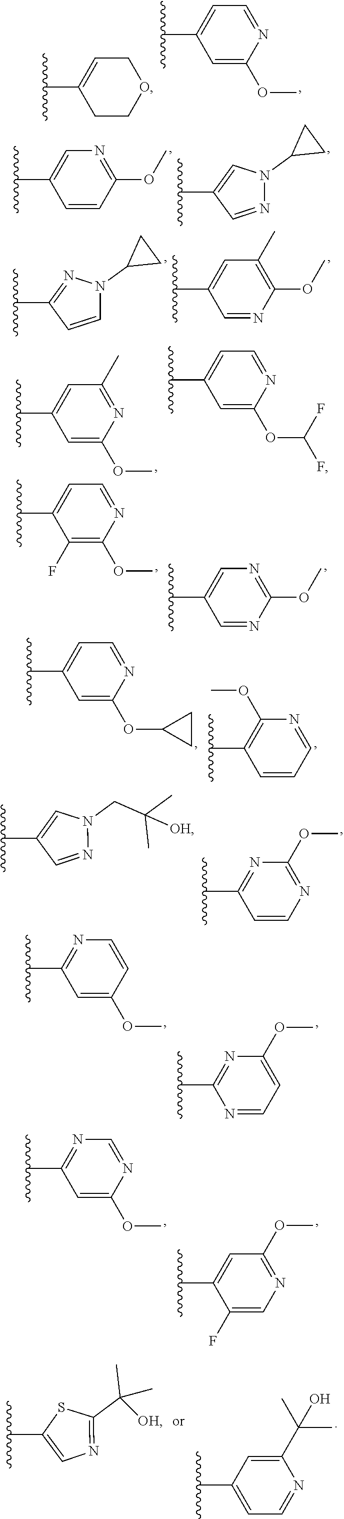

In some embodiments, R^{1a} is

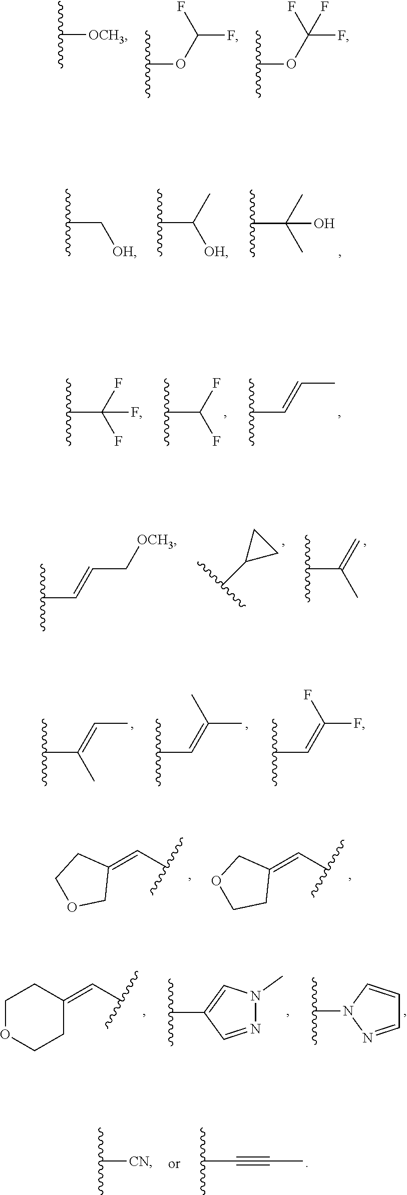

In some embodiments, R^{1a} is as selected from one of the substituents of Table 1 or Table 1a.

In some embodiments, R¹¹ is

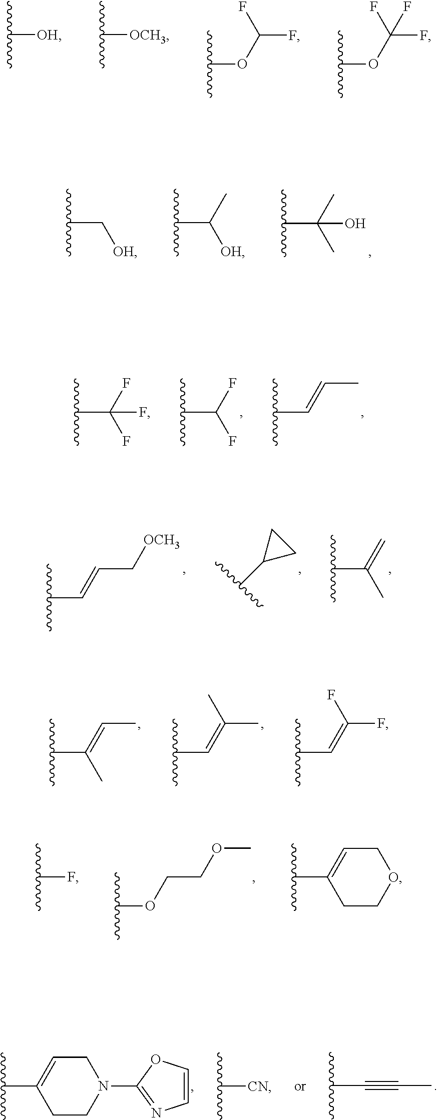

As described generally above, each R^{1b} is independently selected from H, halogen, CN, OH, C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy, wherein said C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy are each independently and optionally substituted with 1-5 halogen, OH, CN, C₁-C₆alkyl, or C₃-C₆cycloalkyl groups.

In some embodiments, R^{1a} and one R^{1b} on adjacent atoms of Ring B, taken together with the adjacent Ring B atoms to which they are attached, form a cyclic group fused to Ring B selected from phenyl, a 5-6 membered heteroaryl (having 1-3 heteroatoms independently selected from nitrogen, oxygen and sulfur), a 4-7 membered saturated or partially unsaturated carbocyclyl, or a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B}.

In some embodiments, R^{1a} and one R^{1b} on adjacent atoms of Ring B, taken together with the adjacent Ring B atoms to which they are attached, form a cyclic group fused to Ring B of phenyl, wherein said cyclic group fused to Ring B is substituted with 0-3 independently selected R^{B}. In some embodiments, R^{1a} and one R^{1b} on adjacent atoms of Ring B, taken together with the adjacent Ring B atoms to which they are attached, form a cyclic group fused to Ring B of a 5-6 membered heteroaryl (having 1-3 heteroatoms independently selected from nitrogen, oxygen and sulfur), wherein said cyclic group fused to Ring B is substituted with 0-3 independently selected R^{B}. In some embodiments, R^{1a} and one R^{1b} on adjacent atoms of Ring B, taken together with the adjacent Ring B atoms to which they are attached, form a cyclic group fused to Ring B of a 4-7 membered saturated or partially unsaturated carbocyclyl, wherein said cyclic group fused to Ring B is substituted with 0-3 independently selected R^{B}. In some embodiments, R¹¹ and one R^{1b} on adjacent atoms of Ring B, taken together with the adjacent Ring B atoms to which they are attached, form a cyclic group fused to Ring B of a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B}.

As described generally above, R² is C(O)N(R)R^{2A}. In some embodiments, R² is C(O)N(R)R^{2A}, wherein R^{2A} is phenyl or bicyclo[1.1.1]pentyl optionally substituted with 1, 2, or 3 substituents independently selected from halogen, C₁-C₄alkyl, or haloC₁-C₄alkyl. In some embodiments, R² is C(O)N(R)R^{2A}, wherein R^{2A} is phenyl optionally substituted with 1, 2, or 3 substituents independently selected from halogen, C₁-C₄alkyl, or haloC₁-C₄alkyl. In some embodiments, R² is C(O)N(R)R^{2A}, wherein R^{2A} is bicyclo[1.1.1]pentyl optionally substituted with 1, 2, or 3 substituents independently selected from halogen, C₁-C₄alkyl, or haloC₁-C₄alkyl.

In some embodiments, R² is

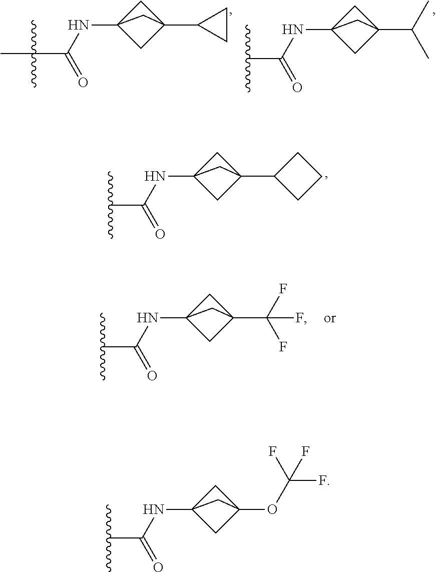

In some embodiments R² is

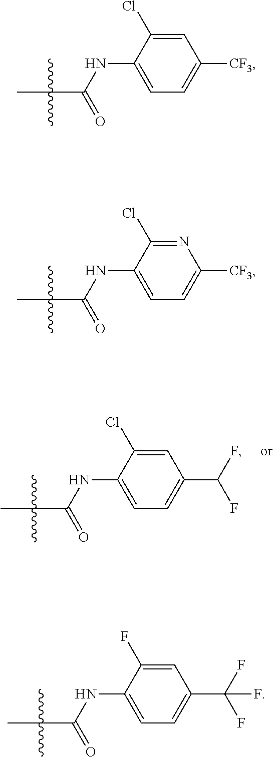 [SMILES: *C(=O)Nc1ccc(C(F)(F)F)cc1Cl ; *C(=O)Nc1ccc(C(F)(F)F)nc1Cl ; *C(=O)Nc1ccc(C(F)F)cc1Cl ; *C(=O)Nc1ccc(C(F)(F)F)cc1F]

In some embodiments R² is

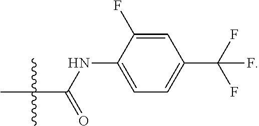 [SMILES: *C(=O)Nc1ccc(C(F)(F)F)cc1F]

In some embodiments R² is

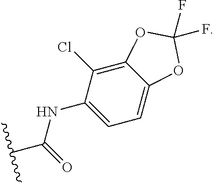 [SMILES: *C(=O)Nc1ccc2c(c1Cl)OC(F)(F)O2]

In some embodiments R² is

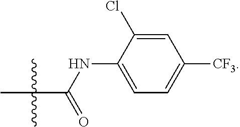 [SMILES: *C(=O)Nc1ccc(C(F)(F)F)cc1Cl]

In some embodiments R² is

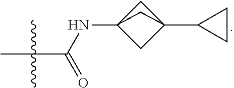 [SMILES: *C(=O)NC12CC(C3CC3)(C1)C2]

In some embodiments, R² is as selected from one of the substituents of Table 1 or Table 1a.

As described generally above, R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆-cycloalkoxy, haloC₃-C₆cycloalkoxy and —SF₅, and wherein two substituents on adjacent atoms of the phenyl or pyridyl, together with said adjacent atoms, optionally form a 4-7 membered carbocyclyl fused to the phenyl or pyridyl, and wherein two substituents on adjacent atoms of the phenyl or pyridyl together with said adjacent atoms optionally form a 4-7 membered heterocyclyl (having 1-4 heteroatoms independently selected from nitrogen, oxygen, and sulfur) fused to the phenyl or pyridyl, wherein said fused 4-7 membered carbocyclyl or fused 4-7 membered heterocyclyl is substituted with 0-5 independently selected halogen or methyl; or R^{2A} is 2-benzimidazolyl, 2-naphthyl, or 3-quinolinyl, each of which is optionally substituted with 1, 2 or 3 substituents independently selected from halogen, C₁₋₄alkyl, and —OH.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two optional substituents on adjacent atoms of the phenyl together with their intervening atoms form a 4-7 membered carbocyclyl fused to the phenyl, and two optional substituents on adjacent atoms of the phenyl together with their intervening atoms form a 4-7 membered heterocyclyl (having 1-4 heteroatoms independently selected from nitrogen, oxygen, and sulfur) fused to the phenyl.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is phenyl optionally substituted with 1, 2, or 3 substituents independently selected from halogen, C₁-C₄alkyl, and haloC₁-C₄alkyl. In some embodiments, R^{2A} is phenyl optionally substituted with a halogen, C₁-C₄alkyl, and haloC₁-C₄alkyl. In some embodiments, R^{2A} is phenyl optionally substituted with two substituents independently selected from halogen, C₁-C₄alkyl, and haloC₁-C₄alkyl. In some embodiments, R^{2A} is phenyl optionally substituted with three substituents independently selected from halogen, C₁-C₄alkyl, and haloC₁-C₄alkyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅, two optional substituents on adjacent atoms of the pyridyl together with their intervening atoms form a 4-7 membered carbocyclyl fused to the pyridyl, and two optional substituents on adjacent atoms of the pyridyl together with their intervening atoms form a 4-7 membered heterocyclyl (having 1-4 heteroatoms independently selected from nitrogen, oxygen, and sulfur) fused to the pyridyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is pyridyl optionally substituted with 1, 2, or 3 substituents independently selected from halogen, C₁-C₄alkyl, and haloC₁-C₄alkyl. In some embodiments, R^{2A} is pyridyl optionally substituted with a halogen, C₁-C₄alkyl, or haloC₁-C₄alkyl. In some embodiments, R^{2A} is pyridyl optionally substituted with 2 substituents independently selected from halogen, C₁-C₄alkyl, and haloC₁-C₄alkyl. In some embodiments, R^{2A} is pyridyl optionally substituted with 3 substituents independently selected from halogen, C₁-C₄alkyl, and haloC₁-C₄alkyl.

In some embodiments, R^{2A} is cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said cubanyl, partially unsaturated monocyclic ring, or saturated or partially unsaturated bridged, fused, or spirocyclic ring are each optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅. In some embodiments, R^{2A} is bicyclo[1.1.1]pentyl optionally substituted with 1, 2, or 3 substituents independently selected from halogen, C₁-C₄alkyl, and haloC₁-C₄alkyl.

In some embodiments, R^{2A} is a saturated or partially unsaturated bridged ring, 5-, 6-, 7-, 8-, 9-, 10-, 11-, or 12-membered ring, which contains 0, 1, 2, 3, or 4 heteroatoms independently selected from nitrogen, oxygen, and sulfur; and wherein said bridged ring is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅.

In some embodiments, R^{2A} is a saturated or partially unsaturated fused ring, 5-, 6-, 7-, 8-, 9-, 10-, 11-, or 12-membered ring, which contains 0, 1, 2, 3, or 4 heteroatoms independently selected from nitrogen, oxygen, and sulfur; and wherein said fused ring is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆cycloalkoxy and —SF₅.

In some embodiments, R^{2A} is a saturated or partially unsaturated spirocyclic 5-, 6-, 7-, 8-, 9-, 10-, 11-, or 12-membered ring, which contains 0, 1, 2, 3, or 4 heteroatoms independently selected from nitrogen, oxygen, and sulfur; and wherein said spirocyclic ring is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆-cycloalkoxy, haloC₃-C₆cycloalkoxy and —SF₅.

In some embodiments, R^{2A} is bicyclo[1.1.1]pentyl optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅. In some embodiments, R^{2A} is bicyclo[1.1.1]pentyl optionally substituted with 1, 2, or 3 substituents independently selected from halogen, C₁-C₄alkyl, and haloC₁-C₄alkyl. In some embodiments, R^{2A} is bicyclo[1.1.1]pentyl optionally substituted with a halogen, C₁-C₄alkyl, or haloC₁-C₄alkyl. In some embodiments, R^{2A} is bicyclo[1.1.1]pentyl optionally substituted with 2 substituents independently selected from halogen, C₁-C₄alkyl, and haloC₁-C₄alkyl. In some embodiments, R^{2A} is bicyclo[1.1.1]pentyl optionally substituted with 3 substituents independently selected from halogen, C₁-C₄alkyl, and haloC₁-C₄alkyl.

In some embodiments, R^{2A} is Ring F selected from the group consisting of:

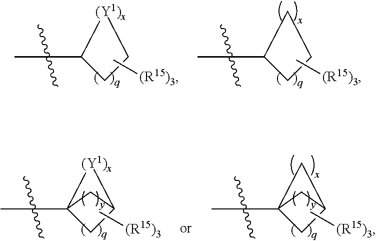

wherein x, y, and q are independently selected from 1, 2 or 3, Y¹ is independently selected from O, NR¹, CHR¹⁵ or CR¹⁵R¹⁵, wherein R¹⁵ is independently selected from H, halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅.

In some embodiments, R^{2A} is Ring F of the following structure

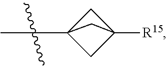

wherein R¹⁵ is selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆cycloalkoxy, haloC₃-C₆-cycloalkoxy and —SF₅.

In some embodiments, R^{2A} is 2-benzimidazolyl, 2-naphthyl, or 3-quinolinyl, each of which is optionally substituted with 1, 2, or 3 substituents independently selected from halogen, C₁₋₄alkyl and, —OH. In some embodiments, R^{2A} is 2-benzimidazolyl optionally substituted with 1, 2, or 3 substituents independently selected from halogen, C₁₋₄alkyl and, —OH. In some embodiments, R^{2A} is 2-naphthyl optionally substituted with 1, 2, or 3 substituents independently selected from halogen, C₁₋₄alkyl and, —OH. In some embodiments, R^{2A} is 3-quinolinyl optionally substituted with 1, 2, or 3 substituents independently selected from halogen, C₁₋₄alkyl and, —OH.

In some embodiments, R^{2A} is phenyl comprising a —CF₃ substituent or pyridyl comprising a —CF₃ substituent.

In some embodiments, R^{2A} is bicyclo[1.1.1]pentyl comprising a —CF₃ substituent or bicyclo[1.1.1]pentyl comprising a —CHF₂ substituent.

In some embodiments, R^{2A} is as selected from one of the substituents of Table 1 or Table 1a.

As described generally above, each R³ is independently selected from:
hydrogen, halo, and OH;
C₁-C₄aliphatic unsubstituted or substituted by 1, 2 or 3 substituents independently selected from halo and OH; and
C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, OR, —C(O)NR¹⁰R¹¹, or N(R)C(O)R; wherein each R^{3A} is independently selected from C₁-C₄alkyl;
   or two R³ substituents on the same ring carbon atom may join, together with the carbon atom to which they are attached, to form a cyclopropyl ring.

In some embodiments, R³ is H. In some embodiments, R³ is methyl. In some embodiments, R³ is methyl and r is 1. In some embodiments, each R³ is halo; OH; C₁-C₄aliphatic unsubstituted or substituted by 1, 2, or 3 substituents independently selected from halo and OH; or C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, OR, —C(O)NR¹⁰R¹¹, or N(R)C(O)R; wherein each R^{3A} is independently selected from C₁-C₄alkyl.

In some embodiments, R³ is as selected from one of the substituents of Table 1 or Table 1a.

As described generally above, each R^{3A} is independently selected from C₁-C₄alkyl. In some embodiments, R^{3A} is —CH₃. In some embodiments, R^{3A} is —CH₂CH₃. In some embodiments, R^{3A} is propyl. In some embodiments, R^{3A} is butyl.

In some embodiments, R^{3A} is as selected from one of the substituents of Table 1 or Table 1a.

As described generally above, R⁴ is phenyl or a first 5-6 membered heteroaryl (having 1-4 heteroatoms independently selected from nitrogen, oxygen, and sulfur) wherein said phenyl or first 5-6 membered heteroaryl is substituted with 0-5 R^{B}; and optionally two adjacent atoms of said phenyl or first 5-6 membered heteroaryl have two substituents that together with said adjacent atoms form a cyclic group fused to the phenyl or first 5-6 membered heteroaryl selected from a 4-7 membered carbocyclyl, a 4-7 membered heterocyclyl (having 1-4 heteroatoms independently selected from nitrogen, oxygen, and sulfur), or a second 5-6 membered heteroaryl (having 1-4 heteroatoms independently selected from nitrogen, oxygen, and sulfur); wherein said fused cyclic group is substituted with 0-3 independently selected R^{B}; or

R⁴ is a C₁-C₄aliphatic, C₁-C₄alkoxy, or C₃-C₆cycloalkyl, each of which is substituted with 0-3 groups independently selected from halogen, —CN, —OH, C₁-C₄alkyl, C₁-C₄alkoxy, and optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur.

In some embodiments, R⁴ is selected from one of a), b), and c):
a) R⁴ is a Ring E that is selected from the group consisting of:

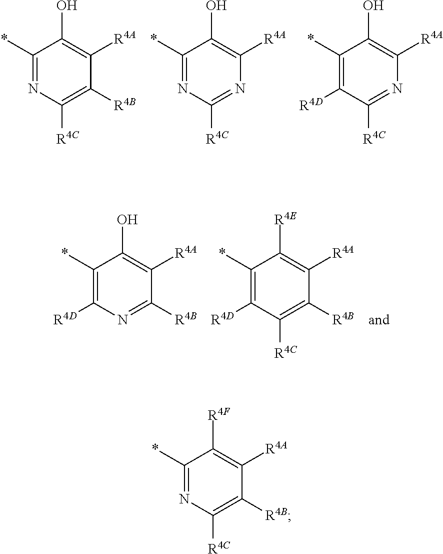

wherein * is a point of attachment to L; and
any substituents that are present on Ring E selected from R^{4A}, R^{4B}, R^{4C}, R^{4D}, R^{4E} and R^{4F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4C}, R^{4D}, R^{4E} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4D}, R^{4E} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4B}, R^{4E} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form a 5-6 membered optionally substituted heteroaryl (having 1-4 heteroatoms independently selected from nitrogen, oxygen, and sulfur) substituted with 0-3 independently selected R^{B}; that is fused to Ring E; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form a 5-6 membered heteroaryl (having 1-4 heteroatoms independently selected from nitrogen, oxygen, and sulfur) substituted with 0-3 independently selected R^{B}; that is fused to Ring E; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is hydrogen, or R¹³ and R¹⁴ combine with the nitrogen atom to which they are attached to form a heterocyclic ring selected from azetidinyl, pyrrolidinyl, or piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; and
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy.

In some embodiments, R⁴ is Ring E of the following structure:

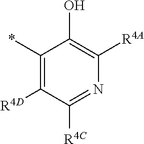

wherein * is a point of attachment to linker L that is bonded to Ring A in Formula I;
and wherein:
R^{4A}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered carbocyclyl) substituted with 0-3 independently selected R^{B}, a 4-7 membered heterocyclyl substituted with 0-3 independently selected R^{B}, or 5-6 membered heteroaryl (having 1-4 heteroatoms independently selected from nitrogen, oxygen, and sulfur) substituted with 0-3 independently selected R^{B}, that is fused to Ring E; and R^{4A} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen and C₁-C₄alkyl optionally substituted with —OH, —OCH₃, or —OCH₂CH₃; and NR¹³R¹⁴, taken in combination form a heterocyclic ring selected from azetidinyl, pyrrolidinyl, or piperidinyl, said heterocyclic ring optionally substituted with —CH₃.
R¹⁴ is hydrogen, or R¹³ and R¹⁴ combine with the nitrogen atom to which they are attached to form a heterocyclic ring selected from azetidinyl, pyrrolidinyl, or piperidinyl, said heterocyclic ring optionally substituted with —CH₃.

In some embodiments, R⁴ is Ring E of the following structure:

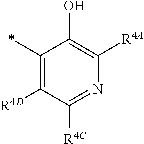

wherein * is a point of attachment to linker L that is bonded to Ring A in Formula I;
and wherein:
R^{4A} is —OCH₃, —OCH₂CH₃, or —OCHF₂;
R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃;
R¹⁴ is hydrogen, or R¹³ and R¹⁴ combine with the nitrogen atom to which they are attached to form a heterocyclic ring selected from azetidinyl, pyrrolidinyl, or piperidinyl, said heterocyclic ring optionally substituted with —CH₃.
or
R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 substituents independently selected from halogen, —OH, —CN, C₁-C₄alkyl, haloC₁-C₄alkyl, C₃-C₆cycloalkyl, and C₁-C₄alkoxy.

In some embodiments, R⁴ is Ring E of the following structure:

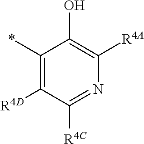

wherein * is a point of attachment to linker L that is bonded to Ring A in Formula I;
and wherein:
R^{4A} is —OCH₃, —OCH₂CH₃, or —OCHF₂;
R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is hydrogen.

In some embodiments, R⁴ is Ring E of the following structure:

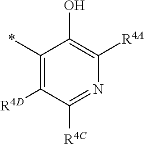

wherein * is a point of attachment to linker L that is bonded to Ring A in Formula I;
and wherein:
R^{4A}, R^{4C}, and R^{4D} are each independently selected from hydrogen; halogen; and C₁-C₄alkyl.

In some embodiments, R⁴ is Ring E of the following structure:

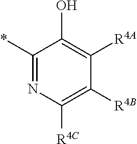

wherein * is a point of attachment to linker L that is bonded to Ring A in Formula I;
and wherein:
R^{4A}, R^{4B}, and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered carbocyclyl or heterocyclyl or 5-6 membered heteroaryl (having 1-4 heteroatoms independently selected from nitrogen, oxygen, and sulfur) that is fused to Ring E; and R^{4C} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; or
R^{4B} and R^{4C}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and R^{4A} is selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is hydrogen.

In some embodiments, R⁴ is Ring E of the following structure:

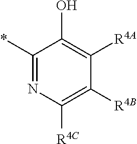

wherein * is a point of attachment to linker L that is bonded to Ring A in Formula I;
and wherein:
R^{4A} and R^{4B}, along with their intervening atoms, join to form 4-7 membered carbocyclyl, 4-7 membered heterocyclyl, or 5-6 membered heteroaryl (having 1-4 heteroatoms independently selected from nitrogen, oxygen, and sulfur) that is fused to Ring E; and
R^{4C} is hydrogen.

In some embodiments, R⁴ is Ring E of the following structure:

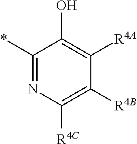

wherein * is a point of attachment to linker L that is bonded to Ring A in Formula I;
and wherein:
R^{4A} and R^{4B}, along with their intervening atoms, join to form 5-membered heterocyclyl (having 1 oxygen atom) that is fused to Ring E; and
R^{4C} is hydrogen.

In some embodiments, R⁴ is Ring E of the following structure:

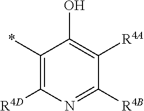

wherein * is a point of attachment linker L that is bonded to Ring A in Formula I;
and wherein:
R^{4A}, R^{4B}, and R^{4D} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form a 4-7 membered carbocyclyl, a 4-7 membered heterocyclyl, a 5-6 membered heteroaryl (having 1-4 heteroatoms independently selected from nitrogen, oxygen, and sulfur) that is fused to Ring E; and R^{4D} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is H.

In some embodiments, R⁴ is Ring E of the following structure:

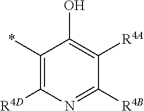

wherein * is a point of attachment to linker L that is bonded to Ring A in Formula I;
and wherein:
R^{4A} and R^{4D} are each hydrogen; and
R^{4B} is C₁-C₄alkyl.

In some embodiments, R⁴ is Ring E of the following structure:

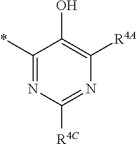

wherein * is a point of attachment linker L that is bonded to Ring A in Formula I;
and wherein:
R^{4A} and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is H.

In some embodiments, R⁴ is Ring E of the following structure:

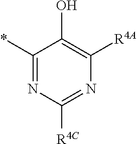

wherein * is a point of attachment to linker L that is bonded to Ring A in Formula I;
and wherein:
R^{4A} and R^{4C} are each independently selected from hydrogen and C₁-C₄alkyl.

In some embodiments, R⁴ is Ring E of the following structure:

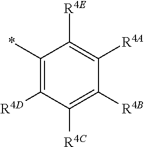

wherein * is a point of attachment to linker L that is bonded to Ring A in Formula I;
and wherein:
R^{4A}, R^{4B}, R^{4C}, R^{4D}, and R^{4E} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered carbocyclyl, 4-7 membered heterocyclyl, or 5-6 membered heteroaryl (having 1-4 heteroatoms independently selected from nitrogen, oxygen, and sulfur) that is fused to Ring E; and R^{4C}, R^{4D}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form 4-7 membered carbocyclyl, 4-7 membered heterocyclyl, 5-6 membered heteroaryl (having 1-4 heteroatoms independently selected from nitrogen, oxygen, and sulfur) that is fused to Ring E; and R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form 5-6 membered heteroaryl (having 1-4 heteroatoms independently selected from nitrogen, oxygen, and sulfur) fused to Ring E; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is H.

In some embodiments, R⁴ is Ring E of the following structure:

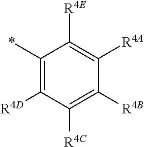

wherein * is a point of attachment to linker L that is bonded to Ring A in Formula I;
and wherein:
R^{4A}, R^{4B}, R^{4C}, R^{4D}, and R^{4E} are each independently selected from hydrogen; halogen; C₁-C₄alkyl; and C₁-C₄alkoxy; or
R^{4C} and R^{4D}, along with their intervening atoms, join to form a 4-7 membered heterocyclyl (having 1-3 nitrogen atoms) fused to Ring E; and R^{4A}, R^{4B}, and R^{4E}are each hydrogen.

In some embodiments, R⁴ is Ring E of the following structure:

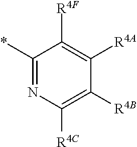

wherein * is a point of attachment to linker L that is bonded to Ring A in Formula I;
and wherein:
R^{4F} and R^{4A}, along with their intervening atoms, join to form 5-6 membered heteroaryl (having 1-4 heteroatoms independently selected from nitrogen, oxygen, and sulfur) fused to Ring E; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen or C₁-C₄alkyl optionally substituted with —OH, —OCH₃, or —OCH₂CH₃; or NR¹³R¹⁴, taken in combination form a heterocyclic ring selected from azetidinyl, pyrrolidinyl, or piperidinyl, said heterocyclic ring optionally substituted with —CH₃; and
R¹⁴ is H.

In some embodiments, R⁴ is Ring E of the following structure:

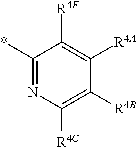

wherein * is a point of attachment to linker L that is bonded to Ring A in Formula I;
and wherein:
R^{4F} and R^{4A}, along with their intervening atoms, join to form 5-6 membered heteroaryl (having 1-2 nitrogen atoms) fused to Ring E; and R^{4B} and R^{4C} are each hydrogen.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from OH, —CH₃, —CHF₂, cyclopropyl, and —OCH₃.

In some embodiments, R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl, and optionally substituted 5-6 membered heterocyclyloxy. In some embodiments, R⁴ is a C₁-C₄alkyl, substituted with 0-3 independently selected halogen, —CN, —OH, C₁-C₄alkyl, and C₁-C₄alkoxy. In some embodiments, R⁴ is a C₁-C₄alkoxy, substituted with 0-3 independently selected halogen, —CN, —OH, C₁-C₄alkyl, and C₁-C₄alkoxy. In some embodiments, R⁴ is a C₃-C₆cycloalkyl, substituted with 0-3 independently selected halogen, —CN, —OH, C₁-C₄alkyl, and C₁-C₄alkoxy.

In some embodiments, R⁴ is an isoxazolyl substituted with —OH or C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms) selected from the group consisting of thiophenyl, imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, 1,2,4-triazolyl, wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy.

In some embodiments, R⁴ is selected from the group consisting of:

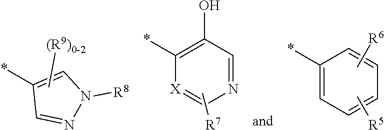

wherein * indicated the point of attachment to L, and R¹, R^{1b}, R², and R³, are as defined herein, both singly and in combination, and wherein:
X is CH, CR⁷, or N;
R⁵ is —OH or halogen;
R⁶ is halogen, C₁₋₄alkyl, or C₁₋₄alkoxy;
each R⁷ is independently hydrogen, halogen, C₁₋₄alkyl, or C₁₋₄alkoxy;
R⁸ is C₁₋₄alkyl;
each of the 0-2 instances of R⁹ is independently a hydrogen or C₁₋₄alkyl.
   In some embodiments:
X is CH or N;
R⁵ is —OH or fluoro;
R⁶ is fluoro, —CH₃, or —OCH₃;
each R⁷ is independently hydrogen, fluoro, —CH₃, or —OCH₃;
R⁸ is —CH₃;
each instance of R⁹ is independently a hydrogen or —CH₃.

In some embodiments, R⁴ is

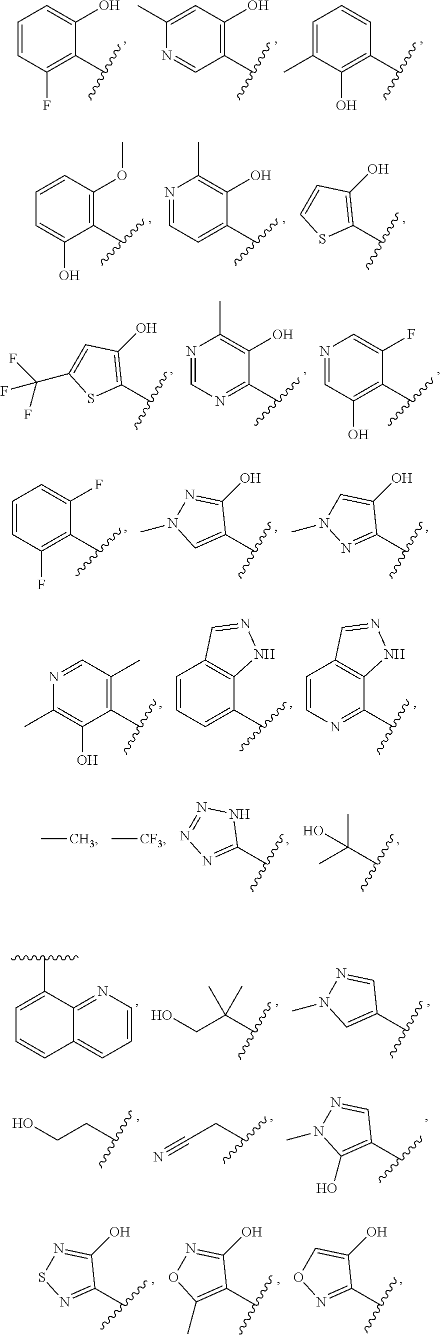

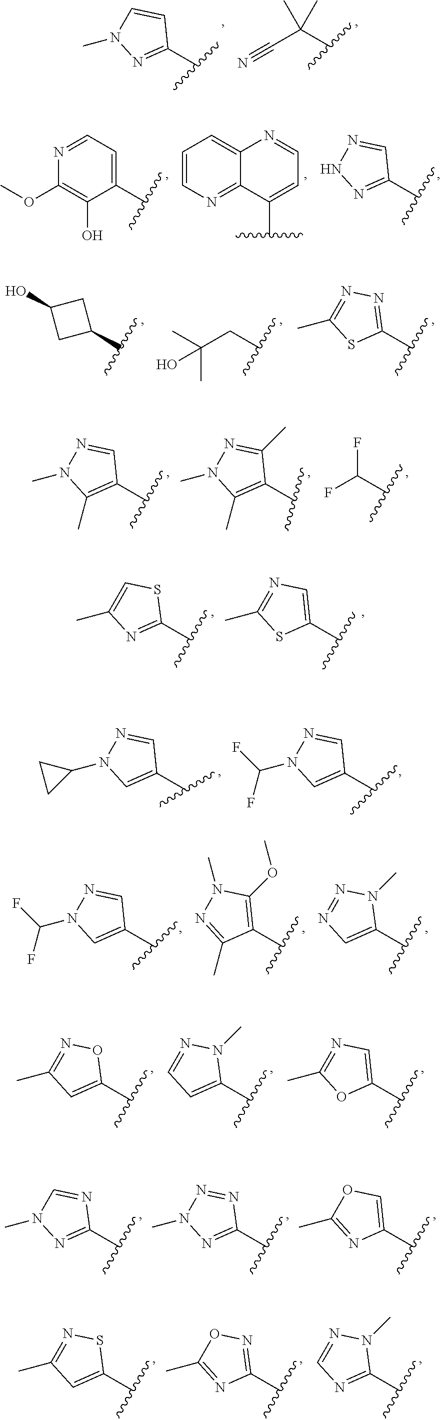

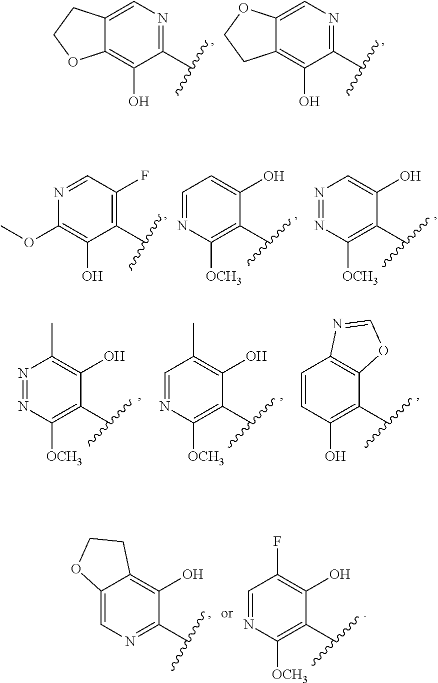

In some embodiments, R⁴ is

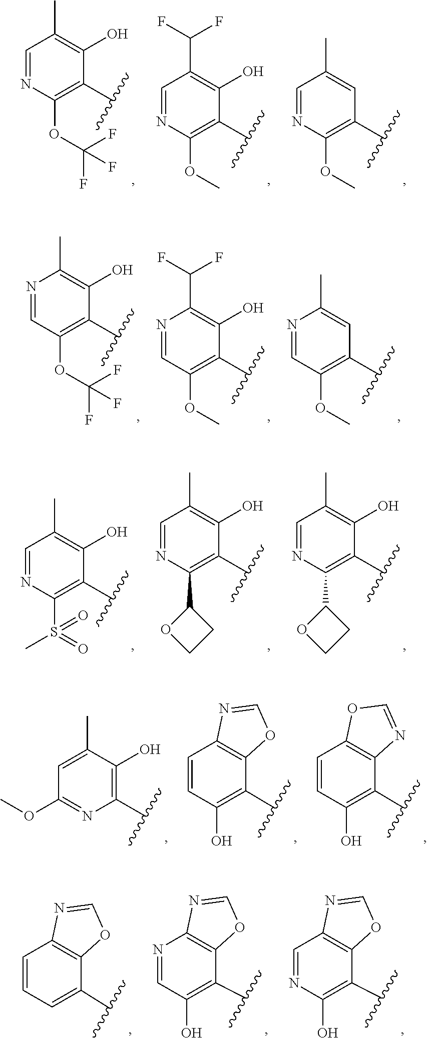

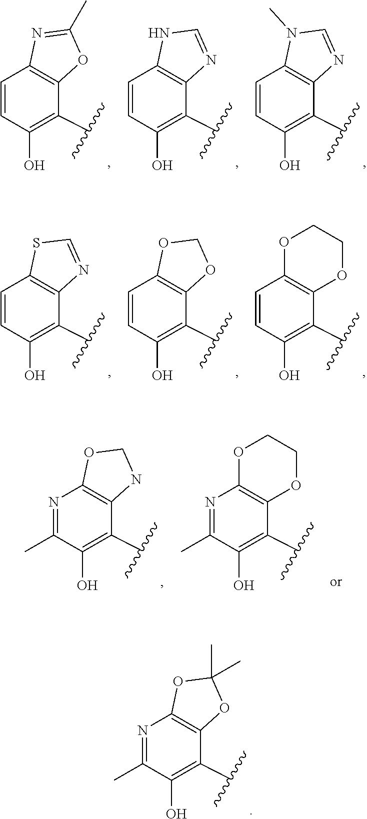

In some embodiments, R⁴ is as shown in a substituent of Table 1 or Table 1a.

As described generally above, R¹⁰ is H, C₁-C₆aliphatic, haloC₁-C₆alkyl, C₃-C₆cycloalkyl, haloC₃-C₆cycloalkyl, —C(O)C₁-C₆alkyl, or a 5-6 membered heteroaryl (having 1-4 heteroatoms independently selected from nitrogen, oxygen, and sulfur); each R¹⁰ except H being optionally substituted with 1 or 2 independently selected R^{B}.

In some embodiments, R¹⁰ is H. In some embodiments, R¹⁰ is C₁-C₆aliphatic, haloC₁-C₆alkyl, C₃-C₆cycloalkyl, haloC₃-C₆cycloalkyl, —C(O)C₁-C₆alkyl, or a 5-6 membered heteroaryl (having 1-4 heteroatoms independently selected from nitrogen, oxygen, and sulfur); each R¹⁰ being optionally substituted with 1 or 2 independently selected R^{B}. In some embodiments, R¹⁰ is C₁-C₆aliphatic, haloC₁-C₆alkyl, C₃-C₆cycloalkyl, haloC₃-C₆cycloalkyl, or —C(O)C₁-C₆alkyl; each R¹⁰ being optionally substituted with 1 or 2 independently selected R. In some embodiments, R¹⁰ is a 5-6 membered heteroaryl (having 1-4 heteroatoms independently selected from nitrogen, oxygen, and sulfur); R¹⁰ being optionally substituted with 1 or 2 independently selected R^{B}.

In some embodiments, R¹⁰ is as shown in a substituent of Table 1 or Table 1a.

As described generally above, R¹¹ is H, C₁-C₆aliphatic, or C₃-C₆cycloalkyl, or R¹⁰ and R¹¹ are taken together with the nitrogen atom to which they are attached to form a 5-6 membered ring optionally substituted with 1, 2, or 3 substituents independently selected from halogen, —OH, —CN, C₁-C₄alkoxy, and haloC₁-C₄alkoxy.

In some embodiments, R¹¹ is H, C₁-C₆aliphatic, or C₃-C₆cycloalkyl. In some embodiments, R¹¹ is H. In some embodiments, R¹¹ is C₁-C₆aliphatic. In some embodiments, R¹¹ is C₃-C₅cycloalkyl. In some embodiments, R¹⁰ and R¹¹ are taken together with the nitrogen atom to which they are attached to form a 5-6 membered ring optionally substituted with 1, 2, or 3 substituents independently selected from halogen, —OH, —CN, C₁-C₄alkoxy, and haloC₁-C₄alkoxy.

In some embodiments, R¹¹ is as shown in a substituent of Table 1 or Table 1a.

As described generally above, R¹² is C₁-C₆aliphatic, C₃-C₆cycloalkyl, or a 5-6 membered heteroaryl (having 1-4 heteroatoms independently selected from nitrogen, oxygen, and sulfur); each R¹² optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy.

In some embodiments, R¹² is C₁-C₆aliphatic optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy. In some embodiments, R¹² is C₁-C₆aliphatic optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy. In some embodiments, R¹² is C₃-C₆cycloalkyl optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy. In some embodiments, R¹² is a 5-6 membered heteroaryl (having 1-4 heteroatoms independently selected from nitrogen, oxygen, and sulfur) optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy. In some embodiments, R¹² is as shown in a substituent of Table 1 or Table 1a.

As described generally above, R^{B} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, haloC₁-C₆alkyl, C₃-C₆cycloalkyl, halo-C₃-C₆cycloalkyl, C₁-C₆alkoxy, halo-C₁-C₆alkoxy, C₃-C₆cycloalkoxy, halo-C₃-C₆cycloalkoxy, C₁-C₆alkylene-O—C₁-C₆alkyl, —CN, —NO₂, oxo, —OR, —SR, NR₂, S(O)₂R, S(O)₂NR₂, S(O)R, S(O)NR₂, C(O)R, C(O)OR, —C(O)NR₂, C(O)N(R)OR, OC(O)R, OC(O)NR₂, —N(R)C(O)OR, N(R)C(O)R, N(R)C(O)NR₂, N(R)C(NR)NR₂, N(R)S(O)₂NR₂, and —N(R)S(O)₂R.

In some embodiments, R^{B} is independently selected at each occurrence from the group consisting of halogen, —OR, or an optionally substituted C₁₋₆aliphatic group. In some embodiments, R^{B} is independently selected at each occurrence from a halogen. In some embodiments, R^{B} is independently selected at each occurrence from —OR. In some embodiments, R^{B} is independently selected at each occurrence from an optionally substituted C₁₋₆aliphatic group.

In some embodiments, R^{B} is as selected from one of the substituents of Table 1 or Table 1a.

As described generally above, each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same atom are taken together with the same atom to form a cyclic group selected from an optionally substituted 4-7 membered saturated ring, a 4-7 membered partially unsaturated ring, or a 5-6 membered heteroaryl ring (having 1-4 heteroatoms independently selected from nitrogen, oxygen, and sulfur); wherein said cyclic group has 0-3 heteroatoms independently selected from nitrogen, oxygen, and sulfur).

In some embodiments, each R is hydrogen. In some embodiments, each R is independently an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur).

In some embodiments, two R groups on the same atom are taken together with the same atom to form a cyclic group selected from an optionally substituted 4-7 membered saturated ring, a 4-7 membered partially unsaturated ring, or a 5-6 membered heteroaryl ring (having 1-4 heteroatoms independently selected from nitrogen, oxygen, and sulfur); wherein said cyclic group has 0-3 heteroatoms independently selected from nitrogen, oxygen, and sulfur). In some embodiments, each R is independently hydrogen or a C₁₋₆ alkyl.

In some embodiments, each R is as selected from one or more of the substituents of Table 1 or Table 1a.

As described generally above, q is 1 or 2. In some embodiments, q is 1. In some embodiments, q is 2.

As described generally above, r is 0, 1, 2, or 3. In some embodiments, r is 1. In some embodiments, r is 2. In some embodiments, r is 3.

In some embodiments, the compound of Formula I is a compound of Formula IIa-Formula IIg:

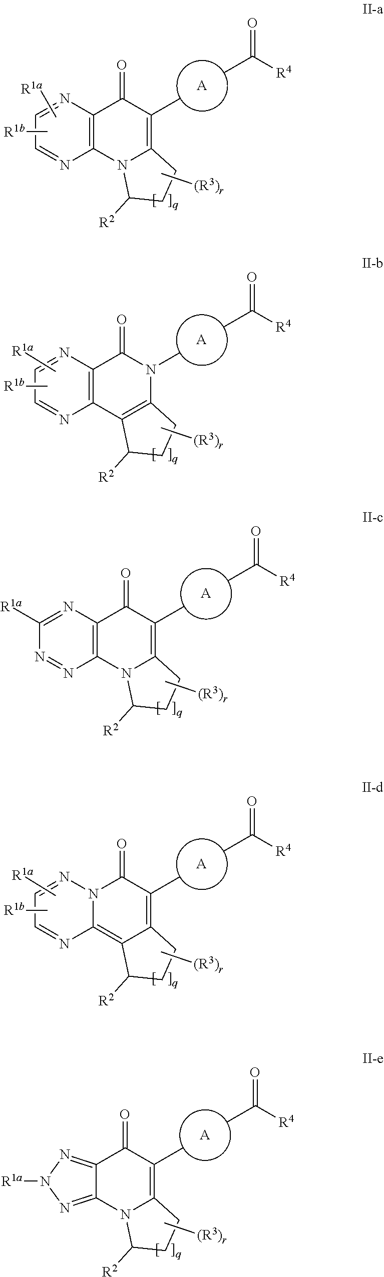

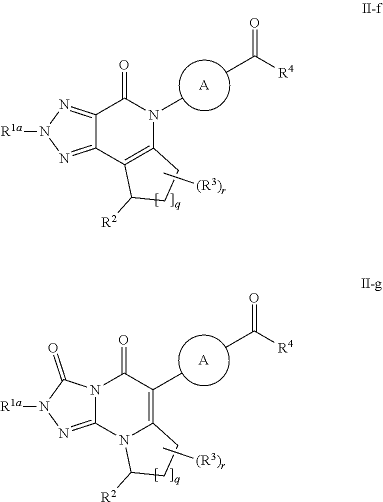

or a pharmaceutically acceptable salt thereof;
wherein R^{1a}, R^{1b}, r, q, Ring A, R², R³, and R⁴, are as defined herein, both singly and in combination.

In some embodiments, the compound of Formula I is a compound of Formula IIIa-Formula IIId:

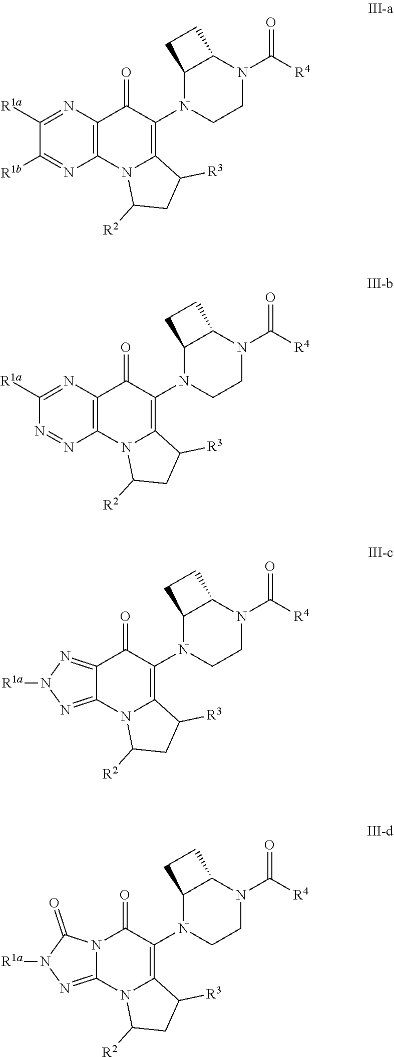

or a pharmaceutically acceptable salt thereof;
wherein R^{1a}, R^{1b}, R², R³, and R⁴, are as defined herein, both singly and in combination.

In some embodiments, the compound of Formula I is a compound of Formula IIa-Formula IIg:

| | |
|---|---|
| 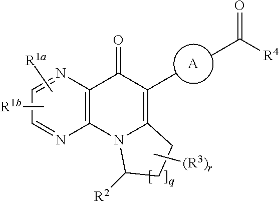 | II-a |
| 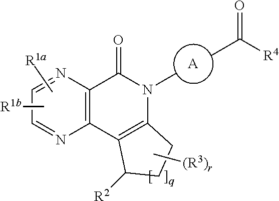 | II-b |
| 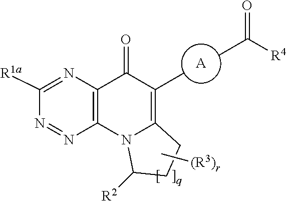 | II-c |
| 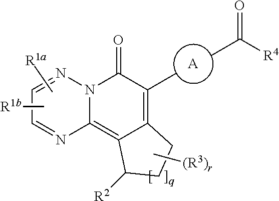 | II-d |
| 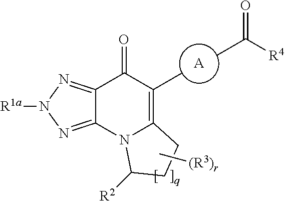 | II-e |
| 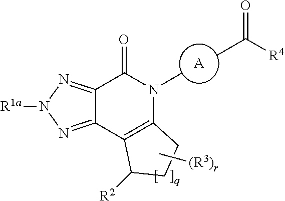 | II-f |
| 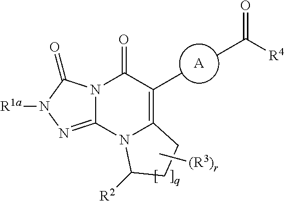 | II-g |

or a pharmaceutically acceptable salt thereof;
wherein R^{1a}, R^{1b}, r, q, Ring A, R², R³, and R⁴, are as defined herein, both singly and in combination and R^{2a} is selected from

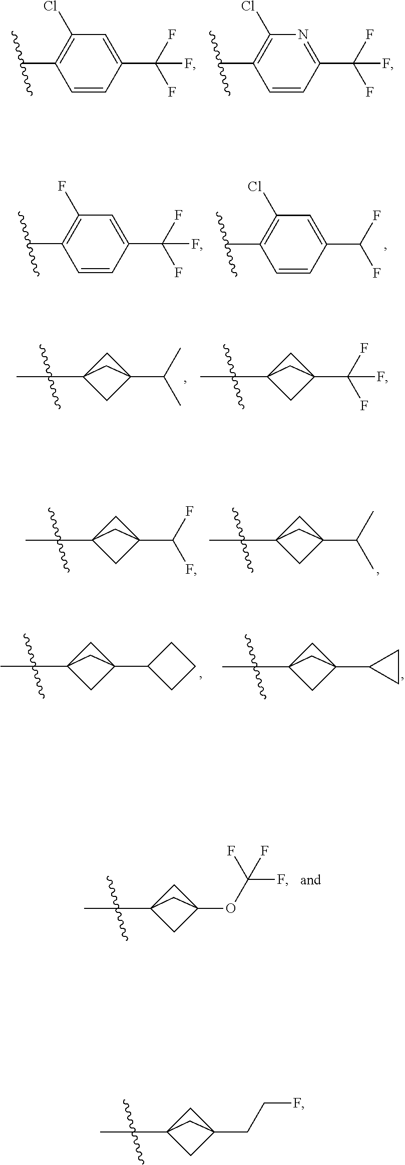

In some embodiments, the compound of Formula I is a compound of Formula IIIa-Formula IIId:

| | |
|---|---|
| 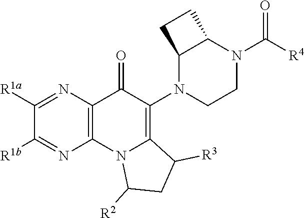 | III-a |
| 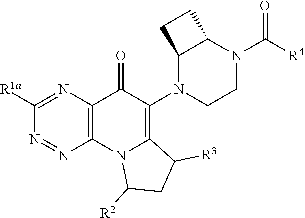 | III-b |
| 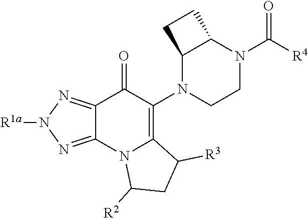 | III-c |
| 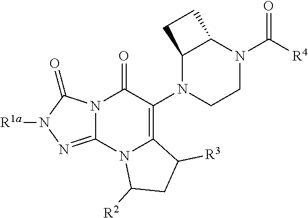 | III-d |

or a pharmaceutically acceptable salt thereof;
wherein R^{1a}, R^{1b}, R², R³, and R⁴, are as defined herein, both singly and in combination and R^{2a} is selected from

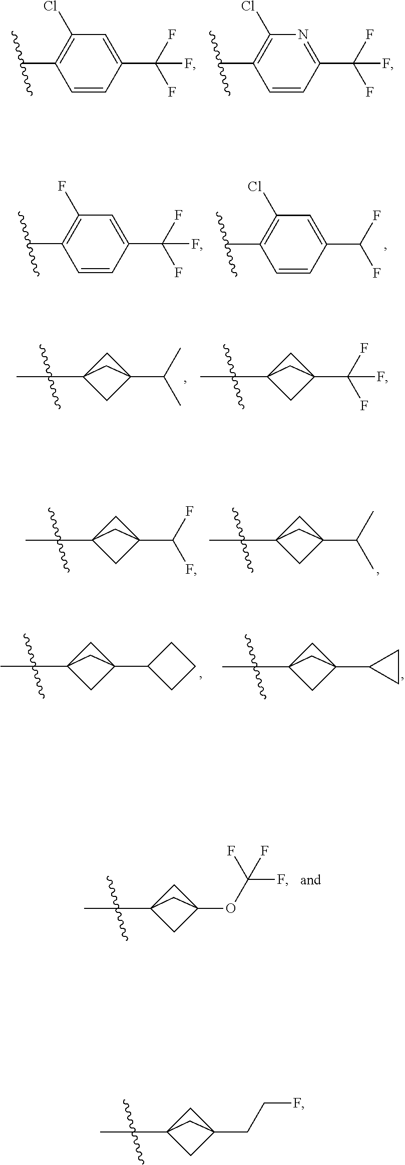

In some embodiments, the compound of Formula I is a compound of Formula IIa-Formula IIg:

| | |
|---|---|
| 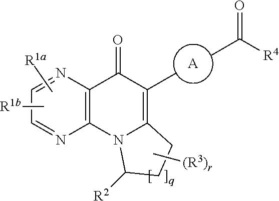 | II-a |
| 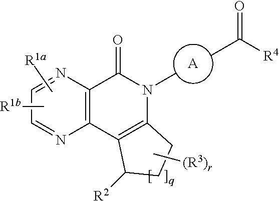 | II-b |
| 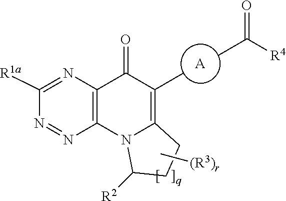 | II-c |
| 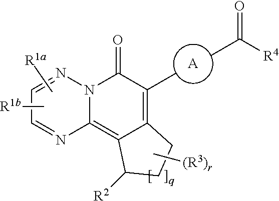 | II-d |
| 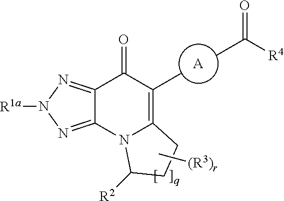 | II-e |
| 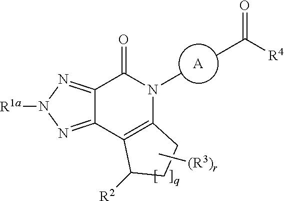 | II-f |
| 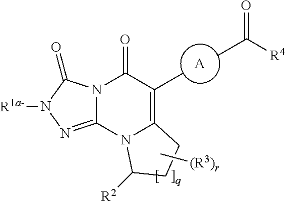 | II-g |

or a pharmaceutically acceptable salt thereof;
wherein R^{1a}, Rib, r, q, Ring A, R², R³, and R⁴, are as defined herein, both singly and in combination, and wherein Ring A is selected from

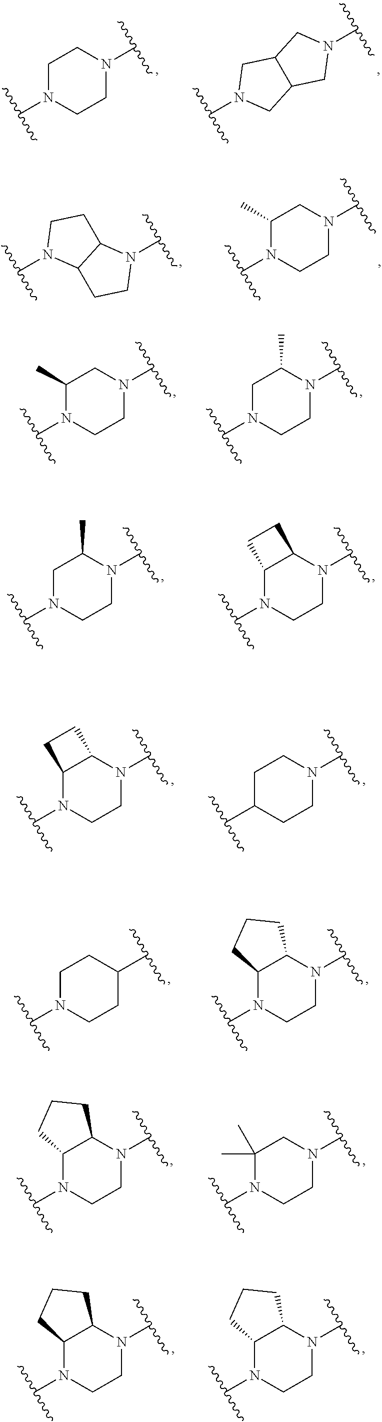

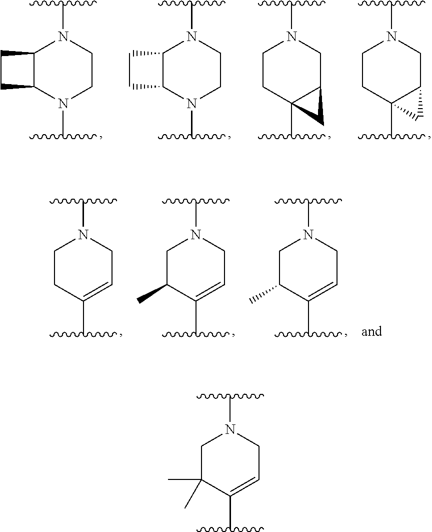

and R^{2a} is selected from

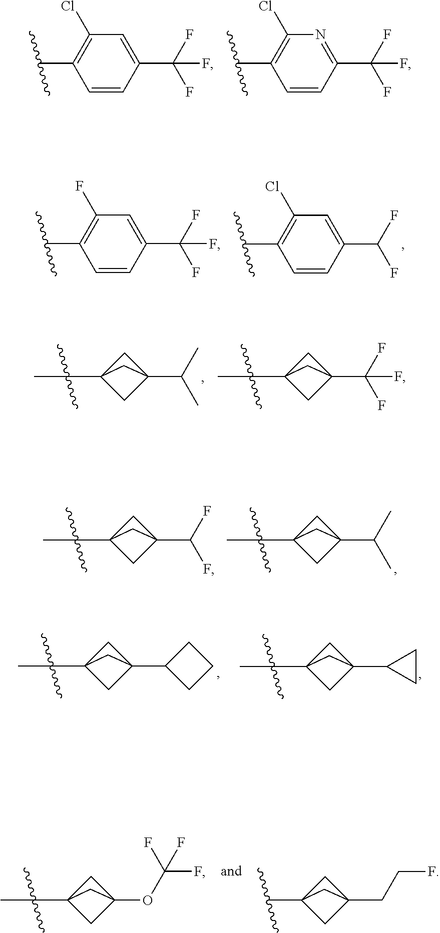

In some embodiments, the compound of Formula I is a compound of Formula IIIa-Formula IIId:

| | |
|---|---|
| 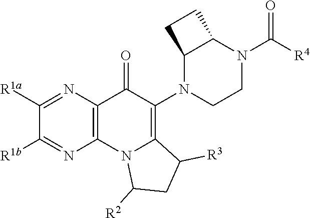 | III-a |
| 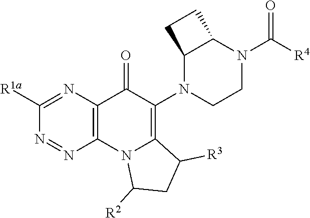 | III-b |
| 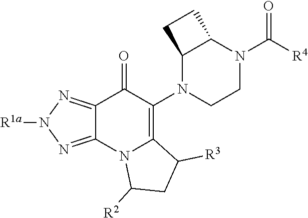 | III-c |
| 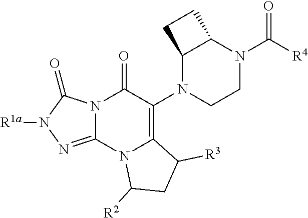 | III-d |

or a pharmaceutically acceptable salt thereof.
wherein R^{1a}, R^{1b}, R², R³, and R⁴, are as defined herein, both singly and in combination, Ring A is selected from

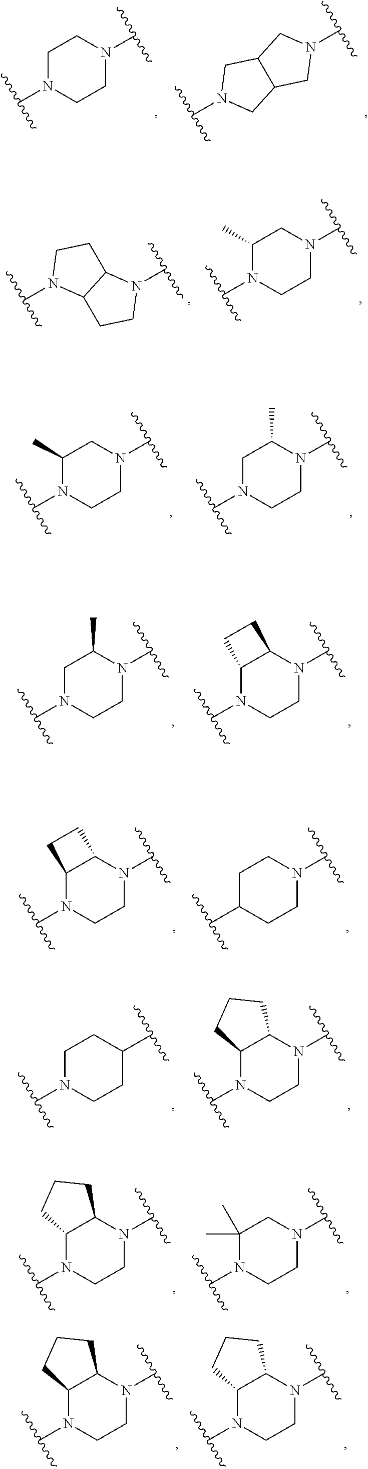

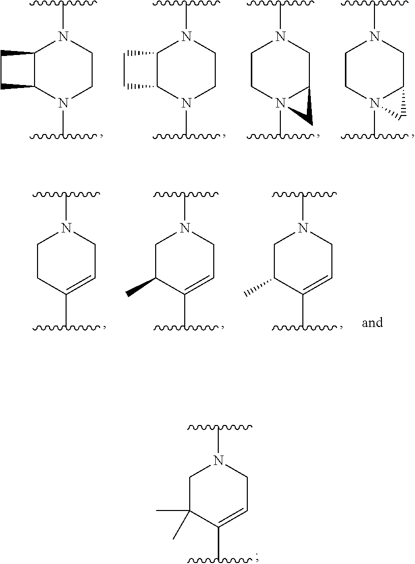

and R^{2a} is selected from

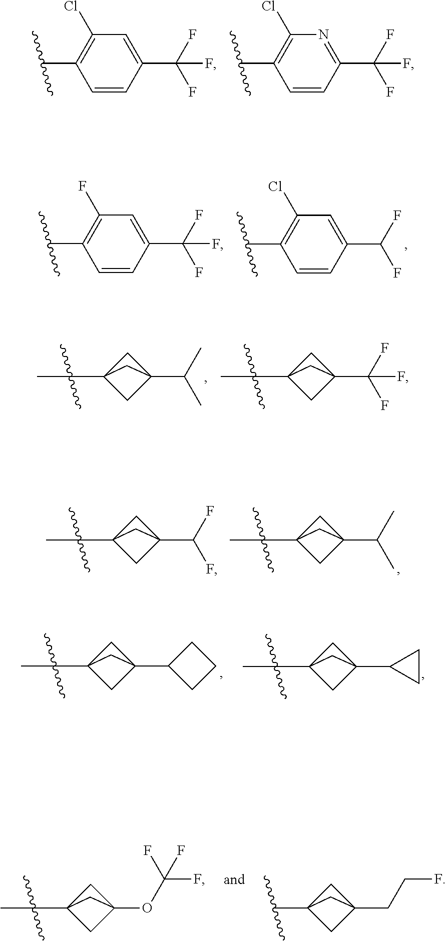

In some embodiments, the compound of Formula I is selected from one of those depicted in Table 1 or Table 1a, or a pharmaceutically acceptable salt thereof. Table 1 or Table 1a, identifies compounds by their IUPAC name and Table 2 or Table 2a lists the same compounds and shows their chemical structure. The chemical names in the present application are generated from the corresponding structures using either CHEMDRAW, or CHEMAXON. In some instances, chemical names generated from the structures may give a different structure when using the “Convert Name to Structure” function in CHEMDRAW. In the event of any discrepancy between Table 1's or Table 1a's name for a compound and Table 2's or Table 2a's structure for that same compound, Table 2's or Table 2a's compound structures will dominate and identify the compound corresponding to each respective compound number (I-#) in Table 1 or Table 1a.

TABLE 1

| No. | IUPAC Name |
|---|---|
| I-1 | N-{3-cyclopropylbicyclo[1.1.1]pentan-1-yl}-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 1) |
| I-2 | N-{3-cyclopropylbicyclo[1.1.1]pentan-1-yl}-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 2) |
| I-3 | N-{3-cyclopropylbicyclo[1.1.1]pentan-1-yl}-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | elindolizine-8-carboxamide (stereoisomer 3) |
| I-4 | N-{3-cyclopropylbicyclo[1.1.1]pentan-1-yl}-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 4) |
| I-5 | N-(2-chloro-4-(trifluoromethyl)phenyl)-5-((1S,6S)-5-(5-hydroxy-6- |
| | methylpyrimidine-4-carbonyl)-2,5-diazabicyclo[4.2.0]octan-2-yl)-2-(2- |
| | methoxypyridin-4-yl)-6-methyl-4-oxo-4,6,7,8-tetrahydro-2H-[1,2,3]triazolo[4,5- |
| | e]indolizine-8-carboxamide (stereoisomer 2) |
| I-6 | N-(2-fluoro-4-(trifluoromethyl)phenyl)-5-((1S,6S)-5-(5-hydroxy-6- |
| | methylpyrimidine-4-carbonyl)-2,5-diazabicyclo[4.2.0]octan-2-yl)-2-(2- |
| | methoxypyridin-4-yl)-6-methyl-4-oxo-4,6,7,8-tetrahydro-2H-[1,2,3]triazolo[4,5- |
| | e]indolizine-8-carboxamide (stereoisomer 2) |
| I-7 | N-(2-chloro-4-(trifluoromethyl)phenyl)-6-((1S,6S)-5-(5-hydroxy-6- |
| | methylpyrimidine-4-carbonyl)-2,5-diazabicyclo[4.2.0]octan-2-yl)-3,7-dimethyl-5- |
| | oxo-5,7,8,9-tetrahydropyrazino[2,3-e]indolizine-9-carboxamide (stereoisomer 4) |
| I-8 | N-(2-chloro-4-(trifluoromethyl)phenyl)-6-((1S,6S)-5-(5-hydroxy-6- |
| | methylpyrimidine-4-carbonyl)-2,5-diazabicyclo[4.2.0]octan-2-yl)-3,7-dimethyl-5- |
| | oxo-5,7,8,9-tetrahydropyrazino[2,3-e]indolizine-9-carboxamide (stereoisomer 3) |
| I-9 | N-(2-chloro-4-(trifluoromethyl)phenyl)-5-((1S,6S)-5-(5-hydroxy-6- |
| | methylpyrimidine-4-carbonyl)-2,5-diazabicyclo[4.2.0]octan-2-yl)-2-(2- |
| | methoxypyridin-4-yl)-6-methyl-4-oxo-4,6,7,8-tetrahydro-2H-[1,2,3]triazolo[4,5- |
| | e]indolizine-8-carboxamide (stereoisomer 3) |
| I-10 | N-(2-fluoro-4-(trifluoromethyl)phenyl)-5-((1S,6S)-5-(5-hydroxy-6- |
| | methylpyrimidine-4-carbonyl)-2,5-diazabicyclo[4.2.0]octan-2-yl)-2-(2- |
| | methoxypyridin-4-yl)-6-methyl-4-oxo-4,6,7,8-tetrahydro-2H-[1,2,3]triazolo[4,5- |
| | e]indolizine-8-carboxamide (stereoisomer 3) |
| I-11 | N-(2-chloro-4-(trifluoromethyl)phenyl)-6-((1S,6S)-5-(5-hydroxy-6- |
| | methylpyrimidine-4-carbonyl)-2,5-diazabicyclo[4.2.0]octan-2-yl)-3,7-dimethyl-5- |
| | oxo-5,7,8,9-tetrahydropyrazino[2,3-e]indolizine-9-carboxamide (stereoisomer 1) |
| I-12 | N-(2-chloro-4-(trifluoromethyl)phenyl)-6-((1S,6S)-5-(5-hydroxy-6- |
| | methylpyrimidine-4-carbonyl)-2,5-diazabicyclo[4.2.0]octan-2-yl)-3,7-dimethyl-5- |
| | oxo-5,7,8,9-tetrahydropyrazino[2,3-e]indolizine-9-carboxamide (stereoisomer 2) |
| I-13 | N-[2-chloro-4-(trifluoromethyl)phenyl]-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 1) |
| I-14 | N-[2-chloro-4-(trifluoromethyl)phenyl]-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 4) |
| I-15 | N-[2-fluoro-4-(trifluoromethyl)phenyl]-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 1) |
| I-16 | N-[2-fluoro-4-(trifluoromethyl)phenyl]-5-[(1S,6S)-5-(5-hydroxy-6- |
| | methylpyrimidine-4-carbonyl)-2,5-diazabicyclo[4.2.0]octan-2-yl]-2-(2- |
| | methoxypyridin-4-yl)-6-methyl-4-oxo-2H,4H,6H,7H,8H-[1,2,3]triazolo[4,5- |
| | e]indolizine-8-carboxamide (stereoisomer 4) |
| I-17 | N-[2-chloro-4-(trifluoromethyl)phenyl]-6-[(1S,6S)-5-(5-hydroxy-6- |
| | methylpyrimidine-4-carbonyl)-2,5-diazabicyclo[4.2.0]octan-2-yl]-2,3,7-trimethyl- |
| | 5-oxo-5H,7H,8H,9H-pyrazino[2,3-e]indolizine-9-carboxamide (stereoisomer 1) |
| I-18 | N-[2-chloro-4-(trifluoromethyl)phenyl]-6-[(1S,6S)-5-(5-hydroxy-6- |
| | methylpyrimidine-4-carbonyl)-2,5-diazabicyclo[4.2.0]octan-2-yl]-2,3,7-trimethyl- |
| | 5-oxo-5H,7H,8H,9H-pyrazino[2,3-e]indolizine-9-carboxamide (stereoisomer 2) |
| I-19 | N-[2-chloro-4-(trifluoromethyl)phenyl]-6-[(1S,6S)-5-(5-hydroxy-6- |
| | methylpyrimidine-4-carbonyl)-2,5-diazabicyclo[4.2.0]octan-2-yl]-2,3,7-trimethyl- |
| | 5-oxo-5H,7H,8H,9H-pyrazino[2,3-e]indolizine-9-carboxamide (stereoisomer 3) |
| I-20 | N-[2-chloro-4-(trifluoromethyl)phenyl]-6-[(1S,6S)-5-(5-hydroxy-6- |
| | methylpyrimidine-4-carbonyl)-2,5-diazabicyclo[4.2.0]octan-2-yl]-2,3,7-trimethyl- |
| | 5-oxo-5H,7H,8H,9H-pyrazino[2,3-e]indolizine-9-carboxamide (stereoisomer 4) |
| I-21 | N-[2-chloro-4-(trifluoromethyl)phenyl]-2-cyclopropyl-5-[(1S,6S)-5-(5-hydroxy- |
| | 6-methylpyrimidine-4-carbonyl)-2,5-diazabicyclo[4.2.0]octan-2-yl]-6-methyl-4- |
| | oxo-2H,4H,6H,7H,8H-[1,2,3]triazolo[4,5-e]indolizine-8-carboxamide |
| | (stereoisomer 1) |
| I-22 | N-[2-chloro-4-(trifluoromethyl)phenyl]-2-cyclopropyl-5-[(1S,6S)-5-(5-hydroxy- |
| | 6-methylpyrimidine-4-carbonyl)-2,5-diazabicyclo[4.2.0]octan-2-yl]-6-methyl-4- |
| | oxo-2H,4H,6H,7H,8H-[1,2,3]triazolo[4,5-e]indolizine-8-carboxamide |
| | (stereoisomer 2) |
| I-23 | N-[2-chloro-4-(trifluoromethyl)phenyl]-2-(3,6-dihydro-2H-pyran-4-yl)-5- |
| | [(1S,6S)-5-(5-hydroxy-6-methylpyrimidine-4-carbonyl)-2,5- |
| | diazabicyclo[4.2.0]octan-2-yl]-6-methyl-4-oxo-2H,4H,6H,7H,8H- |
| | [1,2,3]triazolo[4,5-e]indolizine-8-carboxamide (Stereoisomer 3) |
| I-24 | N-[2-chloro-4-(trifluoromethyl)phenyl]-2-(3,6-dihydro-2H-pyran-4-yl)-5- |
| | [(1S,6S)-5-(5-hydroxy-6-methylpyrimidine-4-carbonyl)-2,5- |
| | diazabicyclo[4.2.0]octan-2-yl]-6-methyl-4-oxo-2H,4H,6H,7H,8H- |
| | [1,2,3]triazolo[4,5-e]indolizine-8-carboxamide (Stereoisomer 2) |
| I-25 | N-[2-chloro-4-(trifluoromethyl)phenyl]-2-(3,6-dihydro-2H-pyran-4-yl)-5- |
| | [(1S,6S)-5-(5-hydroxy-6-methylpyrimidine-4-carbonyl)-2,5- |
| | diazabicyclo[4.2.0]octan-2-yl]-6-methyl-4-oxo-2H,4H,6H,7H,8H- |
| | [1,2,3]triazolo[4,5-e]indolizine-8-carboxamide (Stereoisomer 1) |
| I-26 | (7R,9R)-N-(4-chloro-2,2-difluoro-2H-1,3-benzodioxol-5-yl)-6-[(1S,6S)-5-(5- |
| | hydroxy-6-methylpyrimidine-4-carbonyl)-2,5-diazabicyclo[4.2.0]octan-2-yl]- |
| | 2,3,7-trimethyl-5-oxo-5H,7H,8H,9H-pyrazino[2,3-e]indolizine-9-carboxamide. |
| I-27 | (6R*,8R**)-N-[2-chloro-4-(trifluoromethyl)phenyl]-2-cyclopropyl-5-[(1S,6S)-5- |
| | (5-hydroxy-6-methylpyrimidine-4-carbonyl)-2,5-diazabicyclo[4.2.0]octan-2-yl]- |
| | 6-methyl-4-oxo-2H,4H,6H,7H,8H-[1,2,3]triazolo[4,5-e]indolizine-8-carboxamide |
| | (stereoisomer 4) |
| I-28 | (6R*,8R**)-N-[2-chloro-4-(trifluoromethyl)phenyl]-2-cyclopropyl-5-[(1S,6S)-5- |
| | (5-hydroxy-6-methylpyrimidine-4-carbonyl)-2,5-diazabicyclo[4.2.0]octan-2-yl]- |
| | 6-methyl-4-oxo-2H,4H,6H,7H,8H-[1,2,3]triazolo[4,5-e]indolizine-8-carboxamide |
| | (stereoisomer 3) |

TABLE 1a

| Compound No. | IUPAC Name |
|---|---|
| I-3a | (7R,9R)-N-(2-chloro-4-(trifluoromethyl)phenyl)-3-cyclopropyl-6- |
| | ((1S,6S)-5-(5-hydroxy-6-methylpyrimidine-4-carbonyl)-2,5- |
| | diazabicyclo[4.2.0]octan-2-yl)-7-methyl-5-oxo-5,7,8,9- |
| | tetrahydropyrazino[2,3-e]indolizine-9-carboxamide |
| I-4a | (7R,9R)-N-(2-chloro-4-(trifluoromethyl)phenyl)-3-(dimethylamino)-6- |
| | ((1S,6S)-5-(5-hydroxy-6-methylpyrimidine-4-carbonyl)-2,5- |
| | diazabicyclo[4.2.0]octan-2-yl)-7-methyl-5-oxo-5,7,8,9- |
| | tetrahydropyrazino[2,3-e]indolizine-9-carboxamide |
| I-5a | (7R,9R)-N-(2-chloro-4-(difluoromethyl)phenyl)-6-((1S,6S)-5-(5- |
| | hydroxy-6-methylpyrimidine-4-carbonyl)-2,5-diazabicyclo[4.2.0]octan- |
| | 2-yl)-3,7-dimethyl-5-oxo-5,7,8,9-tetrahydropyrazino[2,3-e]indolizine-9- |
| | carboxamide |
| I-6a | (7R,9R)-6-((1S,6S)-5-(5-hydroxy-6-methylpyrimidine-4-carbonyl)-2,5- |
| | diazabicyclo[4.2.0]octan-2-yl)-3,7-dimethyl-5-oxo-N-(spiro[3.3]heptan- |
| | 2-yl)-5,7,8,9-tetrahydropyrazino[2,3-e]indolizine-9-carboxamide |
| I-7a | (7R,9R)-6-((1S,6S)-5-(5-hydroxy-6-methylpyrimidine-4-carbonyl)-2,5- |
| | diazabicyclo[4.2.0]octan-2-yl)-N-(3-isopropylbicyclo[1.1.1]pentan-1- |
| | yl)-3,7-dimethyl-5-oxo-5,7,8,9-tetrahydropyrazino[2,3-e]indolizine-9- |
| | carboxamide |
| I-8a | (7R,9R)-N-(3-cyclopropylbicyclo[1.1.1]pentan-1-yl)-6-((1S,6S)-5-(5- |
| | hydroxy-6-methylpyrimidine-4-carbonyl)-2,5-diazabicyclo[4.2.0]octan- |
| | 2-yl)-3,7-dimethyl-5-oxo-5,7,8,9-tetrahydropyrazino[2,3-e]indolizine-9- |
| | carboxamide |
| I-9a | (7R,9R)-N-(3-cyclobutylbicyclo[1.1.1]pentan-1-yl)-6-((1S,6S)-5-(5- |
| | hydroxy-6-methylpyrimidine-4-carbonyl)-2,5-diazabicyclo[4.2.0]octan- |
| | 2-yl)-3,7-dimethyl-5-oxo-5,7,8,9-tetrahydropyrazino[2,3-e]indolizine-9- |
| | carboxamide |
| I-10a | (7R,9R)-N-(2-chloro-4-(trifluoromethyl)phenyl)-6-((4aS,7aS)-4-(5- |
| | hydroxy-6-methylpyrimidine-4-carbonyl)octahydro-1H- |
| | cyclopenta[b]pyrazin-1-yl)-3,7-dimethyl-5-oxo-5,7,8,9- |
| | tetrahydropyrazino[2,3-e]indolizine-9-carboxamide |
| I-11a | (7R,9R)-N-(2-chloro-4-(trifluoromethyl)phenyl)-6-(4-(5-hydroxy-6- |
| | methylpyrimidine-4-carbonyl)-2,2-dimethylpiperazin-1-yl)-3,7- |
| | dimethyl-5-oxo-5,7,8,9-tetrahydropyrazino[2,3-e]indolizine-9- |
| | carboxamide |
| I-12a | (7R,9R)-N-(2-chloro-4-(trifluoromethyl)phenyl)-6-((1S,6S)-5-(4- |
| | hydroxy-2-methoxy-5-methylnicotinoyl)-2,5-diazabicyclo[4.2.0]octan- |
| | 2-yl)-3,7-dimethyl-5-oxo-5,7,8,9-tetrahydropyrazino[2,3-e]indolizine-9- |
| | carboxamide |
| I-18a | (7R,9R)-N-(3-cyclopropylbicyclo[1.1.1]pentan-1-yl)-2-(3,6-dihydro- |
| | 2H-pyran-4-yl)-6-((1S,6S)-5-(5-hydroxy-6-methylpyrimidine-4- |
| | carbonyl)-2,5-diazabicyclo[4.2.0]octan-2-yl)-7-methyl-5-oxo-5,7,8,9- |
| | tetrahydropyrrolo[1,2-c][1,2,4]triazolo[1,5-a]pyrimidine-9-carboxamide |
| I-19a | (7R,9R)-N-(3-cyclopropylbicyclo[1.1.1]pentan-1-yl)-6-((1S,6S)-5-(5- |
| | hydroxy-6-methylpyrimidine-4-carbonyl)-2,5-diazabicyclo[4.2.0]octan- |
| | 2-yl)-2-(2-methoxypyridin-4-yl)-7-methyl-5-oxo-5,7,8,9- |
| | tetrahydropyrrolo[1,2-c][1,2,4]triazolo[1,5-a]pyrimidine-9-carboxamide |
| I-27a | (7R,9R)-2-cyclopropyl-N-(3-cyclopropylbicyclo[1.1.1]pentan-1-yl)-6- |
| | ((1S,6S)-5-(5-hydroxy-6-methylpyrimidine-4-carbonyl)-2,5- |
| | diazabicyclo[4.2.0]octan-2-yl)-7-methyl-5-oxo-5,7,8,9- |
| | tetrahydropyrrolo[1,2-c][1,2,4]triazolo[1,5-a]pyrimidine-9-carboxamide |
| I-28a | (7R,9R)-N-(2-chloro-4-(trifluoromethyl)phenyl)-2-cyclopropyl-6- |
| | ((1S,6S)-5-(5-hydroxy-6-methylpyrimidine-4-carbonyl)-2,5- |
| | diazabicyclo[4.2.0]octan-2-yl)-7-methyl-5-oxo-5,7,8,9- |
| | tetrahydropyrrolo[1,2-c][1,2,4]triazolo[1,5-a]pyrimidine-9-carboxamide |
| I-29a | (6R,8R)-N-(2-chloro-4-(trifluoromethyl)phenyl)-2-(3,6-dihydro-2H- |
| | pyran-4-yl)-5-((1S,6S)-5-(5-hydroxy-6-methylpyrimidine-4-carbonyl)- |
| | 2,5-diazabicyclo[4.2.0]octan-2-yl)-6-methyl-4-oxo-4,6,7,8-tetrahydro- |
| | 2H-[1,2,3]triazolo[4,5-e]indolizine-8-carboxamide |
| I-31a | (6R,8R)-N-(2-chloro-4-(trifluoromethyl)phenyl)-2-cyclopropyl-5- |
| | ((1S,6S)-5-(5-hydroxy-6-methylpyrimidine-4-carbonyl)-2,5- |
| | diazabicyclo[4.2.0]octan-2-yl)-6-methyl-4-oxo-4,6,7,8-tetrahydro-2H- |
| | [1,2,3]triazolo[4,5-e]indolizine-8-carboxamide |
| I-32a | (6R,8R)-N-(2-chloro-4-(difluoromethyl)phenyl)-2-(3,6-dihydro-2H- |
| | pyran-4-yl)-5-((1S,6S)-5-(5-hydroxy-6-methylpyrimidine-4-carbonyl)- |
| | 2,5-diazabicyclo[4.2.0]octan-2-yl)-6-methyl-4-oxo-4,6,7,8-tetrahydro- |
| | 2H-[1,2,3]triazolo[4,5-e]indolizine-8-carboxamide |
| I-38a | (6R,8R)-2-cyclopropyl-N-(3-cyclopropylbicyclo[1.1.1]pentan-1-yl)-5- |
| | ((1S,6S)-5-(5-hydroxy-6-methylpyrimidine-4-carbonyl)-2,5- |
| | diazabicyclo[4.2.0]octan-2-yl)-6-methyl-4-oxo-4,6,7,8-tetrahydro-2H- |
| | [1,2,3]triazolo[4,5-e]indolizine-8-carboxamide |
| I-39a | (6R,8R)-N-(3-cyclopropylbicyclo[1.1.1]pentan-1-yl)-2-(3,6-dihydro- |
| | 2H-pyran-4-yl)-5-((1S,6S)-5-(5-hydroxy-6-methylpyrimidine-4- |
| | carbonyl)-2,5-diazabicyclo[4.2.0]octan-2-yl)-6-methyl-4-oxo-4,6,7,8- |
| | tetrahydro-2H-[1,2,3]triazolo[4,5-e]indolizine-8-carboxamide |
| I-40a | (7R,9R)-N-(4-chloro-2,2-difluorobenzo[d][1,3]dioxol-5-yl)-6-((1S,6S)- |
| | 5-(5-hydroxy-6-methylpyrimidine-4-carbonyl)-2,5- |
| | diazabicyclo[4.2.0]octan-2-yl)-3,7-dimethyl-5-oxo-5,7,8,9- |
| | tetrahydropyrazino[2,3-e]indolizine-9-carboxamide |
| I-41a | (7R,9R)-N-(4-chloro-2,2-difluorobenzo[d][1,3]dioxol-5-yl)-6-((1S,6S)- |
| | 5-(5-hydroxy-6-methylpyrimidine-4-carbonyl)-2,5- |
| | diazabicyclo[4.2.0]octan-2-yl)-3,7-dimethyl-5-oxo-5,7,8,9- |
| | tetrahydropyrazino[2,3-e]indolizine-9-carboxamide |
| I-42a | (7R,9R)-N-(4-chloro-2,2-difluorobenzo[d][1,3]dioxol-5-yl)-3- |
| | (dimethylamino)-6-((1S,6S)-5-(5-hydroxy-6-methylpyrimidine-4- |
| | carbonyl)-2,5-diazabicyclo[4.2.0]octan-2-yl)-7-methyl-5-oxo-5,7,8,9- |
| | tetrahydropyrazino[2,3-e]indolizine-9-carboxamide |
| I-43a | (7R,9R)-N-(4-chloro-2,2-difluorobenzo[d][1,3]dioxol-5-yl)-3- |
| | cyclopropyl-6-((1S,6S)-5-(5-hydroxy-6-methylpyrimidine-4-carbonyl)- |
| | 2,5-diazabicyclo[4.2.0]octan-2-yl)-7-methyl-5-oxo-5,7,8,9- |
| | tetrahydropyrazino[2,3-e]indolizine-9-carboxamide |

### 4. Pharmaceutical Compositions, Methods of Treatment and Uses of Compounds

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and
e) absorbents, colorants, flavors and sweeteners.

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween® 80 or Cremophor EL®). Cyclodextrins are established as safe solubilizing agents. Compounds with a high solubility in natural oils may be solubilized in parenteral fat emulsions.

There is also provided a pharmaceutical composition comprising a compound of Formula I as described herein, or a pharmaceutically acceptable salt thereof, and one or more pharmaceutically acceptable carriers.

### Uses

The compounds of Formula I of the present invention in free form or in pharmaceutically acceptable salt form, exhibit valuable pharmacological properties, e.g. WRN inhibiting properties, e.g. as indicated in vitro tests as provided in the next sections, and are therefore indicated for therapy, or for use as research chemicals, e.g. as a chemical probe, and as tool compounds.

Also provided is a compound of Formula I, as described herein. Said compound can be used as a research chemical, a compound herein comprising an added biotin moiety, for example a tool compound or chemical probe, in particular for research on WRN. In another embodiment there is provided the use of a compound of Formula I, as described herein, as a research chemical, for example tool compound or chemical probe, in particular for research on WRN.

There is also provided a compound of Formula I as described herein, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of Formula I as described herein, or a pharmaceutically acceptable salt thereof, may be useful in the treatment of a cancer that is characterized as microsatellite instability-high (MSI-H) or mismatch repair deficient (dMMR).

There is also provided a compound of Formula I as described herein, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of Formula I as described herein, or a pharmaceutically acceptable salt thereof,
inhibiting WRN in a subject, wherein the method comprises administering to the subject a therapeutically effective amount of the compound of Formula I as described herein, or a pharmaceutically acceptable salt thereof,
treating a disorder or disease which can be treated by WRN inhibition in a subject, comprising administering to the subject a therapeutically effective amount of the compound of Formula I as described herein, or a pharmaceutically acceptable salt thereof,
treating cancer in a subject, comprising administering to the subject a therapeutically effective amount of the compound of Formula I as described herein, or a pharmaceutically acceptable salt thereof,
treating cancer in a subject, comprising administering a compound of Formula I as described herein, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of Formula I as described herein, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition,
   wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is intended to represent unlabeled forms as well as isotopically labeled forms of the compounds, in addition to the deuteration specifically claimed in Formula I. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as 3H, 11C, 13C, 14C, 15N, 18F 31P, 32P, 35S, 36CI, 123I, 124I, and 125I, respectively. Accordingly it should be understood that the invention includes compounds that incorporate one or more of any of the aforementioned isotopes, including for example, radioactive isotopes, such as 3H and 14C, or those into which non-radioactive isotopes, such as 2H and 13C are present. Such isotopically labelled compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

A “compound of the present invention” or a “compound of Formula I” includes a zwitterion thereof, a non-zwitterion thereof (non-charged form), or a pharmaceutically acceptable salt of said zwitterionic or non-zwitterionic form thereof. “Zwitterion” or “zwitterionic form” means a compound containing both positive and negatively charged functional groups.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“WRN inhibitor” or “WRN helicase inhibitor” as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

“Disease or condition mediated by WRN” includes a disease or condition, such as cancer, which is treated by WRN inhibition. In particular this can include cancers characterized as microsatellite instability-high (MSI-H) or mismatch repair deficient (dMMR).

“Microsatellite unstable cancer,” “microsatellite instability-high cancer,” “microsatellite high cancer” and “MSI-high cancer,” “MSIhi” and “MSI-H” when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

Microsatellite instability can be found in colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The terms “synthetic lethality,” and “synthetically lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In some embodiments, the methods of the invention comprise administration of a therapeutically effective amount of a compound herein.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate, a rat or a mouse. In yet other embodiments, the subject is a human.

As used herein, the term “inhibit,” “inhibition” or “inhibiting” refers to the reduction or suppression of a given condition, symptom, or disorder, or disease, or a significant decrease in the baseline activity of a biological activity or process.

As used herein, the term “treat,” “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent,” “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

“May join” means joins or does not join.

“May be replaced by deuterium” means is replaced by deuterium, or is not replaced by deuterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of Formula I that contain groups capable of acting as donors and/or acceptors for hydrogen bonds may be capable of forming co-crystals with suitable co-crystal formers. These co-crystals may be prepared from compounds of Formula I by known co-crystal forming procedures. Such procedures include grinding, heating, co-subliming, co-melting, or contacting in solution compounds of Formula I with the co-crystal former under crystallization conditions and isolating co-crystals thereby formed. Suitable co-crystal formers include those described in WO 2004/078163. Hence the invention further provides co-crystals comprising a compound of Formula I.

Furthermore, the compounds of the present invention, including their salts, can also be obtained in the form of their hydrates, or include other solvents used for their crystallization.

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of Formula I, or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage.

The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of Formula I and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

There is also provided a combination comprising a compound of Formula I as described herein, or a pharmaceutically acceptable salt thereof, as described herein, and one or more additional therapeutically active agents. The additional therapeutic agent is, for example, a chemical compound, peptide, antibody, antibody fragment or nucleic acid, which is therapeutically active or enhances the therapeutic activity when administered to a patient in combination with a compound of the present disclosure. In particular, an additional therapeutically active agent is:
an anti-cancer agent,
a chemotherapy,
chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate,
liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEXO), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular fluorouracil (5-FU) and irinotecan (Camptosar®).
a PD-1 inhibitor,
an anti-PD-1 antibody molecule,
a PD-1 inhibitor selected from spartalizumab (Novartis), nivolumab (Bristol-Myers Squibb), pembrolizumab (Merck & Co), pidilizumab (CureTech), MED10680 (Medimmune), cemiplimab (REGN2810, Regeneron), dostarlimab (TSR-042, Tesaro), PF-06801591 (Pfizer), tislelizumab (BGB-A317, Beigene), BGB-108 (Beigene), INCSHR1210 (Incyte), balstilimab (AGEN2035, Agenus), sintilimab (InnoVent), toripalimab (Shanghai Junshi Bioscience), camrelizumab (Jiangsu Hengrui Medicine Co.), AMP-224 (Amplimmune), penpulimab (Akeso Biopharma Inc), zimberelimab (Arcus Biosciences Inc), and prolgolimab (Biocad Ltd),
spartalizumab, or
tislelizumab (BGB-A317, Beigene).

In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In a further embodiment, the PD-1 inhibitor is an anti-PD-1 antibody molecule as described in US 2015/0210769, published on Jul. 30, 2015, entitled “Antibody Molecules to PD-1 and Uses Thereof.”

In another embodiment, there is provided a combination of a compound of Formula I or a pharmaceutically acceptable salt thereof, and a chemotherapy, and a PD-1 inhibitor. In particular, the chemotherapy and PD-1 inhibitor are selected from those described above. In some embodiments, the PD-1 inhibitor is pembrolizumab, nivolumab, cemiplimab, dostarlimab, or retifanlimab.

The above-mentioned compounds, which can be used in combination with a compound of the present invention, can be prepared and administered as described in the art, such as in the documents cited above.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of Formula I and the other therapeutic agent(s) together in the same pharmaceutical composition, or the compound of the present invention and the other therapeutic agent(s) in separate form, e.g. in the form of a kit.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of the present invention.

The invention also provides a compound of the present invention for use in treating a disease or condition mediated by WRN, wherein the compound of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of the present invention.

The invention also provides the use of a compound of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### 5. General Synthetic Methods of Making Compounds of the Disclosure

Compounds of the present invention can generally be prepared by conventional techniques known to those skilled in the art or by processes analogous to those described in the accompanying synthetic schemes.

Referring to Scheme A below, an appropriately functionalized starting material comprising Ring B may be functionalized with a β-diketone having a leaving group (LG) at the a position. A reagent comprising the Ring A moiety is then reacted via nucleophilic substitution to displace said LG and establish Ring A to the growing compound. Ring C may be established via intramolecular cyclization/condensation to form the pyridinone characteristic of Ring C. Ring D may be formed by functionalizing Ring C with an optionally substituted butene moiety, dihydroxylation the alkene of said butene, followed by intramolecular cyclization to form Ring D. Further functional group manipulations apparent to those having ordinary skill in the art may be employed to reach the final compounds.

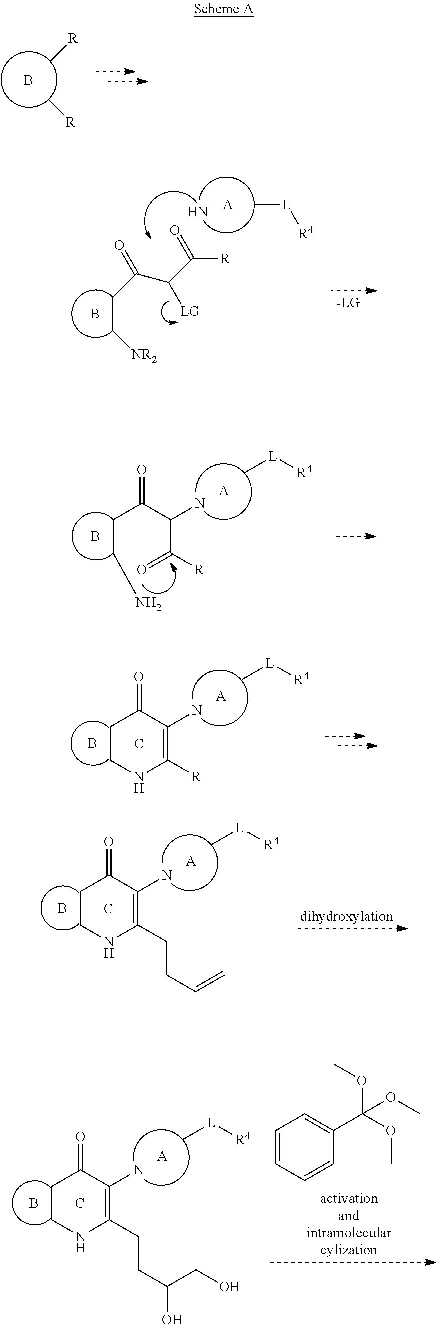

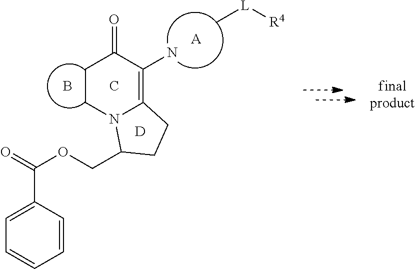

Certain compounds of the disclosure include a Ring B that is a 1,2,3-triazole. In that case, said triazole may be formed as illustrated in Scheme B below (specific reaction conditions are disclosed in Example 1 below). The compound syntheses may be finished analogously to the procedure of Scheme A above.

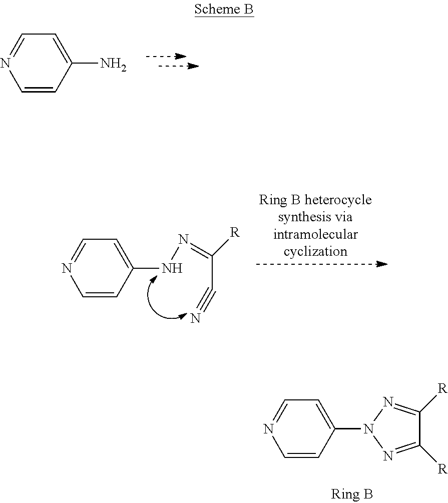

### EXAMPLES

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### List of Abbreviations

K₂CO₃: potassium carbonate
NaOH: sodium hydroxide
OsO₄: Osmium(VIII) oxide
NMO: 4-Methylmorpholine N-oxide
BF₃·Et₂O: boron trifluoride etherate
TPAP: tetra-n-propylammonium perruthenate
HATU: 2-(7-Azabenzotriazol-1-yl)-N,N,N′,N′-tetramethyluronium hexafluorophosphate
t-BuOH: tertiary alcohol
Na₂SO₃: sodium sulfite
AcOH: acetic acid
ACN: acetonitrile
THF: tetrahydrofuran
FA: formic acid
Na₂SO₄: sodium sulfate
EtOAc: ethyl acetate
DCM: dichloromethane
pH: potential of hydrogen
Boc₂O: di-tert-butyl dicarbonate
NH₄Cl: ammonium chloride
TFA: trifluoroacetic acid
NBS: N-bromosuccinimide
DIEA: N,N-diisopropylethylamine
LiHMDS: Lithium Hexamethyldisilazide
M: molar concentration
δ: Chemical shift
Cu(OAc)₂: Copper(II) acetate
HCl: Hydrochloride acid
EtOH: Ethanol
PMB: p-Methoxybenzyl
Wt %—weight percent
LiOH·H₂O: Lithium hydroxide monohydrate
CDI: 1,1′-Carbonyldiimidazole
NaHMDS: Sodium bis(trimethylsilyl)amide
SEM: 2-(trimethylsilyl)ethoxymethyl chloride
TEMPO: 2,2,6,6-tetramethylpiperidinyloxy
OTf: trifluoromethanesulfonate
OMs: mesylate
OTs: tosylate
SFC: supercritical fluid chromatography
T3P: propanephosphonic acid anhydride
DIBALH: diisobutylaluminium hydride
DMP: Dess-Martin Periodinane
EDCI: N-(3-dimethylaminopropyl)-N′-ethylcarbodiimide hydrochloride
(COCl)₂: Oxalyl chloride
TsOH-H₂O: 4-methylbenzenesulfonic acid monohydrate
TsOH: 4-Methylbenzenesulfonic acid
H₃PO₄: phosphoric acid
POCl₃: Phosphoryl chloride
ppm: parts per million
LCMS: liquid chromatography-mass spectrometry
HPLC: high-performance liquid chromatography
NMR: nuclear magnetic resonance
CDCl₃: deuterated chloroform
H₂O: water
DCM: dichloromethane
MeOH: methanol
DMF: N,N-dimethyl formamide
PE: petroleum ether
PPh₃: triphenyl phosphine
NH₄HCO₃: ammonium bicarbonate
Boc: tert-butyloxycarbonyl
Et₃N: triethylamine
N₂: nitrogen
Pd(dppf)Cl₂: bis(diphenylphosphino)ferrocene]dichloropalladium(II), complex
K₃PO₄: tripotassium phosphate
pH: potential of hydrogen
TLC: thin layer chromatography
CuCN: Copper(I) cyanide
DMA: Dimethylacetamide
NaIO₄: sodium periodate
NaHCO₃: Sodium hydrogen carbonate
NaBH₄: sodium borohydride
Cs₂CO₃: dicesium carbonate
CuI Copper(I) iodide
Pd(PPh₃)₂Cl₂: dichloropalladium triphenylphosphane
Rose Bengal: dipotassium 4,5,6,7-tetrachloro-2′,4′,5′,7′-tetraiodo-3-oxospiro[2-benzofuran-1,9′-xanthene]-3′,6′-diolate
Pd(PPh₃)₄: Tetrakis(triphenylphosphine)palladium(0)
NaH: Sodium Hydride
K₂OsO₄-2H₂O: potassium osmate (VI) dihydrate
DAST: Diethylaminosulfur trifluoride
LiOH: Lithium Hydroxide
K₂CO₃: Potassium carbonate, anhydrous
Pd(dppf)Cl₂—CH₂Cl₂: [1,1′-Bis(diphenylphosphino)ferrocene]dichloropalladium(II), complex with dichloromethane
O₂: Oxygen
DMSO: Dimethyl sulfoxide
LED: light emitting diode
LDA: Lithium diisopropylamide
NH₃: ammonia
CO: carbon monoxide
CD₃OD: deuterated methanol
MeMgBr: methylmagnesium bromide
HBr: hydrobromic acid
HI: Hydriodic acid
DMSO-d₆: deuterated dimethyl sulfoxide
NMP: N-methylpyrrolidone
DEA: Diethylamine
BOP: (Benzotriazollyloxy)tris(dimethylamino)phosphonium hexafluophosphate
KI: potassium iodide
MTBE: methyl tertiary butyl ether
m-CPBA: meta-chloroperoxybenzoic acid
DME: diethylene glycol dimethyl ether
IPA: isopropanol
C₂Cl₆: hexachloroethane
n-BuLi: n-butyllithium
DPPA: diphenylphosphoryl azide
RuPhos-Pd-G3 (CAS: 1445085-77-7): (2′-Amino-2-biphenylyl)(methanesulfonato-κO)palladium-dicyclohexyl(2′,6′-diisopropoxy-2-biphenylyl)phosphine
AgPF₆: silver hexafluorophosphate
(NH₄)₂S₂O₈: ammonium persulfate
T4P: 2,4,6-tributyl-1,3,5,2,4,6-trioxatriphosphinane 2,4,6-trioxide

### Example 1: General Synthetic Methods of Producing Compounds of the Disclosure

Compounds of the present invention can generally be prepared by conventional techniques known to those skilled in the art or by processes analogous to those described in the accompanying synthetic schemes.

Referring to Scheme 1 below, pyridin-4-amine 1 is reacted with ethyl 2-cyanoacetate, NaNO₂, HCl and water to furnish 2. 2 undergoes Cu-mediated heterocycle synthesis to form 3. 3 may then be reacted with 3-methylhex-5-en-2-one to form 4. α-Bromination of 4 furnishes 5. Nucleophilic substitution of the bromine residue of 5 with the appropriate piperazine in step 5 furnishes compound 6. 6 cyclizes and is deprotected under treatment with TFA to form the pyridinone Ring C of the present genus and is again protected with a Boc protecting group to form compound 7. Compound 7 undergoes vicinal syn dihydroxylation of the alkene to from diol 8. Lewis-acid promoted cyclization using trimethyl orthobenzoate yields ester 9. Hydrolysis of ester 9 yields alcohol 10. Oxidation of the primary alcohol of 10 to the acid to form 11, followed by pyridinium ion 12 formation with MeOTf, followed by removal of the pyridine moiety from the compound 12 by treatment with piperazine furnishes 13. Copper-catalyzed coupling of 13 with boronic acid derivatives followed by hydrolysis of the ester group leads to a free carboxylic acid 15, wherein said free carboxylic acid 15 is subjected to amide synthesis with the appropriate amine and separation of isomers to produce 16. Boc deprotection of 16 followed by amide coupling with sodium 5-hydroxy-6-methylpyrimidine-4-carboxylate produces compounds of the disclosure such as compounds of general formula 18.

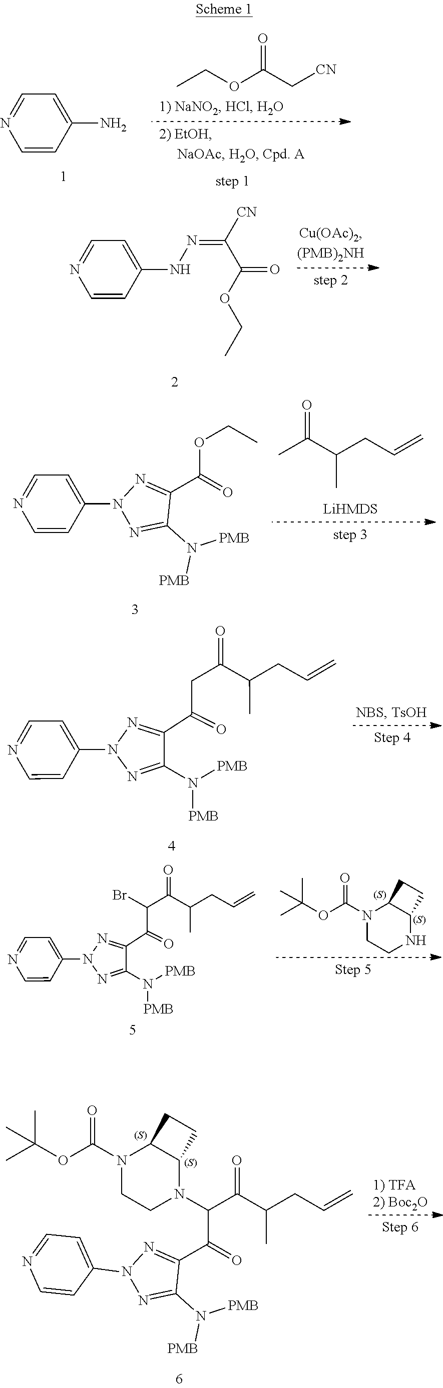

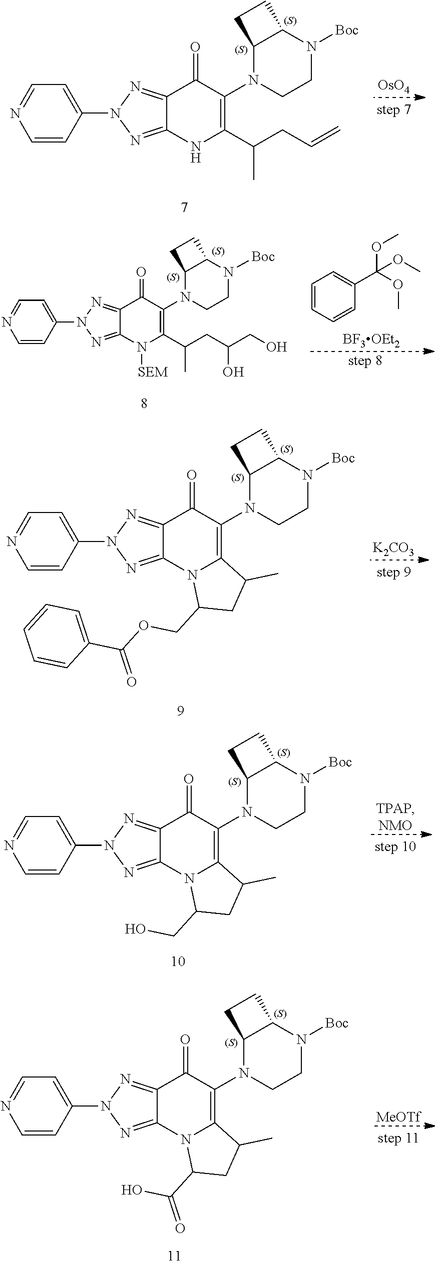

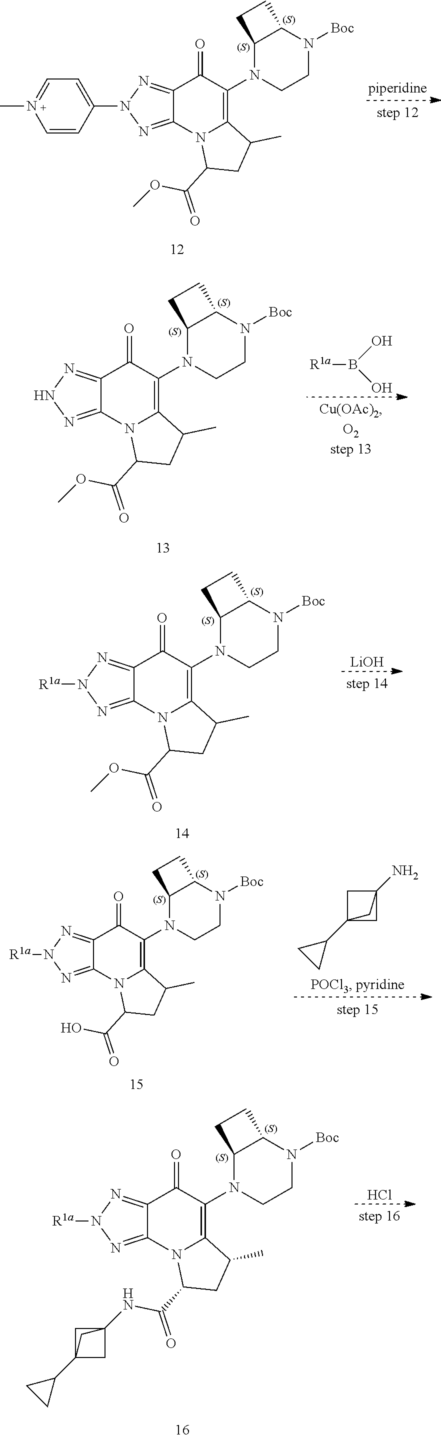

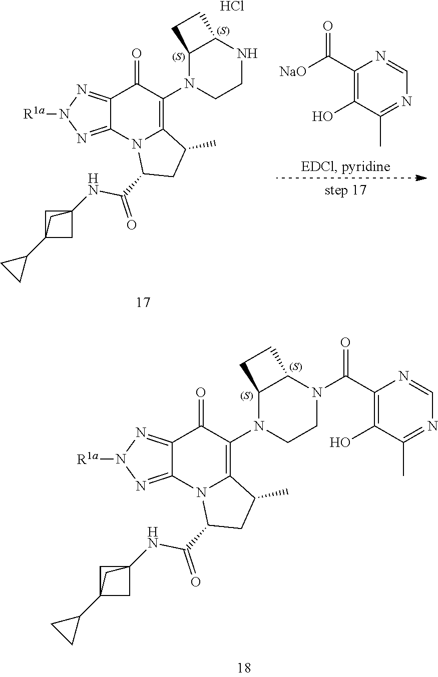

Referring to Scheme 2 below, compound of general formula 21 may be prepared via a method alternative to that in Scheme 1. Steps 1 and 2 proceed as described in Scheme 1. However, in Step 3, intermediate 3 is reacted with 2-butanone in the presence of base (to form enolate of the ketone) to produce β-diketone 4. Alfa-bromination of 4 followed by nucleophilic substitution of said bromine with the appropriate bicyclic piperazine (analogous to Scheme 1) produces 6. TFA promoted deprotection and cyclization of 6 to form the pyridinone (Ring C) followed by Boc protection of piperazine NH produces 7 which is then SEM protected at the pyridone NH group to yield 8. Deprotonation of 8 followed by alkylation with allyl iodide provides alkene 9. Cleavage of SEM protecting group yields 10. Vicinal syn dihydroxylation of 10 followed by Lewis-acid promoted cyclization yields 12. Completion of the synthesis analogous to the procedure described in Scheme 1 furnishes compounds of general formula 21.

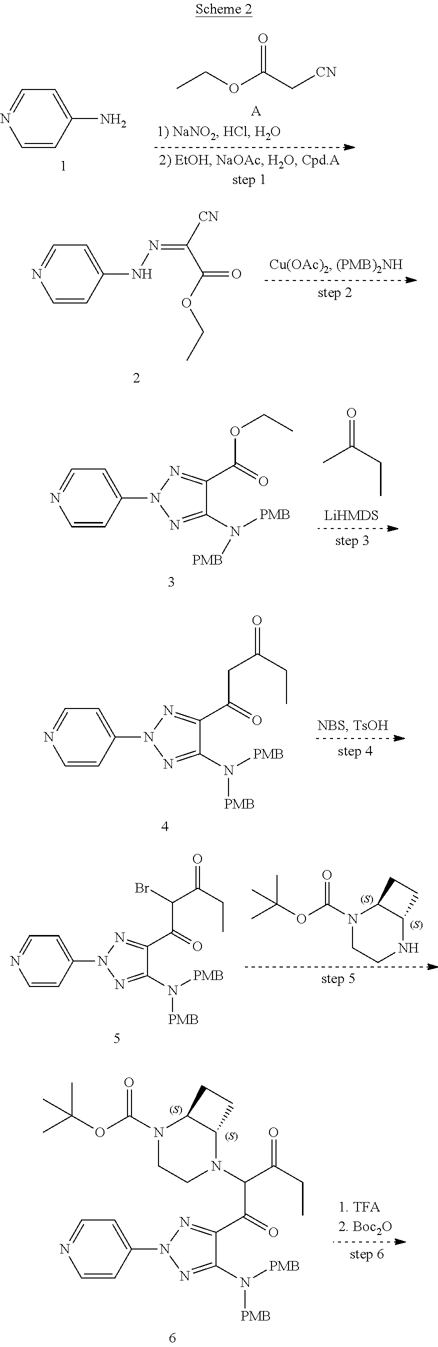

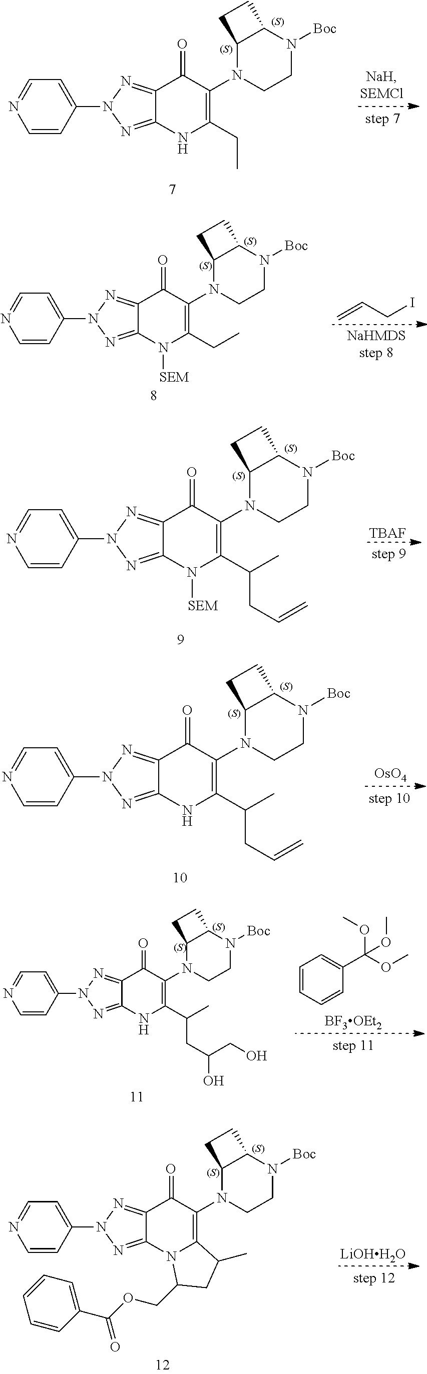

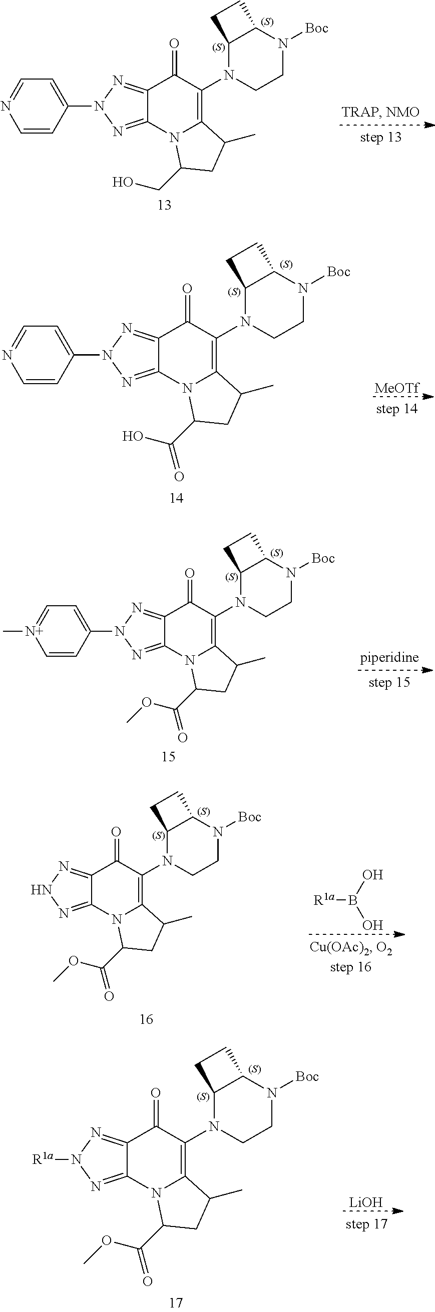

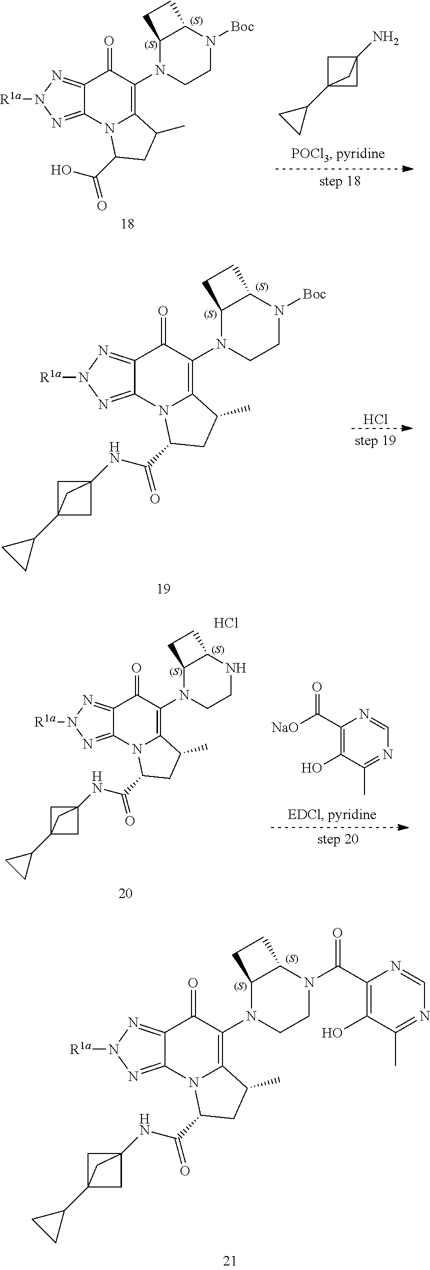

Referring to Scheme 3 below, pyridin-4-amine 1 is reacted with ethyl 2-cyanoacetate, NaNO₂, HCl and water to furnish 2. 2 undergoes Cu-mediated heterocycle synthesis to form 3. 3 may then be reacted with 3-methylhex-5-en-2-one to form 4. α-Bromination of 4 furnishes 5. Nucleophilic substitution of the bromine residue of 5 with the appropriate piperazine in step 5 furnishes compound 6. 6 cyclizes and is deprotected under treatment with TFA to form the pyridinone Ring C of the present genus and is again protected with a Boc protecting group to form compound 7. Compound 7 undergoes vicinal syn dihydroxylation of the alkene to from diol 8. Lewis-acid promoted cyclization using trimethyl orthobenzoate yields ester 9. Hydrolysis of ester 9 yields alcohol 10. Oxidation of the primary alcohol of 10 to the carboxylic acid leads to compound 11. Carboxylic acid 11 is subjected to amide synthesis with the appropriate amine producing 12. Boc deprotection of 12 followed by amide coupling with sodium 5-hydroxy-6-methylpyrimidine-4-carboxylate produces compounds of the disclosure such as compounds of general formula 14. Single stereoisomers of 14 are readily obtained by means of HPLC or SFC separation of stereoisomeric mixtures of 14 or synthesis intermediates such as 11, 12 or 13 using chiral or non-chiral stationary phase as known to the person skilled in the art.

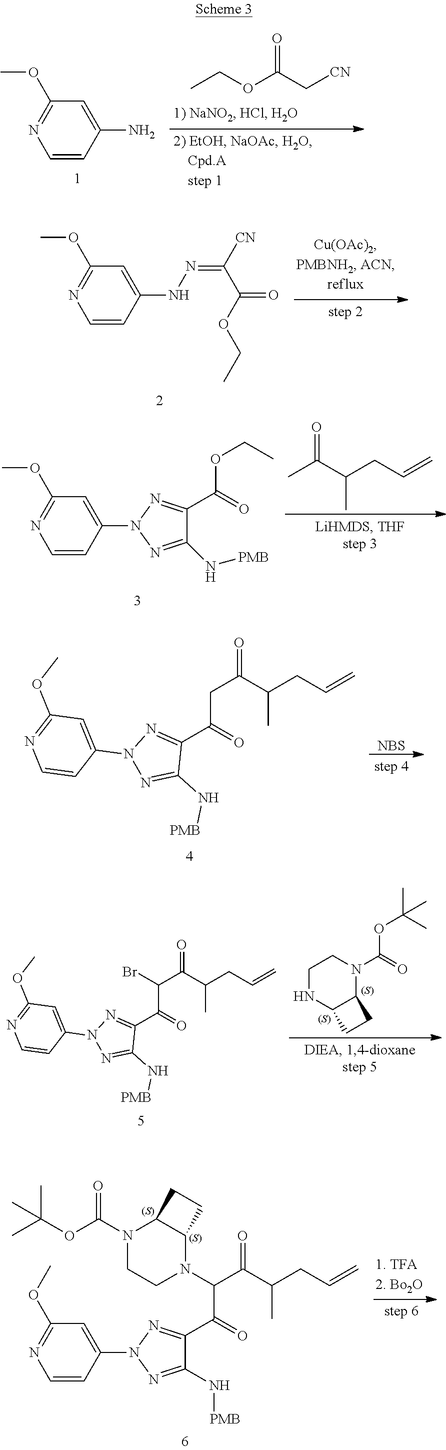

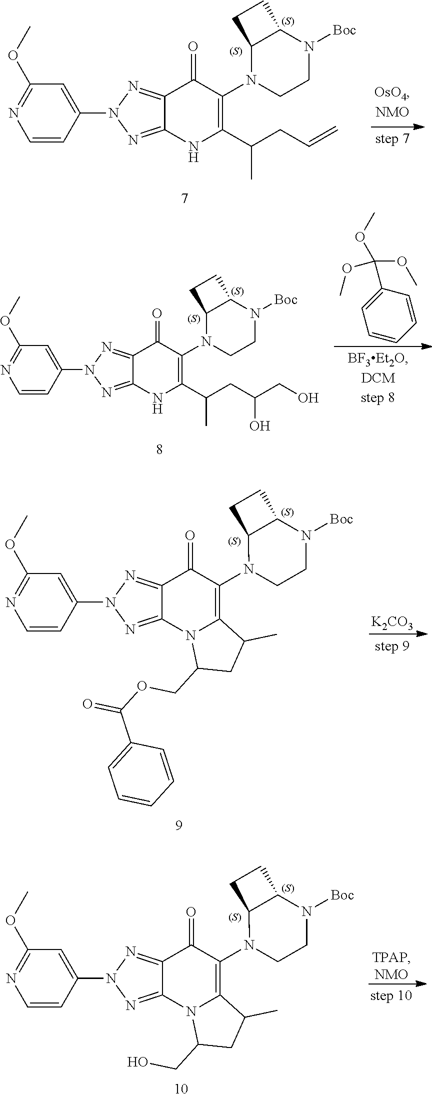

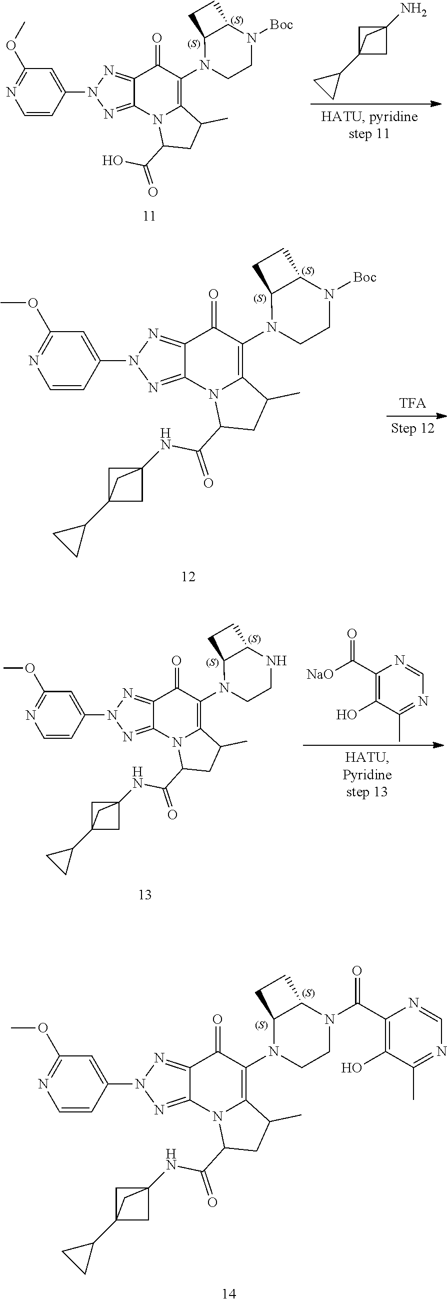

Referring to Scheme 4, compounds of general formula 15 may be prepared as illustrated therein. Suzuki coupling of 1 with appropriate boronic acid or boronate ester produces 2 which when contacted with the enolate formed by 3-methylhex-5-en-2-one and LDA, displaces methanol to form β-diketone 3. α-Bromination of 3 followed by nucleophilic displacement of the bromine with the appropriate piperazine provides 5. NaOMe promoted cyclization forms the pyridinone Ring C of the core in compound 6. 6 undergoes vicinal syn dihydroxylation of the alkene to from diol 7. The N-PMB group is cleaved by treatment with TFA and the Boc-protecting group is reinstalled to give ester 8. Ester cleavage yields diol 9. Lewis-acid promoted cyclization of 9 using trimethyl orthobenzoate yields ester 10. Hydrolysis of ester 10 yields alcohol 11. Oxidation of the primary alcohol of 11 to the acid to form 12, followed by POCl₃ facilitated amidation with appropriately functionalized primary amine furnishes 13. 13 is Boc-deprotected to furnish 14 which is then subjected to amide coupling with sodium 5-hydroxy-6-methylpyrimidine-4-carboxylate to produce compounds of the disclosure such as compounds of general formula 15. Single stereoisomers of 15 are readily obtained by means of HPLC or SFC separation of stereoisomeric mixtures of 15 or synthesis intermediates such as 12, 13 or 14 using chiral or non-chiral stationary phase as known to the person skilled in the art.

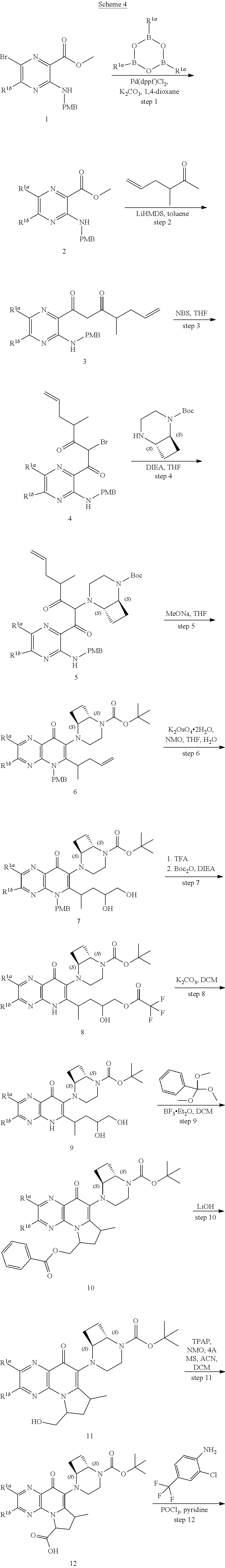

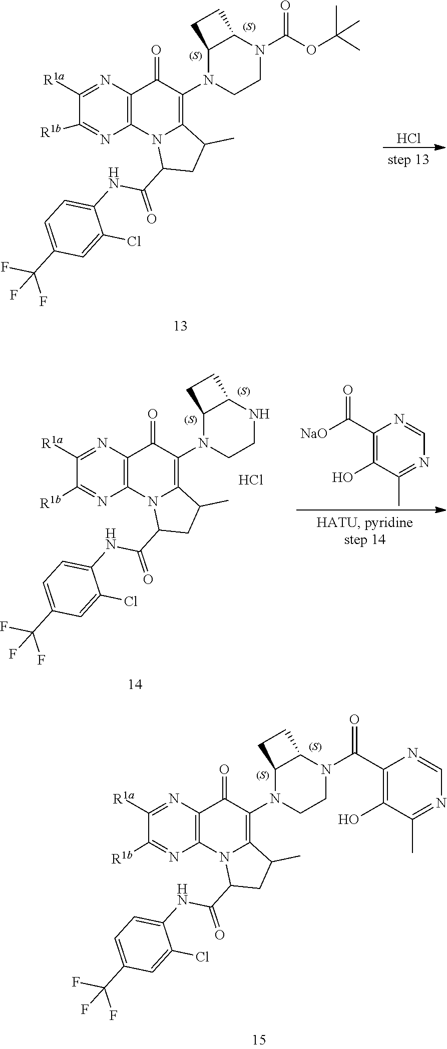

Compounds such as I-18a may be prepared as shown in Scheme 5 below, by making modifications to said procedure known to those of ordinary skill in the art. Referring to Scheme 5, Compound D (see intermediate synthesis section below) and hydrazinecarboximidamide may be reacted to form Ring C, the appropriately substituted 2-imino-2,3-dihydropyrimidin-4(1H)-one 1. Heterocycle synthesis to form triazolyl Ring B of 2 is achieved by reaction of Compound C with 1 in the presence of FeCl₃. α-Bromination of the ester side chain of 2 followed by intramolecular nucleophilic substitution at said α-carbon produces Ring D seen in product 3.3 is chirally resolved to produce enantiopure material 3′. 3′ is subjected to chemistry described in the above Schemes to produce 8 which is chirally resolved to furnish I-18a.

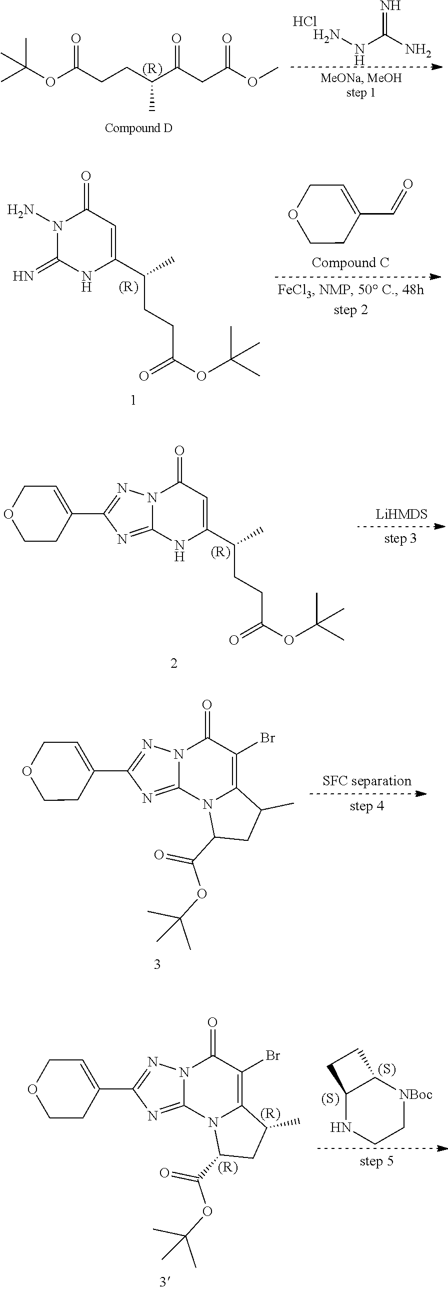

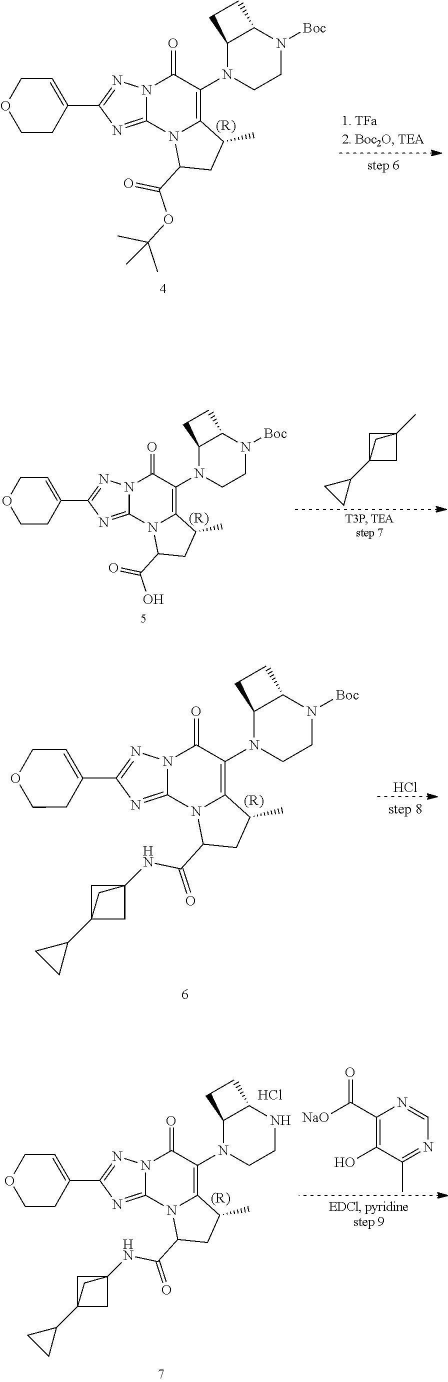

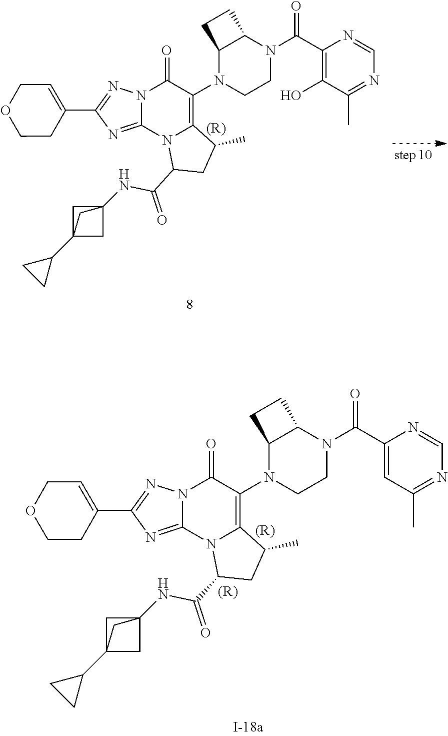

### Example 2: Starting Material Compound Synthesis

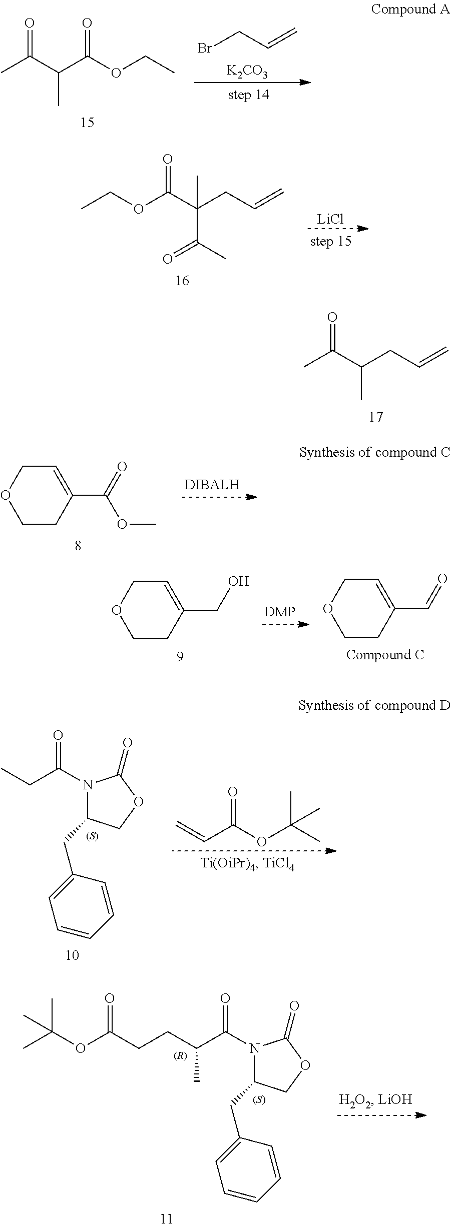

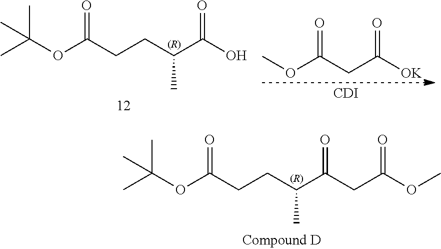

### Example 3: Synthesis of Compounds of the Disclosure

### Synthesis of N-{3-cyclopropylbicyclo[1.1.1]pentan-1-yl}-5-[(1S,6S)-5-(5-hydroxy-6-methylpyrimidine-4-carbonyl)-2,5-diazabicyclo[4.2.0]octan-2-yl]-2-(2-methoxypyridin-4-yl)-6-methyl-4-oxo-2H,4H,6H,7H,8H-[1,2,3]triazolo[4,5-e]indolizine-8-carboxamide (Example I-1 as Stereoisomer 1) and N-{3-cyclopropylbicyclo[1.1.1]pentan-1-yl}-5-[(1S,6S)-5-(5-hydroxy-6-methylpyrimidine-4-carbonyl)-2,5-diazabicyclo[4.2.0]octan-2-yl]-2-(2-methoxypyridin-4-yl)-6-methyl-4-oxo-2H,4H,6H,7H,8H-[1,2,3]triazolo[4,5-e]indolizine-8-carboxamide (Example I-2 as Stereoisomer 2)

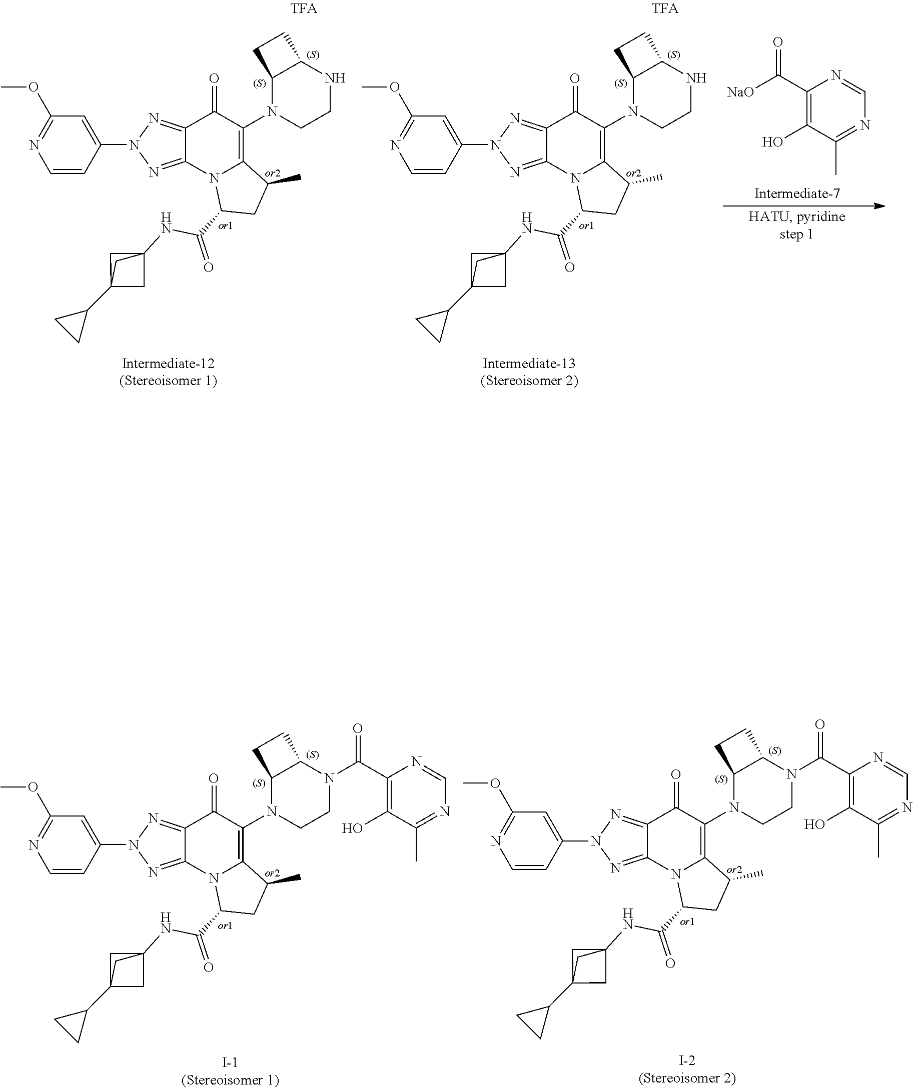

Step 1. To a solution of 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (mixture of Intermediate 12 (Stereoisomer 1) and Intermediate 13 (Stereoisomer 2) obtained as described in the “Synthesis of intermediates” section of this document) (100 mg, 180 mol, 1.0 eq) and sodium 5-hydroxy-6-methylpyrimidine-4-carboxylate (Intermediate-7) (95 mg, 539 mol, 3.0 eq) in pyridine (2 mL) was added HATU (137 mg, 359 mol, 2.0 eq), and the resulting mixture was stirred at room temperature for 10 min. The reaction mixture was diluted with water (10 mL) and extracted with EtOAc (10 mL*3). The combined organic layers were washed with brine (5 mL), dried over anhydrous Na₂SO₄, filtered and concentrated in vacuo. The residue was purified by reverse phase HPLC (C18 column, water (0.1% FA)-ACN) to afford stereoisomer 1 and stereoisomer 2. Example I-1 as stereoisomer 1: LCMS: 693.4 [M+H]⁺. ¹H NMR (400 MHz, CDCl₃) δ 12.45-11.42 (m, 1H), 8.60 (s, 1H), 8.28 (d, 1H), 7.67 (dd, 1H), 7.47 (d, 1H), 6.58 (br s, 1H), 6.16-4.11 (m, 3H), 4.08-3.88 (m, 4H), 3.86-3.62 (m, 2H), 3.57-3.26 (m, 2H), 2.72 (ddd, 1H), 2.55 (s, 3H), 2.39-2.09 (m, 2H), 1.89 (s, 6H), 1.85-1.77 (m, 1H), 1.58 (br d, 3H), 1.55-1.41 (m, 2H), 0.96-0.85 (m, 1H), 0.46-0.34 (m, 2H), 0.18-0.05 (m, 2H). Example I-2 as stereoisomer 2: LCMS: 693.4 [M+H]⁺. ¹H NMR (400 MHz, CDCl₃) δ 12.46-11.72 (m, 1H), 8.61 (s, 1H), 8.30 (d, 1H), 7.69 (dd, 1H), 7.49 (d, 1H), 6.58 (br s, 1H), 5.82-4.54 (m, 2H), 4.33-3.79 (m, 6H), 3.78-3.55 (m, 2H), 3.54-3.38 (m, 1H), 2.82 (td, 1H), 2.55 (s, 3H), 2.32 (br d, 2H), 1.89 (s, 6H), 1.85-1.80 (m, 1H), 1.54 (br d, 3H), 1.50-1.28 (m, 2H), 1.00-0.80 (m, 1H), 0.49-0.30 (m, 2H), 0.20-0.01 (m, 2H).

### Synthesis of N-{3-cyclopropylbicyclo[1.1.1]pentan-1-yl}-5-[(1S,6S)-5-(5-hydroxy-6-methylpyrimidine-4-carbonyl)-2,5-diazabicyclo[4.2.0]octan-2-yl]-2-(2-methoxypyridin-4-yl)-6-methyl-4-oxo-2H,4H,6H,7H,8H-[1,2,3]triazolo[4,5-e]indolizine-8-carboxamide (Example I-3 as Stereoisomer 3)

The example I-3 was prepared through the same procedure as described for I-1 and I-2 starting from intermediate Intermediate-14 (Stereoisomer 3). LCMS: 693.4 [M+H]⁺. ¹H NMR (400 MHz, CDCl₃) δ 12.46-11.74 (m, 1H), 8.62 (s, 1H), 8.30 (d, 1H), 7.69 (dd, 1H), 7.49 (d, 1H), 6.43 (s, 1H), 5.75-4.60 (m, 2H), 4.58-4.17 (m, 1H), 4.16-3.90 (m, 5H), 3.89-3.79 (m, 1H), 3.77-3.59 (m, 1H), 3.58-3.34 (m, 1H), 2.80-2.66 (m, 1H), 2.55 (s, 3H), 2.39-2.09 (m, 2H), 1.90 (s, 6H), 1.82-1.72 (m, 1H), 1.54 (br d, 3H), 1.49-1.33 (m, 2H), 0.99-0.84 (m, 1H), 0.48-0.35 (m, 2H), 0.16-0.08 (m, 2H).

### Synthesis of N-{3-cyclopropylbicyclo[1.1.1]pentan-1-yl}-5-[(1S,6S)-5-(5-hydroxy-6-methylpyrimidine-4-carbonyl)-2,5-diazabicyclo[4.2.0]octan-2-yl]-2-(2-methoxypyridin-4-yl)-6-methyl-4-oxo-2H,4H,6H,7H,8H-[1,2,3]triazolo[4,5-e]indolizine-8-carboxamide (Example I-4 as Stereoisomer 4)

The example I-4 was prepared through the same procedure as described for I-1 and I-2 starting from intermediate Intermediate-15 (Stereoisomer 4). LCMS: 693.4 [M+H]⁺. ¹H NMR (400 MHz, CDCl₃) δ 12.46-11.43 (m, 1H), 8.60 (s, 1H), 8.30 (d, 1H), 7.69 (dd, 1H), 7.49 (d, 1H), 6.57 (br s, 1H), 6.04-4.57 (m, 2H), 4.53-4.14 (m, 1H), 4.12-3.84 (m, 4H), 3.80-3.58 (m, 2H), 3.55-3.23 (m, 2H), 2.81 (dt, 1H), 2.56 (s, 3H), 2.43-2.15 (m, 2H), 1.90 (s, 6H), 1.84-1.73 (m, 1H), 1.60 (br d, 3H), 1.54-1.45 (m, 2H), 0.98-0.86 (m, 1H), 0.46-0.36 (m, 2H), 0.11 (q, 2H).

### Synthesis of N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-3,7-dimethyl-5-oxo-5,7,8,9-tetrahydropyrazino[2,3-e]indolizine-9-carboxamide (Example I-11 as Stereoisomer 1) and N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-3,7-dimethyl-5-oxo-5,7,8,9-tetrahydropyrazino[2,3-e]indolizine-9-carboxamide (Example I-7 as Stereoisomer 4)

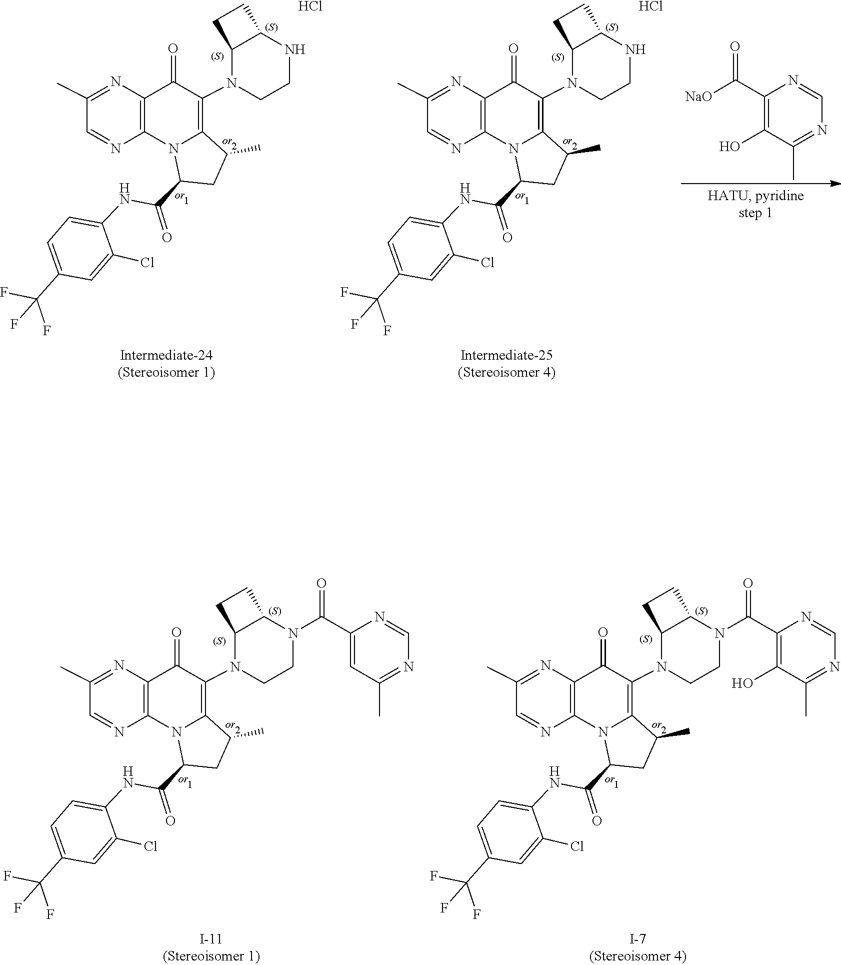

Step 1. To a solution of 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (mixture of Intermediate-24 (Stereoisomer 1) and Intermediate-25 (Stereoisomer 4) obtained as described in the “Synthesis of intermediates” section of this document infra (119 mg, 204 mol, 1.0 eq) and sodium 5-hydroxy-6-methylpyrimidine-4-carboxylate (72 mg, 408 mol, 2.0 eq) in pyridine (2 mL) was added HATU (116 mg, 306 mol, 1.5 eq), and the resulting mixture was stirred at room temperature for 1 h. The reaction mixture was diluted with water (10 mL) and extracted with EtOAc (10 mL*3). The combined organic layers were washed with brine (5 mL), dried over anhydrous Na₂SO₄, filtered and concentrated in vacuo. The residue was purified by reverse phase HPLC to afford stereoisomer 1 and stereoisomer 4. Prep-HPLC method: column: Phenomenex luna C18 150*25 mm*10 μm; Mobile Phase: Phase A for water (0.225% FA), Phase B for ACN; gradient:55%-65% B over 10 min; Flow rate: 25 mL/min; Retention Time: Peak1 0.910 min, Peak2 0.928 min. Example I-11 as Stereoisomer 1, first eluting peak: LCMS: 683.3 [M+H]⁺. ¹H NMR (400 MHz, CDCl₃) δ 12.79-11.31 (m, 1H), 8.83 (s, 1H), 8.60 (s, 1H), 8.48 (s, 1H), 8.46 (d, 1H), 7.66 (d, 1H), 7.57-7.48 (m, 1H), 5.74-4.66 (m, 2H), 4.64-4.29 (m, 1H), 4.25-3.97 (m, 1H), 3.92 (dq, 1H), 3.87-3.65 (m, 1H), 3.65-3.32 (m, 2H), 2.97-2.83 (m, 1H), 2.75 (s, 3H), 2.55 (s, 3H), 2.29 (dt, 2H), 1.73 (br d, 4H), 1.59-1.38 (m, 2H). Example I-7 as Stereoisomer 4, second eluting peak: LCMS: 683.3 [M+H]⁺. ¹H NMR (400 MHz, CDCl₃) δ 12.67-11.54 (m, 1H), 9.00 (br s, 1H), 8.61 (s, 1H), 8.56-8.49 (m, 2H), 7.66 (d, 1H), 7.53 (br d, 1H), 5.79-4.57 (m, 2H), 4.47-4.04 (m, 2H), 4.03-3.84 (m, 2H), 3.83-3.62 (m, 1H), 3.57-3.33 (m, 1H), 2.88 (dt, 1H), 2.77 (s, 3H), 2.55 (s, 3H), 2.52-2.25 (m, 2H), 1.73 (br d, 1H), 1.63 (d, 3H), 1.55-1.33 (m, 2H).

### Synthesis of N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-3,7-dimethyl-5-oxo-5,7,8,9-tetrahydropyrazino[2,3-e]indolizine-9-carboxamide (Example I-12 as Stereoisomer 2) and N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-3,7-dimethyl-5-oxo-5,7,8,9-tetrahydropyrazino[2,3-e]indolizine-9-carboxamide (Example I-8 as Stereoisomer 3)

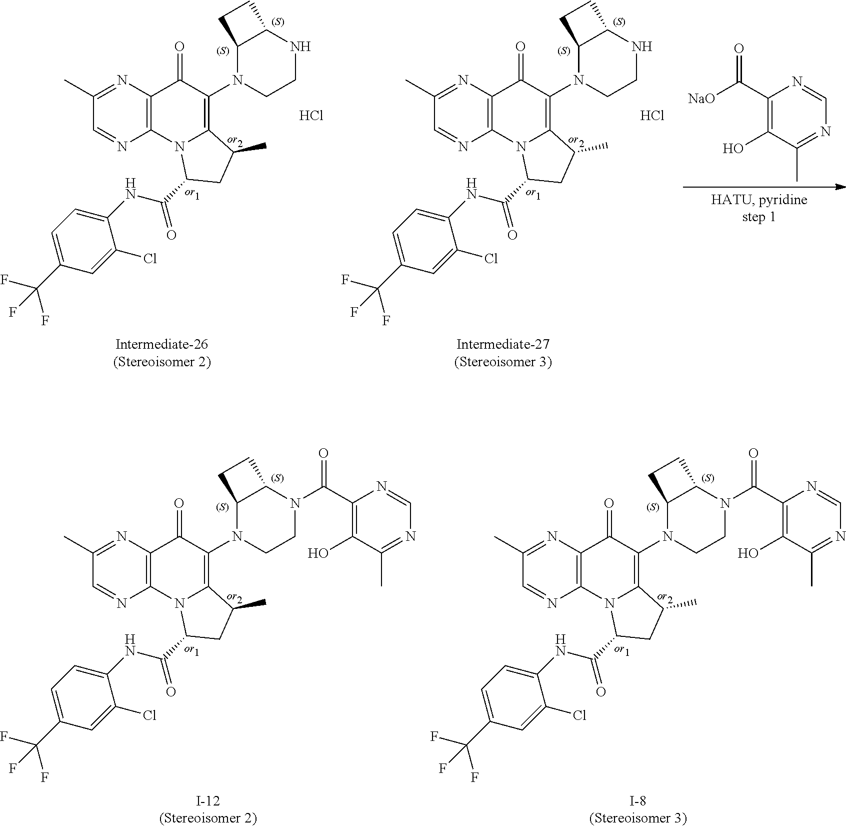

Step 1. To a solution of 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (mixture of Intermediate-2 (Stereoisomer 2) and Intermediate-27 (Stereoisomer 3) obtained as described in the “Synthesis of intermediates” section of this document) (134 mg, 230 mol, 1.0 eq) and sodium 5-hydroxy-6-methylpyrimidine-4-carboxylate (91 mg, 461 mol, 2.0 eq) in pyridine (2 mL) was added HATU (123 mg, 323 mol, 1.4 eq), and the resulting mixture was stirred at room temperature for 1 h. The reaction mixture was diluted with water (10 mL) and extracted with EtOAc (10 mL*3). The combined organic layers were washed with brine (5 mL), dried over anhydrous Na₂SO₄, filtered and concentrated in vacuo. The residue was purified by reverse phase HPLC (C18 column, water (0.1% FA)-ACN) to afford a mixture of stereoisomer 2 and stereoisomer 3. The mixture was then separated by chiral SFC Instrument: Thar 80 system; Column: DAICEL CHIRALPAK AD (250 mm*30 mm, 10 μm); Mobile Phase: Phase A for Supercritical CO₂, Phase B for i-PrOH (0.1% NH₃H₂O); Isocratic elution: 35% Phase B in Supercritical CO₂; Flow rate: 75 g/min; Retention Time: Peak1 5.37 min, Peak2 8.29 min; cycle time: 7.2 min; Back Pressure: 100 bar to keep the CO₂ in Supercritical flow; UV: 220 nm to afford stereoisomer 2 and stereoisomer 3. Example I-12 as Stereoisomer 2 (peak 1 in chiral SFC separation): LCMS: 683.3 [M+H]⁺. ¹H NMR (400 MHz, CDCl₃) δ 12.61-11.66 (s, 1H), 8.88 (br s, 1H), 8.63 (br s, 1H), 8.52 (s, 1H), 8.49 (d, 1H), 7.66 (s, 1H), 7.52 (br d, 1H), 5.78-4.69 (m, 2H), 4.66-4.25 (m, 1H), 4.22-3.89 (m, 3H), 3.89-3.50 (m, 1H), 3.48-3.33 (m, 1H), 2.90 (ddd, 1H), 2.78 (s, 3H), 2.55 (s, 3H), 2.39-2.19 (m, 2H), 2.00-1.66 (m, 4H), 1.53-1.38 (m, 2H). Example I-8 as Stereoisomer 3 (peak 2 in chiral SFC separation): LCMS: 683.3 [M+H]⁺. ¹H NMR (400 MHz, CDCl₃) δ 9.08 (br s, 1H), 8.60 (br s, 1H), 8.56-8.50 (m, 2H), 7.66 (s, 1H), 7.54 (br d, 1H), 5.77-4.71 (m, 2H), 4.66-4.28 (m, 1H), 4.25-3.97 (m, 1H), 3.94-3.69 (m, 2H), 3.66-3.31 (m, 2H), 2.92-2.81 (m, 1H), 2.78 (s, 3H), 2.59-2.51 (m, 4H), 2.42-2.22 (m, 1H), 1.77-1.67 (m, 4H), 1.55-1.42 (m, 2H).

### Synthesis of N-[2-chloro-4-(trifluoromethyl)phenyl]-6-[(1S,6S)-5-(5-hydroxy-6-methylpyrimidine-4-carbonyl)-2,5-diazabicyclo[4.2.0]octan-2-yl]-2,3,7-trimethyl-5-oxo-5H,7H,8H,9H-pyrazino[2,3-e]indolizine-9-carboxamide (Example I-17 as Stereoisomer 1) and N-[2-chloro-4-(trifluoromethyl)phenyl]-6-[(1S,6S)-5-(5-hydroxy-6-methylpyrimidine-4-carbonyl)-2,5-diazabicyclo[4.2.0]octan-2-yl]-2,3,7-trimethyl-5-oxo-5H,7H,8H,9H-pyrazino[2,3-e]indolizine-9-carboxamide (Example I-18 as Stereoisomer 2)

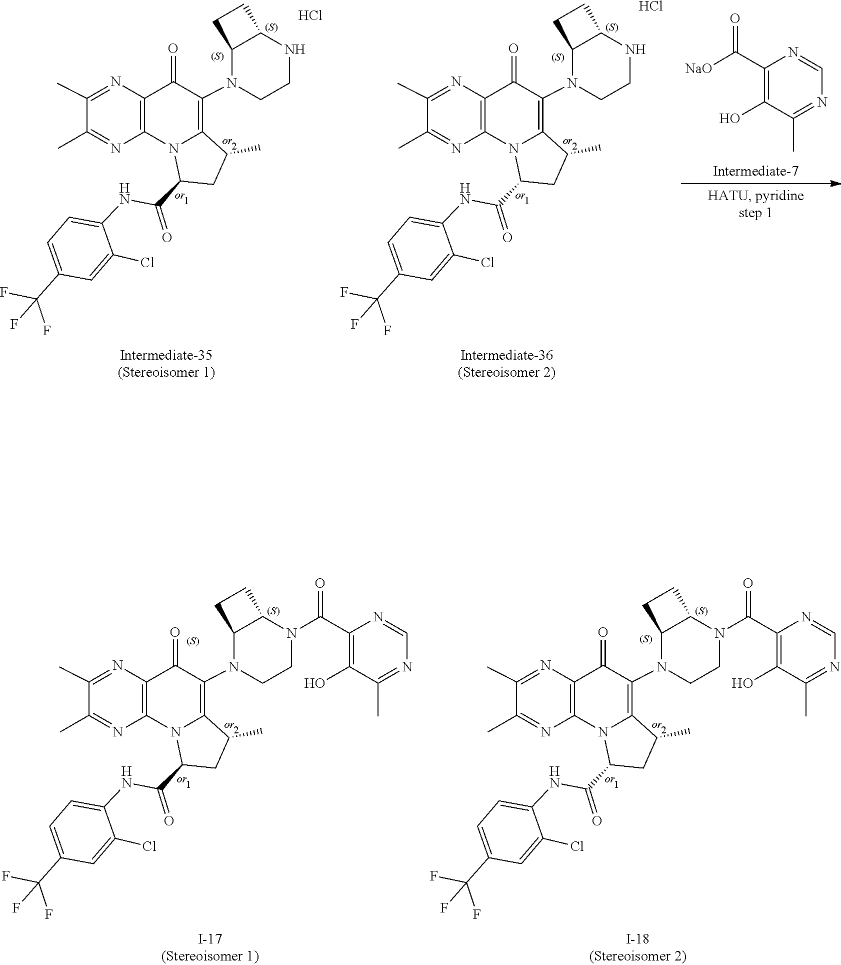

Step 1. To a solution of 6-((1S,6S)-2,5-diazabicyclo[4.2.0]octan-2-yl)-N-(2-chloro-4-(trifluoromethyl)phenyl)-2,3,7-trimethyl-5-oxo-5,7,8,9-tetrahydropyrazino[2,3-e]indolizine-9-carboxamide hydrochloride (a mixture of Intermediate-35 (Stereoisomer 1) and Intermediate-36 (Stereoisomer 2) obtained as described in the “Synthesis of intermediates” section of this document) (717 mg, 1.28 mmol, 1.0 eq) and sodium 5-hydroxy-6-methylpyrimidine-4-carboxylate (Intermediate-7) (450 mg, 2.56 mmol, 2.0 eq) in pyridine (10 mL) was added HATU (583 mg, 1.53 mmol, 1.2 eq), and the resulting mixture was stirred at room temperature for 0.5 h. The reaction mixture was diluted with water (30 mL) and extracted with EtOAc (50 mL*3). The combined organic layers were washed with brine (20 mL), dried over anhydrous Na₂SO₄, filtered and concentrated under reduced pressure. The residue was purified by reverse phase HPLC to afford the title compound as stereoisomer 1 (first eluting) and stereoisomer 2 (second eluting). Prep-HPLC method: column: Phenomenex luna C18 150*25 mm*10 μm; Mobile Phase: Phase A for water (0.225% FA), Phase B for ACN; gradient: hold 40% B for 0.9 min, to 55% B over 0.1 min, to 65% B over 10 min, to 100% B over 0.1 min, hold 100% B for 4 min, to 5% B over 0.1 min, hold 5% B for 1 min; Flow rate: 25 mL/min; UV 220 nm/254 nm; Retention Time: Peak1 8.10 min, Peak2 9.80 min. Example I-17 as Stereoisomer 1, first eluting peak: LCMS: 697.2 [M+H]⁺. ¹H NMR (400 MHz, CDCl₃) δ 12.45-11.55 (m, 1H), 8.60 (s, 1H), 8.53 (br s, 1H), 8.43 (d, 1H), 7.67 (d, 1H), 7.55 (dd, 1H), 5.55 (dd, 1H), 4.82-4.40 (m, 2H), 4.23-3.72 (m, 3H), 3.65-3.34 (m, 2H), 2.84 (td, 1H), 2.69 (s, 3H), 2.60 (s, 3H), 2.55 (s, 3H), 2.38-2.14 (m, 2H), 1.77-1.71 (m, 4H), 1.61-1.32 (m, 2H). Example I-18 as Stereoisomer 2, second eluting peak: LCMS: 697.2 [M+H]⁺. ¹H NMR (400 MHz, CDCl₃) δ 12.75-11.60 (m, 1H), 8.61 (s, 1H), 8.57 (br s, 1H), 8.45 (d, 1H), 7.66 (d, 1H), 7.54 (dd, 1H), 5.62 (dd, 1H), 5.12-4.70 (m, 1H), 4.36-4.07 (m, 2H), 4.03-3.84 (m, 2H), 3.79-3.40 (m, 2H), 2.87 (td, 1H), 2.71 (s, 3H), 2.63 (s, 3H), 2.61 (s, 3H), 2.55-2.30 (m, 2H), 2.03-1.90 (m, 1H), 1.61 (br d, 3H), 1.55-1.31 (m, 2H).

### Synthesis of N-(2-fluoro-4-(trifluoromethyl)phenyl)-5-((1S,6S)-5-(5-hydroxy-6-methylpyrimidine-4-carbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxamide (Example I-6 as Stereoisomer 2)

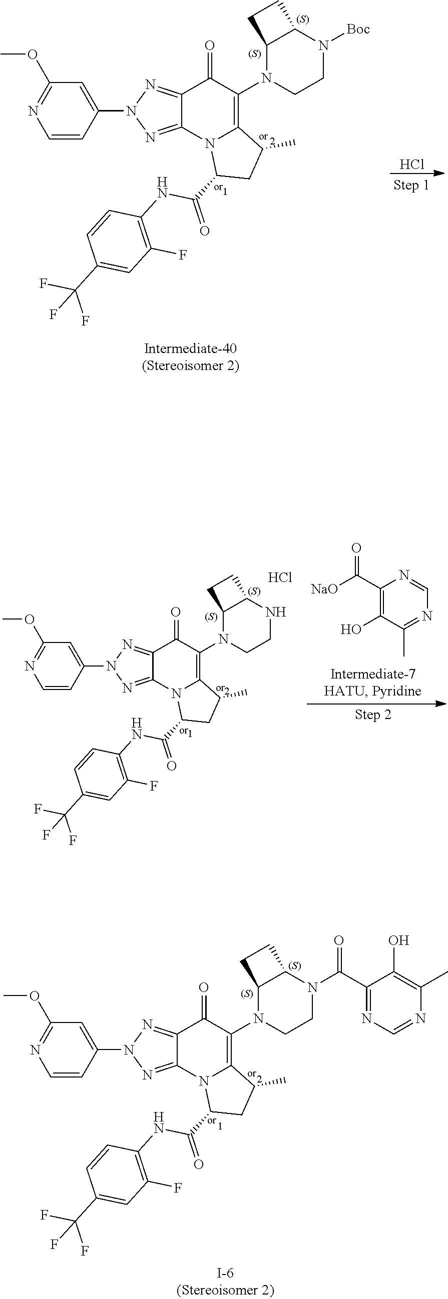

Step 1: To a solution of tert-butyl (1S,6S)-5-(8-((2-fluoro-4-(trifluoromethyl)phenyl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Intermediate-40 as Stereoisomer 2) (70 mg, 98 mol, 1.0 eq) in 1,4-dioxane (1 mL) was added a solution of HCl in 1,4-dioxane (4 M, 2 mL), and the resulting mixture was stirred at room temperature for 0.5 h. The reaction mixture was concentrated under reduced pressure to afford 5-((1S,6S)-2,5-diazabicyclo[4.2.0]octan-2-yl)-N-(2-fluoro-4-(trifluoromethyl)phenyl)-2-(2-methoxypyridin-4-yl)-6-methyl-4-oxo-4,6,7,8-tetrahydro-2H-[1,2,3]triazolo[4,5-e]indolizine-8-carboxamide hydrochloride (stereoisomer 2), which was used into the next step without further purification. LCMS: 613.3 [M+H]⁺.

Step 2: To a solution of 5-((1S,6S)-2,5-diazabicyclo[4.2.0]octan-2-yl)-N-(2-fluoro-4-(trifluoromethyl)phenyl)-2-(2-methoxypyridin-4-yl)-6-methyl-4-oxo-4,6,7,8-tetrahydro-2H-[1,2,3]triazolo[4,5-e]indolizine-8-carboxamide hydrochloride (Stereoisomer 2) (60 mg, 92 mol, 1.0 eq) and sodium 5-hydroxy-6-methylpyrimidine-4-carboxylate (Intermediate-7) (33 mg, 185 mol, 2.0 eq) in pyridine (1 mL) was added HATU (52 mg, 138 mol, 1.5 eq), and the resulting mixture was stirred at room temperature for 0.5 h. The reaction mixture was diluted with H₂O (20 mL) and extracted with EtOAc (20 mL*2). The combined organic layers were dried over anhydrous Na₂SO₄ and concentrated under reduced pressure. The residue was purified by reverse phase HPLC (C18 column, water (0.1% FA)-ACN) to afford N-(2-fluoro-4-(trifluoromethyl)phenyl)-5-((1S,6S)-5-(5-hydroxy-6-methylpyrimidine-4-carbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxamide (Stereoisomer 2). LCMS: 749.3 [M+H]⁺. ¹H NMR (400 MHz, CDCl₃) δ 12.95-10.49 (m, 1H), 8.62 (s, 1H), 8.55-8.49 (m, 1H), 8.46 (s, 1H), 8.29 (d, 1H), 7.70 (dd, 1H), 7.66 (d, 1H), 7.56 (d, 1H), 7.50 (d, 1H), 5.34 (dd, 2H), 4.07 (s, 2H), 4.00 (s, 3H), 3.82 (s, 2H), 3.79-3.62 (m, 1H), 3.58-3.38 (m, 1H), 3.09-2.91 (m, 1H), 2.56 (s, 4H), 2.45-2.18 (m, 1H), 2.13-1.83 (m, 1H), 1.58 (d, 5H).

### Synthesis of N-(2-fluoro-4-(trifluoromethyl)phenyl)-5-((1S,6S)-5-(5-hydroxy-6-methylpyrimidine-4-carbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxamide (Example I-15 as Stereoisomer 1)

Example I-15 (Stereoisomer 1) was synthesized by analogy to the 2-step procedure described for example I-6 starting from Intermediate-39 (Stereoisomer 1). LCMS: 749.3 [M+H]+¹H NMR (400 MHz, CDCl₃) δ 12.95-10.66 (m, 1H), 8.61 (s, 1H), 8.49 (d, 1H), 8.36 (s, 1H), 8.26 (d, 1H), 7.71-7.67 (m, 1H), 7.67-7.63 (m, 1H), 7.58-7.53 (m, 1H), 7.43 (d, 1H), 5.30 (dd, 2H), 4.58-4.25 (m, 1H), 3.98 (s, 4H), 3.88-3.65 (m, 2H), 3.62-3.28 (m, 2H), 3.00-2.81 (m, 1H), 2.56 (s, 3H), 2.47-2.24 (m, 2H), 2.00-1.86 (m, 1H), 1.69 (d, 3H), 1.59-1.44 (m, 2H).

### Synthesis of N-(2-fluoro-4-(trifluoromethyl)phenyl)-5-((1S,6S)-5-(5-hydroxy-6-methylpyrimidine-4-carbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxamide (Example I-10 as Stereoisomer 3)

Example I-10 (Stereoisomer 3) was synthesized by analogy to the 2-step procedure described for example I-6 starting from Intermediate-41 (Stereoisomer 3). LCMS: 749.3 [M+H]⁺. ¹H NMR (400 MHz, CDCl₃) δ 12.90-11.16 (m, 1H), 8.62 (s, 1H), 8.54-8.45 (m, 1H), 8.40-8.33 (m, 1H), 8.31-8.22 (m, 1H), 7.67 (s, 2H), 7.59-7.53 (m, 1H), 7.49-7.42 (m, 1H), 5.31 (dd, 2H), 4.50-4.20 (m, 1H), 3.99 (s, 4H), 3.93-3.85 (m, 1H), 3.79-3.63 (m, 1H), 3.59-3.32 (m, 1H), 2.90 (m, 1H), 2.56 (s, 3H), 2.45-2.27 (m, 2H), 2.04-1.93 (m, 2H), 1.65 (d, 3H), 1.55-1.38 (m, 2H)

### Synthesis of N-(2-fluoro-4-(trifluoromethyl)phenyl)-5-((1S,6S)-5-(5-hydroxy-6-methylpyrimidine-4-carbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxamide (Example I-16 as Stereoisomer 4)

Example I-16 (Stereoisomer 4) was synthesized by analogy to the 2-step procedure described for example I-6 starting from Intermediate-42 (Stereoisomer 4). LCMS: 749.3 [M+H]⁺. ¹H NMR (400 MHz, CDCl₃) δ 12.33-11.28 (m, 1H), 8.61 (s, 1H), 8.56-8.51 (m, 1H), 8.50-8.40 (m, 1H), 8.29 (d, 1H), 7.73-7.69 (m, 1H), 7.67-7.63 (m, 1H), 7.59-7.54 (m, 1H), 7.53-7.50 (m, 1H), 5.30 (d, 2H), 4.53-4.19 (m, 1H), 4.01 (s, 4H), 3.84-3.64 (m, 2H), 3.59-3.28 (m, 2H), 3.07-2.89 (m, 1H), 2.68-2.46 (m, 4H), 2.40 (s, 1H), 1.96-1.77 (m, 1H), 1.65 (s, 3H), 1.57-1.46 (m, 2H)

### Synthesis of (7R,9R)—N-(4-chloro-2,2-difluoro-2H-1,3-benzodioxol-5-yl)-6-[(1S,6S)-5-(5-hydroxy-6-methylpyrimidine-4-carbonyl)-2,5-diazabicyclo[4.2.0]octan-2-yl]-2,3,7-trimethyl-5-oxo-5H,7H,8H,9H-pyrazino[2,3-e]indolizine-9-carboxamide

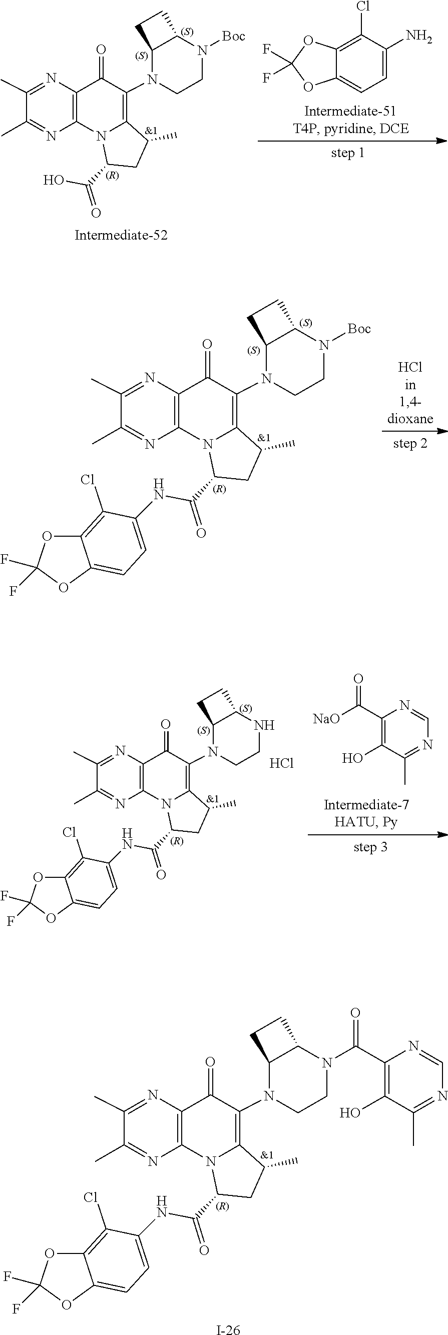

Step 1: To a solution of (7RS,9R)-6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid (Intermediate-52) (300 mg, 620 mol, 1.0 eq) and 4-chloro-2,2-difluoro-1,3-benzodioxol-5-amine trifluoroacetate (Intermediate-51) (300 mg, 1.45 mmol, 2.33 eq) in DCE (5 mL) was added pyridine (736 mg, 9.31 mmol, 15 eq) and T4P (671 mg, 1.86 mmol, 3.0 eq) and the resulting mixture was stirred at 80° C. for 0.5 h. The reaction mixture was diluted with water (10 mL) and extracted with EtOAc (20 mL×3). The combined organic layers were washed with brine (15 mL), dried over Na₂SO₄, filtered and the filtrate concentrated under reduced pressure. The residue was purified by silica gel chromatography (Eluent of EtOAc/PE) to afford tert-butyl (1S,6S)-5-((7RS,9R)-9-((4-chloro-2,2-difluorobenzo[d][1,3]dioxol-5-yl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (mixture of 2 stereoisomers). LCMS: 673.1 [M+H]⁺.

Step 2: To a solution of HCl in 1,4-dioxane (2 M, 12 mL) was added a mixture of tert-butyl (1S,6S)-5-[(7R,9R)-9-[(4-chloro-2,2-difluoro-1,3-benzodioxol-5-yl)carbamoyl]-2,3,7-trimethyl-5-oxo-8,9-dihydro-7H-pyrazino[2,3-e]indolizin-6-yl]-2,5-diazabicyclo[4.2.0]octane-2-carboxylate and tert-butyl (1S,6S)-5-((7S,9R)-9-((4-chloro-2,2-difluorobenzo[d][1,3]dioxol-5-yl) carbamoyl)-2,3,7-trimethyl-5-oxo-5,7,8,9-tetrahydropyrazino [2,3-e]indolizin-6-yl)-2,5-diazabicyclo[4.2.0]octane-2-carboxylate (400 mg, 594 mol, 1.0 eq), and it was stirred at room temperature for 0.5 h. The reaction mixture was concentrated under reduced pressure to afford (7RS,9R)-6-((1S,6S)-2,5-diazabicyclo[4.2.0]octan-2-yl)-N-(4-chloro-2,2-difluorobenzo[d][1,3]dioxol-5-yl)-2,3,7-trimethyl-5-oxo-5,7,8,9-tetrahydropyrazino[2,3-e]indolizine-9-carboxamide hydrochloride (mixture of 2 stereoisomers), which was used into the next step without further purification. LCMS: 573.1 [M+H]⁺.

Step 3: To a solution of sodium 5-hydroxy-6-methylpyrimidine-4-carboxylate (Intermediate-7) (277 mg, 1.58 mmol, 3.0 eq) in DCM (2 mL) and pyridine (2 mL) was added HATU (499 mg, 1.31 mmol, 2.5 eq) and the resulting mixture was stirred at room temperature for 0.5 h. (7RS,9R)-6-((1S,6S)-2,5-diazabicyclo[4.2.0]octan-2-yl)-N-(4-chloro-2,2-difluorobenzo[d][1,3]dioxol-5-yl)-2,3,7-trimethyl-5-oxo-5,7,8,9-tetrahydropyrazino[2,3-e]indolizine-9-carboxamide hydrochloride (mixture of 2 stereoisomers) (320 mg, 525 mol, 1.0 eq) was added, and the resulting mixture was stirred at room temperature for 1.5 h. The reaction mixture was diluted with water (10 mL) and extracted with EtOAc (20 mL×3). The combined organic layers were washed with brine (15 mL), dried over Na₂SO₄, filtered and the filtrate concentrated under reduced pressure. The residue was purified by reverse phase HPLC (column: Phenomenex luna C18 150×40 mm, 15 μm; mobile phase: H₂O (0.225% FA)/ACN; gradient: 42%-72% B over 15.0 min, retention time: peak 1: 10.5 min (undesired); peak 2: 12.5 min (desired peak) to afford (7R,9R)—N-(4-chloro-2,2-difluoro-2H-1,3-benzodioxol-5-yl)-6-[(1S,6S)-5-(5-hydroxy-6-methylpyrimidine-4-carbonyl)-2,5-diazabicyclo[4.2.0]octan-2-yl]-2,3,7-trimethyl-5-oxo-5H,7H,8H,9H-pyrazino[2,3-e]indolizine-9-carboxamide. LCMS: 709.2 [M+H]⁺. ¹H NMR (400 MHz, CDCl₃): δ 11.82 (br s, 1H), 8.61 (br s, 1H), 8.39 (br s, 1H), 7.90 (br d, 1H), 6.99 (d, 1H), 5.59 (br d, 1H), 4.54-4.02 (m, 2H), 4.02-3.77 (m, 2H), 3.77-3.57 (m, 1H), 3.55-3.42 (m, 1H), 2.96-2.79 (m, 1H), 2.71 (s, 3H), 2.64 (s, 3H), 2.55 (s, 3H), 2.44 (br d, 1H), 2.39-2.18 (m, 1H), 1.76-1.68 (m, 1H), 1.60 (br d, 3H), 1.46 (br d, 2H), 1.36-1.09 (m, 1H).

### Example 4: Synthesis of Intermediates

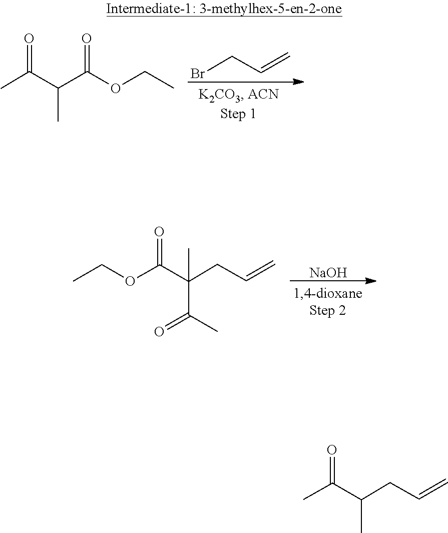

Step 1. To a solution of ethyl 2-methyl-3-oxobutanoate (100 g, 693.64 mmol, 1.0 eq) and K₂CO₃ (191.73 g, 1.39 mol, 2.0 eq) in ACN (1500 mL) was added 3-bromoprop-1-ene (92.30 g, 763.00 mmol, 1.1 eq) and the resulting mixture was stirred at 70° C. for 48 h. The reaction mixture was filtered and the filtrate was concentrated under reduced pressure. The residue was purified by silica gel chromatography (EtOAc/PE) to afford ethyl 2-acetyl-2-methylpent-4-enoate. LCMS: 185.1 [M+H]⁺.

Step 2. To a mixture of ethyl 2-acetyl-2-methylpent-4-enoate (15.60 g, 84.68 mmol, 1.0 eq) in H₂O (150 mL) and 1,4-dioxane (15 mL) was added NaOH (12.53 g, 313.30 mmol, 3.7 eq), and the resulting mixture was stirred at 105° C. overnight. The reaction mixture was extracted with DCM (100 mL*3). The combined organic layers were washed with brine (50 mL), dried over anhydrous Na₂SO₄, filtered and concentrated under reduced pressure to afford 3-methylhex-5-en-2-one, which was used into the next step without further purification. ¹H NMR (400 MHz, CDCl₃) δ 5.76-5.67 (m, 1H), 5.29 (s, 1H), 5.05-4.99 (m, 1H), 2.60-2.55 (m, 1H), 2.40-2.35 (m, 1H), 2.12 (s, 3H), 2.10-2.07 (m, 1H), 1.08 (d, 3H).

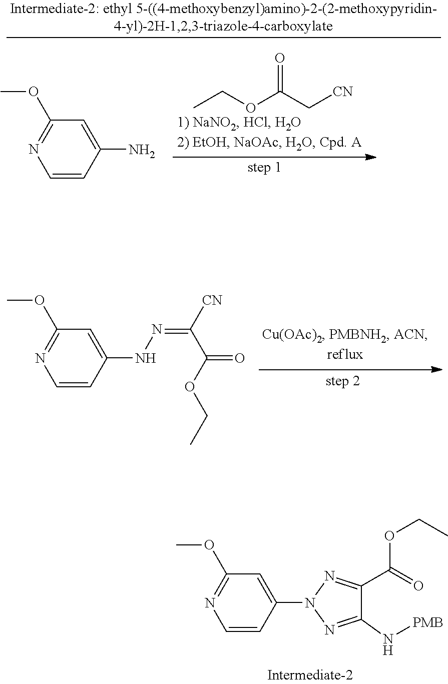

Step 1. To a HCl aqueous solution (4M, 150 mL) was added 2-methoxypyridin-4-amine (10.00 g, 80.55 mmol, 1.0 eq) at 0° C., followed by adding a solution of NaNO₂ (8.34 g, 120.83 mmol, 1.5 eq) in H₂O (100 mL) dropwise 0° C. After addition, the resulting mixture was stirred at 0° C. for 0.5 h. Then, a solution of ethyl 2-cyanoacetate (10.02 g, 88.61 mmol, 1.1 eq) and NaOAc (39.65 g, 483.32 mmol, 6.0 eq) in H₂O (150 mL) and EtOH (100 mL) was added to the above reaction mixture at 0° C. The resulting mixture was stirred at room temperature for 2 h. The reaction mixture was filtered and the filter cake was dried in vacuum to afford ethyl 2-cyano-2-[(2-methoxy-4-pyridyl)hydrazono]acetate, which was used into the next step without further purification. LCMS: 249.1 [M+H]⁺.

Step 2: To a solution of ethyl 2-cyano-2-(2-(2-methoxypyridin-4-yl)hydrazineylidene)acetate (140.00 g, 563.98 mmol, 1 eq) in ACN (1400 mL) was added (4-methoxyphenyl)methanamine (85.10 g, 620.37 mmol, 1.1 eq), the resulting mixture was stirred at 90° C. for 0.5 h. Copper acetate monohydrate (112.60 g, 563.98 mmol, 1 eq) was added, and the resulting mixture was stirred at 90° C. for overnight under O₂ balloon. The reaction solution was filtered and washed with EtOAc (500 mL*2). The filtrate was concentrated under reduced pressure to give a residue. The residue was purified by silica gel chromatography (Eluent of EtOAc/PE) to give crude product. The crude product was triturated with PE:EtOAc (10:1, 1.1 L) at room temperature for 20 min, filtered and the filter cake was dried under reduced pressure to afford ethyl 5-((4-methoxybenzyl)amino)-2-(2-methoxypyridin-4-yl)-2H-1,2,3-triazole-4-carboxylate. LCMS: 384.2 [M+H]⁺. ¹H NMR (400 MHz, DMSO-d₆) δ=8.26 (d, 1H), 7.45 (dd, 1H), 7.35 (d, 2H), 7.12 (d, 1H), 6.92-6.83 (m, 2H), 6.70 (t, 1H), 4.50-4.29 (m, 4H), 3.90 (s, 3H), 3.71 (s, 3H), 1.33 (t, 3H).

### Intermediate-4: tert-butyl (1S,6S)-5-(2-(2-methoxypyridin-4-yl)-7-oxo-5-(pent-4-en-2-yl)-4,7-dihydro-2H-[1,2,3]triazolo[4,5-b]pyridin-6-yl)-2,5-diazabicyclo[4.2.0]octane-2-carboxylate and Intermediate-5: 5-((1S,6S)-5-(tert-butoxycarbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxylic acid

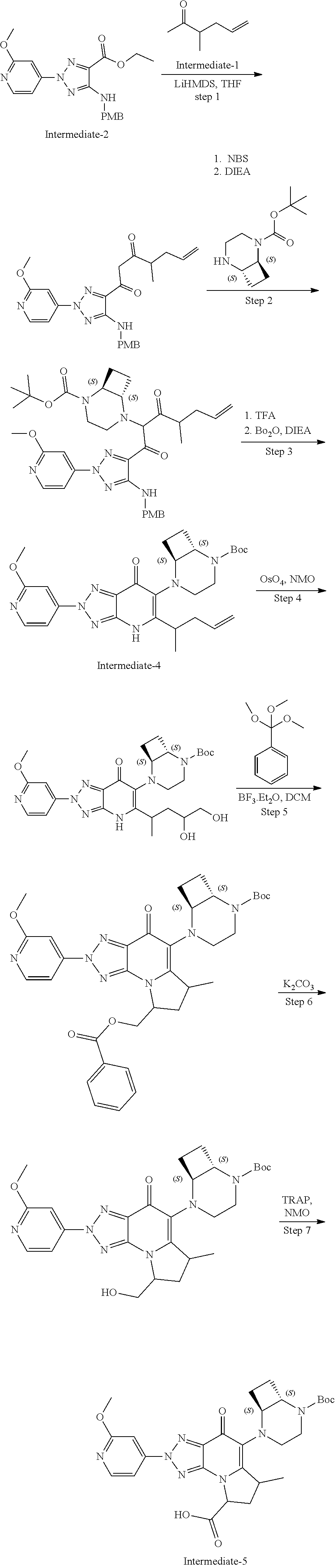

Step 1. To a solution of ethyl 5-((4-methoxybenzyl)amino)-2-(2-methoxypyridin-4-yl)-2H-1,2,3-triazole-4-carboxylate (Intermediate-2) (11.00 g, 28.69 mmol, 1.0 eq) and 3-methylhex-5-en-2-one (Intermediate-1) (25.29 g, 63.12 mmol, 2.2 eq) in toluene (200 mL) was added LiHMDS (1 M in THF, 63.12 mL, 2.2 eq) at 0° C. After addition, the resulting mixture was stirred at 60° C. for 1 h. The reaction was quenched with saturated NH₄Cl (200 mL) and extracted with EtOAc (100 mL*3). The combined organic layers were washed with brine (50 mL), dried over anhydrous Na₂SO₄, filtered and concentrated in vacuo. The residue was purified by silica gel chromatography (EtOAc/PE) to afford 1-(5-((4-methoxybenzyl)amino)-2-(2-methoxypyridin-4-yl)-2H-1,2,3-triazol-4-yl)-4-methylhept-6-ene-1,3-dione. LCMS: 450.2 [M+H]⁺.

Step 2. To a solution of 1-(5-((4-methoxybenzyl)amino)-2-(2-methoxypyridin-4-yl)-2H-1,2,3-triazol-4-yl)-4-methylhept-6-ene-1,3-dione (22.5 g, 50.06 mmol, 1.0 eq) in THF (220 mL) was added NBS (8.91 g, 50.06 mmol, 1.0 eq) at 0° C., and the resulting mixture was stirred at 0° C. for 1 h. The reaction mixture was used into the next step without work-up and purification. To the reaction mixture was added tert-butyl (1S,6S)-2,5-diazabicyclo[4.2.0]octane-2-carboxylate (15.91 g, 74.94 mmol, 1.5 eq) and DIEA (19.37 g, 149.89 mmol, 26.11 mL, 3.0 eq), the resulting mixture was stirred at 40° C. for 2 h. The reaction was diluted with water (150 mL) and extracted with EtOAc (100 mL*3). The combined organic layers were washed with brine (50 mL), dried over anhydrous Na₂SO₄, filtered and concentrated in vacuo. The residue was purified by silica gel chromatography (EtOAc/PE) to afford tert-butyl (1S,6S)-5-(1-(5-((4-methoxybenzyl)amino)-2-(2-methoxypyridin-4-yl)-2H-1,2,3-triazol-4-yl)-4-methyl-1,3-dioxohept-6-en-2-yl)-2,5-diazabicyclo[4.2.0]octane-2-carboxylate. LCMS: 660.5 [M+H]⁺.

Step 3. A mixture of tert-butyl (1S,6S)-5-(1-(5-((4-methoxybenzyl)amino)-2-(2-methoxypyridin-4-yl)-2H-1,2,3-triazol-4-yl)-4-methyl-1,3-dioxohept-6-en-2-yl)-2,5-diazabicyclo[4.2.0]octane-2-carboxylate (19 g, 28.80 mmol, 1 eq) in TFA (200 mL) was stirred at 70° C. for 2 h and then concentrated in vacuo. The residue was dissolved into DCM (150 mL), then DIEA (29.68 g, 229.66 mmol, 8.0 eq) and (Boc)₂O (6.27 g, 28.71 mmol, 1.0 eq) was added. The resulting mixture was stirred at room temperature for 1 h. The reaction was diluted with water (150 mL) and extracted with DCM (100 mL*3). The combined organic layers were washed with brine (50 mL), dried over anhydrous Na₂SO₄, filtered and concentrated in vacuo. The residue was purified by silica gel chromatography (EtOAc/PE) to afford tert-butyl (1S,6S)-5-(2-(2-methoxypyridin-4-yl)-7-oxo-5-(pent-4-en-2-yl)-4,7-dihydro-2H-[1,2,3]triazolo[4,5-b]pyridin-6-yl)-2,5-diazabicyclo[4.2.0]octane-2-carboxylate. LCMS: 522.4 [M+H]⁺.

Step 4. To a solution of tert-butyl (1S,6S)-5-(2-(2-methoxypyridin-4-yl)-7-oxo-5-(pent-4-en-2-yl)-4,7-dihydro-2H-[1,2,3]triazolo[4,5-b]pyridin-6-yl)-2,5-diazabicyclo[4.2.0]octane-2-carboxylate (Intermediate-4) (14.90 g, 28.57 mmol, 1.0 eq) and NMO (3.68 g, 31.42 mmol, 1.1 eq) in a mixed solvent of THF (150 mL), t-BuOH (30 mL) and H₂O (30 mL) was added OsO₄ (726.22 mg, 2.86 mmol, 0.1 eq). The resulting mixture was stirred at room temperature for 4 h. The reaction mixture was quenched with 10% Na₂SO₃ (200 mL) and extracted with DCM (100 mL*2). The combined organic layers were washed with brine, dried over anhydrous Na₂SO₄, filtered and concentrated under reduced pressure. The residue was purified by silica gel chromatography (EtOAc/PE) to afford tert-butyl (1S,6S)-5-(5-(4,5-dihydroxypentan-2-yl)-2-(2-methoxypyridin-4-yl)-7-oxo-4,7-dihydro-2H-[1,2,3]triazolo[4,5-b]pyridin-6-yl)-2,5-diazabicyclo[4.2.0]octane-2-carboxylate. LCMS: 556.3 [M+H]⁺.

Step 5. To a solution of tert-butyl (1S,6S)-5-(5-(4,5-dihydroxypentan-2-yl)-2-(2-methoxypyridin-4-yl)-7-oxo-4,7-dihydro-2H-[1,2,3]triazolo[4,5-b]pyridin-6-yl)-2,5-diazabicyclo[4.2.0]octane-2-carboxylate (500 mg, 899.89 mol, 1.0 eq) and (trimethoxymethyl)benzene (246 mg, 1.35 mmol, 1.5 eq) in DCM (5 mL) was added BF₃·Et₂O (13 mg, 90 mol, 0.1 eq), and the resulting mixture was stirred at room temperature for 1 h. The reaction mixture was quenched by saturated NaHCO₃ (20 mL), and then extracted with DCM (20 mL*3). The combined organic layers were washed with brine (20 mL), dried over anhydrous Na₂SO₄, filtered and concentrated under reduced pressure. The residue was purified by silica gel chromatography (EtOAc/PE) to afford tert-butyl (1S,6S)-5-(8-((benzoyloxy)methyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate. LCMS: 642.3 [M+H]⁺.

Step 6. To a solution of tert-butyl (1S,6S)-5-(8-((benzoyloxy)methyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (500 mg, 779 mol, 1.0 eq) in MeOH (1 mL), THF (2 mL) and H₂O (1 mL) was added K₂CO₃ (323.05 mg, 2.34 mmol, 3.0 eq), and the resulting mixture was stirred at 60° C. for 1 h. The reaction was diluted with water (20 mL) and extracted with EtOAc (30 mL*3). The combined organic layers were washed with brine (20 mL), dried over anhydrous Na₂SO₄, filtered and concentrated in vacuo to afford tert-butyl (1S,6S)-5-(8-(hydroxymethyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate, which was used into the next step without further purification. LCMS: 538.2 [M+H]⁺.

Step 7. To a mixture of tert-butyl (1S,6S)-5-(8-(hydroxymethyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (300 mg, 558 mol, 1.0 eq), NMO (131 mg, 1.12 mmol, 2.0 eq) and 4 Å molecular sieves (100 mg) in DCM (1.5 mL) and MeCN (1.5 mL) was added a solution of TPAP (196 mg, 558.03 mol, 1.0 eq) in DCM (0.25 mL) and MeCN (0.25 mL) at 0° C. After addition, the resulting mixture was stirred at room temperature for 20 min. The reaction mixture was adjusted to pH 3-4 with aqueous HCl solution (1 N), and then diluted with water (20 mL) and extracted with EtOAc (20 mL*3). The combined organic layers were washed with brine (10 mL), dried over anhydrous Na₂SO₄, filtered and concentrated in vacuo. The residue was purified by reverse phase HPLC (C18 column, water (0.1% FA)-ACN) to afford 5-((1S,6S)-5-(tert-butoxycarbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxylic acid. LCMS: 552.4 [M+H]⁺.

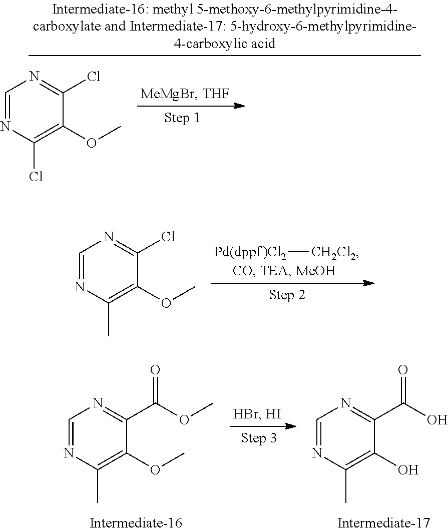

Step 1: To a mixture of 4,6-dichloro-5-methoxypyrimidine (30.00 g, 167.6 mmol, 1.0 eq) in THF (300 mL) was added a 3 M solution of MeMgBr (61.45 mL, 184.4 mmol, 1.1 eq) in diethyl ether dropwise at 0° C. and then the mixture was stirred at 5° C. for 1 h. The resulting mixture was poured into H₂O (200 mL) and extracted with EtOAc (100 mL*3). The combined organic phase was washed with brine (100 mL), dried over anhydrous Na₂SO₄, filtered and the filtrate was concentrated in vacuum. The residue was purified by flash silica gel chromatography (eluent of EtOAc/PE) to afford 4-chloro-5-methoxy-6-methylpyrimidine. LCMS: 159.1 [M+H]⁺.

Step 2: To a mixture of 4-chloro-5-methoxy-6-methylpyrimidine (22.00 g, 138.7 mmol, 1.0 eq) in MeOH (250 mL) was added Pd(dppf)Cl₂—CH₂Cl₂ (6.80 g, 8.32 mmol, 0.06 eq) and TEA (28.1 g, 278 mmol, 2.0 eq). The reaction was purged with CO (50 psi) and stirred at 50° C. overnight. The resulting mixture was filtered, and the filtrate was concentrated under reduced pressure. The residue was purified by flash silica gel chromatography (eluent of EtOAc/PE) to afford methyl 5-methoxy-6-methylpyrimidine-4-carboxylate. LCMS: 183.1 [M+H]⁺.

Step 3: A mixture of methyl 5-methoxy-6-methylpyrimidine-4-carboxylate (Intermediate-16) (16.00 g, 87.83 mmol, 1.0 eq) in HBr solution (aq.) (68.5 mL, 48 wt %) was stirred at 50° C. overnight. Then HI solution (aq.) (67.2 mL, 56 wt %) was added and stirred at 50° C. for 6 h. The reaction mixture was cooled to room temperature and basified with 50% NaOH solution (aq.) to pH 9 at 0° C., then adjusted to pH 7 with 2 M HCl solution (aq.) at 0° C. The mixture was filtered, the filter cake was dried in vacuum to afford 5-hydroxy-6-methylpyrimidine-4-carboxylic acid, which was used in the next step without further purification. LCMS: 155.1 [M+H]⁺. ¹H NMR (400 MHz, DMSO-d₆) δ ppm 15.46 (br s, 1H), 8.37 (s, 1H), 2.34 (s, 3H).

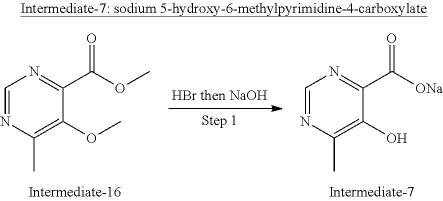

Step 1. To a mixture of methyl 5-methoxy-6-methyl-pyrimidine-4-carboxylate (Intermediate-16) (210 g, 1.15 mol, 1 eq) was added aqueous HBr solution (850 mL, 48 wt %) at 20° C. After addition, the mixture was stirred at 50° C. for 16 hr, and then aqueous HI solution (600 mL, 56 wt %) was added at 50° C. The resulting mixture was stirred at 50° C. for 6 hr. The reaction mixture was filtered. The filtrate was adjusted to pH 8-9 with aqueous NaOH solution (30% in water) at 0˜5° C. The mixture was filtered, the filter cake was dried under reduced pressure to afford sodium 5-hydroxy-6-methylpyrimidine-4-carboxylate, which was used into next step directly without further purification. ¹H NMR (400 MHz, CD₃OD) δ ppm 8.47 (s, 1H), 2.48 (s, 3H).

### Intermediate-8: tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 1); Intermediate-9: tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 2); Intermediate-10: tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 3); Intermediate-11: tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 4)

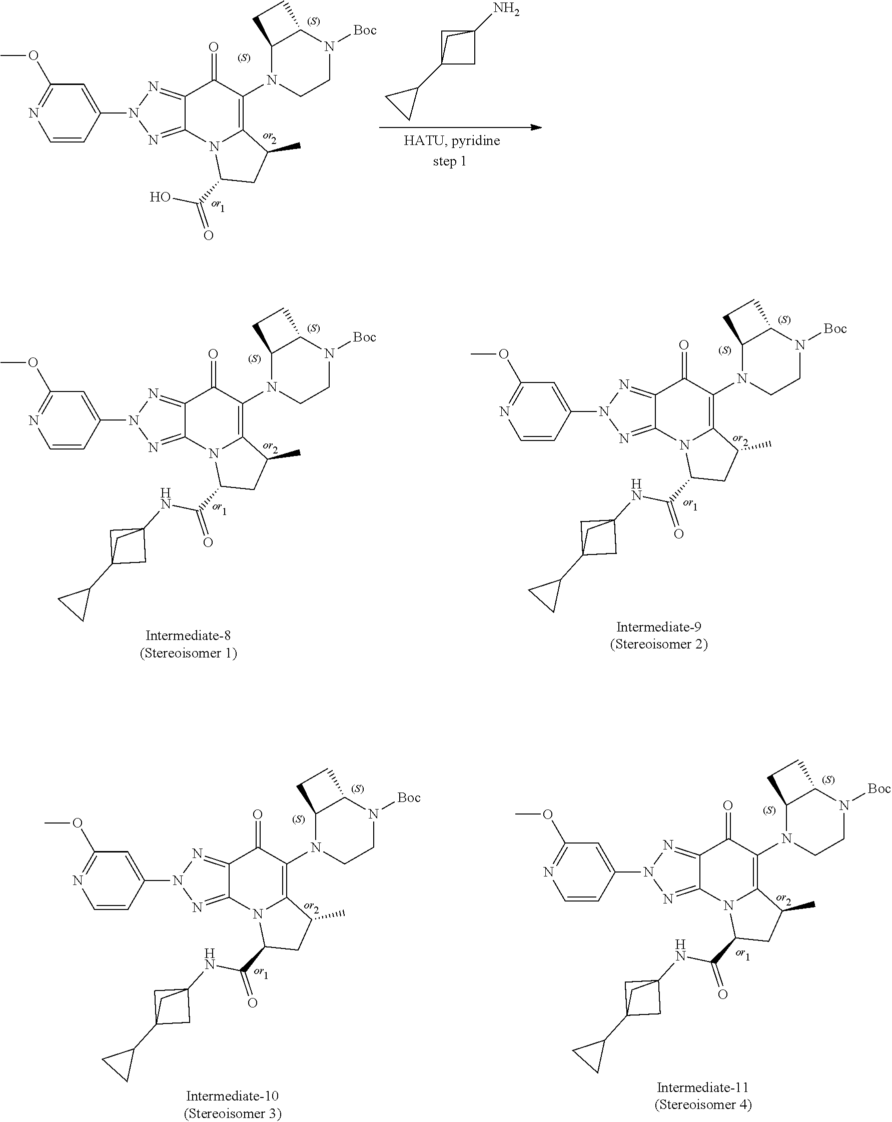

Step 11. To a solution of 5-((1S,6S)-5-(tert-butoxycarbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxylic acid (Intermediate-5) (580 mg, 1.05 mmol, 1.0 eq) and 3-cyclopropylbicyclo[1.1.1]pentan-1-amine (130 mg, 1.05 mmol, 1.0 eq) in pyridine (5 mL) was added HATU (400 mg, 1.05 mmol, 1.0 eq), the resulting mixture was stirred at room temperature for 0.5 h. The reaction mixture was diluted with water (30 mL) and extracted with EtOAc (30 mL*3). The combined organic layers were washed with brine (15 mL), dried over anhydrous Na₂SO₄, filtered and concentrated in vacuo. The residue was purified by silica gel chromatography (EtOAc/PE) to afford three fractions of tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate. The three fractions were used in the following steps separately. Fraction 1: Intermediate 8 (Stereoisomer 1)+Intermediate 9 (Stereoisomer 2); Fraction 2: Intermediate 10 (Stereoisomer 3); Fraction 3: Intermediate 11 (Stereoisomer 4). LCMS: 657.4 [M+H]⁺.

### Intermediate-12: 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Stereoisomer 1) and Intermediate-13: 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Stereoisomer 2)

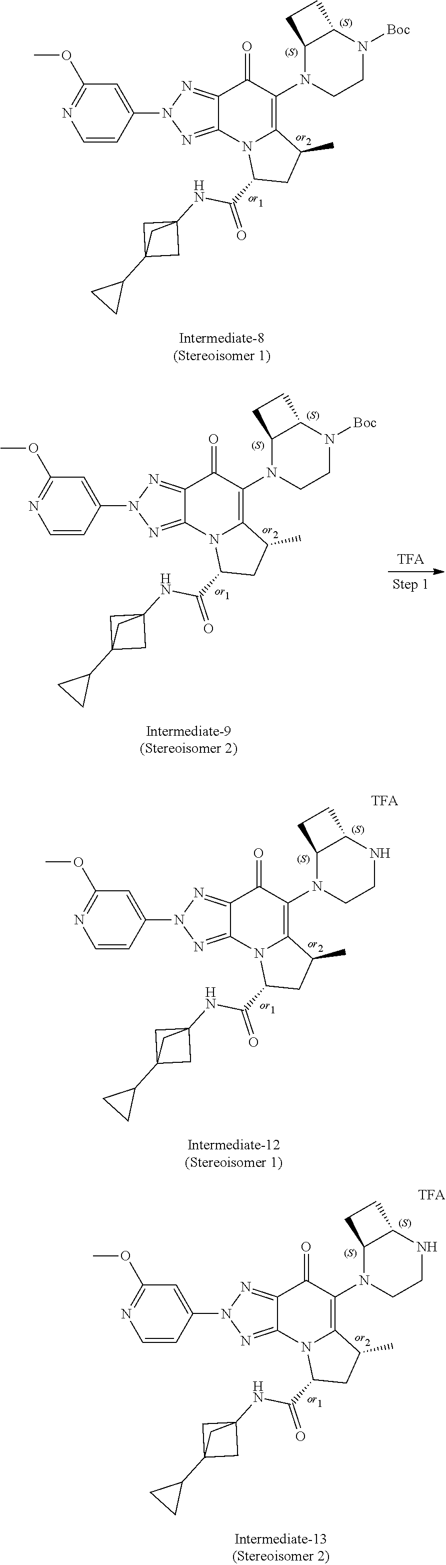

Step 1. To a solution of tert-butyl (1S,6S)-5-(8-((3-cyclopropylbicyclo[1.1.1]pentan-1-yl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Fraction 1: Mixture of Intermediate-8 (Stereoisomer 1)+Intermediate-9 (Stereoisomer 2), 120 mg, 183 mol, 1.0 eq) in DCM (1 mL) was added TFA (1 mL), the resulting mixture was stirred at room temperature for 1 h. The reaction mixture was concentrated in vacuo to afford 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Intermediate 12, Stereoisomer 1)+5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Intermediate 13, Stereoisomer 2)), which was used into the next step without further purification. LCMS: 557.4 [M+H]⁺.

Intermediate-14: 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Stereoisomer 3)

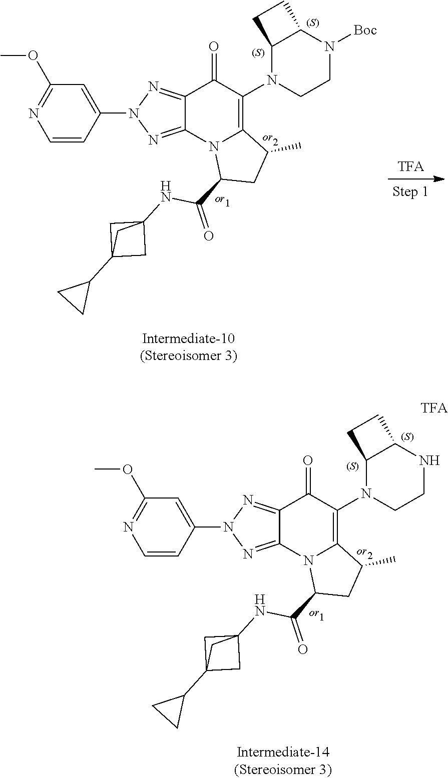

Intermediate-14 (Stereoisomer 3) was prepared by analogy to the procedure described for Intermediate-12 and Intermediate-13 using Intermediate-10 (Stereoisomer 3) as starting material in step 1. LCMS: 557.4 [M+H]⁺.

### Intermediate-15: 5-((1S,6S)-2,5-diazabicyclo[4.2.0]octan-2-yl)-N-(3-cyclopropylbicyclo[1.1.1]pentan-1-yl)-2-(2-methoxypyridin-4-yl)-6-methyl-4-oxo-4,6,7,8-tetrahydro-2H-[1,2,3]triazolo[4,5-e]indolizine-8-carboxamide trifluoroacetate (Stereoisomer 4)

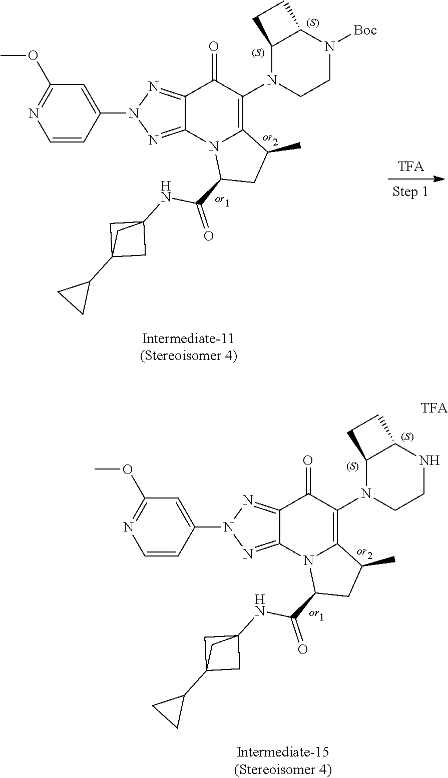

Intermediate-15 (stereoisomer 4) was prepared by analogy to the procedure described for Intermediate-12 and Intermediate-13 using Intermediate-11 (Stereoisomer 4) as starting material in step 1. LCMS: 557.4 [M+H]⁺.

### Intermediate-18: 5-hydroxy-6-methylpyrimidine-4-carbonylchloride

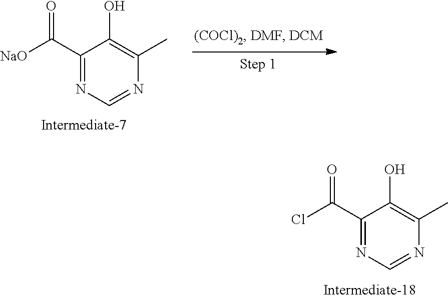

Step 1. To a solution of sodium 5-hydroxy-6-methylpyrimidine-4-carboxylate (Intermediate-7) (90 mg, 584 mol, 1.0 eq) in DCM (1 mL) was added oxalyl dichloride (148 mg, 1.17 mmol, 2.0 eq) and one drop of DMF. The resulting mixture was stirred at room temperature for 30 min and then concentrated in vacuo to afford 5-hydroxy-6-methylpyrimidine-4-carbonyl chloride, which was used into the next step without further purification.

### Intermediate-28: tert-butyl (1S,6S)-5-(5-(4-methoxybenzyl)-2-methyl-8-oxo-6-(pent-4-en-2-yl)-5,8-dihydropyrido[2,3-b]pyrazin-7-yl)-2,5-diazabicyclo[4.2.0]octane-2-carboxylate

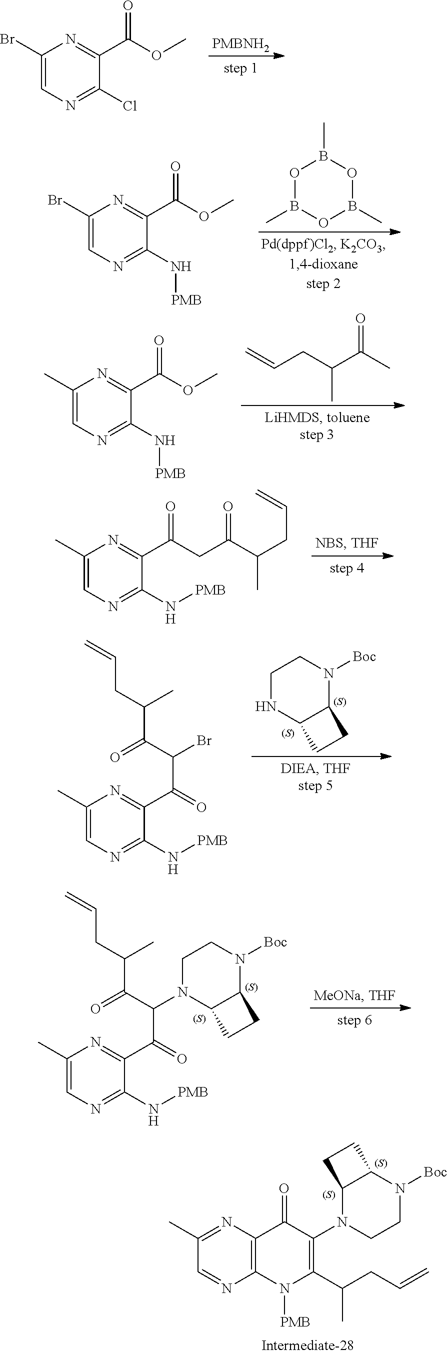

Step 1: To a solution of methyl 6-bromo-3-chloropyrazine-2-carboxylate (52.00 g, 206.79 mmol, 1.0 eq) in 1,4-dioxane (500 mL) was added DIEA (40.09 g, 310.18 mmol, 1.5 eq) and bis(4-methoxybenzyl)amine (31.20 g, 227.47 mmol, 1.1 eq), the resulting mixture was stirred at 100° C. overnight. The reaction mixture was concentrated under reduced pressure to remove solvent. The residue was diluted with H₂O (1500 mL) and extracted with EtOAc (1000 mL*2). The combined organic layers were washed with brine (1000 mL), dried over anhydrous Na₂SO₄, filtered and concentrated under reduced pressure. The crude product was triturated with (PE:EtOAc=10:1, 440 mL) to afford methyl 6-bromo-3-((4-methoxybenzyl)amino)pyrazine-2-carboxylate. LCMS: 352.0 [M+H]⁺. ¹H NMR (400 MHz, CDCl₃) δ ppm 8.35 (s, 1H), 8.31 (br s, 1H), 7.29 (d, 2H), 6.90 (d, 2H), 4.65 (d, 2H), 3.97 (s, 3H), 3.82 (s, 3H).

Step 2. To a solution of methyl 6-bromo-3-((4-methoxybenzyl)amino)pyrazine-2-carboxylate (10.00 g, 28.39 mmol, 1.0 eq) and 2,4,6-trimethyl-1,3,5,2,4,6-trioxatriborinane (14.26 g, 56.79 mmol, 2.0 eq) in 1,4-dioxane (150 mL) was added Pd(dppf)Cl₂ (2.08 g, 2.84 mmol, 0.1 eq) and K₂CO₃ (11.77 g, 85.18 mmol, 3.0 eq). The resulting mixture was stirred under N₂ atmosphere at 110° C. overnight. The reaction mixture was diluted with H₂O (100 mL) and extracted with EtOAc (100 mL*3). The combined organic layers were washed with brine (100 mL), dried over anhydrous Na₂SO₄, filtered and the filtrate was concentrated under reduced pressure. The residue was purified by silica gel chromatography (EtOAc/PE) to afford methyl 3-((4-methoxybenzyl)amino)-6-methylpyrazine-2-carboxylate. LCMS: 288.2 [M+H]⁺.

Step 3. To a solution of methyl 3-((4-methoxybenzyl)amino)-6-methylpyrazine-2-carboxylate (2.50 g, 8.70 mmol, 1.0 eq) and 3-methylhex-5-en-2-one (8.26 g, 19.14 mmol, 2.2 eq) in toluene (60 mL) was added LiHMDS (1 M in THF, 19.14 mL, 2.2 eq) at 0° C., and the resulting mixture was stirred at 60° C. for 0.5 h. The reaction mixture was quenched with saturated NH₄Cl (50 mL) and extracted with EtOAc (50 mL*3). The combined organic layers were washed with brine (30 mL), dried over anhydrous Na₂SO₄, filtered and the filtrate was concentrated under reduced pressure. The residue was purified by silica gel chromatography (EtOAc/PE) to afford 1-(3-((4-methoxybenzyl)amino)-6-methylpyrazin-2-yl)-4-methylhept-6-ene-1,3-dione. LCMS: 368.2 [M+H]⁺.

Step 4. To a solution of 1-(3-((4-methoxybenzyl)amino)-6-methylpyrazin-2-yl)-4-methylhept-6-ene-1,3-dione (3.50 g, 9.53 mmol, 1.0 eq) in THF (30 mL) was added NBS (1.70 g, 9.53 mmol, 1.0 eq), and the resulting mixture was stirred at 0° C. for 0.5 h. The reaction mixture comprising 2-bromo-1-(3-((4-methoxybenzyl)amino)-6-methylpyrazin-2-yl)-4-methylhept-6-ene-1,3-dione was used into the next step directly without further workup or purification. LCMS: 446.1 [M+H]⁺.

Step 5. To the reaction mixture of step 3 was added DIEA (3.65 g, 28.59 mmol, 3.0 eq) and tert-butyl (1S,6S)-2,5-diazabicyclo[4.2.0]octane-2-carboxylate (4.05 g, 19.06 mmol, 2.0 eq), and the resulting mixture was stirred at room temperature overnight. The reaction mixture was diluted with H₂O (50 mL) and extracted with EtOAc (50 mL*3). The combined organic layers were washed with brine (30 mL), dried over anhydrous Na₂SO₄, filtered and the filtrate was concentrated under reduced pressure. The residue was purified by silica gel chromatography (EtOAc/PE) to afford tert-butyl (1S,6S)-5-(1-(3-((4-methoxybenzyl)amino)-6-methylpyrazin-2-yl)-4-methyl-1,3-dioxohept-6-en-2-yl)-2,5-diazabicyclo[4.2.0]octane-2-carboxylate. LCMS: 578.5 [M+H]⁺.

Step 6. To a solution of tert-butyl (1S,6S)-5-(1-(3-((4-methoxybenzyl)amino)-6-methylpyrazin-2-yl)-4-methyl-1,3-dioxohept-6-en-2-yl)-2,5-diazabicyclo[4.2.0]octane-2-carboxylate (3.50 g, 6.06 mmol, 1.0 eq) in THF (30 mL) was added NaOMe (6.55 g, 121.17 mmol, 20.0 eq), and the resulting mixture was stirred at 35° C. for 1 h. The reaction mixture was diluted with H₂O (50 mL) and extracted with EtOAc (50 mL*3). The combined organic layers were washed with brine (30 mL), dried over anhydrous Na₂SO₄, filtered and the filtrate was concentrated under reduced pressure. The residue was purified by silica gel chromatography (EtOAc/PE) to afford tert-butyl (1S,6S)-5-(5-(4-methoxybenzyl)-2-methyl-8-oxo-6-(pent-4-en-2-yl)-5,8-dihydropyrido[2,3-b]pyrazin-7-yl)-2,5-diazabicyclo[4.2.0]octane-2-carboxylate. LCMS: 560.3 [M+H]⁺.

### Intermediate-19: 6-((1S,6S)-5-(tert-butoxycarbonyl)-2,5-diazabicyclo[4.2.0]octan-2-yl)-3,7-dimethyl-5-oxo-5,7,8,9-tetrahydropyrazino[2,3-e]indolizine-9-carboxylic acid

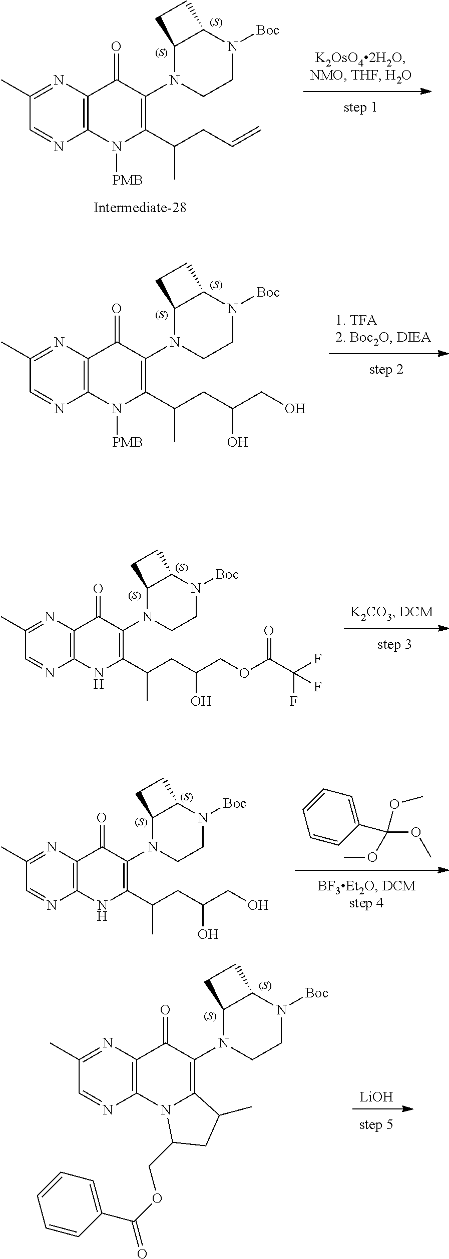

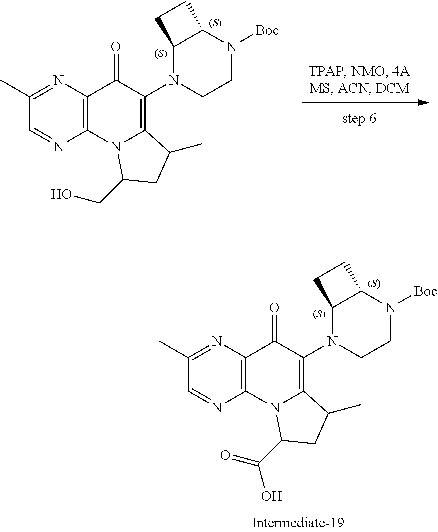

Step 1. To a solution of tert-butyl (1S,6S)-5-(5-(4-methoxybenzyl)-2-methyl)-8-oxo-6-(pent-4-en-2-yl)-5,8-dihydropyrido[2,3-b]pyrazin-7-yl)-2,5-diazabicyclo[4.2.0]octane-2-carboxylate (Intermediate-28) (2.90 g, 5.18 mmol, 1.0 eq) in a mixed solvent of THF (30 mL) and H₂O (6 mL) was added NMO (1.52 g, 12.95 mmol, 2.5 eq) and K₂OsO₄·2H₂O (191 mg, 518 mol, 0.1 eq), and the resulting mixture was stirred at room temperature overnight. The reaction mixture was quenched with saturated Na₂SO₃ (100 mL) and extracted with EtOAc (70 mL*3). The combined organic layers were washed with brine, dried over anhydrous Na₂SO₄, filtered and concentrated under reduced pressure. The residue was purified by silica gel chromatography (EtOAc/PE) to afford tert-butyl (1S,6S)-5-(6-(4,5-dihydroxypentan-2-yl)-5-(4-methoxybenzyl)-2-methyl-8-oxo-5,8-dihydropyrido[2,3-b]pyrazin-7-yl)-2,5-diazabicyclo[4.2.0]octane-2-carboxylate. LCMS: 594.5 [M+H]⁺.

Step 2. A solution of tert-butyl (1S,6S)-5-(6-(4,5-dihydroxypentan-2-yl)-5-(4-methoxybenzyl)-2-methyl-8-oxo-5,8-dihydropyrido[2,3-b]pyrazin-7-yl)-2,5-diazabicyclo[4.2.0]octane-2-carboxylate (2.4 g, 4.04 mmol, 1 eq) in TFA (30 mL) was stirred at 60° C. for 2 h and then it was concentrated in vacuo. The residue was dissolved into DCM (30 mL) and adjusted to pH 8 with DIEA, then Boc₂O (970 mg, 4.45 mmol, 1.1 eq) was added. The resulting mixture was stirred at room temperature for 1 h. The reaction mixture was diluted with H₂O (30 mL) and extracted with EtOAc (30 mL*3). The combined organic layers were washed with brine (20 mL), dried over anhydrous Na₂SO₄, filtered and the filtrate was concentrated under reduced pressure to afford tert-butyl (1S,6S)—S-(6-(4-hydroxy-5-(2,2,2-trifluoroacetoxy)pentan-2-yl)-2-methyl-8-oxo-5,8-dihydropyrido[2,3-b]pyrazin-7-yl)-2,5-diazabicyclo[4.2.0]octane-2-carboxylate, which was used into the next step without further purification. LCMS: 570.4 [M+H]⁺.

Step 3. Synthesis of tert-butyl (1S,6S)-5-(6-(4,5-dihydroxypentan-2-yl)-2-methyl-8-oxo-5,8-dihydropyrido[2,3-b]pyrazin-7-yl)-2,5-diazabicyclo[4.2.0]octane-2-carboxylate To a solution of tert-butyl (1S,6S)-5-(6-(4-hydroxy-5-(2,2,2-trifluoroacetoxy)pentan-2-yl)-2-methyl-8-oxo-5,8-dihydropyrido[2,3-b]pyrazin-7-yl)-2,5-diazabicyclo[4.2.0]octane-2-carboxylate (2.5 g, 3.76 mmol, 1.0 eq) in DCM (50 mL) was added saturated K₂CO₃ aqueous solution (10 mL), the mixture was stirred at room temperature for 10 min. The reaction mixture was diluted with H₂O (20 mL) and extracted with DCM (30 mL*5). The combined organic layers were washed with brine (20 mL), dried over anhydrous Na₂SO₄, filtered and the filtrate was concentrated under reduced pressure. The residue was purified by silica gel chromatography (EtOAc/PE) to afford the title compound. LCMS: 474.4 [M+H]⁺.

Step 4. To a solution of tert-butyl (1S,6S)-5-(6-(4,5-dihydroxypentan-2-yl)-2-methyl-8-oxo-5,8-dihydropyrido[2,3-b]pyrazin-7-yl)-2,5-diazabicyclo[4.2.0]octane-2-carboxylate (2.00 g, 4.22 mmol, 1.0 eq) in DCM (18 mL) was added BF₃·Et₂O (60 mg, 422 mol, 0.1 eq) and (trimethoxymethyl)benzene (1.15 g, 6.33 mmol, 1.5 eq), and the resulting mixture was stirred at room temperature overnight. The reaction mixture was quenched by saturated NaHCO₃ (30 mL), and then extracted with DCM (30 mL*3). The combined organic layers were washed with brine (20 mL), dried over anhydrous Na₂SO₄, filtered and concentrated under reduced pressure. The residue was purified by silica gel chromatography (EtOAc/PE) to afford tert-butyl (1S,6S)-5-(9-((benzoyloxy)methyl)-3,7-dimethyl-5-oxo-5,7,8,9-tetrahydropyrazino[2,3-e]indolizin-6-yl)-2,5-diazabicyclo[4.2.0]octane-2-carboxylate. LCMS: 560.5 [M+H]⁺.

Step 5. To a solution of tert-butyl (1S,6S)-5-(9-((benzoyloxy)methyl)-3,7-dimethyl-5-oxo-5,7,8,9-tetrahydropyrazino[2,3-e]indolizin-6-yl)-2,5-diazabicyclo[4.2.0]octane-2-carboxylate (1.60 g, 2.86 mmol, 1.0 eq) in a mixed solvent of THF (32 mL) and MeOH (16 mL) was added a solution of LiOH·H₂O (180 mg, 4.29 mmol, 1.5 eq) in H₂O (16 mL), and the resulting mixture was stirred for 10 min. The reaction was diluted with water (30 mL) and extracted with EtOAc (30 mL*3). The combined organic layers were washed with brine (30 mL), dried over anhydrous Na₂SO₄, filtered and concentrated in vacuo to afford tert-butyl (1S,6S)-5-(9-(hydroxymethyl)-3,7-dimethyl-5-oxo-5,7,8,9-tetrahydropyrazino[2,3-e]indolizin-6-yl)-2,5-diazabicyclo[4.2.0]octane-2-carboxylate, which was used into the next step without further purification. LCMS: 456.3 [M+H]⁺.

Step 6. To a solution of tert-butyl (1S,6S)-5-(9-(hydroxymethyl)-3,7-dimethyl-5-oxo-5,7,8,9-tetrahydropyrazino[2,3-e]indolizin-6-yl)-2,5-diazabicyclo[4.2.0]octane-2-carboxylate (1.55 g, 3.40 mmol, 1.0 eq), NMO (797 mg, 6.80 mmol, 2.0 eq) and 4 A molecular sieve (400 mg) in DCM (20 mL) and ACN (20 mL) was added a solution of TPAP (1.79 g, 5.10 mmol, 1.5 eq) in DCM (4 mL) and ACN (4 mL). After addition, the resulting mixture was stirred at room temperature for 10 min. The reaction mixture was adjusted to pH 3-4 with aqueous HCl solution (1 N), and then diluted with water (30 mL) and extracted with EtOAc (30 mL*5). The combined organic layers were washed with brine (10 mL), dried over anhydrous Na₂SO₄, filtered and concentrated in vacuo. The residue was purified by silica gel chromatography (EtOAc/PE) to afford 6-((1S,6S)-5-(tert-butoxycarbonyl)-2,5-diazabicyclo[4.2.0]octan-2-yl)-3,7-dimethyl-5-oxo-5,7,8,9-tetrahydropyrazino[2,3-e]indolizine-9-carboxylic acid. LCMS: 470.2 [M+H]⁺.

### Intermediate-20: tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Stereoisomer 1); Intermediate-21: tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Stereoisomer 2); Intermediate-22: tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Stereoisomer 3); Intermediate-23: tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Stereoisomer 4)

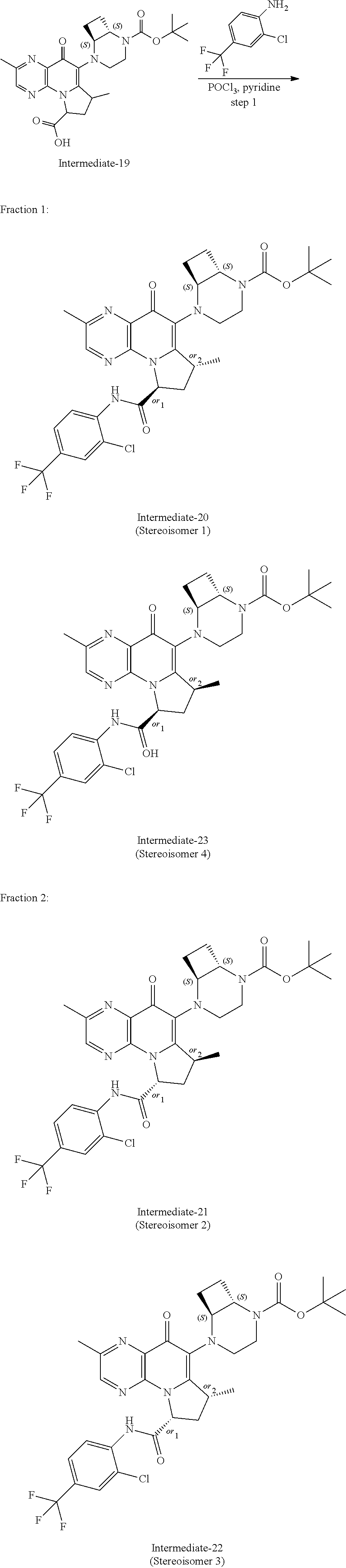

Step 1. To a solution of 6-((1S,6S)-5-(tert-butoxycarbonyl)-2,5-diazabicyclo[4.2.0]octan-2-yl)-3,7-dimethyl-5-oxo-5,7,8,9-tetrahydropyrazino[2,3-e]indolizine-9-carboxylic acid (Intermediate-19) (500 mg, 1.06 mmol, 1.0 eq) and 2-chloro-4-(trifluoromethyl)aniline (521 mg, 2.66 mmol, 2.5 eq) in DCM (7 mL) and pyridine (7 mL) was added POCl₃ (245 mg, 1.60 mmol, 1.5 eq) at 0° C., and the resulting mixture was stirred at 0° C. for 1 h. The reaction mixture was diluted with water (30 mL) and extracted with EtOAc (30 mL*3). The combined organic layers were washed with brine (15 mL), dried over anhydrous Na₂SO₄, filtered and concentrated in vacuo. The residue was purified by silica gel chromatography (EtOAc/PE) to afford two fractions of tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate. The two fractions were used in the following steps separately. Fraction 1: Mixture of Intermediate-20 (Stereoisomer 1) and Intermediate-23 (Stereoisomer 4): LCMS: 647.2 [M+H]⁺. Fraction 2: Mixture of Intermediate-21 (Stereoisomer 2) and Intermediate-22 (Stereoisomer 3): LCMS: 647.2 [M+H]⁺.

### Intermediate-24: 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (Stereoisomer 1) and Intermediate-25: 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (Stereoisomer 4)

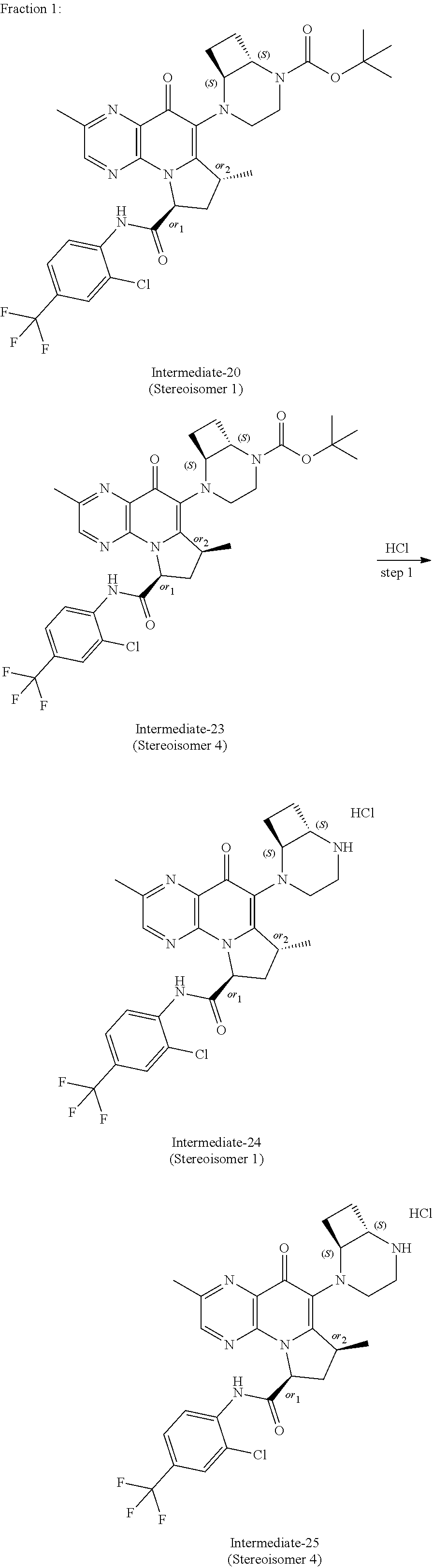

Step 13. A solution of tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Fraction 1, mixture of Intermediate-20 (Stereoisomer 1) and Intermediate-23 (Stereoisomer 4)) (150 mg, 232 mol, 1 eq) in HCl/1,4-dioxane (2 M, 4 mL) was stirred at room temperature for 30 min. The reaction mixture was concentrated under reduced pressure to afford 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (mixture of stereoisomer 1 and stereoisomer 4), which was used into the next step without further purification. LCMS: 547.1 [M+H]⁺.

### Intermediate-26: 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (Stereoisomer 2); and Intermediate-27: 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (Stereoisomer 3)

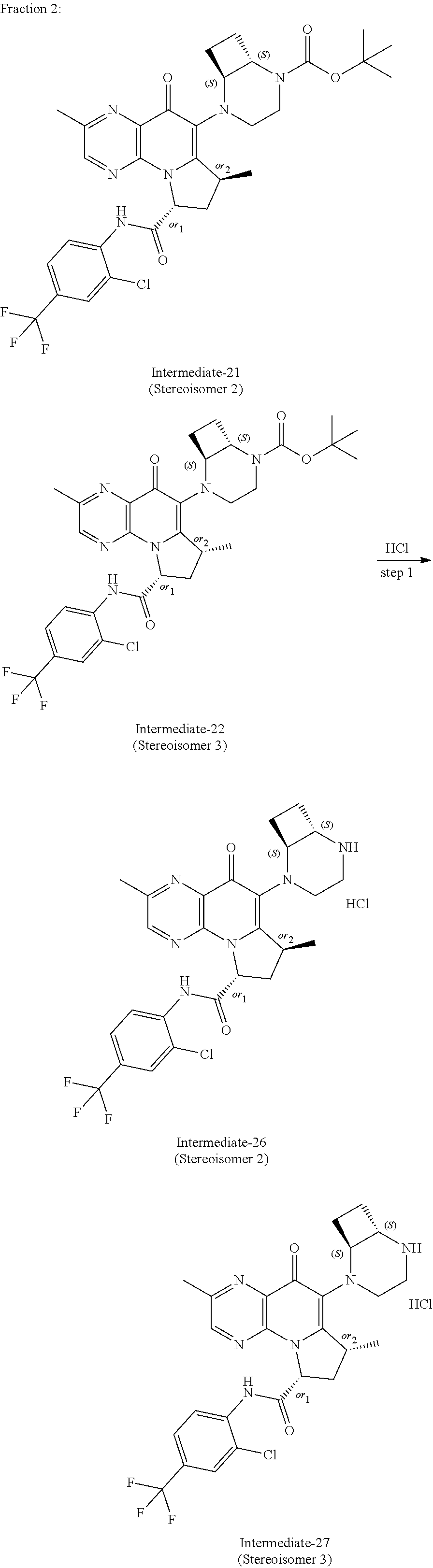

Step 1. A solution of tert-butyl (1S,6S)-5-(9-((2-chloro-4-(trifluoromethyl)phenyl)carbamoyl)-3,7-dimethyl-5-oxo-5,7,8,9-tetrahydropyrazino[2,3-e]indolizin-6-yl)-2,5-diazabicyclo[4.2.0]octane-2-carboxylate (Fraction 2: Mixture of Intermediate-21 (Stereoisomer 2) and Intermediate-22 (Stereoisomer 3)) (150 mg, 232 mol, 1 eq) in HCl/1,4-dioxane (2 M, 4 mL) was stirred at room temperature for 30 min. The reaction mixture was concentrated under reduced pressure to afford 6-((1S,6S)-2,5-diazabicyclo[4.2.0]octan-2-yl)-N-(2-chloro-4-(trifluoromethyl)phenyl)-3,7-dimethyl-5-oxo-5,7,8,9-tetrahydropyrazino[2,3-e]indolizine-9-carboxamide hydrochloride (mixture of stereoisomer 2 and stereoisomer 3), which was used into the next step without further purification. LCMS: 547.1 [M+H]⁺.

### Intermediate-29: tert-butyl (1S,6S)-5-(2,3-dimethyl-8-oxo-6-(pent-4-en-2-yl)-5,8-dihydropyrido[2,3-b]pyrazin-7-yl)-2,5-diazabicyclo[4.2.0]octane-2-carboxylate

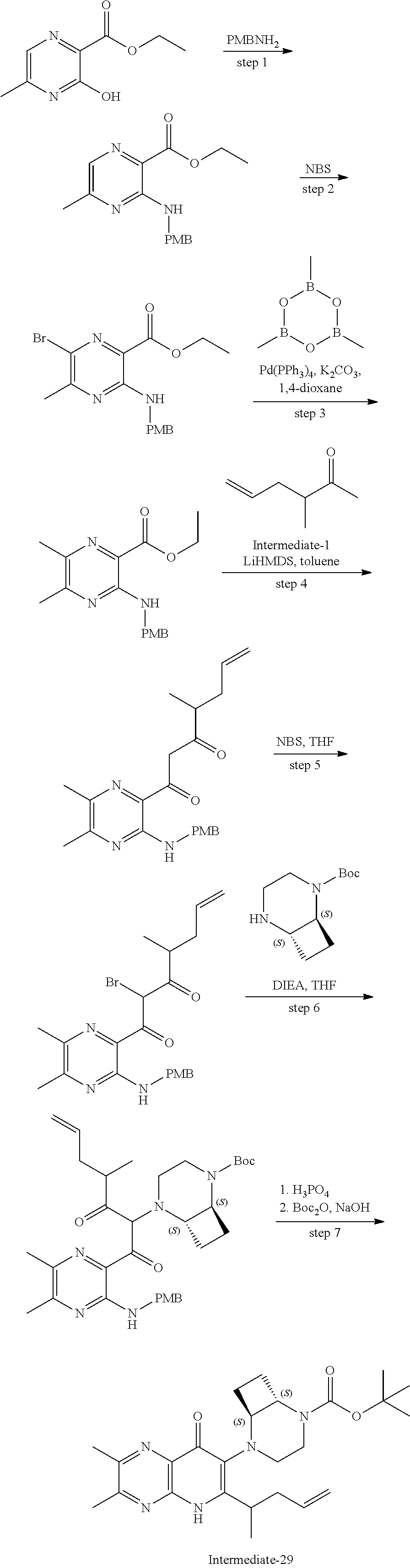

Step 1. To a solution of ethyl 3-hydroxy-5-methylpyrazine-2-carboxylate (100.00 g, 548.92 mmol, 1.0 eq) in DMF (1000 mL) was added DIEA (106.42 g, 823.38 mmol, 1.5 eq) and BOP (267.05 g, 603.81 mmol, 1.1 eq), and the resulting mixture was stirred at 45° C. for 4 h. Then, (4-methoxyphenyl)methanamine (90.36 g, 658.70 mmol, 1.2 eq) was added and the mixture was stirred at 45° C. for overnight. The reaction mixture was diluted with H₂O (2 L) and extracted with EtOAc (1.5 L*3). The combined organic layers were washed with brine (500 mL), dried over anhydrous Na₂SO₄, filtered and the filtrate was concentrated under reduced pressure. The residue was purified by silica gel chromatography (EtOAc/PE) to afford ethyl 3-((4-methoxybenzyl)amino)-5-methylpyrazine-2-carboxylate. LCMS: 302.2 [M+H]⁺.

Step 2. To a solution of ethyl 3-((4-methoxybenzyl)amino)-5-methylpyrazine-2-carboxylate (40.00 g, 132.74 mmol, 1.0 eq) in DMF (400 mL) was added NBS (23.63 g, 132.74 mmol, 1.0 eq) at 0° C., and the resulting mixture was stirred at room temperature for 1 h. The reaction mixture was diluted with H₂O (1 L) and extracted with EtOAc (500 mL*3). The combined organic layers were washed with brine (300 mL), dried over anhydrous Na₂SO₄, filtered and the filtrate was concentrated under reduced pressure to afford ethyl 6-bromo-3-((4-methoxybenzyl)amino)-5-methylpyrazine-2-carboxylate, which was used into the next step without further purification. LCMS: 380.0 [M+H]⁺.

Step 3. To a solution of ethyl 6-bromo-3-((4-methoxybenzyl)amino)-5-methylpyrazine-2-carboxylate (50.50 g, 132.81 mmol, 1.0 eq) and 2,4,6-trimethyl-1,3,5,2,4,6-trioxatriborinane (66.69 g, 265.62 mmol, 2.0 eq) in 1,4-dioxane (505 mL) was added K₂CO₃ (55.07 g, 398.44 mmol, 3.0 eq) and Pd(PPh₃)₄ (15.35 g, 13.28 mmol, 0.1 eq), the resulting mixture was stirred at 110° C. overnight under N₂ atmosphere. The reaction mixture was allowed to cool down to room temperature, diluted with H₂O (1000 mL) and extracted with EtOAc (500 mL*3). The combined organic layers were washed with brine (200 mL), dried over anhydrous Na₂SO₄, filtered and the filtrate was concentrated under reduced pressure. The residue was purified by silica gel chromatography (EtOAc/PE) to afford ethyl 3-((4-methoxybenzyl)amino)-5,6-dimethylpyrazine-2-carboxylate. LCMS: 316.1 [M+H]⁺.

Step 4. To a solution of ethyl 3-((4-methoxybenzyl)amino)-5,6-dimethylpyrazine-2-carboxylate (18.90 g, 59.93 mmol, 1.0 eq) and 3-methylhex-5-en-2-one (Intermediate-1) (29.58 g, 131.85 mmol, 2.2 eq) in toluene (200 mL) was added LiHMDS (1 M in THF, 131.85 mL, 2.2 eq) at 0° C., and then the resulting mixture was heated to 60° C. and stirred at 60° C. for 1 h. The reaction mixture was poured into saturated aqueous NH₄Cl solution (200 mL) and then extracted with EtOAc (200 mL*3). The combined organic layers were washed with brine (200 mL), dried over anhydrous Na₂SO₄, filtered and the filtrate was concentrated under reduced pressure. The residue was purified by silica gel chromatography (EtOAc/PE) to afford 1-(3-((4-methoxybenzyl)amino)-5,6-dimethylpyrazin-2-yl)-4-methylhept-6-ene-1,3-dione. LCMS: 382.2 [M+H]⁺.

Step 5. To a solution of 1-(3-((4-methoxybenzyl)amino)-5,6-dimethylpyrazin-2-yl)-4-methylhept-6-ene-1,3-dione (20.66 g, 54.16 mmol, 1.0 eq) in THF (210 mL) was added NBS (10.12 g, 56.87 mmol, 1.05 eq) at 0° C., and the resulting mixture was stirred at room temperature for 1 h to afford 2-bromo-1-(3-((4-methoxybenzyl)amino)-5,6-dimethylpyrazin-2-yl)-4-methylhept-6-ene-1,3-dione, the reaction solution was used into the next step without further workup or purification. LCMS: 462.2 [M+H]⁺.

Step 6. To the reaction mixture of step 4 was added DIEA (14.04 g, 108.32 mmol, 2.0 eq) and tert-butyl (1S,6S)-2,5-diazabicyclo[4.2.0]octane-2-carboxylate (17.29 g, 81.24 mmol, 1.5 eq), and the resulting mixture was stirred at room temperature overnight. The reaction mixture was diluted with H₂O (500 mL) and extracted with EtOAc (250 mL*3). The combined organic layers were washed with brine (100 mL), dried over anhydrous Na₂SO₄, filtered and the filtrate was concentrated under reduced pressure. The residue was purified by silica gel chromatography (EtOAc/PE) to afford tert-butyl (1S,6S)-5-(1-(3-((4-methoxybenzyl)amino)-5,6-dimethylpyrazin-2-yl)-4-methyl-1,3-dioxohept-6-en-2-yl)-2,5-diazabicyclo[4.2.0]octane-2-carboxylate. LCMS: 592.6 [M+H]⁺.

Step 7. To a solution of tert-butyl (1S,6S)-5-(1-(3-((4-methoxybenzyl)amino)-5,6-dimethylpyrazin-2-yl)-4-methyl-1,3-dioxohept-6-en-2-yl)-2,5-diazabicyclo[4.2.0]octane-2-carboxylate (29.40 g, 49.68 mmol, 1.0 eq) in H₂O (140 mL) was added H₃PO₄ (140 mL), and the resulting mixture was stirred at 80° C. for 2 h. The reaction mixture was basified with aqueous NaOH solution (5 M) to pH 9. Then THF (500 mL) and Boc₂O (20.51 g, 93.96 mmol, 2.0 eq) were added, and the mixture was stirred at room temperature for 15 min. The reaction mixture was extracted with EtOAc (300 mL*3). The combined organic layers were washed with brine (100 mL), dried over anhydrous Na₂SO₄, filtered and the filtrate was concentrated under reduced pressure. The residue was purified by silica gel chromatography (EtOAc/PE) to afford tert-butyl (1S,6S)-5-(2,3-dimethyl-8-oxo-6-(pent-4-en-2-yl)-5,8-dihydropyrido[2,3-b]pyrazin-7-yl)-2,5-diazabicyclo[4.2.0]octane-2-carboxylate (Intermediate-29). LCMS: 454.2 [M+H]⁺.

### Intermediate 30: 6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid

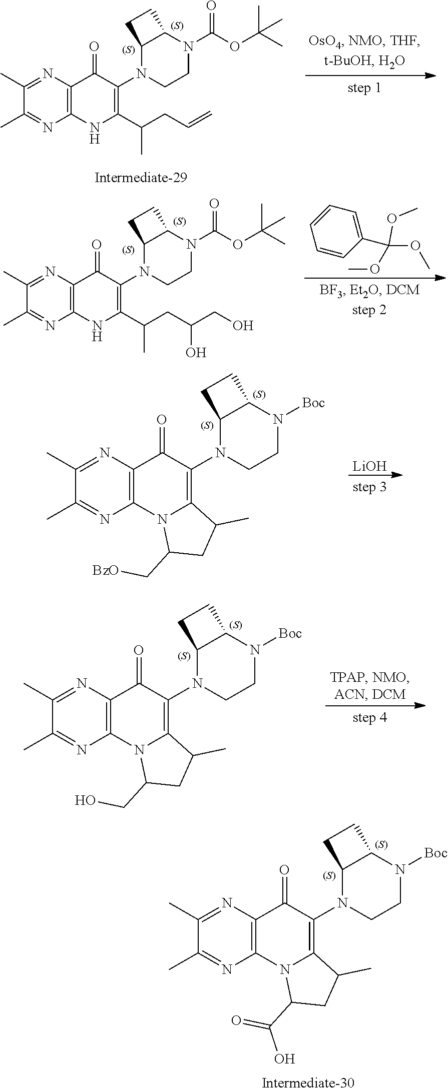

Step 1. To a solution of tert-butyl (1S,6S)-5-(2,3-dimethyl-8-oxo-6-(pent-4-en-2-yl)-5,8-Idihydropyrido[2,3-b]pyrazin-7-yl)-2,5-diazabicyclo[4.2.0]octane-2-carboxylate (Intermediate-29) (9.20 g, 20.28 mmol, 1.0 eq) in THF (60 mL), t-BuOH (10 mL) and H₂O (10 mL) was added NMO (2.61 g, 22.31 mmol, 1.1 eq) and OsO₄ (495 mg, 1.95 mmol, 0.1 eq), and the resulting mixture was stirred at room temperature for 1 h. The reaction mixture was quenched with saturated Na₂SO₃ solution (200 mL) and then extracted with EtOAc (200 mL*3). The combined organic layers were washed with brine (100 mL), dried over anhydrous Na₂SO₄, filtered and the filtrate was concentrated under reduced pressure. The residue was purified by silica gel chromatography (MeOH/DCM) to afford tert-butyl (1S,6S)-5-(6-(4,5-dihydroxypentan-2-yl)-2,3-dimethyl-8-oxo-5,8-dihydropyrido[2,3-b]pyrazin-7-yl)-2,5-diazabicyclo[4.2.0]octane-2-carboxylate. LCMS: 488.3 [M+H]⁺.

Step 2. To a solution of tert-butyl (1S,6S)-5-(6-(4,5-dihydroxypentan-2-yl)-2,3-dimethyl-8-oxo-5,8-dihydropyrido[2,3-b]pyrazin-7-yl)-2,5-diazabicyclo[4.2.0]octane-2-carboxylate (5.46 g, 11.20 mmol, 1.0 eq) and (trimethoxymethyl)benzene (3.06 g, 16.80 mmol, 1.5 eq) in DCM (60 mL) was added BF₃·Et₂O (477 mg, 3.36 mmol, 0.3 eq), and the resulting mixture was stirred at room temperature for 1.5 h. The reaction mixture was quenched by saturated NaHCO₃ (100 mL) and then extracted with DCM (100 mL*3). The combined organic layers were washed with brine (50 mL), dried over anhydrous Na₂SO₄, filtered and concentrated under reduced pressure. The residue was purified by silica gel chromatography (EtOAc/PE) to afford tert-butyl (1S,6S)-5-(9-((benzoyloxy)methyl)-2,3,7-trimethyl-5-oxo-5,7,8,9-tetrahydropyrazino[2,3-e]indolizin-6-yl)-2,5-diazabicyclo[4.2.0]octane-2-carboxylate. LCMS: 574.3 [M+H]⁺.

Step 3. To a solution of tert-butyl (1S,6S)-5-(9-((benzoyloxy)methyl)-2,3,7-trimethyl-5-oxo-5,7,8,9-tetrahydropyrazino[2,3-e]indolizin-6-yl)-2,5-diazabicyclo[4.2.0]octane-2-carboxylate (4.43 g, 7.72 mmol, 1.0 eq) in THF (40 mL), MeOH (20 mL) and H₂O (20 mL) was added LiOH·H₂O (486 mg, 11.58 mmol, 1.5 eq) at 0° C., and the resulting mixture was stirred at room temperature for 0.5 h. The reaction mixture was diluted with H₂O (50 mL) and extracted with EtOAc (50 mL*3). The combined organic layers were washed with brine (30 mL), dried over anhydrous Na₂SO₄, filtered and the filtrate was concentrated under reduced pressure to afford tert-butyl (1S,6S)-5-(9-(hydroxymethyl)-2,3,7-trimethyl-5-oxo-5,7,8,9-tetrahydropyrazino[2,3-e]indolizin-6-yl)-2,5-diazabicyclo[4.2.0]octane-2-carboxylate, which was used in the next step without further purification. LCMS: 470.3 [M+H]⁺.

Step 4. To a solution of tert-butyl (1S,6S)-5-(9-(hydroxymethyl)-2,3,7-trimethyl-5-oxo-5,7,8,9-tetrahydropyrazino[2,3-e]indolizin-6-yl)-2,5-diazabicyclo[4.2.0]octane-2-carboxylate (3.60 g, 7.67 mmol, 1.0 eq) and NMO (1.80 g, 15.33 mmol, 2.0 eq) in DCM (20 mL) and MeCN (20 mL) was added a solution of TPAP (2.69 g, 7.67 mmol, 1 eq) in DCM (2 mL) and MeCN (2 mL) at 0° C., and the resulting mixture was stirred at room temperature for 0.5 h. The reaction mixture was adjusted to pH 3-4 with aqueous HCl solution (1 N), and then diluted with water (50 mL) and extracted with DCM (50 mL*3). The combined organic layers were washed with brine (30 mL), dried over anhydrous Na₂SO₄, filtered and concentrated under reduced pressure to afford 6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid (Intermediate-30), which was used into the next step without further purification. LCMS: 484.2 [M+H]⁺.

### Intermediate-31: tert-butyl (1S,6S)-5-(9-((2-chloro-4-(trifluoromethyl)phenyl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (Stereoisomer 1), Intermediate-32: tert-butyl (1S,6S)-5-(9-((2-chloro-4-(trifluoromethyl)phenyl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (Stereoisomer 2), Intermediate-33: tert-butyl (1S,6S)-5-(9-((2-chloro-4-(trifluoromethyl)phenyl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (Stereoisomer 3), Intermediate-34: tert-butyl (1S,6S)-5-(9-((2-chloro-4-(trifluoromethyl)phenyl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (Stereoisomer 4)

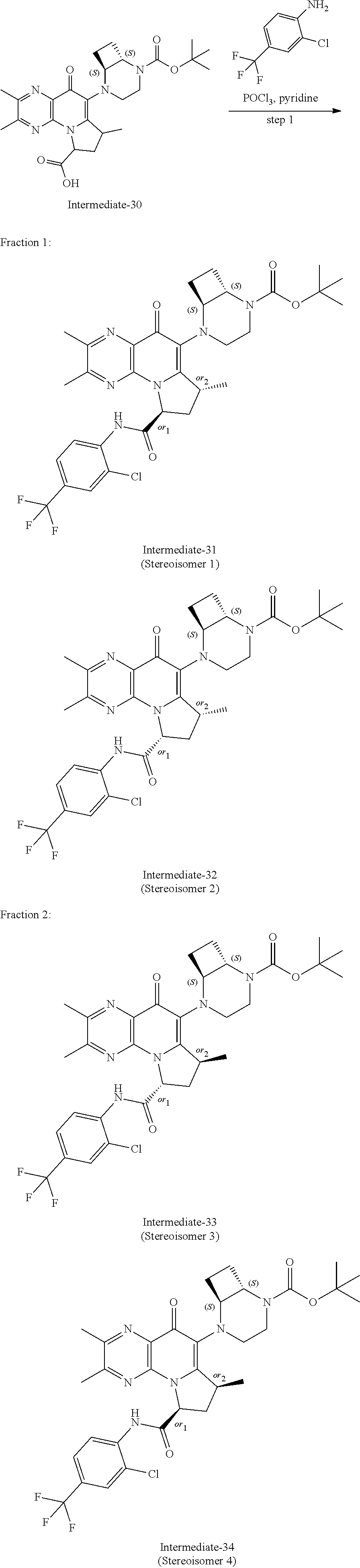

Step 1. To a solution of 6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid (Intermediate-30) (1.86 g, 9.51 mmol, 2.5 eq) in DCM (23 mL) and pyridine (23 mL) was added POCl₃ (875 mg, 5.71 mmol, 1.5 eq) at 0° C., and the resulting mixture was stirred at 0° C. for 10 min. The reaction mixture was diluted with water (50 mL) and extracted with EtOAc (50 mL*3). The combined organic layers were washed with brine (30 mL), dried over anhydrous Na₂SO₄, filtered and concentrated under reduced pressure. The residue was purified by silica gel chromatography (EtOAc/PE) to afford two fractions of the title compound. The two fractions were used in the following steps separately.

Fraction 1: mixture of Intermediate-31 (Stereoisomer 1) and Intermediate-32 (Stereoisomer 2). LCMS: 661.3 [M+H]⁺.

Fraction 2: mixture of Intermediate-33 (Stereoisomer 3) and Intermediate-34 (Stereoisomer 4). LCMS: 661.3 [M+H]⁺.

### Intermediate-35: 6-((1S,6S)-2,5-diazabicyclo[4.2.0]octan-2-yl)-N-(2-chloro-4-(trifluoromethyl)phenyl)-2,3,7-trimethyl-5-oxo-5,7,8,9-tetrahydropyrazino[2,3-e]indolizine-9-carboxamide hydrochloride (Stereoisomer 1) and Intermediate-36: 6-((1S,6S)-2,5-diazabicyclo[4.2.0]octan-2-yl)-N-(2-chloro-4-(trifluoromethyl)phenyl)-2,3,7-trimethyl-5-oxo-5,7,8,9-tetrahydropyrazino[2,3-e]indolizine-9-carboxamide hydrochloride (Stereoisomer 2)

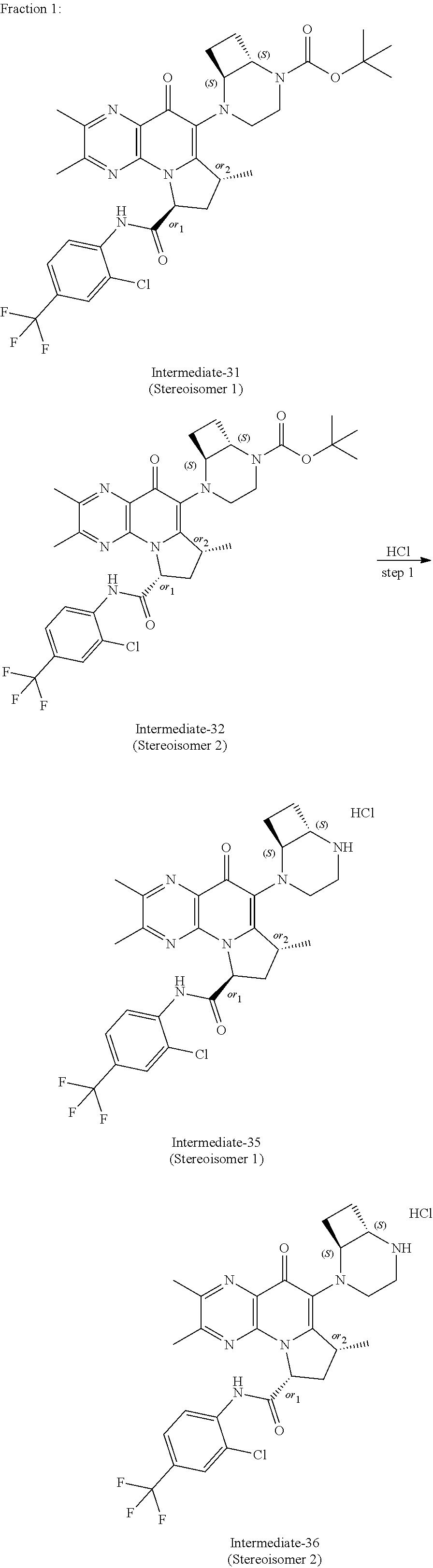

Step 1. To a 4M solution of HCl in 1,4-dioxane (10 mL) was added tert-butyl (1S,6S)-5-(9-((2-chloro-4-(trifluoromethyl)phenyl)carbamoyl)-2,3,7-trimethyl-5-oxo-5,7,8,9-tetrahydropyrazino[2,3-e]indolizin-6-yl)-2,5-diazabicyclo[4.2.0]octane-2-carboxylate (Fraction 1: mixture of Intermediate-31 (Stereoisomer 1) and Intermediate-32 (Stereoisomer 2)) (845 mg, 1.28 mmol, 1.0 eq), and it was stirred at room temperature for 0.5 h. The resulting mixture was concentrated in vacuo to afford the title compound as a mixture of Intermediate-35 (Stereoisomer 1) and Intermediate-36 (Stereoisomer 2), which was used into the next step without further purification. LCMS: 561.3 [M+H]⁺.

### Intermediate-37: 6-((1S,6S)-2,5-diazabicyclo[4.2.0]octan-2-yl)-N-(2-chloro-4-(trifluoromethyl)phenyl)-2,3,7-trimethyl-5-oxo-5,7,8,9-tetrahydropyrazino[2,3-e]indolizine-9-carboxamide hydrochloride (Stereoisomer 3) and Intermediate 38: 6-((1S,6S)-2,5-diazabicyclo[4.2.0]octan-2-yl)-N-(2-chloro-4-(trifluoromethyl)phenyl)-2,3,7-trimethyl-5-oxo-5,7,8,9-tetrahydropyrazino[2,3-e]indolizine-9-carboxamide hydrochloride (Stereoisomer 4)

A mixture of Intermediate-37 and Intermediate-38 was prepared through the same procedure as described for the mixture of Intermediate-35 and Intermediate-36 starting from Fraction 2: mixture of Intermediate-33 (Stereoisomer 3) and Intermediate-34 (Stereoisomer 4). LCMS: 561.3 [M+H]⁺.

### Intermediate-39: tert-butyl (1S,6S)-5-(8-((2-fluoro-4-(trifluoromethyl)phenyl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 1), Intermediate-40: tert-butyl (1S,6S)-5-(8-((2-fluoro-4-(trifluoromethyl)phenyl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 2), Intermediate-41: tert-butyl (1S,6S)-5-(8-((2-fluoro-4-(trifluoromethyl)phenyl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 3), and Intermediate-42: tert-butyl (1S,6S)-5-(8-((2-fluoro-4-(trifluoromethyl)phenyl)carbamoyl)-2-(2-methoxypyridin-4-yl)-6-methyl-4-oxo-4,6,7,8-tetrahydro-2H-[1,2,3]triazolo[4,5-e]indolizin-5-yl)-2,5-diazabicyclo[4.2.0]octane-2-carboxylate (Stereoisomer 4)

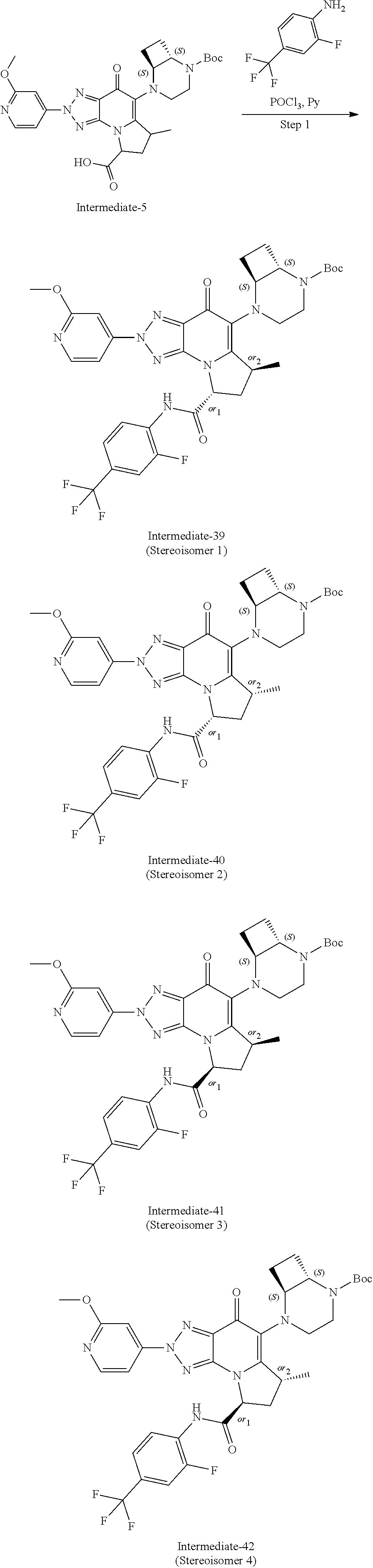

Step 1. To a solution of 5-((1S,6S)-5-(tert-butoxycarbonyl)-2,5-diazabicyclo[4.2.0]octan-2-yl)-2-(2-methoxypyridin-4-yl)-6-methyl-4-oxo-4,6,7,8-tetrahydro-2H-[1,2,3]triazolo[4,5-e]indolizine-8-carboxylic acid (Intermediate-5) (500 mg, 906 mol, 1.0 eq) and 2-fluoro-4-(trifluoromethyl)aniline (406 mg, 2.27 mmol, 2.5 eq) in pyridine (5 mL) and DCM (5 mL) was added POCl₃ (208 mg, 1.36 mmol, 1.5 eq) at 0° C., and the resulting mixture was stirred at 0° C. for 10 min. The reaction mixture was quenched with H₂O (20 mL) and extracted with EtOAc (20 mL*2). The combined organic layers were washed with brine (20 mL), dried over anhydrous Na₂SO₄, filtered and concentrated under reduced pressure. The residue was purified by silica gel chromatography (Eluent of EtOAc/PE) and then prep-TLC(PE/EtOAc) to give the title compounds as single stereoisomers. Each of the stereoisomers was used in the next steps separately. LCMS: 713.4 [M+H]⁺.

### Intermediate-50: methyl (2R,4R)-4-methyl-5-oxopyrrolidine-2-carboxylate

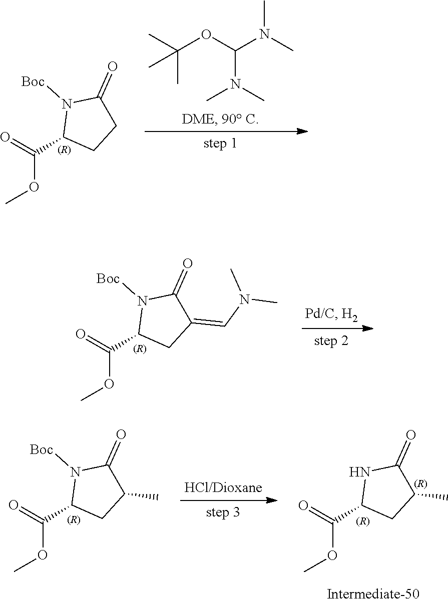

Step 1: To a solution of 1-(tert-butyl) 2-methyl (R)-5-oxopyrrolidine-1,2-dicarboxylate (200.00 g, 822.18 mmol, 1.0 eq) in DME (2000 mL) was added 1-tert-butoxy-N,N,N′,N′-tetramethylmethanediamine (214.94 g, 1.23 mol, 1.5 eq) and the reaction mixture was stirred at 95° C. overnight. The reaction mixture was concentrated under reduced pressure to give a residue. The residue was triturated with MBTE (200 mL) for 15 min at room temperature and then filtered. The filter cake was dried under reduced pressure to afford 1-(tert-butyl) 2-methyl (R)-4-((dimethylamino)methylene)-5-oxopyrrolidine-1,2-dicarboxylate, which was used into the next step without further purification. ¹H NMR (400 MHz, CDCl₃) δ=7.12 (s, 1H), 4.56-4.52 (m, 1H), 3.74 (s, 3H), 3.27-3.20 (m, 1H), 3.00 (s, 6H), 2.90-2.86 (m, 1H), 1.48 (s, 9H) ppm.

Step 2: The solution of 1-(tert-butyl) 2-methyl (R,Z)-4-((dimethylamino)methylene)-5-oxopyrrolidine-1,2-dicarboxylate (100.00 g, 335.19 mmol, 1.0 eq) and AcOH (40.26 g, 670.39 mmol, 2.0 eq) in IPA (4 L) was passed through the flow chemistry equipment (fixed bed with granular catalyst 5% Pd/C; H₂ back pressure: 0.5 Mpa; flow rate of H₂: 90 mL/min; flow rate of SM: 30.3 mL/min; temperature: 80° C., residence time 0.38 min). The resulting mixture was collected and concentrated under reduced pressure to afford 1-(tert-butyl) 2-methyl (2R,4R)-4-methyl-5-oxopyrrolidine-1,2-dicarboxylate, which was used into the next step without further purification. LCMS: 158.2 [M+H−100]+.

Step 3: Synthesis of To 1-(tert-butyl) 2-methyl (2R,4R)-4-methyl-5-oxopyrrolidine-1,2-dicarboxylate (55 g, 213.77 mmol, 1 eq) was added a solution of HCl in 1,4-dioxane (4 M, 550 mL) and the resulting mixture was stirred at room temperature for 0.5 hr. The reaction mixture was concentrated under reduced pressure to afford methyl (2R,4R)-4-methyl-5-oxopyrrolidine-2-carboxylate, which was used in the next step without further purification. LCMS: 158.2 [M+H]⁺.

### Intermediate-51: Synthesis of 4-chloro-2,2-difluorobenzo[d][1,3]dioxol-5-amine trifluoroacetate

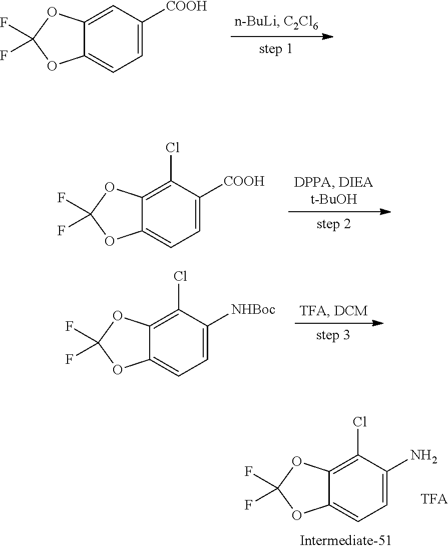

Step 1: To a solution of 2,2-difluoro-1,3-benzodioxole-5-carboxylic acid (3.50 g, 17.32 mmol, 1.0 eq) in THF (66 mL) was added n-BuLi (2.5 M in hexane, 18.70 mL, 2.7 eq) at −78° C. After addition was complete the reaction mixture was warmed up to 0° C. and stirred for 1 h. The reaction mixture was cooled again to −78° C. and 1,1,1,2,2,2-hexachloroethane (12.30 g, 51.95 mmol, 3.0 eq) was added. The resulting mixture was stirred at −78 for 40 min, and then slowly warmed up to room temperature and stirred for 40 min. The reaction mixture was poured into saturated aqueous citric acid solution (100 mL) and extracted with EtOAc (50 mL×3), then the combined organic layers were washed with brine (50 mL), dried with anhydrous Na₂SO₄, filtered and the filtrate was concentrated under reduced pressure. The residue was purified by silica gel chromatography (Eluent of EtOAc/PE) to afford 4-chloro-2,2-difluorobenzo[d][1,3]dioxole-5-carboxylic acid. ¹H NMR (400 MHz, DMSO) δ 7.81 (d, 1H), 7.49 (d, 1H).

Step 2: To the solution of 4-chloro-2,2-difluoro-1,3-benzodioxole-5-carboxylic acid (800 mg, 3.38 mmol, 1.0 eq) in t-BuOH (10 mL) was added DPPA (1.12 g, 4.06 mmol, 1.2 eq) and DIEA (655.62 mg, 5.07 mmol, 883.58 μL, 1.5 eq) and the resulting mixture was stirred at 80° C. overnight. The reaction mixture was filtered and the filtrate was concentrated under reduced pressure. The residue was purified by silica gel chromatography (Eluent of EtOAc/PE) to afford tert-butyl (4-chloro-2,2-difluorobenzo[d][1,3]dioxol-5-yl)carbamate. ¹H NMR (400 MHz, DMSO) δ 8.99 (s, 1H), 7.42-7.33 (m, 1H), 7.30-7.20 (m, 1H), 1.45 (s, 9H).

Step 3: To a solution of tert-butyl N-(4-chloro-2,2-difluoro-1,3-benzodioxol-5-yl) carbamate (470 mg, 1.53 mmol, 1.0 eq) in DCM (6 mL) was added TFA (2 mL), and the resulting mixture was stirred at room temperature overnight. The reaction mixture was adjusted to pH 8 with saturated aqueous NaHCO₃ solution, then extracted with EtOAc (20 mL×3). The combined organic layers were washed with brine (15 mL), dried over Na₂SO₄, filtered and the filtrate was concentrated under reduced pressure to afford 4-chloro-2,2-difluorobenzo[d][1,3]dioxol-5-amine trifluoroacetate which was used in the next step without further purification. LCMS: 208.0 [M+H]⁺.

### Intermediate 52: (7RS,9R)-6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid (Mixture of 2 Stereoisomers)

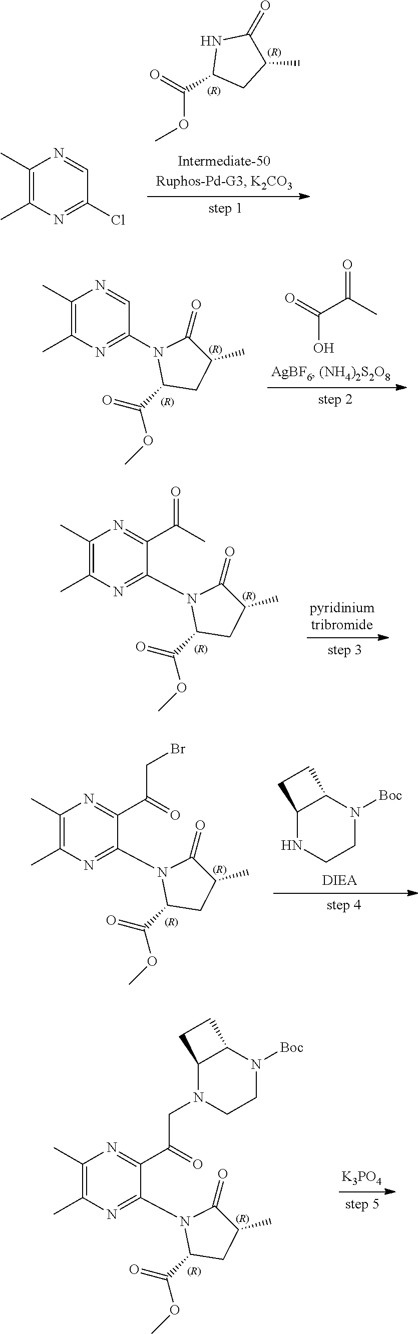

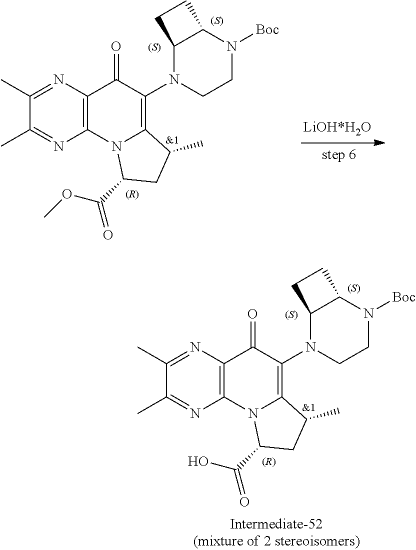

Step 1: To the solution of 5-chloro-2,3-dimethyl-pyrazine (19.00 g, 133.25 mmol, 1.0 eq) and methyl (2R,4R)-4-methyl-5-oxopyrrolidine-2-carboxylate (Intermediate-50) (31.41 g, 199.88 mmol, 1.5 eq) in 1,4-dioxane (200 mL) was added the K₂CO₃ (55.25 g, 399.76 mmol, 3 eq) and RuPhos-Pd-G3 (CAS: 1445085-77-7) (11.14 g, 13.33 mmol, 0.1 eq) and the resulting mixture was stirred at 100° C. overnight under N₂ atmosphere. The reaction mixture was filtered and the filtrate was concentrated under reduced pressure. The residue was purified by silica gel chromatography (Eluent of EtOAc/PE) to afford methyl (2R,4R)-1-(5,6-dimethylpyrazin-2-yl)-4-methyl-5-oxopyrrolidine-2-carboxylate. LCMS: 264.1 [M+H]⁺.

Step 2. To the solution of methyl (2R,4R)-1-(5,6-dimethylpyrazin-2-yl)-4-methyl-5-oxo-pyrrolidine-2-carboxylate (22.00 g, 83.56 mmol, 1.0 eq), ammonium persulfate (38.14 g, 167.12 mmol, 2.0 eq), 2-oxopropanoic acid (22.07 g, 250.67 mmol, 3.0 eq) and AgPF₆ (2.11 g, 8.36 mmol, 0.1 eq) in DCM (250 mL) and H₂O (250 mL) was added the TFA (28.58 g, 250.67 mmol, 3.0 eq) at room temperature and the resulting mixture was stirred at 40° C. overnight. The reaction mixture was filtered, the filtrate was extracted with DCM (200 mL×2). The combined organic phase was washed with brine (200 mL), dried over anhydrous Na₂SO₄, filtered and the filtrate was concentrated under reduced pressure. The residue was purified by silica gel chromatography (Eluent of EtOAc/PE) to afford methyl (2R,4R)-1-(3-acetyl-5,6-dimethylpyrazin-2-yl)-4-methyl-5-oxopyrrolidine-2-carboxylate. LCMS: 306.2 [M+H]⁺.

Step 3: To the solution of methyl (2R,4R)-1-(3-acetyl-5,6-dimethyl-pyrazin-2-yl)-4-methyl-5-oxo-pyrrolidine-2-carboxylate (10.00 g, 32.75 mmol, 1.0 eq) in HBr (33weight % in AcOH, 100 mL) was added pyridinium tribromide (12.57 g, 39.30 mmol, 1.2 eq), and the resulting mixture was stirred at room temperature for 1 h. The reaction mixture was diluted with H₂O (200 mL) and MTBE (100 mL), then basified with saturated aqueous NaHCO₃ solution (200 mL). The organic phase was separated, and the aqueous phase was extracted with MTBE (50 mL). The combined organic phase was washed with brine (100 mL), dried over anhydrous Na₂SO₄, filtered and the filtrate was concentrated under reduced pressure to afford methyl (2R,4R)-1-(3-(2-bromoacetyl)-5,6-dimethylpyrazin-2-yl)-4-methyl-5-oxopyrrolidine-2-carboxylate, which was used into the next step without further purification. LCMS: 384.0 [M+H]⁺.

Step 4: To the solution of methyl (2R,4R)-1-[3-(2-bromoacetyl)-5,6-dimethyl-pyrazin-2-yl]-4-methyl-5-oxo-pyrrolidine-2-carboxylate (11.00 g, 28.63 mmol, 1.0 eq) and tert-butyl (1S,6S)-2,5-diazabicyclo[4.2.0]octane-2-carboxylate (9.12 g, 42.94 mmol, 1.5 eq) in DCM (100 mL) was added DIEA (7.40 g, 57.26 mmol, 2.0 eq) and the resulting mixture was stirred at room temperature for 1.5 h. The reaction mixture was concentrated under reduced pressure and the residue was purified by silica gel chromatography (Eluent of EtOAc/PE) to afford tert-butyl (1S,6S)-5-(2-(3-((3R,5R)-5-(methoxycarbonyl)-3-methyl-2-oxopyrrolidin-1-yl)-5,6-dimethylpyrazin-2-yl)-2-oxoethyl)-2,5-diazabicyclo[4.2.0]octane-2-carboxylate. LCMS: 516.4 [M+H]⁺.

Step 5: To the solution of tert-butyl (1S,6S)-5-[2-[3-[(3R,5R)-5-methoxycarbonyl-3-methyl-2-oxo-pyrrolidin-1-yl]-5,6-dimethyl-pyrazin-2-yl]-2-oxo-ethyl]-2,5-diazabicyclo[4.2.0]octane-2 carboxylate (3.00 g, 5.82 mmol, 1.0 eq) in toluene (30 mL) was added K₃PO₄ (3.71 g, 17.46 mmol, 3.0 eq), and the resulting mixture was stirred at 120° C. for 2 h. The reaction mixture was filtered and the filtrate was concentrated under reduced pressure to afford methyl (7RS,9R)-6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylate (Mixture of 2 stereoisomers), which was used in the next step without further purification. LCMS: 498.5 [M+H].

Step 6: To the solution of methyl (7RS,9R)-6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylate) (1.7:1, 2.70 g, 5.43 mmol, 1.0 eq) in THF (10 mL), MeOH (10 mL) and H₂O (10 mL) was added LiOH×H₂O (455 mg, 10.85 mmol, 2.0 eq) and the resulting mixture was stirred at room temperature for 1 h. The reaction mixture was acidified with aqueous HCl (1 N, 20 mL) and then extracted EtOAc (10 mL×3). The combined organic phase was washed with brine (20 mL), dried with anhydrous Na₂SO₄, filtered and the filtrate was concentrated under reduced pressure to afford (7RS,9R)-6-((1S,6S)-5-(tert-butoxycarbonyl)-2,5-diazabicyclo[4.2.0]octan-2-yl)-2,3,7-trimethyl-5-oxo-5,7,8,9-tetrahydropyrazino[2,3-e]indolizine-9-carboxylic acid (Mixture of 2 stereoisomers) which was used in the next step without further purification. LCMS: 484.3 [M+H]⁺.

### Compounds of the Disclosure

The compounds of the disclosure are shown below in Table 2 or Table 2a along with characterization data (Table 2) or the theoretical mass (Table 2a).

TABLE 2

| | | | Mass | Retention | Chiral | Retention |
|---|---|---|---|---|---|---|
| | | LCMS | Observed | time | analytical | time |
| No. | Compound | method | [g/mol] | [min] | method | [min] |
| I-1 | 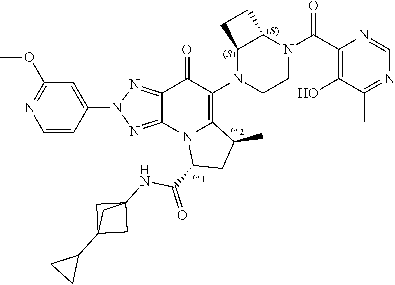 | LCMS 2 | 693.4 | 2.327 | Chiral SFC method 1 | 1.99 |
| I-2 | 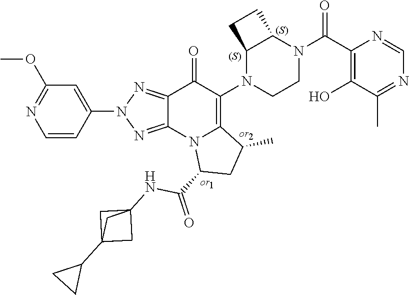 | LCMS 2 | 693.4 | 2.405 | Chiral SFC method 1 | 2.21 |
| I-3 | 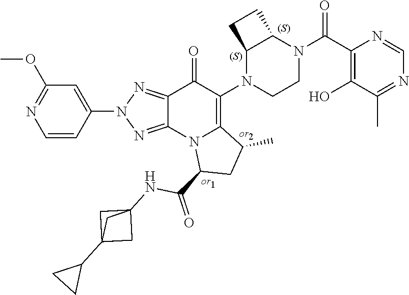 | LCMS 2 | 693.4 | 2.384 | Chiral SFC method 1 | 2.37 |
| I-4 | 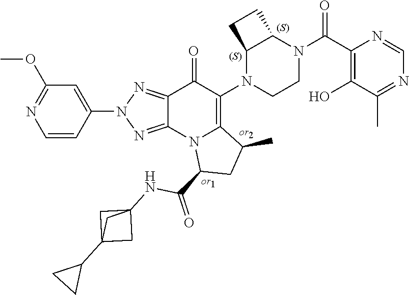 | LCMS 2 | 693.4 | 2.379 | Chiral SFC method 1 | 2.55 |
| I-5 | 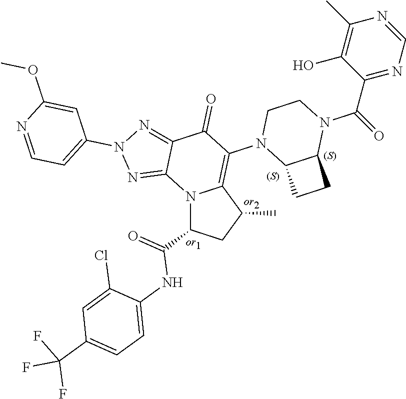 | LCMS 2 | 765.3 | 2.54 | Chiral SFC method 3 | 1.15 |
| I-6 | 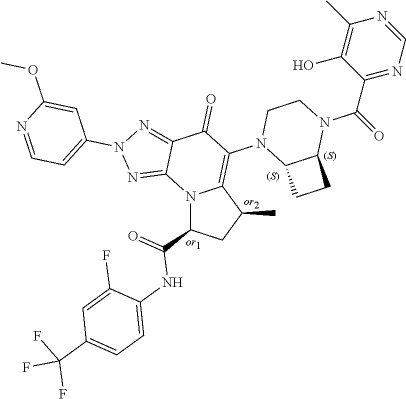 | LCMS 2 | 749.3 | 2.5 | Chiral SFC method 4 | 0.73 |
| I-7 | 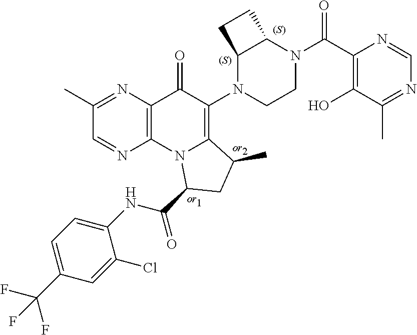 | LCMS 2 | 683.3 | 2.31 | Chiral SFC method 2 | 1.94 |
| I-8 | 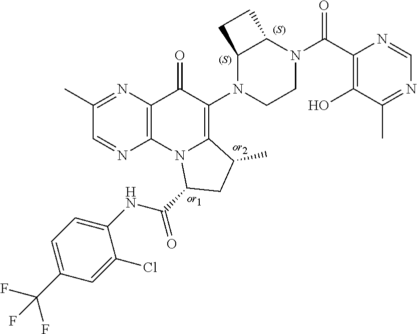 | LCMS 1 | 683.35 | 0.6 | Chiral SFC method 2 | 1.84 |
| I-9 | 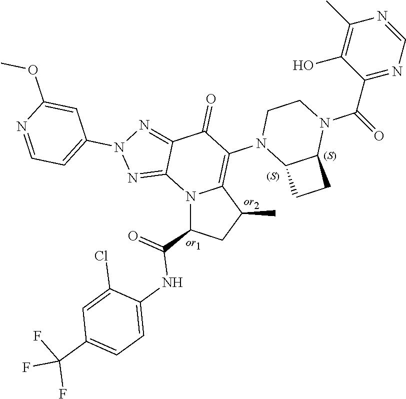 | LCMS 2 | 765.3 | 2.53 | Chiral SFC method 3 | 1.41 |
| I-10 | 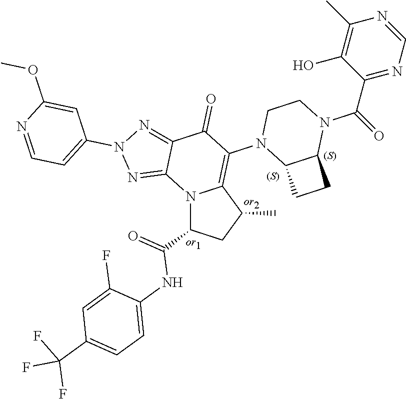 | LCMS 2 | 749.3 | 2.48 | Chiral SFC method 4 | 0.91 |
| I-11 | 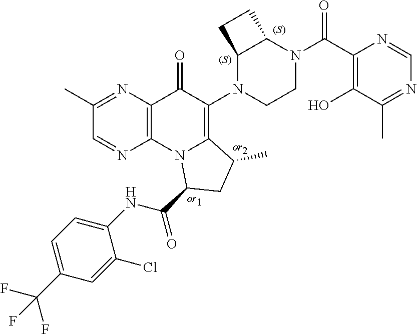 | LCMS 2 | 683.3 | 2.25 | Chiral SFC method 2 | 1.58 |
| I-12 | 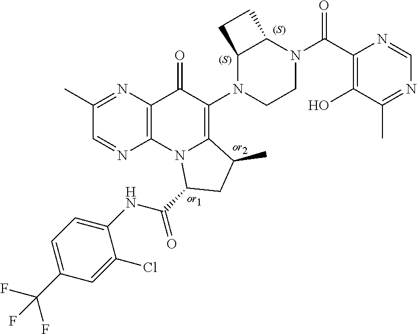 | LCMS 1 | 683.4 | 0.61 | Chiral SFC method 2 | 1.72 |
| I-13 | 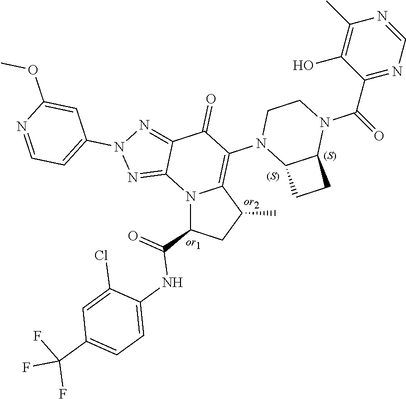 | LCMS 2 | 765.2 | 2.47 | Chiral SFC method 3 | 0.80 |
| I-14 | 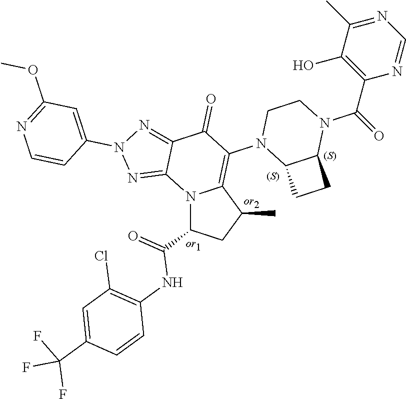 | LCMS 2 | 765.3 | 2.55 | Chiral SFC method 3 | 1.80 |
| I-15 | 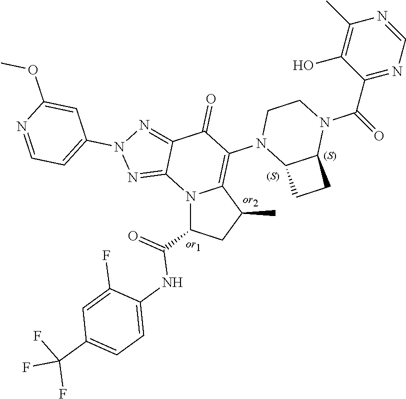 | LCMS 2 | 749.3 | 2.43 | Chiral SFC method 4 | 0.52 |
| I-16 | 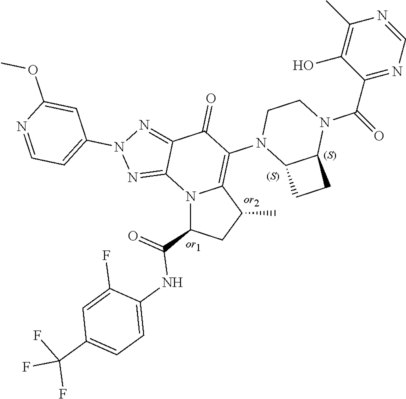 | LCMS 2 | 749.3 | 2.49 | Chiral SFC method 4 | 1.04 |
| I-17 | 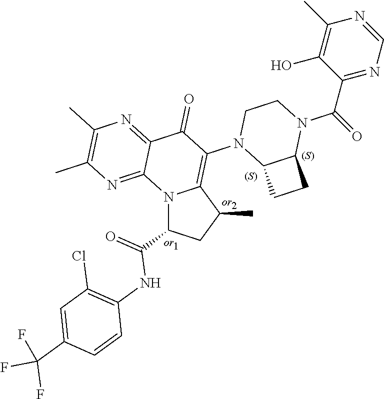 | LCMS 1 | 697.3 | 0.92 | Chiral HPLC method 5 | 1.49 |
| I-18 | 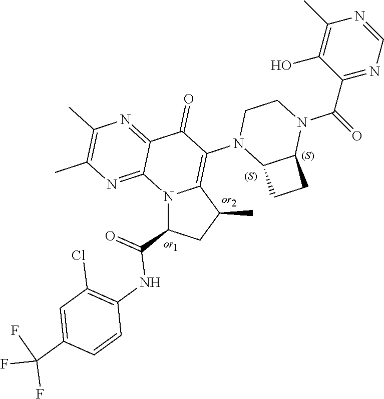 | LCMS 1 | 697.3 | 0.94 | Chiral HPLC method 5 | 1.90 |
| I-19 | 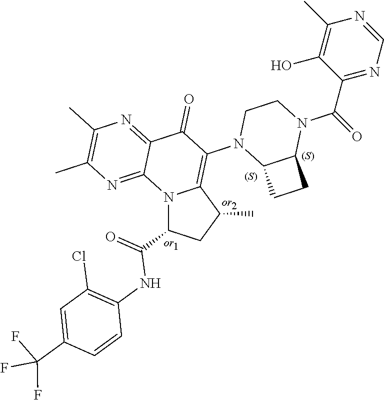 | LCMS 1 | 697.3 | 0.56 | Chiral HPLC method 5 | 4.82 |
| I-20 | 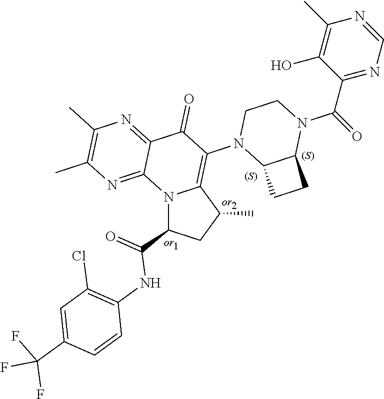 | LCMS 1 | 697.3 | 0.56 | Chiral HPLC method 5 | 6.64 |
| I-21 | 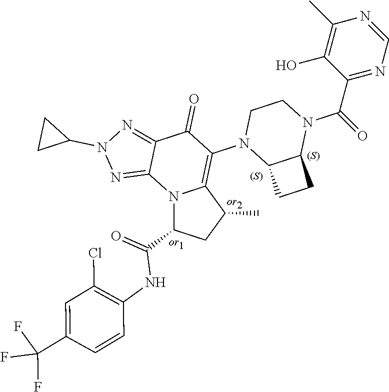 | LCMS 3 | 698.4 | 0.53 | Chiral SFC method 6 | 1.98 |
| I-22 | 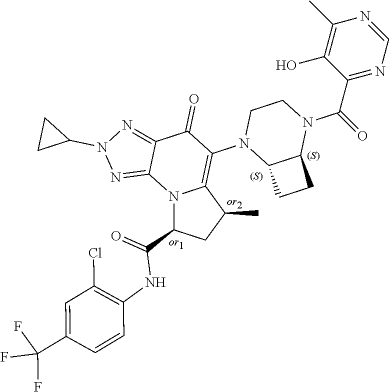 | LCMS 1 | 698.2 | 0.57 | Chiral SFC method 6 | 2.05 |
| I-23 | 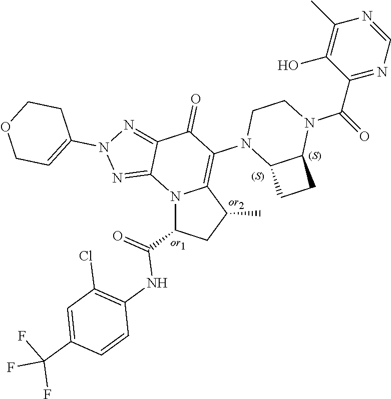 | LCMS 1 | 740.4 | 0.557 | Chiral SFC method 7 | 2.11 |
| I-24 | 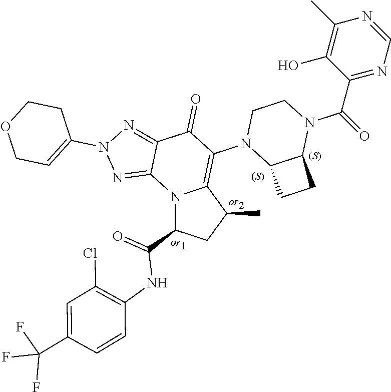 | LCMS 1 | 740.4 | 0.550 | Chiral SFC method 7 | 1.97 |
| I-25 | 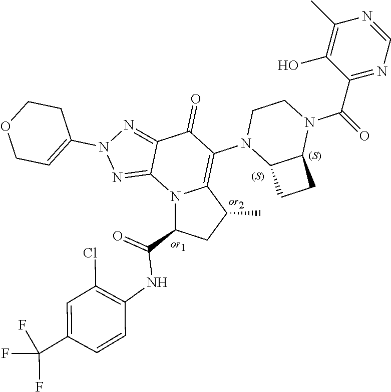 | LCMS 1 | 740.4 | 0.558 | Chiral SFC method 7 | 1.84 |
| I-26 | 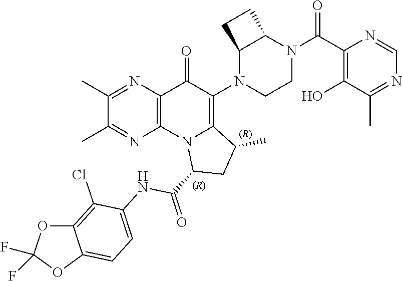 | LCMS 1 | 0.562 | 709.2 | Chiral SFC method 8 | 1.08 |
| I-27 | 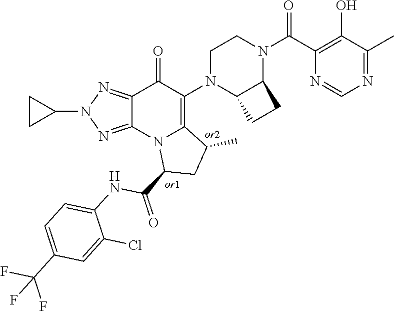 [SMILES: Cc1ncnc(C(=O)N2CCN(c3c4n(c5nn(C6CC6)nc5c3=O)[C@H](C(=O)Nc3ccc(C(F)(F)F)cc3Cl)C[C@H]4C)[C@H]3CC[C@@H]32)c1O] | LCMS 1 | 0.624 | 698.2 | Chiral SFC method 6 | 1.84 |
| I-28 | 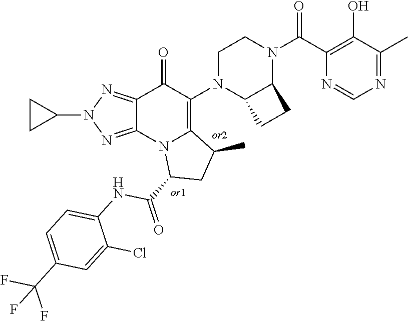 [SMILES: Cc1ncnc(C(=O)N2CCN(c3c4n(c5nn(C6CC6)nc5c3=O)[C@@H](C(=O)Nc3ccc(C(F)(F)F)cc3Cl)C[C@@H]4C)[C@H]3CC[C@@H]32)c1O] | LCMS 1 | 0.566 | 698.3 | Chiral SFC method 6 | 2.16 |

Further compounds of the disclosure are shown below in Table 2a along with the theoretical mass.

TABLE 2a

| | | (Mass theoretical) |
|---|---|---|
| No. | Compound | [g/mol] |
| I-3a | 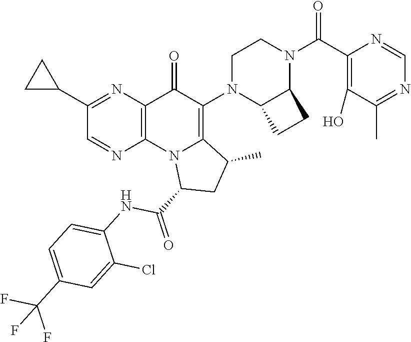 [SMILES: Cc1ncnc(C(=O)N2CCN(c3c4n(c5ncc(C6CC6)nc5c3=O)[C@@H](C(=O)Nc3ccc(C(F)(F)F)cc3Cl)C[C@H]4C)[C@H]3CC[C@@H]32)c1O] | 708.2 |
| I-4a | 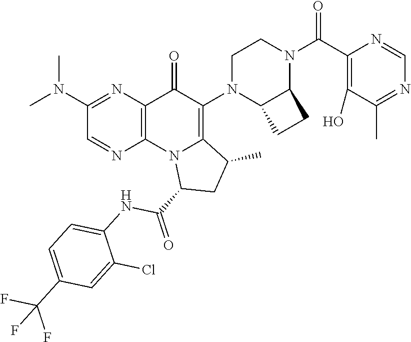 [SMILES: Cc1ncnc(C(=O)N2CCN(c3c4n(c5ncc(N(C)C)nc5c3=O)[C@@H](C(=O)Nc3ccc(C(F)(F)F)cc3Cl)C[C@H]4C)[C@H]3CC[C@@H]32)c1O] | 711.2 |
| I-5a | 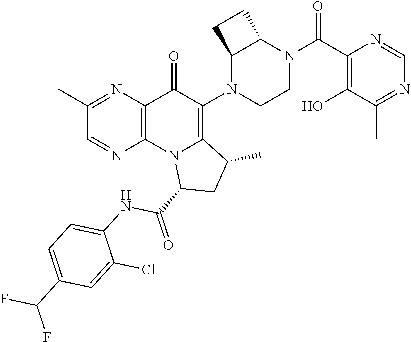 [SMILES: Cc1cnc2c(n1)c(=O)c(N1CCN(C(=O)c3ncnc(C)c3O)[C@H]3CC[C@@H]31)c1n2[C@@H](C(=O)Nc2ccc(C(F)F)cc2Cl)C[C@H]1C] | 664.2 |
| I-6a | 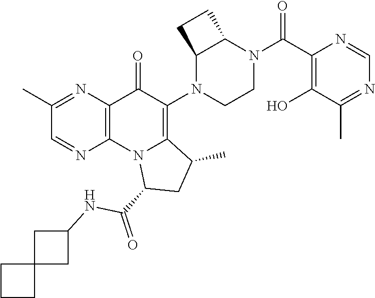 [SMILES: Cc1cnc2c(n1)c(=O)c(N1CCN(C(=O)c3ncnc(C)c3O)[C@H]3CC[C@@H]31)c1n2[C@@H](C(=O)NC2CC3(CCC3)C2)C[C@H]1C] | 598.3 |
| I-7a | 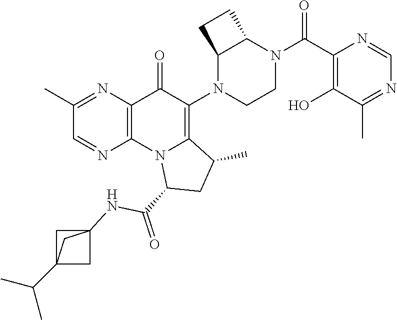 [SMILES: Cc1cnc2c(n1)c(=O)c(N1CCN(C(=O)c3ncnc(C)c3O)[C@H]3CC[C@@H]31)c1n2[C@@H](C(=O)NC23CC(C(C)C)(C2)C3)C[C@H]1C] | 612.3 |
| I-8a | 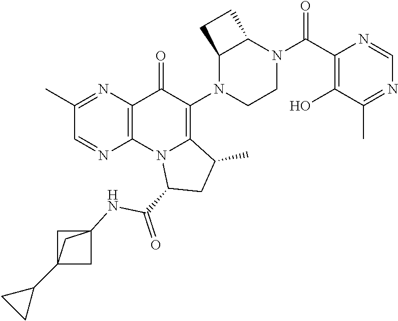 [SMILES: Cc1cnc2c(n1)c(=O)c(N1CCN(C(=O)c3ncnc(C)c3O)[C@H]3CC[C@@H]31)c1n2[C@@H](C(=O)NC23CC(C4CC4)(C2)C3)C[C@H]1C] | 610.3 |
| I-9a | 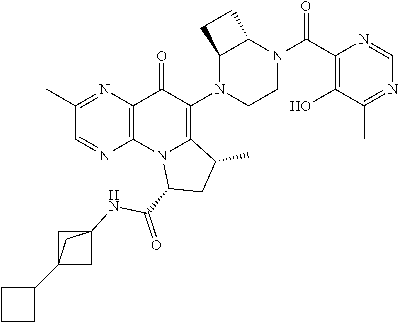 [SMILES: Cc1cnc2c(n1)c(=O)c(N1CCN(C(=O)c3ncnc(C)c3O)[C@H]3CC[C@@H]31)c1n2[C@@H](C(=O)NC23CC(C4CCC4)(C2)C3)C[C@H]1C] | 624.3 |
| I-10a | 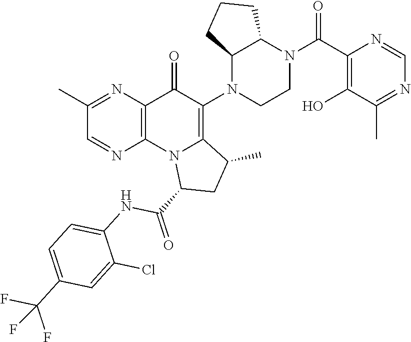 [SMILES: Cc1cnc2c(n1)c(=O)c(N1CCN(C(=O)c3ncnc(C)c3O)[C@H]3CCC[C@@H]31)c1n2[C@@H](C(=O)Nc2ccc(C(F)(F)F)cc2Cl)C[C@H]1C] | 696.2 |
| I-11a | 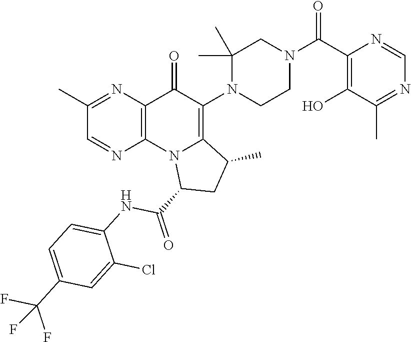 [SMILES: Cc1cnc2c(n1)c(=O)c(N1CCN(C(=O)c3ncnc(C)c3O)CC1(C)C)c1n2[C@@H](C(=O)Nc2ccc(C(F)(F)F)cc2Cl)C[C@H]1C] | 684.2 |
| I-12a | 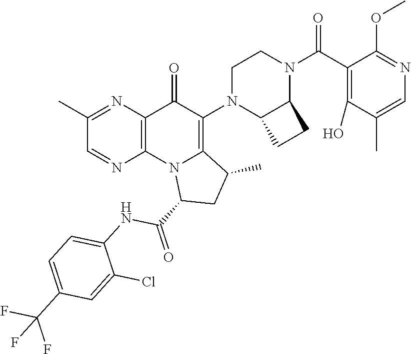 [SMILES: COc1ncc(C)c(O)c1C(=O)N1CCN(c2c3n(c4ncc(C)nc4c2=O)[C@@H](C(=O)Nc2ccc(C(F)(F)F)cc2Cl)C[C@H]3C)[C@H]2CC[C@@H]21] | 711.2 |
| I-18a | 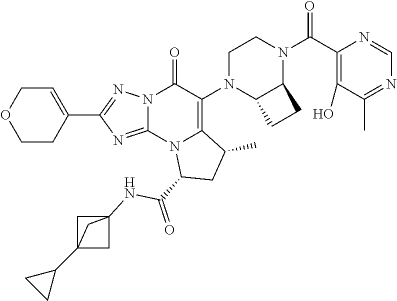 [SMILES: Cc1ncnc(C(=O)N2CCN(c3c4n(c5nc(C6=CCOCC6)nn5c3=O)[C@@H](C(=O)NC35CC(C6CC6)(C3)C5)C[C@H]4C)[C@H]3CC[C@@H]32)c1O] | 667.3 |
| I-19a | 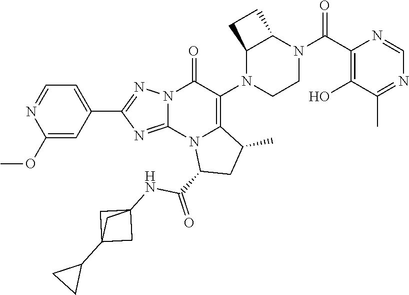 [SMILES: COc1cc(-c2nc3n4c(c(N5CCN(C(=O)c6ncnc(C)c6O)[C@H]6CC[C@@H]65)c(=O)n3n2)[C@H](C)C[C@@H]4C(=O)NC23CC(C4CC4)(C2)C3)ccn1] | 692.3 |
| I-27a | 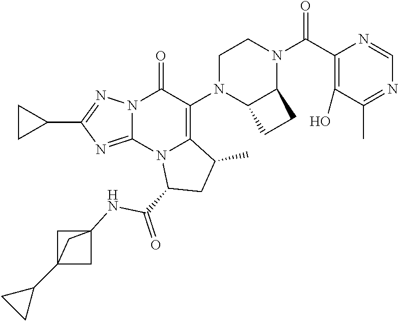 [SMILES: Cc1ncnc(C(=O)N2CCN(c3c4n(c5nc(C6CC6)nn5c3=O)[C@@H](C(=O)NC35CC(C6CC6)(C3)C5)C[C@H]4C)[C@H]3CC[C@@H]32)c1O] | 625.3 |
| I-28a | 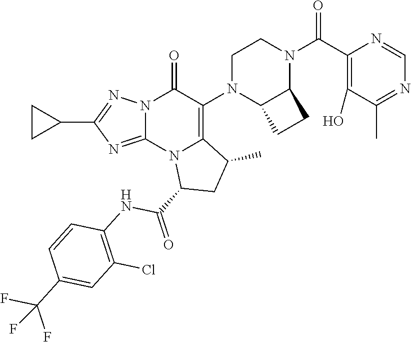 [SMILES: Cc1ncnc(C(=O)N2CCN(c3c4n(c5nc(C6CC6)nn5c3=O)[C@@H](C(=O)Nc3ccc(C(F)(F)F)cc3Cl)C[C@H]4C)[C@H]3CC[C@@H]32)c1O] | 697.2 |
| I-29a | 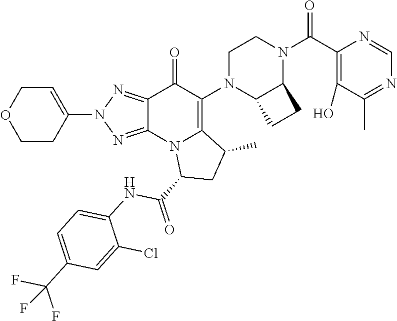 [SMILES: Cc1ncnc(C(=O)N2CCN(c3c4n(c5nn(C6=CCOCC6)nc5c3=O)[C@@H](C(=O)Nc3ccc(C(F)(F)F)cc3Cl)C[C@H]4C)[C@H]3CC[C@@H]32)c1O] | 739.2 |
| I-31a | 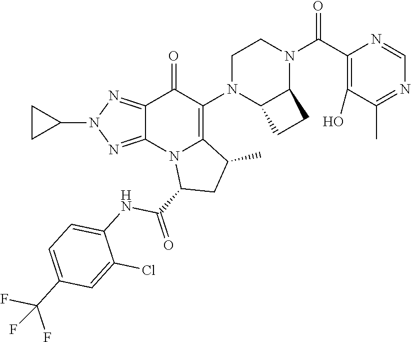 [SMILES: Cc1ncnc(C(=O)N2CCN(c3c4n(c5nn(C6CC6)nc5c3=O)[C@@H](C(=O)Nc3ccc(C(F)(F)F)cc3Cl)C[C@H]4C)[C@H]3CC[C@@H]32)c1O] | 697.2 |
| I-32a | 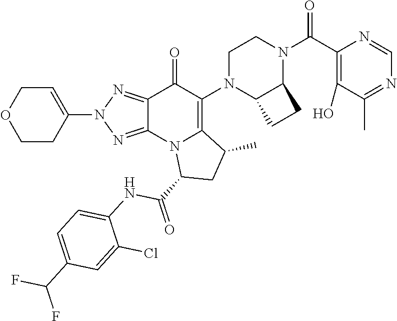 [SMILES: Cc1ncnc(C(=O)N2CCN(c3c4n(c5nn(C6=CCOCC6)nc5c3=O)[C@@H](C(=O)Nc3ccc(C(F)F)cc3Cl)C[C@H]4C)[C@H]3CC[C@@H]32)c1O] | 721.2 |
| I-38a | 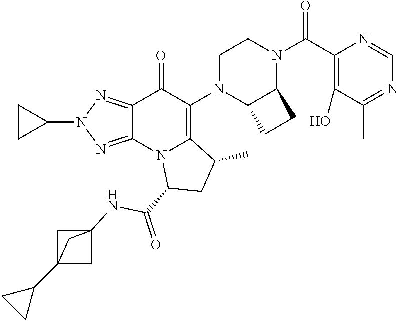 [SMILES: Cc1ncnc(C(=O)N2CCN(c3c4n(c5nn(C6CC6)nc5c3=O)[C@@H](C(=O)NC35CC(C6CC6)(C3)C5)C[C@H]4C)[C@H]3CC[C@@H]32)c1O] | 625.3 |
| I-39a | 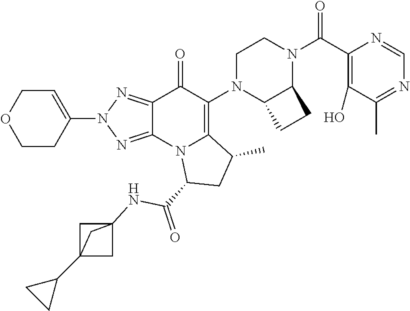 [SMILES: Cc1ncnc(C(=O)N2CCN(c3c4n(c5nn(C6=CCOCC6)nc5c3=O)[C@@H](C(=O)NC35CC(C6CC6)(C3)C5)C[C@H]4C)[C@H]3CC[C@@H]32)c1O] | 667.3 |
| I-40a | 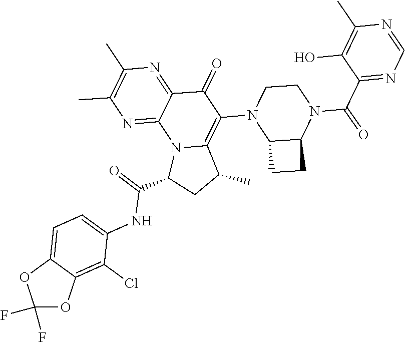 [SMILES: Cc1nc2c(=O)c(N3CCN(C(=O)c4ncnc(C)c4O)[C@H]4CC[C@@H]43)c3n(c2nc1C)[C@@H](C(=O)Nc1ccc2c(c1Cl)OC(F)(F)O2)C[C@H]3C] | 708.2 |
| I-41a | 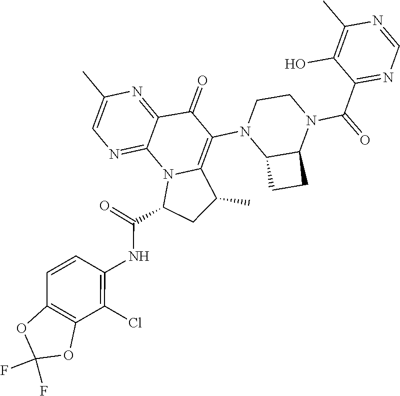 [SMILES: Cc1cnc2c(n1)c(=O)c(N1CCN(C(=O)c3ncnc(C)c3O)[C@H]3CC[C@@H]31)c1n2[C@@H](C(=O)Nc2ccc3c(c2Cl)OC(F)(F)O3)C[C@H]1C] | 694.2 |
| I-42a | 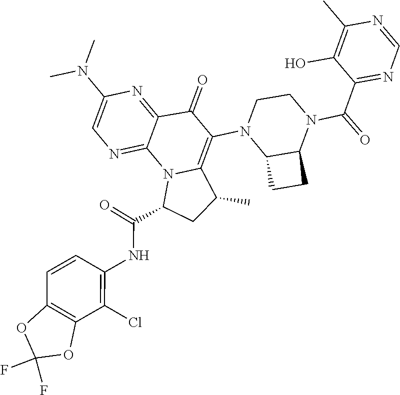 [SMILES: Cc1ncnc(C(=O)N2CCN(c3c4n(c5ncc(N(C)C)nc5c3=O)[C@@H](C(=O)Nc3ccc5c(c3Cl)OC(F)(F)O5)C[C@H]4C)[C@H]3CC[C@@H]32)c1O] | 723.2 |
| I-43a | 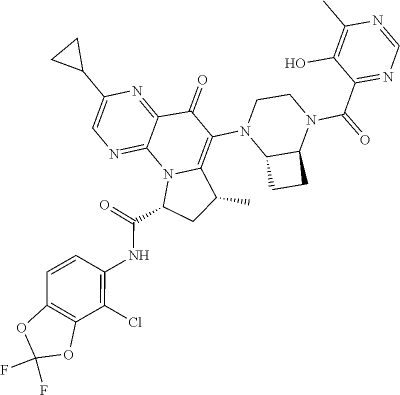 [SMILES: Cc1ncnc(C(=O)N2CCN(c3c4n(c5ncc(C6CC6)nc5c3=O)[C@@H](C(=O)Nc3ccc5c(c3Cl)OC(F)(F)O5)C[C@H]4C)[C@H]3CC[C@@H]32)c1O] | 720.2 |

### LCMS Methods

LCMS 1

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 5 to 95% B in 0.5 min, 95% B for 0.3 min, |
| | 95 to 5% B in 0.01 min, 5% B for 0.24 min |
| Flow Rate | 1.5 mL/min |
| Column Temperature | 50° C. |

LCMS 2

| | |
|---|---|
| Column | 3.0 × 30 mm, 5.0 μm |
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 5 to 95% B in 3.0 min, 95% B for 0.5 min, |
| | 95 to 5% B in 0.01 min, 5% B for 0.49 min |
| Flow Rate | 0.8 mL/min |
| Column Temperature | 50° C. |
| Column | 3.0 × 30 mm, 5.0 μm |

LCMS 3

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 5 to 95% B in 0.45 min, 95% B for 0.25 min, |
| | 95 to 5% B in 0.01 min, 5% B for 0.19 min |
| Flow Rate | 1.5 mL/min |
| Column Temperature | 50° C. |
| Column | 3.0 × 30 mm, 5.0 μm |

Chiral SFC method 1:

| Instrument | SHIMADZU LC-30AD | | |
|---|---|---|---|
| column | Chiralpak IK-3 50 × 4.6 mm I.D., particle size 3 μm | | |
| Mobile Phase | Phase A for CO₂ | Phase B for IPA + ACN 2:1 (0.05% DEA) | |
| | Time(min) | A(%) | B(%) |
| Gradient | 0.00 | 80 | 20 |
| | 1.80 | 40 | 60 |
| | 2.70 | 40 | 60 |
| | 2.71 | 80 | 20 |
| | 3.00 | 80 | 20 |

| | |
|---|---|
| Flow rate | 3.0 mL/min |
| Column Temp | 35° C. |
| Back pressure | 100 Bar |
| UV | 220 nm |

Chiral SFC method 2:

| Instrument | SHIMADZU LC-30AD | | |
|---|---|---|---|
| Column | Chiralcel OD-3 50 × 4.6 mm I.D., | | |
| | particle size 3 μm | | |
| Mobile Phase | Phase A for CO₂ | Phase B for IPA (0.05% DEA) | |
| | Time(min) | A(%) | B(%) |
| Gradient | 0.00 | 95 | 10 |
| | 1.80 | 40 | 60 |
| | 2.70 | 40 | 60 |
| | 2.71 | 90 | 10 |
| | 3.00 | 90 | 10 |

| | |
|---|---|
| Flow rate | 3.0 mL/min |
| Column Temp | 35° C. |
| Back pressure | 100 Bar |
| UV | 220 nm |

Chiral SFC method 3:

| Instrument | SHIMADZU LC-30AD | | |
|---|---|---|---|
| column | Chiralpak IK-3 50 × 4.6 mm I.D., particle size 3 μm | | |
| Mobile Phase | Phase A for CO₂ | Phase B for MeOH + ACN 2:1 (0.05% DEA) | |
| | Time(min) | A(%) | B(%) |
| Gradient | 0.00 | 40 | 60 |
| | 3.00 | 40 | 60 |

| | |
|---|---|
| Flow rate | 3.0 mL/min |
| Column Temp | 35° C. |
| Back pressure | 100 Bar |
| UV | 220 nm |

Chiral SFC method 4:

| Instrument | SHIMADZU LC-30AD | | |
|---|---|---|---|
| column | Chiralpak IK-3 50 × 4.6 mm I.D., particle size 3 μm | | |
| Mobile Phase | Phase A for CO₂ | Phase B for EtOH + ACN 2:1 (0.05% DEA) | |
| | Time(min) | A(%) | B(%) |
| Gradient | 0.00 | 40 | 60 |
| | 3.00 | 40 | 60 |

| | |
|---|---|
| Flow rate | 3.0 mL/min |
| Column Temp | 35° C. |
| Back pressure | 100 Bar |
| UV | 220 nm |

Chiral HPLC method 5:

| Instrument | Agilent 1260 | | |
|---|---|---|---|
| Column | Chiralpak IC-3 50 × 4.6 mm I.D., particle size 3 μm | | |
| Mobile Phase | Phase A for Hexane (0.05% DEA) | Phase B for EtOH (0.05% DEA) | |
| | Time(min) | A(%) | B(%) |
| Gradient | 0.00 | 35 | 65 |
| | 15.0 | 35 | 65 |

| | |
|---|---|
| Flow rate | 1.0 mL/min |
| Column Temp | 35° C. |
| UV | 220 nm |

Chiral SFC method 6:

| Instrument | SHIMADZU LC-30AD | | |
|---|---|---|---|
| column | (S, S)Whelk-O1 50*4.6 mm I.D., 3.5 μm | | |
| Mobile Phase | Phase A for CO₂ | Phase B for AtOH + ACN 2:1 (0.05% DEA) | |
| | Time (min) | A(%) | B(%) |
| Gradient | 0.00 | 80 | 20 |
| | 1.80 | 40 | 60 |
| | 2.70 | 40 | 60 |
| | 2.71 | 80 | 20 |
| | 3.00 | 80 | 20 |

| | |
|---|---|
| Flow rate | 3.0 mL/min |
| Column Temp | 35° C. |
| Back pressure | 100 bar |
| UV | 220 nm |

Chiral SFC method 7:

| Instrument | SHIMADZU LC-30ADsf | | |
|---|---|---|---|
| column | Chiralcel OD-3 50 × 4.6 mm I.D., particle size 3 μm | | |
| Mobile Phase | Phase A for CO₂ | Phase B for ethanol | |
| | | (0.05% DEA) | |
| | Time (min) | A(%) | B(%) |
| Gradient | 0.00 | 95 | 5 |
| | 1.80 | 60 | 50 |
| | 2.70 | 60 | 50 |
| | 2.71 | 95 | 5 |
| | 3.00 | 95 | 5 |

| | |
|---|---|
| Flow rate | 3.0 mL/min |
| Column Temp | 35° C. |
| Back pressure | 10 MPa |
| UV | 220 nm |

Chiral SFC method 8:

| Instrument | SHIMADZU LC-30AD | | |
|---|---|---|---|
| Column | Chiralcel OD-3 50 × 4.6 mm I.D., particle size 3 μm | | |
| Mobile Phase | Phase A for CO₂ | Phase B for IPA + ACN | |
| | | (0.05% DEA) | |
| | Time(min) | A(%) | B(%) |
| Gradient | 0.00 | 95 | 10 |
| | 1.80 | 40 | 60 |
| | 2.70 | 40 | 60 |
| | 2.71 | 90 | 10 |
| | 3.00 | 90 | 10 |

| | |
|---|---|
| Flow rate | 4.0 mL/min |
| Column Temp | 35° C. |
| Back pressure | 100 Bar |
| UV | 220 nm |

Bovine skin gelatin (BSG), dimethyl sulfoxide (DMSO), Pluronic F-127 and tris(2-carboxyethyl)phosphine hydrochloride solution (TCEP) were purchased from Sigma-Aldrich (St. Louis, MO) at the highest level of purity possible. Bicine buffer solution was purchased from Alfa Aesar (Tewksbury, MA) and compound NSC-617145 was purchased from Tocris (Minneapolis, MN). DNA duplex was synthesized at BGI (Shenzhen, China) and was composed of strand 1 with the sequence 5′-GCACTGGCCGTCGTTTTACGGTCG-3′ (SEQ ID NO.: 1) and strand 2 with the sequence 5′-TCCAAGTAAAACGACGGCCAGTGC-3′ (SEQ ID NO.: 2). DNA strands were annealed by heating to 95° C. for 5 minutes followed by slow cooling to room temperature. Compounds in 100% DMSO (0.1 μl) were spotted into a 384-well white polystyrene Optiplate-384 (Perkin Elmer; Waltham, MA) assay plate using a LabCyte Echo 550 (Agilent; Santa Clara, CA). DMSO (0.1 μl) was added to columns 12, rows A-H and column 24, rows I-P for the maximum signal control. Compound NSC-617145 (0.1 μl) was added to columns 12, rows I-P and 24, rows A-H for the minimum signal control (100% inhibition). Compounds/DMSO were preincubated for 15 minutes at 25° C. with 5 μl 2×WRN (BV08), prepared as described below, in assay buffer containing 20 mM Bicine (pH=7.5), 1 mM MgCl₂, 10 mM KCl, 0.1% Pluronic F-127, 0.005% BSG, 1 mM TCEP. The reaction was initiated by the addition of 5 μl 2× substrate mixture in assay buffer and incubated for 60 minutes at 25° C. The final concentrations of the assay components were 0.15 nM WRN, 5 μM ATP, and 0.1 nM DNA duplex. The final DMSO concentration was 1% and the reference compound concentration (NSC-617145) used for the minimal signal control was 20 μM. The reaction was stopped by the addition of the ADP-Glo Kit components (Promega; Madison, WI) as directed and the relative luminescence units (RLU) were read on an Envision 2104 (Perkin Elmer; Waltham, MA). % inhibition calculation:

$$\% INH = ( RLU MAX - RLU sample ) / ( RLU MAX - RLU MIN ) ) \times 100$$

Where RLU=relative luminescence units, sample=signal in sample well, and MIN and MAX are the respective minimum and maximum signal controls.

### Four-Parameter IC₅₀ Fit Equation:

$$Y = Bottom + ( Top - Bottom ) / ( 1 + ( IC 50 / X ) \hat{} Hill Slope )$$

Where top and bottom are normally allowed to float but may be fixed at 100 or 0 respectively in a 3-parameter fit. Y is the % inhibition and X is the compound concentration.

### Example 3: WRN Protein Production

Molecular Biology and virus production. The DNA encoding human Werner helicase (Uniprot Q14191, amino acids 517-1235 with L1074F point mutation) was generated with codon-optimization for E. coli expression and subcloned into the pFastBac vector with a TEV cleavable 8×His tag (SEQ ID NO: 5) (WRN-BV08). The baculovirus from the expression plasmid WRN-BV08 was generated from transfection and amplification following the manufacturer's instructions.

Gene sequence of WRN-BV08
[pFastBac1-WRN-(517-1235 L1074F)-TEV-8His]
(SEQ ID NO.: 3)
ATGAACGAGGGCGAAGAAGACGACGACAAGGACTTCCTGTGGCCT
GCCCCTAACGAAGAACAAGTGACATGCCTGAAGATGTACTTCGGA
CACAGTAGCTTCAAGCCTGTGCAATGGAAGGTCATCCACTCCGTG
CTGGAAGAAAGAAGGGACAACGTGGCTGTGATGGCTACCGGATAC
GGTAAGTCCCTGTGCTTCCAGTACCCTCCCGTGTACGTGGGCAAG
ATCGGTCTGGTGATCTCCCCTCTGATCTCTCTGATGGAGGACCAG
GTGCTGCAATTGAAGATGTCCAACATCCCCGCTTGCTTCCTGGGT
TCCGCTCAAAGTGAGAACGTGCTGACAGACATCAAGCTGGGCAAG
TACCGCATCGTGTACGTGACCCCTGAGTACTGCTCCGGTAACATG
GGTCTGCTGCAACAGCTGGAGGCTGACATCGGAATCACCCTGATC
GCTGTGGACGAGGCTCACTGCATCTCCGAGTGGGGACACGACTTC
CGCGACTCCTTCCGTAAGCTGGGATCCTTGAAGACCGCTCTCCCT
ATGGTGCCTATCGTGGCCCTGACCGCCACTGCTTCCTCCTCCATC
CGCGAGGACATCGTGCGTTGCCTGAACCTGCGCAACCCTCAGATC
ACTTGCACCGGTTTCGACCGCCCTAACTTGTACCTCGAGGTGCGT
CGCAAGACCGGTAACATCCTCCAGGACCTGCAGCCTTTCCTGGTC
AAGACCTCCTCCCACTGGGAATTTGAGGGCCCTACCATCATCTAC
TGCCCTTCCCGCAAGATGACCCAGCAAGTCACCGGCGAGCTGCGC
AAGCTCAACCTCTCCTGCGGTACCTACCACGCTGGTATGTCCTTC
TCCACCCGCAAGGACATCCACCACCGCTTCGTCCGTGACGAAATC
CAATGCGTCATCGCTACCATCGCTTTCGGAATGGGCATCAACAAG
GCTGACATCCGCCAGGTGATCCACTACGGCGCCCCCAAGGACATG
GAATCCTACTACCAGGAAATCGGTCGCGCCGGTCGCGACGGTCTG
CAGTCTTCCTGTCACGTGCTGTGGGCCCCCGCTGACATCAACCTG
AACCGCCACCTGCTGACCGAAATCCGCAACGAGAAGTTCCGCCTG
TACAAGCTCAAGATGATGGCTAAGATGGAGAAGTACCTGCACTCC
TCCCGCTGTCGCCGTCAGATCATCCTCTCCCACTTCGAGGACAAG
CAAGTGCAAAAGGCTAGCCTGGGTATCATGGGCACCGAAAAGTGT
TGTGACAACTGCCGCTCCCGCCTCGACCACTGCTACTCCATGGAC
GACAGCGAGGACACCTCCTGGGACTTCGGTCCTCAAGCTTTCAAG
CTCTTGTCCGCTGTGGACATCCTGGGCGAGAAGTTCGGTATCGGT
CTCCCCATCCTCTTCCTGCGTGGTAGCAACTCCCAACGCCTGGCT
GACCAGTACCGCCGCCACTCCCTCTTCGGTACCGGTAAGGACCAG
ACCGAGTCCTGGTGGAAGGCTTTCTCTCGCCAACTGATCACCGAA
GGTTTCCTGGTGGAGGTGTCCCGCTACAACAAGTTCATGAAGATC
TGCGCTCTCACTAAGAAGGGAAGGAACTGGCTGCACAAGGCTAAC
ACTGAGTCCCAATCCCTCATCCTGCAGGCTAACGAGGAGCTGTGC
CCTAAGAAGTTCCTGCTGCCTTCCTCCAAGACCGTGTCCTCCGGA
ACAAAGGAACACTGCTACAACCAAGTCCCTGTGGAGCTCTCCACC
GAGAAGAAGTCCAACCTGGAGAAGCTGTACAGCTACAAGCCTTGC
GACAAGATCAGCTCCGGTTCCAACATCAGCAAGAAGTCCATCATG
GTGCAATCCCCTGAAAAGGCCTACTCCAGCTCCCAACCTGTCATC
TCCGCTCAAGAGCAAGAGACCCAGATCGTGCTGTACGGTAAGCTG
GTCGAAGCCCGCCAAAAGCACGCTAACAAGATGGACGTCCCTCCC
GCTATCCTCGCCACCAACAAGATCCTCGTGGATATGGCTAAGATG
CGCCCCACCACCGTCGAGAACGTGAAGCGCATCGACGGTGTCTCC
GAGGGTAAGGCCGCTATGCTGGCTCCTCTGCTGGAAGTGATCAAG
CACTTCTGCCAGACCAACTCCGTGCAGACCGACCTGTTCAGTAGT
GAGAACCTGTACTTCCAAGGCCACCATCATCATCATCATCACCAC
TAA
Protein sequence of WRN-BV08
[pFastBac1-WRN-(517-1235 L1074F)-TEV-8His]
(SEQ ID NO.: 4)
MNEGEEDDDKDFLWPAPNEEQVTCLKMYFGHSSFKPVQWKVIHSV
LEERRDNVAVMATGYGKSLCFQYPPVYVGKIGL VISPLISLMED
QVLQLKMSNIPACFLGSAQSENVLTDIKLGKYRIVYVTPEYCSGN
MGLLQQLEADIGITLIAVDEAHCISEWGHDFRDSFRKLGSLKTAL
PMVPIVALTATASSSIREDIVRCLNLRNPQITCTGFDRPNLYLEV
RRKTGNILQDLQPFLVKTSSHWEFEGPTIIYCPSRKMTQQVTGEL
RKLNLSCGTYHAGMSFSTRKDIHHRFVRDEIQCVIATIAFGMGIN
KADIRQVIHYGAPKDMESYYQEIGRAGRDGLQSSCHVLWAPADIN
LNRHLLTEIRNEKFRLYKLKMMAKMEKYLHSSRCRRQIILSHFED
KQVQKASLGIMGTEKCCDNCRSRLDHCYSMDDSEDTSWDFGPQAF
KLLSAVDILGEKFGIGLPILFLRGSNSQRLADQYRRHSLFGTGKD
QTESWWKAFSRQLITEGFLVEVSRYNKFMKICALTKKGRNWLHKA
NTESQSLILQANEELCPKKFLLPSSKTVSSGTKEHCYNQVPVELS
TEKKSNLEKLYSYKPCDKISSGSNISKKSIMVQSPEKAYSSSQPV
ISAQEQETQIVLYGKLVEARQKHANKMDVPPAILATNKILVDMAK
MRPTTVENVKRIDGVSEGKAAMLAPLLEVIKHFCQTNSVQTDLFS
SENLYFQGHHHHHHHH

Sf9 cells grown in SF900II media were infected with 1:200 WRN-BV08 P2 virus and incubated for protein expression for 72 h at 27° C. The WRN protein was purified using the following protocol. The cell pellets were thawed and resuspended in buffer A (50 mM Tris, pH 7.5, 500 mM NaCl, 1 mM TCEP, 10% Glycerol) supplemented with 0.5% CHAPS, 1 mM PMSF, 1p g/ml Leupeptin, 1 μg/ml Pepstatin, and the Pierce Universal Nuclease and cocktail tablet. Cleared lysates were loaded onto a Ni Sepharose™ excel column and washed with buffer A and bound protein was eluted with buffer A supplemented with 300 mM imidazole. The eluted protein was dialyzed against buffer A and digested by His-tagged TEV (1:5 ratio) overnight at 4° C. ZnCl₂ was added into the sample at final 15 μM before loading onto a second Ni Sepharose™ excel column. Untagged WRN protein was eluted from the column with buffer A supplemented with 20 mM imidazole, dialyzed overnight into buffer B (50 mM Tris, pH 7.5, 1 mM TCEP, 10% Glycerol) supplemented with 150 mM NaCl and loaded onto a Heparin column. Proteins were eluted with a step gradient of buffer B supplemented with 150 mM, 200 mM, 300 mM and 500 mM NaCl. WRN containing fractions were pooled and concentrated prior to loading on to size exclusion chromatography using a HiLoad 16/600 Superdex™ 200 pg column (GE Healthcare) in buffer C (20 mM HEPES, pH 7.5, 250 mM NaCl, 0.25 mM TCEP, 2.5% Glycerol).

The resultant IC₅₀ results obtained for the tested compounds are shown below in Table 3. Compounds with an IC₅₀ less than or equal to 0.005 μM are designated as “A.” Compounds with an IC₅₀ greater than 0.005 μM and less than or equal to 0.05 μM are designated as “B.” Compounds with an IC₅₀ greater than 0.05 μM and less than or equal to 0.1 μM are designated as “C.” Compounds with an IC₅₀ greater than 0.1 μM or equal to 0.5 μM are designated as “D.” Compounds with an IC₅₀ greater than 0.5 μM are designated as “E.” and “-” (not tested).

TABLE 3

| Cmpd. No. | ADP-Glo_hWRN_IC50 [μM] |
|---|---|
| I-1 | B |
| I-2 | B |
| I-3 | C |
| I-4 | D |
| I-5 | A |
| I-6 | A |
| I-7 | A |
| I-8 | D |
| I-9 | D |
| I-10 | D |
| I-11 | A |
| I-12 | E |
| I-13 | A |
| I-14 | B |
| I-15 | A |
| I-16 | D |
| I-17 | A |
| I-18 | A |
| I-19 | C |
| I-20 | E |
| I-21 | A |
| I-22 | D |
| I-23 | A |
| I-24 | E |
| I-25 | E |
| I-26 | — |
| I-27 | A |
| I-28 | B |

### Example 4: Method for Determining Effect on p21 Induction in Cells

The colon carcinoma cell line HCT 116 was obtained from ATCC and cultured in growth medium consisting of Mccoy's 5A Medium (Gibco 16600108) supplemented with 1000 FBS (Transgene FS201-02) and 100 units/mL penicillin-streptomycin (Gibco 15140122) and maintained at 37° C. under 500 CO₂. On the day of seeding, 2,000 cells in 30 μL of culture media were plated per well to Poly-D-Lysine 384 Well Black Clear Plates (Biocoat 356663) and incubated overnight at 37° C. under 5% CO₂. The following day, compounds were serially diluted in DMSO for a total of 11 test concentrations. The typical starting concentration of cpds was 10 μM with 2-fold dilutions. Next, 150 nL of diluted compound was added in duplicate to the assay plate, using an Echo 655 (Labcyte). The plate was centrifuged at 500 RPM for 1 min and then incubated at 37° C. under 5% CO₂ for 24 h. After 24 h, medium was removed, and cells were fixed by adding 40 L of 4% paraformaldehyde solution to each well and incubated for 20 min at room temperature. The plate was then washed 4 times with 100 μL per well of wash buffer (PBS with 0.1% Tween-20) using a microplate washer. Next, 30 μL of ice-cold methanol was added to each well and the plate was incubated at −20° C. for 10 min. The plate was washed 4 times with 100 μL per well of wash buffer by a microplate washer, then 30 μL per well of blocking buffer (Intercept PBS blocking buffer (LI-COR 927-70001) with 0.05% Tween-20) was added and the plate was incubated at room temperature with shaking for 2 h. Next, to each test well, 20 μL of primary antibody solution (p21 Waf1/CIP (12D1) Rabbit mAb (Cell Signaling Technologies 2947) diluted 1:1000 and GAPDH (D4C₆R) Mouse mAb (Cell Signaling Technologies 97166) diluted 1:2000 in blocking buffer) was added and the plate was placed at 4° C., overnight. The following day, the plate was washed 5 times with 100 μL per well of wash buffer using a microplate washer for 5 min. 20 μL per well of secondary antibody (IRDye 680CW Goat anti-Mouse IgG (H+L) (LI-COR 926-68070) diluted 1: 2000 in Blocking Buffer and IRDye 800CW Goat anti-Rabbit IgG (H+L) (LI-COR 926-32211) diluted 1: 2000 in Blocking Buffer) was then added and the plate was stored for 2 h in the dark at room temperature with shaking. The plate was then washed 4 times with 100 μL per well of wash buffer again using a microplate washer. Finally, the p21 signal and the GAPDH signal were quantified using a LI-COR Odyssey CLx Imager machine reading at 800 nm and 700 nm, respectively. Each plate contained DMSO control (low control) and an internal reference WRN inhibitor (high control) respectively. For quantitation, the 800 nm/700 nm ratio was calculated for each well to give fold p21 induction and then percent activation for each compound well was calculated as follows (100×(ratio cpd well-ratio low control)/(ratio high control−ratio low control)). EC50 values for each compound were generated after non-linear regression curve fitting using commercially available software. The resultant EC₅₀ results obtained for the tested compounds are shown below in Table 4. Compounds with an EC₅₀ less than or equal to 0.50 μM are designated as “A.” Compounds with an EC₅₀ greater than 0.50 μM and less than or equal to 2.00 μM are designated as “B.” Compounds with an EC₅₀ greater than 2.00 μM and less than or equal to 5.00 μM are designated as “C.” Compounds with an EC₅₀ greater than 5.00 μM are designated as “D.”

TABLE 4

| Cmpd. No. | P21_EC50 [μM] |
|---|---|
| I-1 | A |
| I-2 | A |
| I-3 | B |
| I-4 | D |
| I-5 | A |
| I-6 | A |
| I-7 | A |
| I-8 | D |
| I-9 | B |
| I-10 | C |
| I-11 | A |
| I-12 | D |
| I-14 | A |
| I-15 | A |
| I-16 | B |
| I-17 | A |
| I-18 | A |
| I-19 | B |
| I-20 | D |
| I-21 | A |
| I-22 | D |
| I-23 | A |
| I-24 | D |
| I-25 | D |
| I-26 | A |
| I-27 | A |
| I-28 | B |

We claim:

1. A compound of Formula I, or a pharmaceutically acceptable salt thereof: 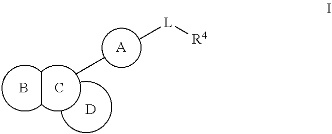
wherein tricyclic Ring BCD is selected from one of the following: 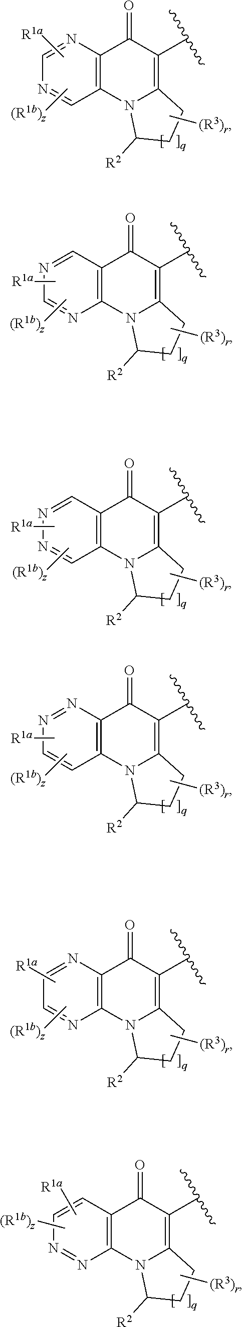 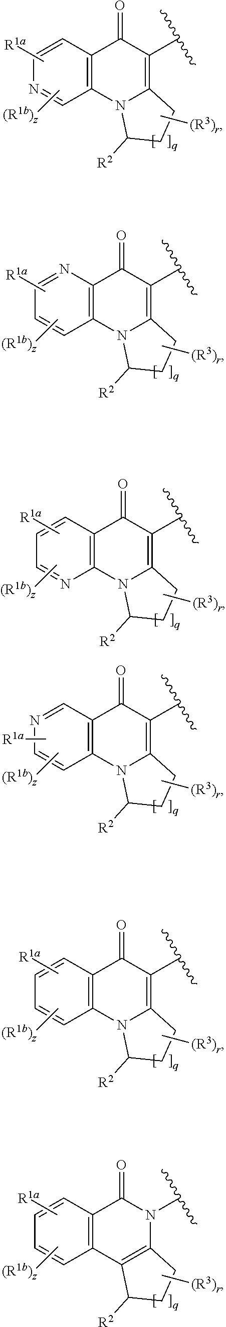 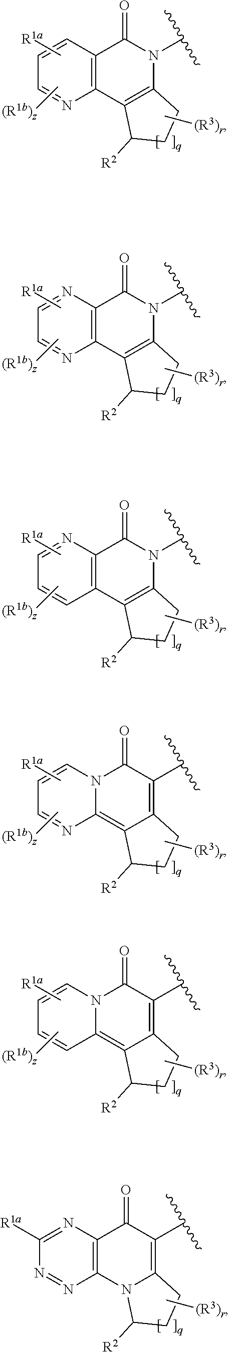 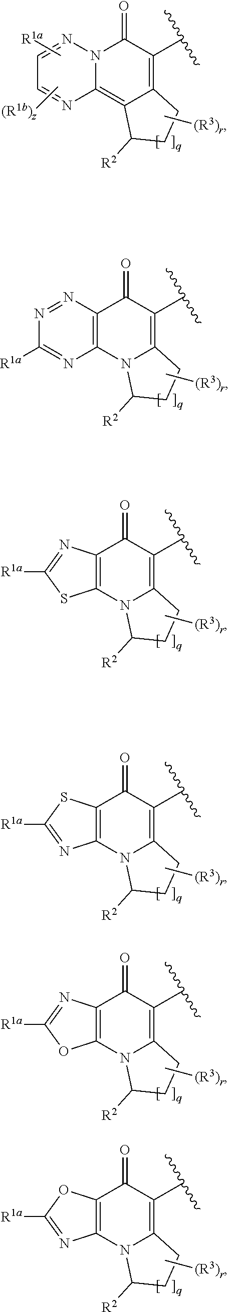 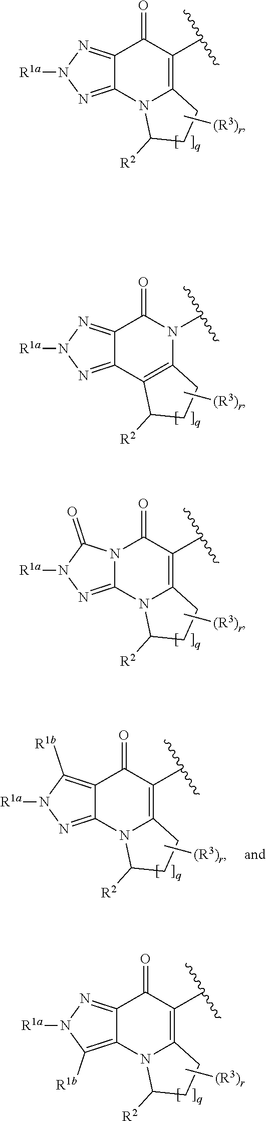
wherein [symbol] denotes the point of attachment to Ring A;
each R^{1b} group is independently selected from H, halogen, CN, OH, C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy, wherein said C₁-C₆aliphatic, C₁-C₆alkoxy, C₃-C₆cycloalkyl, C₁-C₆alkylene-O—C₁-C₆alkyl, haloC₁-C₆alkyl, haloC₁-C₆alkoxy, and C₃-C₆cycloalkoxy are each independently and optionally substituted with 1-5 halogen, OH, CN, C₁-C₆alkyl, or C₃-C₆cycloalkyl groups; wherein z is 0, 1, or 2;
Ring A is:
a) a 4-7 membered saturated or partially unsaturated bivalent monocyclic carbocyclyl or 4-7 membered saturated or partially unsaturated bivalent heterocyclyl ring (having 1-4 heteroatoms independently selected from nitrogen, oxygen, and sulfur); or
b) a 4-12 membered saturated or partially unsaturated bivalent bicyclic ring system that is fused, bridged, or spirocyclic selected from carbocyclyl or heterocyclyl (having 1-4 heteroatoms independently selected from nitrogen, oxygen, and sulfur);
wherein Ring A is substituted with 0-4 independently selected R^{B} substituents;
-L- is a linker selected from —C(O)—, —S(O)—, —S(O)₂—, and 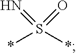
R^{1a} is selected from:
a) a 5-6 membered heteroaryl (having 1-4 heteroatoms independently selected from nitrogen, oxygen, and sulfur) optionally substituted with 1-3 groups independently selected from halogen, C₁-C₆aliphatic, C₃-C₆cycloalkyl, haloC₁-C₆alkyl, C₁-C₆alkoxy, and C₃-C₆cycloalkoxy, wherein said 5-6 membered heteroaryl is further substituted with 0-3 independently selected R^{B};
b) a 4-7 membered saturated or partially unsaturated heterocyclyl (having 1-2 heteroatoms independently selected from nitrogen, oxygen, and sulfur), optionally substituted with 1 or 2 groups independently selected from C₁-C₆aliphatic, C₃-C₆cycloalkyl, C₁-C₆alkoxy, C₃-C₆cycloalkoxy, and —OR, wherein said 4-7 membered saturated or partially unsaturated heterocyclyl is further substituted with 0-3 independently selected R^{B}, and two R^{B} along with their intervening atoms optionally join to form a 3-5 membered carbocyclyl;
c) a 4-12 membered saturated or partially unsaturated bicyclic ring system that is fused, bridged, or spirocyclic selected from carbocyclyl or heterocyclyl (having 1-4 heteroatoms independently selected from nitrogen, oxygen, and sulfur), wherein said carbocyclyl or heterocyclyl is substituted with 0-3 independently selected R^{B}; and
d) H, halogen, C₁-C₆aliphatic, C₃-C₇cycloalkyl, C₁-C₆alkylene-O—C₁-C₆alkyl, CN, —OR, —OR¹⁰, —NR¹⁰R¹¹, —C(O)NR¹⁰R¹¹, —CH₂NR¹⁰R¹¹, or —SO₂R¹², wherein said C₁-C₆aliphatic, C₃-C₇cycloalkyl, or C₁-C₆alkylene-O—C₁-C₆alkyl is substituted with 0-5 independently selected R^{B};
or R^{1a} and one R^{1b} on adjacent atoms of Ring B, taken together with the adjacent Ring B atoms to which they are attached, form a cyclic group fused to Ring B selected from phenyl, a 5-6 membered heteroaryl (having 1-3 heteroatoms independently selected from nitrogen, oxygen and sulfur), a 4-7 membered saturated or partially unsaturated carbocyclyl, or a 4-7 membered saturated or partially unsaturated heterocyclyl (having 1-3 heteroatoms independently selected from nitrogen, oxygen and sulfur), wherein said cyclic group fused to Ring B is substituted with 0-3 independently selected R^{B};
R² is selected from C(O)N(R)R^{2A};
R^{2A} is phenyl, pyridyl, cubanyl, a saturated or partially unsaturated 4-8 membered monocyclic ring, a saturated or partially unsaturated bridged, fused, or spirocyclic 5-, 6-, 7-, 8-, 9-, 10-, 11-, or 12-membered ring, wherein said saturated or partially unsaturated monocyclic ring, or saturated or partially unsaturated bridged, fused, or spirocyclic ring contains 0, 1, 2, 3, or 4 heteroatoms independently selected from nitrogen, oxygen, and sulfur; and wherein said phenyl, pyridyl, cubanyl, saturated or partially unsaturated monocyclic ring, or saturated or partially unsaturated bridged, fused, or spirocyclic ring are each optionally substituted with 1, 2, or 3 substituents independently selected from halogen, C₁-C₄aliphatic, haloC₁-C₄alkyl, C₃-C₆cycloalkyl, haloC₃-C₆cycloalkyl, —OH, —CN, C₁-C₄alkoxy, haloC₁-C₄alkoxy, C₃-C₆-cycloalkoxy, haloC₃-C₆cycloalkoxy and —SF₅, and wherein two substituents on adjacent atoms of the phenyl or pyridyl, together with said adjacent atoms, optionally form a 4-7 membered carbocyclyl fused to the phenyl or pyridyl, and wherein two substituents on adjacent atoms of the phenyl or pyridyl together with said adjacent atoms optionally form a 4-7 membered heterocyclyl (having 1-4 heteroatoms independently selected from nitrogen, oxygen, and sulfur) fused to the phenyl or pyridyl, wherein said fused 4-7 membered carbocyclyl or fused 4-7 membered heterocyclyl is substituted with 0-5 independently selected halogen or methyl; or
R^{2A} is 2-benzimidazolyl, 2-naphthyl, or 3-quinolinyl, each of which is optionally substituted with 1, 2, or 3 substituents independently selected from halogen, C₁₋₄alkyl, and —OH;
each R³ is independently selected from:
hydrogen, halo, and OH;
C₁-C₄aliphatic unsubstituted or substituted by 1, 2 or 3 substituents independently selected from halo and OH; and
C₃-C₅cycloalkyl, C₁-C₄alkoxy, —NHR^{3A}, —N(R^{3A})₂, or C₁-C₄alkylthio, each of which, besides hydrogen, is optionally substituted with —OH, 1-5 independently selected halogen, OR, —C(O)NR¹⁰R¹¹, or N(R)C(O)R; wherein each R^{3A} is independently selected from C₁-C₄alkyl;
or two R³ substituents on the same ring carbon atom may join, together with the carbon atom to which they are attached, to form a cyclopropyl ring;
R⁴ is phenyl or a first 5-6 membered heteroaryl (having 1-4 heteroatoms independently selected from nitrogen, oxygen, and sulfur) wherein said phenyl or first 5-6 membered heteroaryl is substituted with 0-5 R^{B}; and optionally two adjacent atoms of said phenyl or first 5-6 membered heteroaryl have two substituents that together with said adjacent atoms form a cyclic group fused to the phenyl or first 5-6 membered heteroaryl selected from a 4-7 membered carbocyclyl, a 4-7 membered heterocyclyl (having 1-4 heteroatoms independently selected from nitrogen, oxygen, and sulfur), or a second 5-6 membered heteroaryl (having 1-4 heteroatoms independently selected from nitrogen, oxygen, and sulfur); wherein said fused cyclic group is substituted with 0-3 independently selected R^{B}; or
R⁴ is a C₁-C₄aliphatic, C₁-C₄alkoxy, or C₃-C₆cycloalkyl, each of which is substituted with 0-3 groups independently selected from halogen, —CN, —OH, C₁-C₄alkyl, C₁-C₄alkoxy, and optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur;
R¹⁰ is H, C₁-C₆aliphatic, haloC₁-C₆alkyl, C₃-C₆cycloalkyl, haloC₃-C₆cycloalkyl, —C(O)C₁-C₆alkyl, or a 5-6 membered heteroaryl (having 1-4 heteroatoms independently selected from nitrogen, oxygen, and sulfur); each R¹⁰ except H is optionally substituted with 1 or 2 independently selected R^{B};
R¹¹ is H, C₁-C₆aliphatic, or C₃-C₆cycloalkyl, or R¹⁰ and R¹¹ are taken together with the nitrogen atom to which they are attached to form a 5-6 membered ring optionally substituted with 1, 2, or 3 substituents independently selected from halogen, —OH, —CN, C₁-C₄alkoxy, and haloC₁-C₄alkoxy;
R¹² is C₁-C₆aliphatic, C₃-C₆cycloalkyl, or a 5-6 membered heteroaryl (having 1-4 heteroatoms independently selected from nitrogen, oxygen, and sulfur); each R¹² is optionally substituted with 1 or 2 groups independently selected from halogen, C₁-C₆aliphatic, haloC₁-C₆alkyl, C₁-C₆alkoxy, C₃-C₆cycloalkyl, and C₃-C₆cycloalkoxy;
R^{B} is independently selected at each occurrence from the group consisting of optionally substituted phenyl, optionally substituted 5-6 membered heteroaryl (having 1-4 heteroatoms independently selected from nitrogen, oxygen, and sulfur), optionally substituted 4-7 membered saturated or partially unsaturated heterocyclyl (having 1-2 heteroatoms independently selected from nitrogen, oxygen, and sulfur), halogen, optionally substituted C₁-C₆aliphatic, haloC₁-C₆alkyl, C₃-C₆cycloalkyl, haloC₃-C₆cycloalkyl, C₁-C₆alkoxy, haloC₁-C₆alkoxy, C₃-C₆cycloalkoxy, haloC₃-C₆cycloalkoxy, C₁-C₆alkylene-O—C₁-C₆alkyl, —CN, —NO₂, oxo, —OR, —SR, NR₂, S(O)₂R, S(O)₂NR₂, S(O)R, S(O)NR₂, C(O)R, C(O)OR, —C(O)NR₂, C(O)N(R)OR, OC(O)R, OC(O)NR₂, —N(R)C(O)OR, N(R)C(O)R, N(R)C(O)NR₂, N(R)C(NR)NR₂, N(R)S(O)₂NR₂, and —N(R)S(O)₂R;
each R is independently hydrogen, or an optionally substituted C₁₋₆aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form a cyclic group selected from an optionally substituted 4-7 membered saturated ring, a 4-7 membered partially unsaturated ring, or a 5-6 membered heteroaryl ring (having 1-4 heteroatoms independently selected from nitrogen, oxygen, and sulfur); wherein said cyclic group has 0-3 heteroatoms independently selected from nitrogen, oxygen, and sulfur);
q is 1 or 2;
r is 0, 1, 2 or 3.

2. The compound of claim 1 selected from the group consisting of:

| | |
|---|---|
| 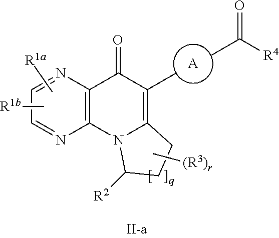 | II-a |
| 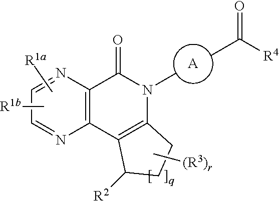 | II-b |
| 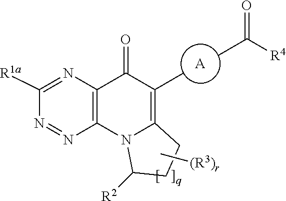 | II-c |
| 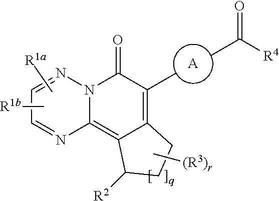 | II-d |
| 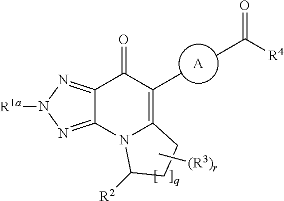 | II-e |
| 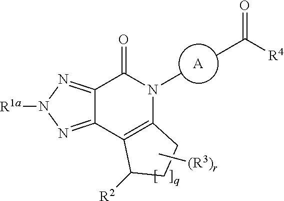 | II-f |
| 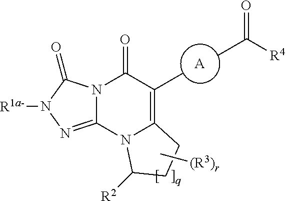 | II-g |

or a pharmaceutically acceptable salt thereof,
wherein R^{1a} is selected from groups a)-d):
a) a 5-6 membered heteroaryl (having 1-4 heteroatoms independently selected from nitrogen, oxygen, and sulfur) optionally substituted with 1 or 2 groups independently selected from C₃-C₆cycloalkyl and C₃-C₆cycloalkoxy, wherein said 5-6 membered heteroaryl is further substituted with 0-3 independently selected R^{B};
b) a 4-6 membered saturated or partially unsaturated heterocyclyl (having 1-2 heteroatoms independently selected from nitrogen, oxygen, and sulfur), said heterocyclyl substituted with 0-2 R^{B} groups independently selected from halogen, oxo, NR₂, optionally substituted C₁-C₄aliphatic, —OR, azetidinyl optionally substituted with 1 or 2 independently selected halogen, and pyrrolidinyl optionally substituted with 1 or 2 independently selected halogen, and two R^{B} along with their intervening atoms optionally join to form a 3-5 membered carbocyclyl;
c) a 6-8 membered saturated or partially unsaturated bridged bicyclic heterocyclyl (having 1-4 heteroatoms independently selected from nitrogen, oxygen, and sulfur), said heterocyclyl substituted with 0-2 R^{B} groups independently selected from halogen, oxo, NR₂, optionally substituted C₁-C₄aliphatic, —OR, azetidinyl optionally substituted with 1 or 2 independently selected halogen, and pyrrolidinyl optionally substituted with 1 or 2 independently selected halogen; and
d) H, halogen, C₁-C₆alkyl, C₂-C₄alkene, C₂-C₄alkyne, CN, —OR¹⁰, —NR¹⁰R¹¹, —C(O)NR¹⁰R¹¹, —CH₂NR¹⁰R¹¹, —SO₂R¹², a 3-7 membered carbocyclyl, wherein said C₁-C₆alkyl, C₂-C₄alkene, C₂-C₄alkyne, or 3-7 membered carbocyclyl may be optionally substituted with 0-3 independently selected R^{B}.

3. The compound of claim 1, or a pharmaceutically acceptable salt thereof, wherein
R⁴ is selected from one of a), b), and c):
a) R⁴ is a Ring E that is selected from the group consisting of: 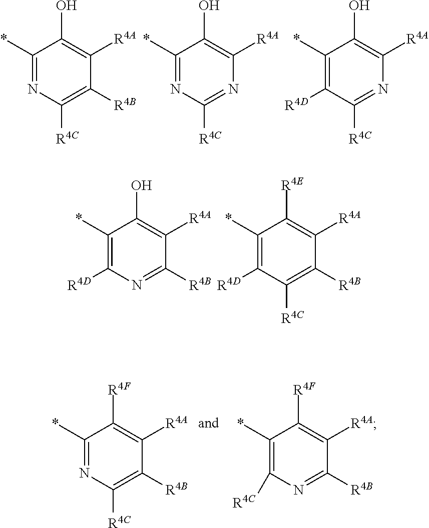
wherein * is a point of attachment to L or —C(O)—;
and:
any substituents that are present on Ring E selected from R^{4A}, R^{4B}, R^{4C}, R^{4D}, R^{4E}, and R^{4F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4B}, R^{4E} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form a 5-6 membered optionally substituted heteroaryl (having 1-4 heteroatoms independently selected from nitrogen, oxygen, and sulfur) substituted with 0-3 independently selected R^{B}; that is fused to Ring E; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form a 5-6 membered heteroaryl (having 1-4 heteroatoms independently selected from nitrogen, oxygen, and sulfur) substituted with 0-3 independently selected R^{B}; that is fused to Ring E; and R^{4B} and R^{4C}are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴;
R¹³ is independently selected at each occurrence from hydrogen and C₁-C₄alkyl optionally substituted with —OH, —OCH₃, or —OCH₂CH₃; and
R¹⁴ is independently selected at each occurrence from hydrogen and C₁-C₄alkyl optionally substituted with —OH, —OCH₃, or —OCH₂CH₃, or R¹³ and R¹⁴ combine with the nitrogen atom to which they are attached to form a heterocyclic ring selected from azetidinyl, pyrrolidinyl, or piperidinyl, said heterocyclic ring optionally substituted with —CH₃;
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; and
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur.

4. The compound of claim 1, or a pharmaceutically acceptable salt thereof, wherein Ring A is selected from 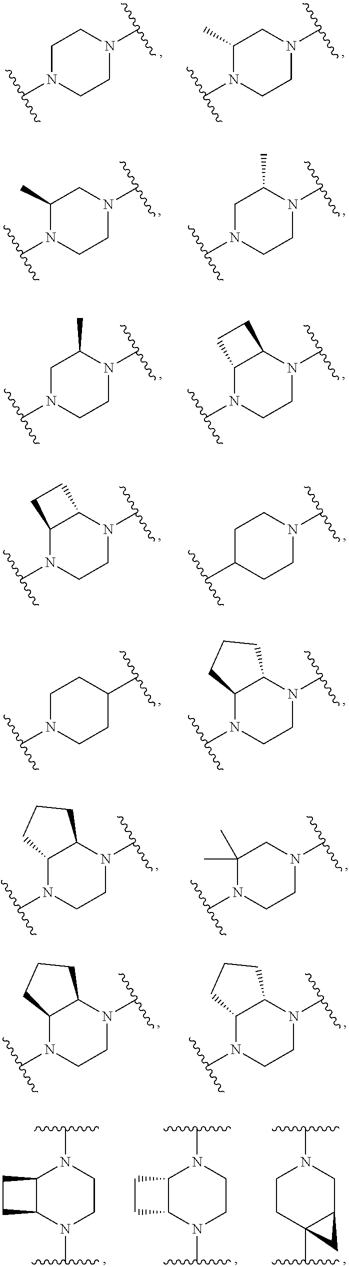 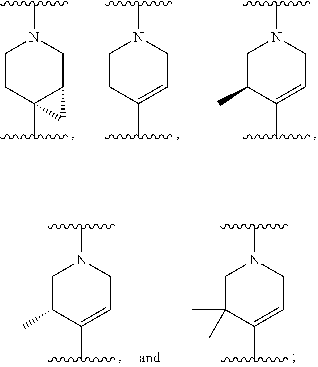
wherein Ring A is substituted with 0-4 independently selected R^{B} substituents.

5. The compound of claim 1 of formula: 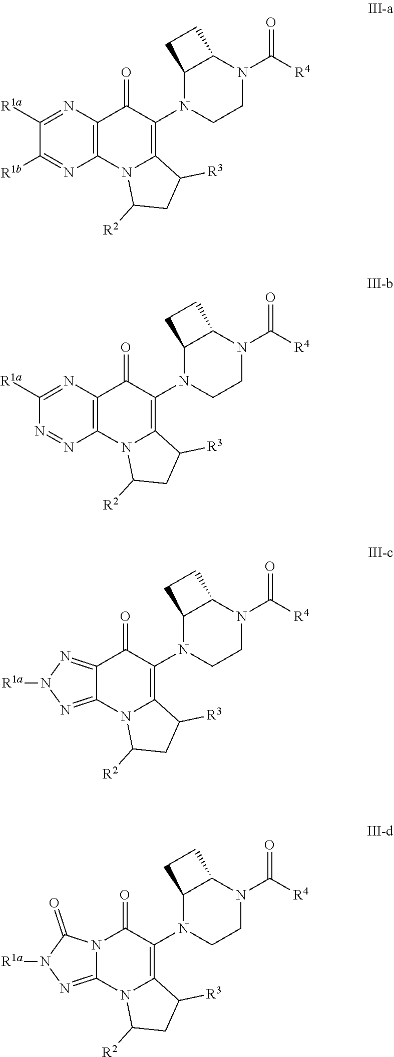
or a pharmaceutically acceptable salt thereof.

6. The compound of claim 5, or a pharmaceutically acceptable salt thereof, wherein
R⁴ is selected from one of a), b), and c):
a) R⁴ is a Ring E that is selected from the group consisting of: 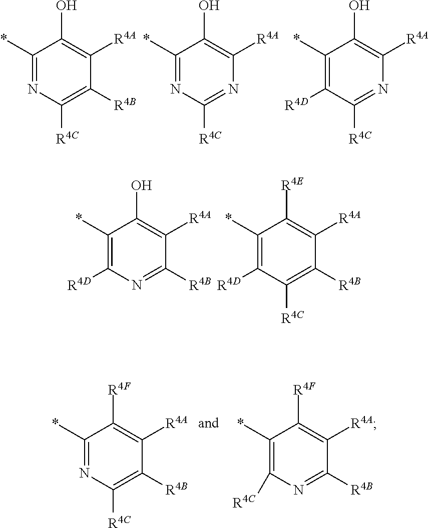
wherein * is a point of attachment to L; and
any substituents that are present on Ring E selected from R^{4A}, R^{4B}, R^{4C}, R^{4D}, R^{4E} and R^{4F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4A} and R^{4B}, along with their intervening atoms, join to form 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4B} and R^{4C}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4C} and R^{4D}, along with their intervening atoms, join to form a 4-7 membered carbocyclyl substituted with 0-3 independently selected R^{B}, a 4-7 membered heterocyclyl substituted with 0-3 independently selected R^{B}, or a 5-6 membered heteroaryl (having 1-4 heteroatoms independently selected from nitrogen, oxygen, and sulfur) substituted with 0-3 independently selected R^{B}; that is fused to Ring E; and any substituents that are present on Ring E selected from R^{4A}, R^{4B}, R^{4E} and R^{4F}are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4E} and R^{4A}, along with their intervening atoms, join to form a 5-6 membered optionally substituted heteroaryl (having 1-4 heteroatoms independently selected from nitrogen, oxygen, and sulfur) substituted with 0-3 independently selected R^{B}; that is fused to Ring E; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; or
R^{4F} and R^{4A}, along with their intervening atoms, join to form a 5-6 membered heteroaryl (having 1-4 heteroatoms independently selected from nitrogen, oxygen, and sulfur) substituted with 0-3 independently selected R^{B}; that is fused to Ring E; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen and C₁-C₄alkyl optionally substituted with —OH, —OCH₃, or —OCH₂CH₃; and
R¹⁴ is independently selected at each occurrence from hydrogen and C₁-C₄alkyl optionally substituted with —OH, —OCH₃, or —OCH₂CH₃, or R¹³ and R¹⁴ combine with the nitrogen atom to which they are attached to form a heterocyclic ring selected from azetidinyl, pyrrolidinyl, or piperidinyl, said heterocyclic ring optionally substituted with —CH₃;
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 groups independently selected from halogen, —OH, —CN, C₁-C₄alkyl, haloC₁-C₄alkyl, C₃-C₆cycloalkyl, and C₁-C₄alkoxy; and
c) R⁴ is a C₁-C₄alkyl, C₁-C₄alkoxy, or C₃-C₆cycloalkyl, each of which is substituted with 0-3 groups independently selected from halogen, —CN, —OH, C₁-C₄alkyl, C₁-C₄alkoxy, optionally substituted 5-6 membered heterocyclyl having 1-3 heteroatoms independently selected from nitrogen, oxygen, and sulfur, and optionally substituted 5-6 membered heterocyclyloxy having 1-3 heteroatoms independently selected from nitrogen, oxygen, and sulfur.

7. The compound of claim 6, wherein R^{1a} is a 5-6 membered heteroaryl (having 1-4 heteroatoms independently selected from nitrogen, oxygen, and sulfur) optionally substituted with 1-3 groups selected from halogen, C₁-C₆alkyl, haloC₁-C₆alkyl, C₁-C₆alkoxy and C₃-C₆cycloalkyl, wherein said 5-6 membered heteroaryl is further substituted with 0-3 independently selected R^{B} and R^{1b} is selected from H, halogen, C₁-C₆alkyl, haloC₁-C₆alkyl, C₁-C₆alkoxy, and haloC₁-C₆alkoxy.

8. The compound of claim 6, wherein R^{1a} is a 4-6 membered saturated or partially unsaturated heterocyclyl (having 1-2 heteroatoms independently selected from nitrogen, oxygen, and sulfur), said heterocyclyl substituted with 0-2 R^{B} groups independently selected from halogen, oxo, NR₂, optionally substituted C₁-C₄aliphatic, —OR, azetidinyl optionally substituted with 1 or 2 independently selected halogen, and pyrrolidinyl optionally substituted with 1 or 2 independently selected halogen, and two R^{B} along with their intervening atoms optionally join to form a 3-5 membered carbocyclyl; and each R^{1b} is independently selected from H, halogen, C₁-C₆alkyl, haloC₁-C₆alkyl, C₁-C₆alkoxy, and haloC₁-C₆alkoxy.

9. The compound of claim 6, wherein R^{1a} is halogen, C₁-C₆alkyl, C₂-C₄alkene, C₂-C₄alkyne, CN, —OR¹⁰, —NR¹⁰R¹¹, —C(O)NR¹⁰R¹¹, —CH₂NR¹⁰R¹¹, —SO₂R¹², a C₃-C₇cycloalkyl, wherein said C₁-C₆alkyl, C₂-C₄alkene, C₂-C₄alkyne, and C₃-C₇cycloalkyl is substituted with 0-3 R^{B} independently selected from halogen, C₃-C₆cycloalkyl, haloC₃-C₆cycloalkyl, —OH, —CN, C₁-C₄alkoxy, and haloC₁-C₄alkoxy; and R^{1b} is selected from H, halogen, C₁-C₆alkyl, haloC₁-C₆alkyl, C₁-C₆alkoxy, and haloC₁-C₆alkoxy.

10-12. (canceled)

13. The compound of claim 1, wherein R^{1a} is selected from the group consisting of:

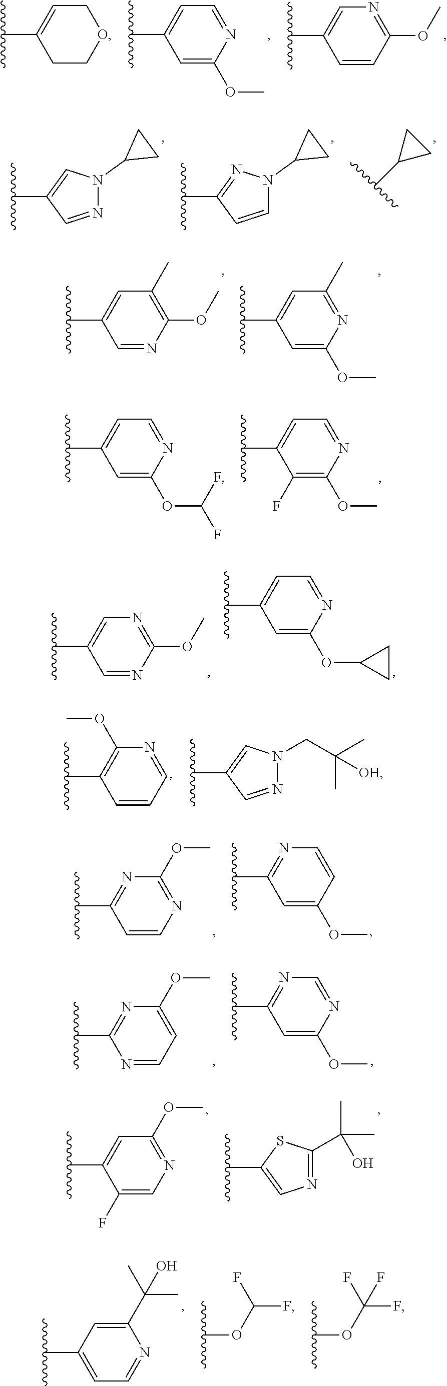

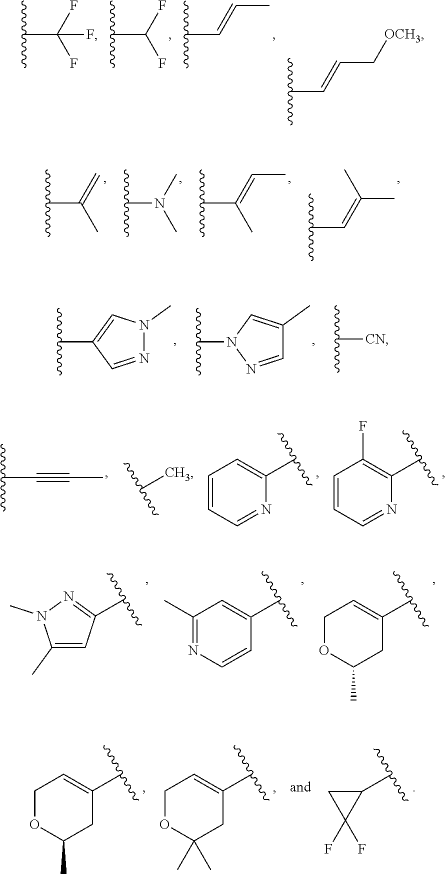

14. The compound of claim 1, wherein R⁴ is Ring E of the following structure: 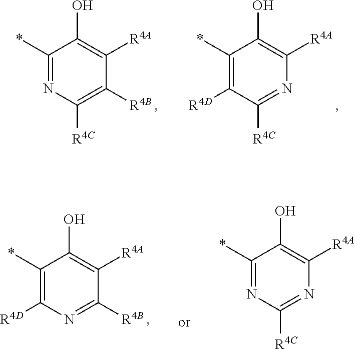
wherein * is a point of attachment to L or —C(O)—;
R^{4A} is hydrogen, —CH₃, —CH₂CH₃, —F, —CF₂H, —CF₃, —OCH₃, —OCF₃, —OCH₂CH₃, or —OCHF₂;
R^{4B}, R^{4C} and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR¹³R¹⁴; and
R¹³ is independently selected at each occurrence from hydrogen or C₁-C₄alkyl optionally substituted with —OH, —OCH₃, or —OCH₂CH₃, and R¹⁴ is H; or NR¹³R¹⁴, taken in combination form a heterocyclic ring selected from azetidinyl, pyrrolidinyl, or piperidinyl, said heterocyclic ring optionally substituted with —CH₃; or
R⁴ is a 5-membered heteroaryl (having 1 heteroatom independently selected from nitrogen, oxygen, and sulfur and 0, 1, 2, or 3 additional ring nitrogen atoms), wherein said heteroaryl is substituted with 0-4 R^{B} independently selected from halogen, —OH, —CN, C₁-C₄alkyl, haloC₁-C₄alkyl, C₃-C₆cycloalkyl, and C₁-C₄alkoxy.

15-16. (canceled)

17. The compound of claim 1, wherein R⁴ is a 5-membered heteroaryl (having 1 heteroatom independently selected from nitrogen, oxygen, and sulfur and 0, 1, 2, or 3 additional ring nitrogen atoms) selected from the group consisting of thiophenyl, imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, and 1,2,4-triazolyl, wherein said heteroaryl is optionally substituted with 0-4 R^{B} independently selected from halogen, —OH, —CN, C₁-C₄alkyl, haloC₁-C₄alkyl, C₃-C₆cycloalkyl, and C₁-C₄alkoxy.

18. The compound of claim 1, wherein R⁴ is

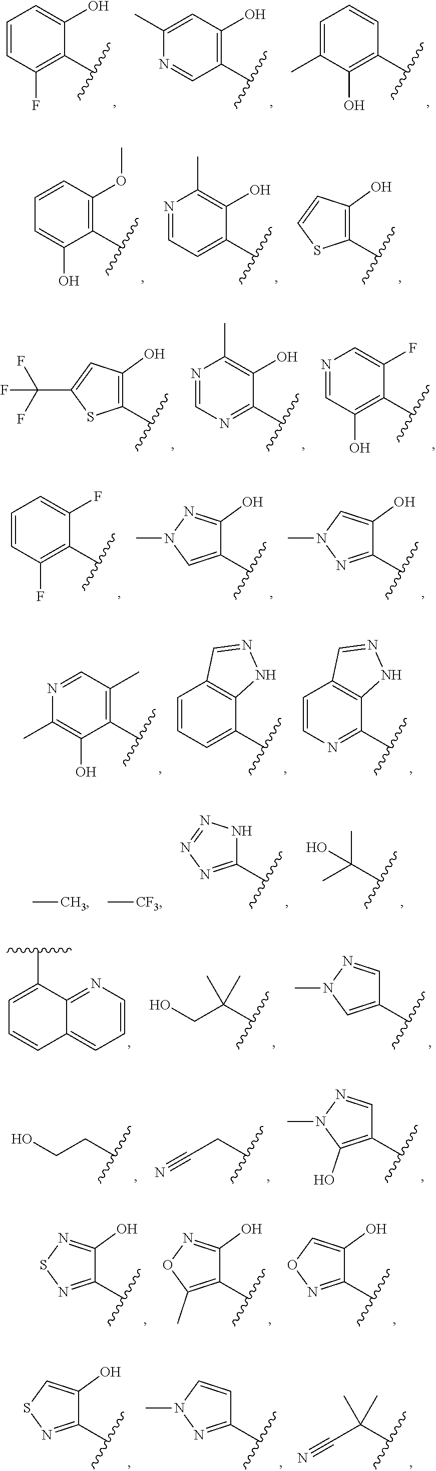

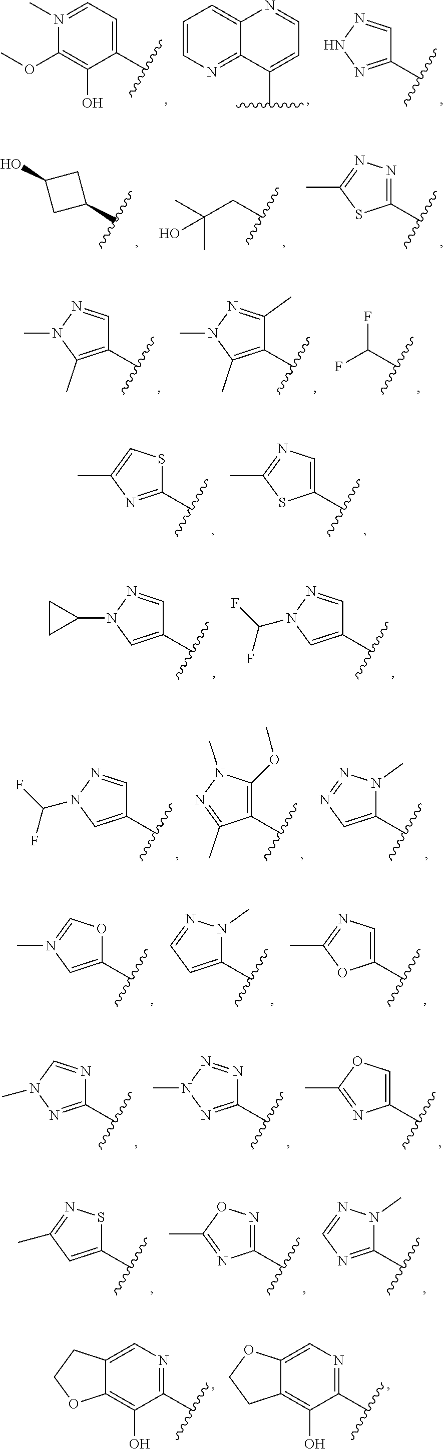

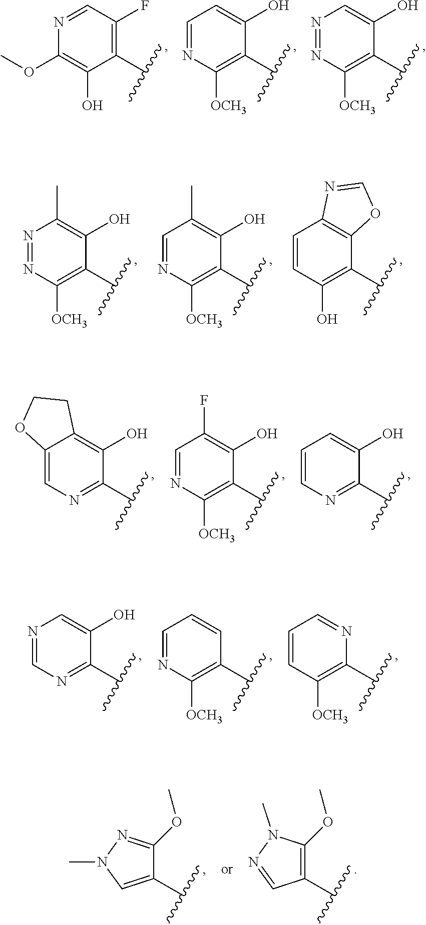

19. (canceled)

20. The compound of claim 1, wherein R² is

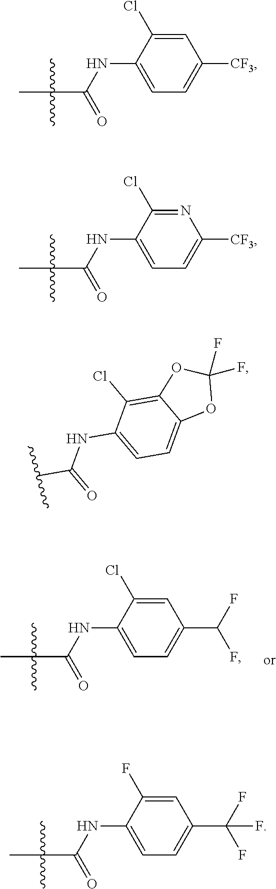 [SMILES: *C(=O)Nc1ccc(C(F)(F)F)cc1Cl ; *C(=O)Nc1ccc(C(F)(F)F)nc1Cl ; *C(=O)Nc1ccc2c(c1Cl)OC(F)(F)O2 ; *C(=O)Nc1ccc(C(F)F)cc1Cl ; *C(=O)Nc1ccc(C(F)(F)F)cc1F]

21. The compound of claim 1, wherein R³ is C₁-C₄alkyl or C₃-C₅cycloalkyl.

22. The compound of claim 1, wherein Ring A and the 0-4 independently selected R^{B} substituents with which Ring A is substituted, is:

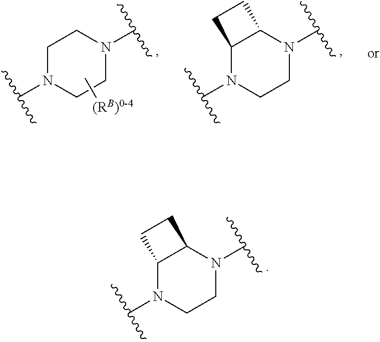

23-24. (canceled)

25. A compound selected from:

| No. | Compound |
|---|---|
| I-1 | 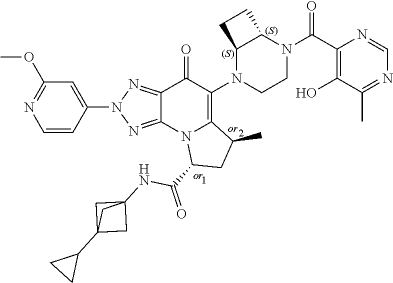 |
| I-2 | 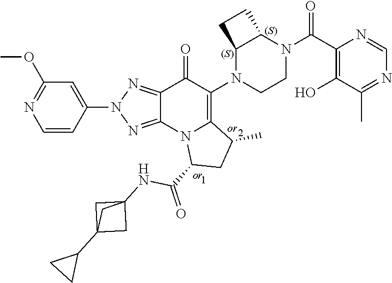 |
| I-3 | 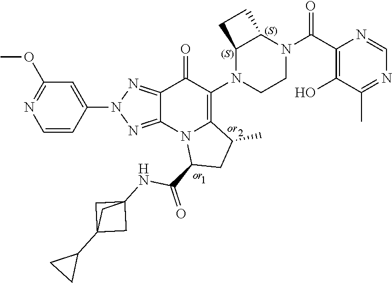 |
| I-4 | 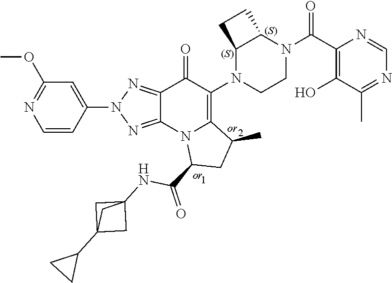 |
| I-5 | 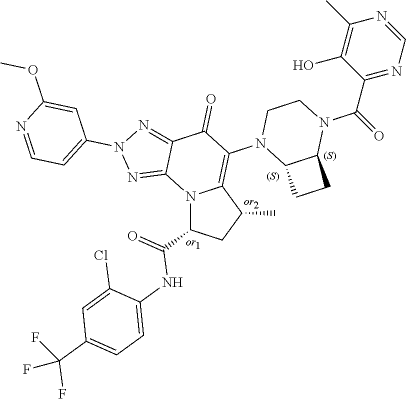 |
| I-6 | 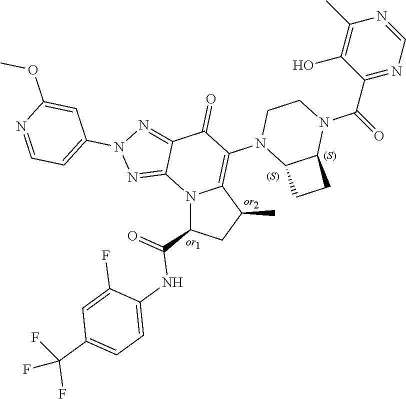 |
| I-7 | 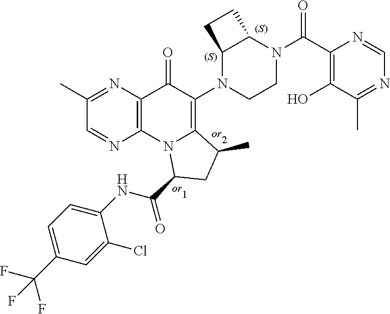 |
| I-8 | 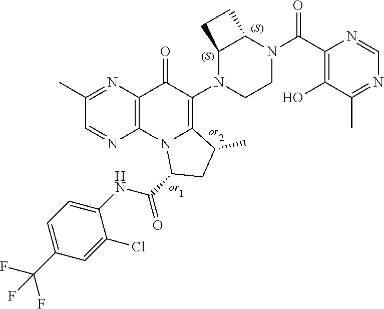 |
| I-9 | 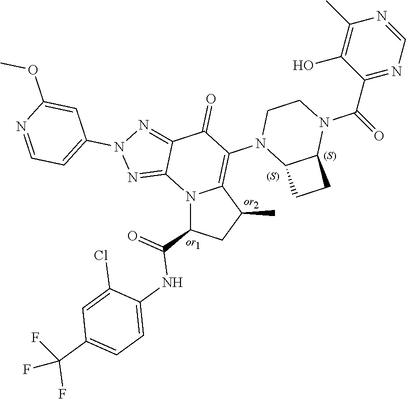 |
| I-10 | 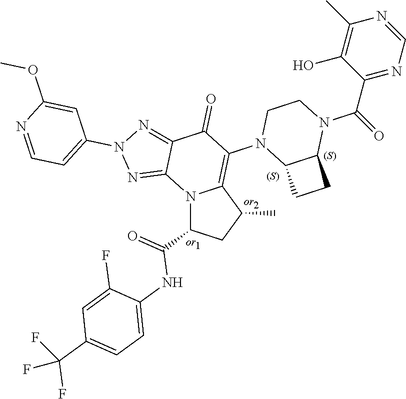 |
| I-11 | 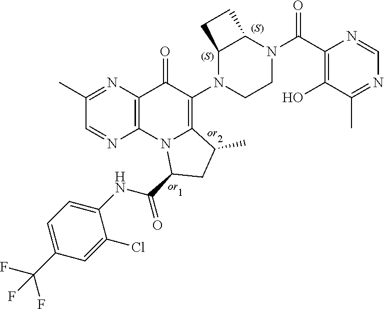 |
| I-12 | 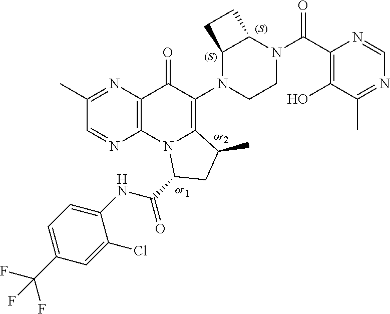 |
| I-13 | 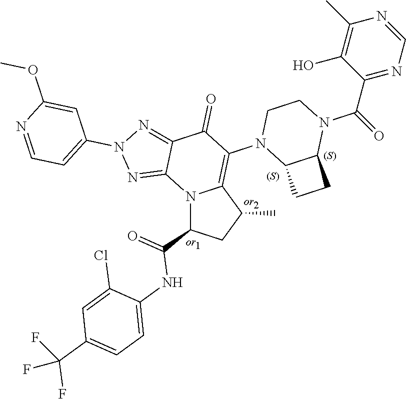 |
| I-14 | 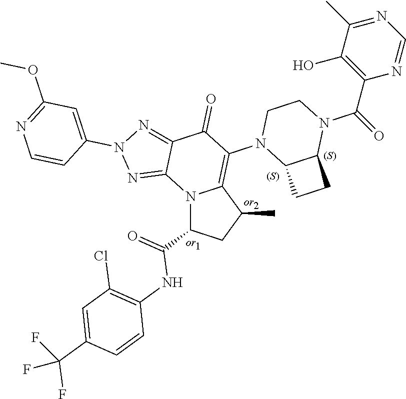 |
| I-15 | 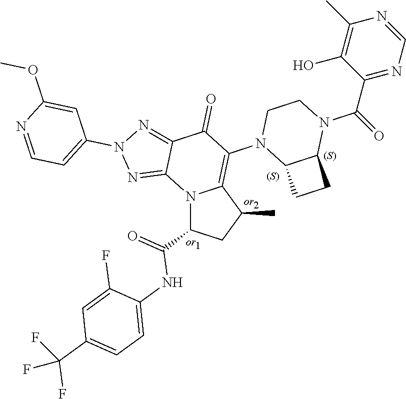 |
| I-16 | 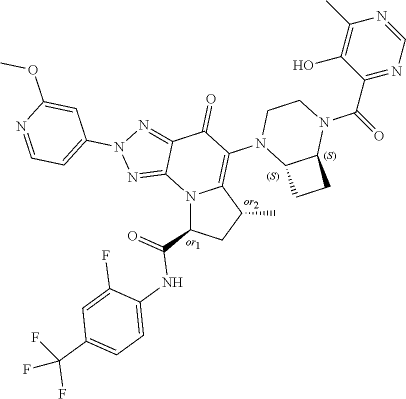 |
| I-17 | 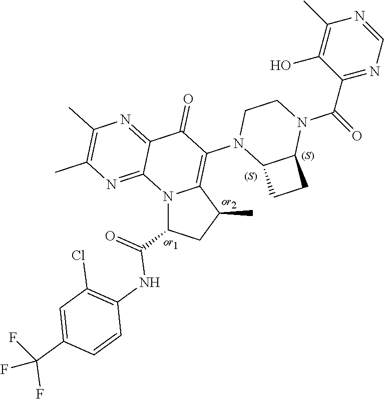 |
| I-18 | 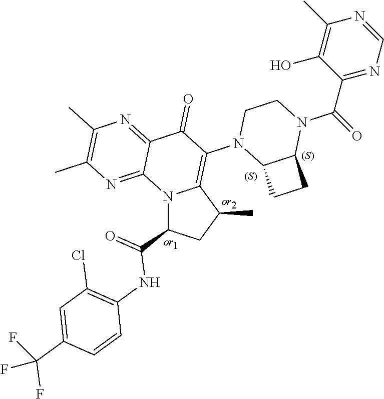 |
| I-19 | 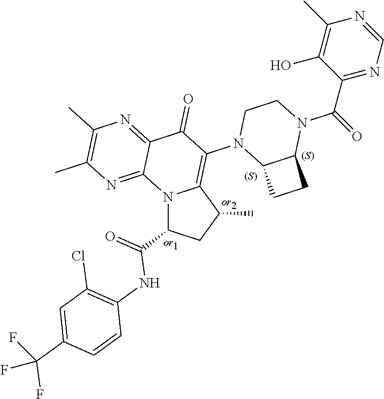 |
| I-20 | 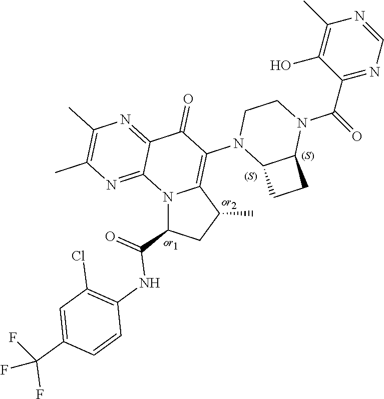 |
| I-21 | 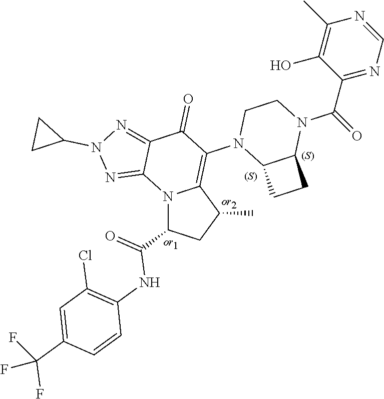 |
| I-22 | 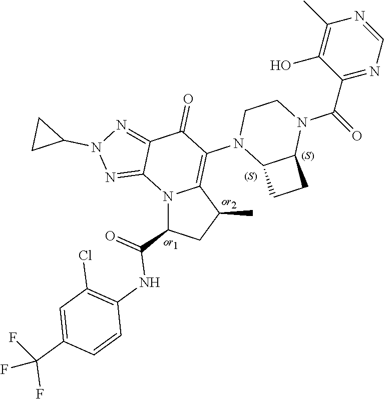 |
| I-23 | 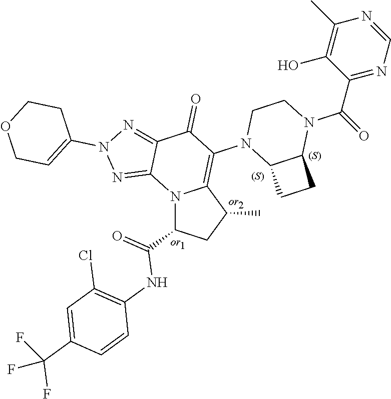 |
| I-24 | 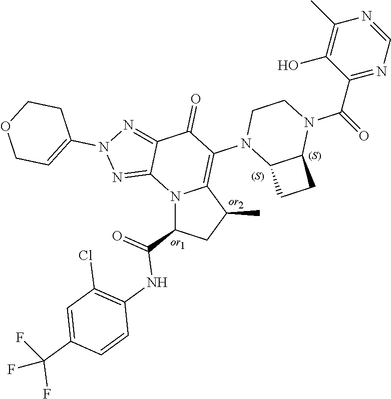 |
| I-25 | 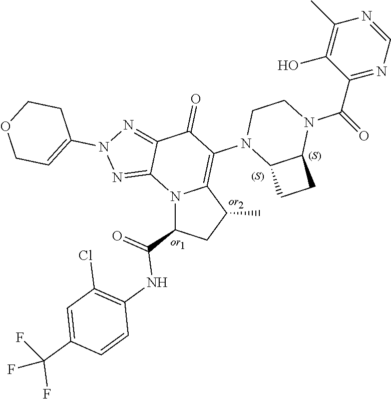 |
| I-26 | 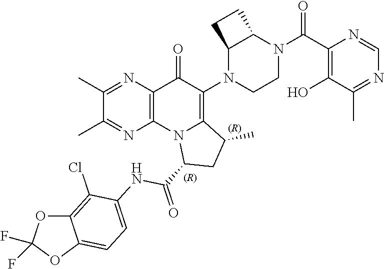 |
| I-27 | 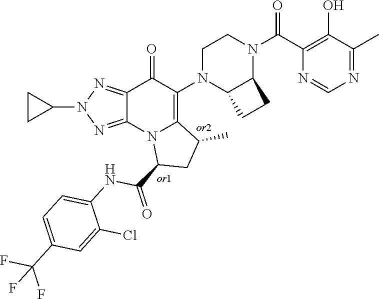 [SMILES: Cc1ncnc(C(=O)N2CCN(c3c4n(c5nn(C6CC6)nc5c3=O)[C@H](C(=O)Nc3ccc(C(F)(F)F)cc3Cl)C[C@H]4C)[C@H]3CC[C@@H]32)c1O] |
| I-28 | 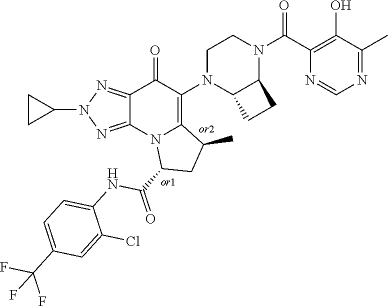 [SMILES: Cc1ncnc(C(=O)N2CCN(c3c4n(c5nn(C6CC6)nc5c3=O)[C@@H](C(=O)Nc3ccc(C(F)(F)F)cc3Cl)C[C@@H]4C)[C@H]3CC[C@@H]32)c1O] |
| I-3a | 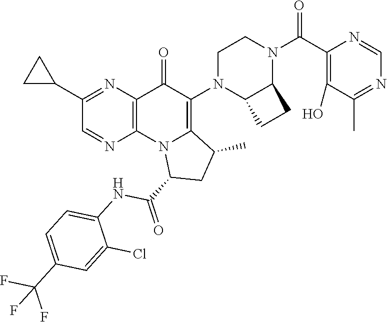 [SMILES: Cc1ncnc(C(=O)N2CCN(c3c4n(c5ncc(C6CC6)nc5c3=O)[C@@H](C(=O)Nc3ccc(C(F)(F)F)cc3Cl)C[C@H]4C)[C@H]3CC[C@@H]32)c1O] |
| I-4a | 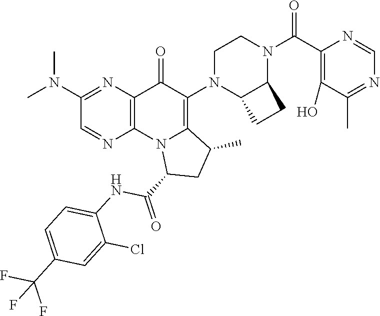 [SMILES: Cc1ncnc(C(=O)N2CCN(c3c4n(c5ncc(N(C)C)nc5c3=O)[C@@H](C(=O)Nc3ccc(C(F)(F)F)cc3Cl)C[C@H]4C)[C@H]3CC[C@@H]32)c1O] |
| I-5a | 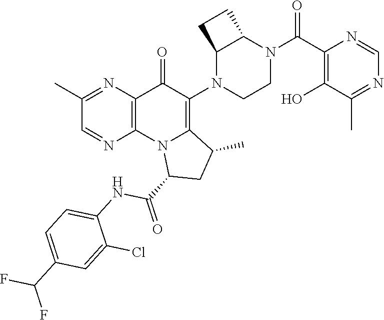 [SMILES: Cc1cnc2c(n1)c(=O)c(N1CCN(C(=O)c3ncnc(C)c3O)[C@H]3CC[C@@H]31)c1n2[C@@H](C(=O)Nc2ccc(C(F)F)cc2Cl)C[C@H]1C] |
| I-6a | 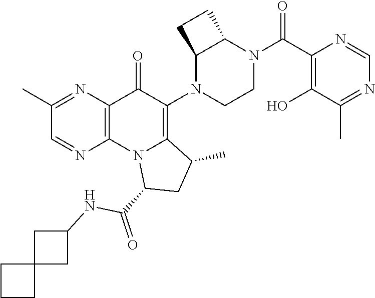 [SMILES: Cc1cnc2c(n1)c(=O)c(N1CCN(C(=O)c3ncnc(C)c3O)[C@H]3CC[C@@H]31)c1n2[C@@H](C(=O)NC2CC3(CCC3)C2)C[C@H]1C] |
| I-7a | 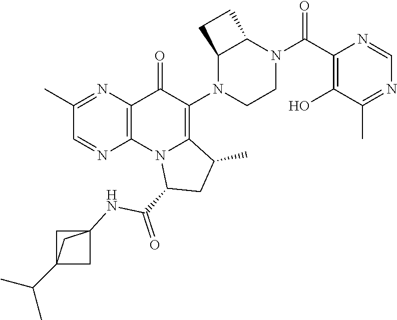 [SMILES: Cc1cnc2c(n1)c(=O)c(N1CCN(C(=O)c3ncnc(C)c3O)[C@H]3CC[C@@H]31)c1n2[C@@H](C(=O)NC23CC(C(C)C)(C2)C3)C[C@H]1C] |
| I-8a | 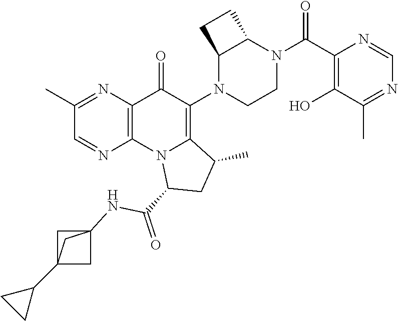 [SMILES: Cc1cnc2c(n1)c(=O)c(N1CCN(C(=O)c3ncnc(C)c3O)[C@H]3CC[C@@H]31)c1n2[C@@H](C(=O)NC23CC(C4CC4)(C2)C3)C[C@H]1C] |
| I-9a | 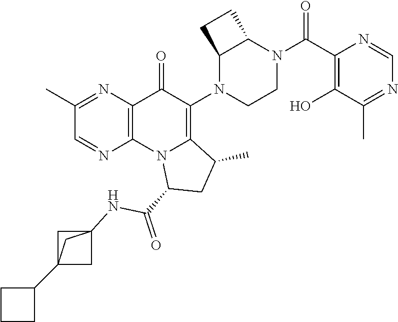 [SMILES: Cc1cnc2c(n1)c(=O)c(N1CCN(C(=O)c3ncnc(C)c3O)[C@H]3CC[C@@H]31)c1n2[C@@H](C(=O)NC23CC(C4CCC4)(C2)C3)C[C@H]1C] |
| I-10a | 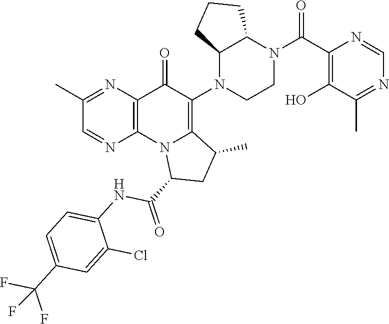 [SMILES: Cc1cnc2c(n1)c(=O)c(N1CCN(C(=O)c3ncnc(C)c3O)[C@H]3CCC[C@@H]31)c1n2[C@@H](C(=O)Nc2ccc(C(F)(F)F)cc2Cl)C[C@H]1C] |
| I-11a | 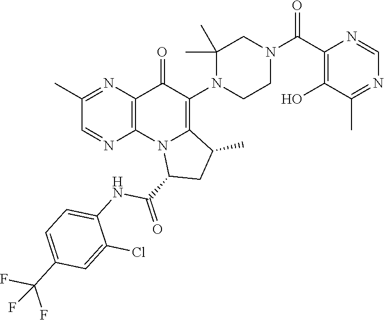 [SMILES: Cc1cnc2c(n1)c(=O)c(N1CCN(C(=O)c3ncnc(C)c3O)CC1(C)C)c1n2[C@@H](C(=O)Nc2ccc(C(F)(F)F)cc2Cl)C[C@H]1C] |
| I-12a | 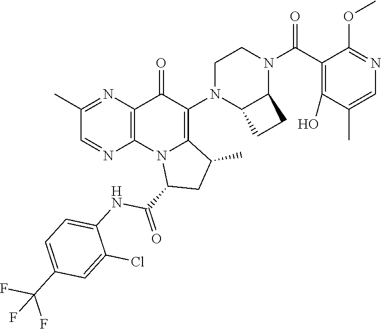 [SMILES: COc1ncc(C)c(O)c1C(=O)N1CCN(c2c3n(c4ncc(C)nc4c2=O)[C@@H](C(=O)Nc2ccc(C(F)(F)F)cc2Cl)C[C@H]3C)[C@H]2CC[C@@H]21] |
| I-18a | 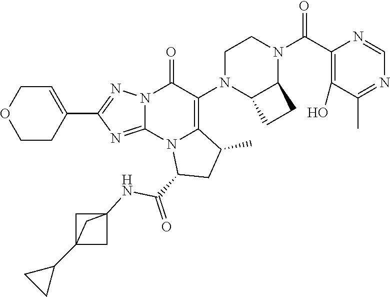 [SMILES: Cc1ncnc(C(=O)N2CCN(c3c4n(c5nc(C6=CCOCC6)nn5c3=O)[C@@H](C(=O)NC35CC(C6CC6)(C3)C5)C[C@H]4C)[C@H]3CC[C@@H]32)c1O] |
| I-19a | 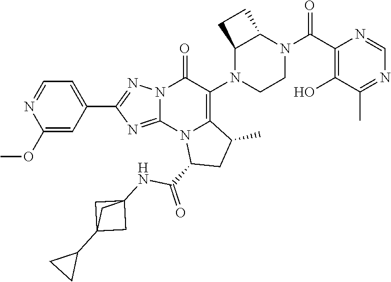 [SMILES: COc1cc(-c2nc3n4c(c(N5CCN(C(=O)c6ncnc(C)c6O)[C@H]6CC[C@@H]65)c(=O)n3n2)[C@H](C)C[C@@H]4C(=O)NC23CC(C4CC4)(C2)C3)ccn1] |
| I-27a | 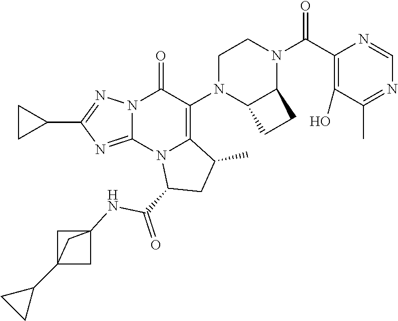 [SMILES: Cc1ncnc(C(=O)N2CCN(c3c4n(c5nc(C6CC6)nn5c3=O)[C@@H](C(=O)NC35CC(C6CC6)(C3)C5)C[C@H]4C)[C@H]3CC[C@@H]32)c1O] |
| I-28a | 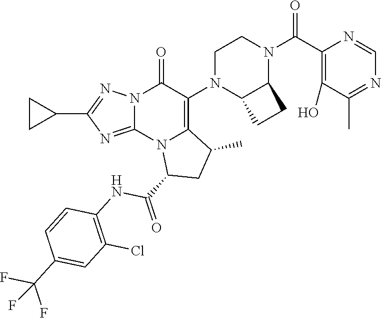 [SMILES: Cc1ncnc(C(=O)N2CCN(c3c4n(c5nc(C6CC6)nn5c3=O)[C@@H](C(=O)Nc3ccc(C(F)(F)F)cc3Cl)C[C@H]4C)[C@H]3CC[C@@H]32)c1O] |
| I-29a | 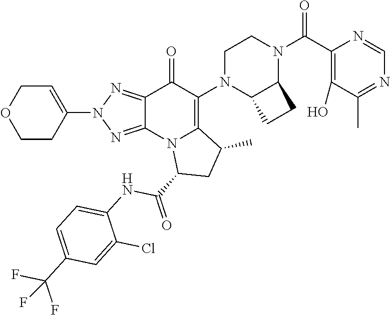 [SMILES: Cc1ncnc(C(=O)N2CCN(c3c4n(c5nn(C6=CCOCC6)nc5c3=O)[C@@H](C(=O)Nc3ccc(C(F)(F)F)cc3Cl)C[C@H]4C)[C@H]3CC[C@@H]32)c1O] |
| I-31a | 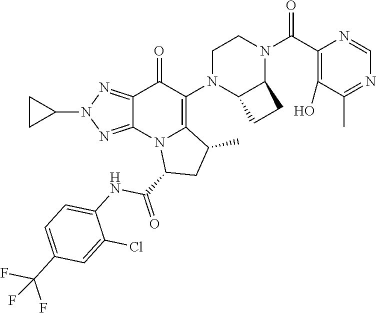 [SMILES: Cc1ncnc(C(=O)N2CCN(c3c4n(c5nn(C6CC6)nc5c3=O)[C@@H](C(=O)Nc3ccc(C(F)(F)F)cc3Cl)C[C@H]4C)[C@H]3CC[C@@H]32)c1O] |
| I-32a | 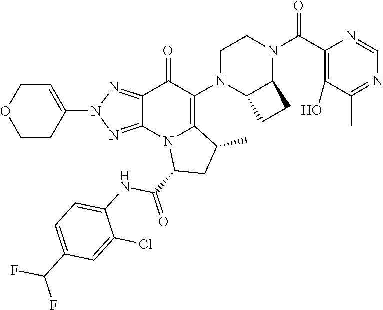 [SMILES: Cc1ncnc(C(=O)N2CCN(c3c4n(c5nn(C6=CCOCC6)nc5c3=O)[C@@H](C(=O)Nc3ccc(C(F)F)cc3Cl)C[C@H]4C)[C@H]3CC[C@@H]32)c1O] |
| I-38a | 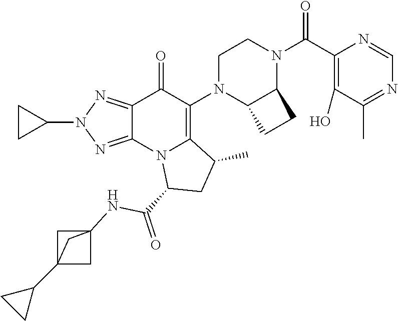 [SMILES: Cc1ncnc(C(=O)N2CCN(c3c4n(c5nn(C6CC6)nc5c3=O)[C@@H](C(=O)NC35CC(C6CC6)(C3)C5)C[C@H]4C)[C@H]3CC[C@@H]32)c1O] |
| I-39a | 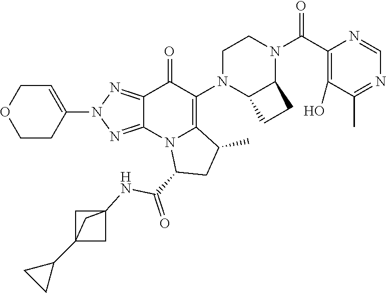 [SMILES: Cc1ncnc(C(=O)N2CCN(c3c4n(c5nn(C6=CCOCC6)nc5c3=O)[C@@H](C(=O)NC35CC(C6CC6)(C3)C5)C[C@H]4C)[C@H]3CC[C@@H]32)c1O] |
| I-40a | 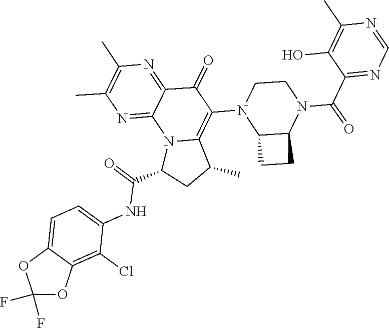 [SMILES: Cc1nc2c(=O)c(N3CCN(C(=O)c4ncnc(C)c4O)[C@H]4CC[C@@H]43)c3n(c2nc1C)[C@@H](C(=O)Nc1ccc2c(c1Cl)OC(F)(F)O2)C[C@H]3C] |
| I-41a | 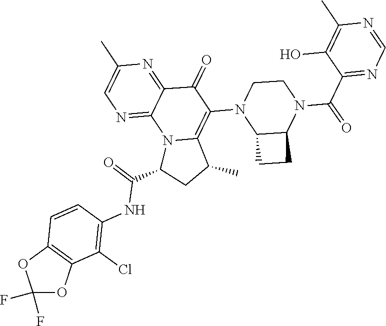 [SMILES: Cc1cnc2c(n1)c(=O)c(N1CCN(C(=O)c3ncnc(C)c3O)[C@H]3CC[C@@H]31)c1n2[C@@H](C(=O)Nc2ccc3c(c2Cl)OC(F)(F)O3)C[C@H]1C] |
| I-42a | 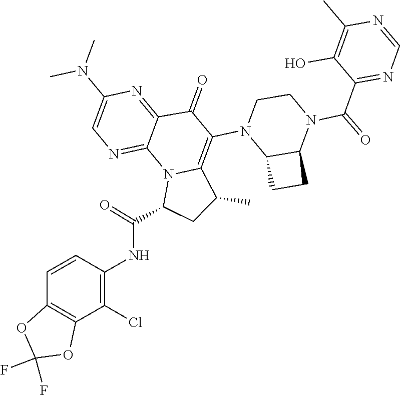 [SMILES: Cc1ncnc(C(=O)N2CCN(c3c4n(c5ncc(N(C)C)nc5c3=O)[C@@H](C(=O)Nc3ccc5c(c3Cl)OC(F)(F)O5)C[C@H]4C)[C@H]3CC[C@@H]32)c1O] |
| I-43a | 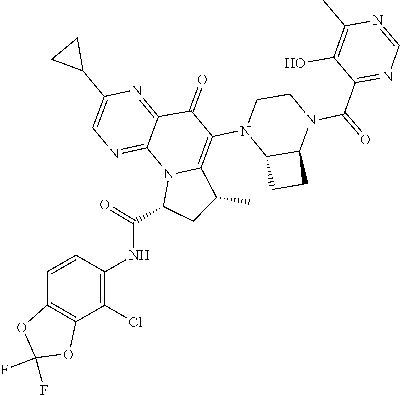 [SMILES: Cc1ncnc(C(=O)N2CCN(c3c4n(c5ncc(C6CC6)nc5c3=O)[C@@H](C(=O)Nc3ccc5c(c3Cl)OC(F)(F)O5)C[C@H]4C)[C@H]3CC[C@@H]32)c1O] |

or a pharmaceutically acceptable salt thereof.

26. A pharmaceutical composition comprising a compound or a pharmaceutically acceptable salt thereof according to claim 1, and one or more pharmaceutically acceptable carriers.

27. A method of treating cancer in a subject, wherein the cancer is characterized as microsatellite instability-high (MSI-H) or mismatch repair deficient (dMMR), comprising administering to the subject a therapeutically effective amount of a compound of claim 1, or a pharmaceutically acceptable salt thereof, or a pharmaceutical composition thereof.

28. (canceled)

29. A method of treating a disorder or disease which can be treated by WRN inhibition in a subject, wherein the method comprises administering to the subject a therapeutically effective amount of the compound according to claim 1, or a pharmaceutically acceptable salt thereof, or a pharmaceutical composition thereof.

30. A method of inhibiting WRN in a subject or modulating WRN activity in a subject, wherein the method comprises administering to the subject a therapeutically effective amount of the compound according to claim 1, or a pharmaceutically acceptable salt thereof, or a pharmaceutical composition thereof.

31. (canceled)

32. The method of claim 29 wherein the disorder or disease is a cancer characterized as microsatellite instability-high (MSI-H) or mismatch repair deficient (dMMR) selected from colorectal, gastric, prostate, endometrial, adrenocortical, uterine, cervical, esophageal, breast, kidney and ovarian cancer.


SEQUENCE LISTING (5 sequences)

SEQ ID NO: 1 - DNA, 24 bp, synthetic construct
  source 1..24: mol_type = other DNA
gcactggccgtcgttttacggtcg

SEQ ID NO: 2 - DNA, 24 bp, synthetic construct
  source 1..24: mol_type = other DNA
tccaagtaaaacgacggccagtgc

SEQ ID NO: 3 - DNA, 2208 bp, synthetic construct
  source 1..2208: mol_type = other DNA
atgaacgagggcgaagaagacgacgacaaggacttcctgtggcctgcccctaacgaagaacaagtgacatgcctgaagatgtacttcggacacagtagcttcaagcctgtgcaatggaaggtcatccactccgtgctggaagaaagaagggacaacgtggctgtgatggctaccggatacggtaagtccctgtgcttccagtaccctcccgtgtacgtgggcaagatcggtctggtgatctcccctctgatctctctgatggaggaccaggtgctgcaattgaagatgtccaacatccccgcttgcttcctgggttccgctcaaagtgagaacgtgctgacagacatcaagctgggcaagtaccgcatcgtgtacgtgacccctgagtactgctccggtaacatgggtctgctgcaacagctggaggctgacatcggaatcaccctgatcgctgtggacgaggctcactgcatctccgagtggggacacgacttccgcgactccttccgtaagctgggatccttgaagaccgctctccctatggtgcctatcgtggccctgaccgccactgcttcctcctccatccgcgaggacatcgtgcgttgcctgaacctgcgcaaccctcagatcacttgcaccggtttcgaccgccctaacttgtacctcgaggtgcgtcgcaagaccggtaacatcctccaggacctgcagcctttcctggtcaagacctcctcccactgggaatttgagggccctaccatcatctactgcccttcccgcaagatgacccagcaagtcaccggcgagctgcgcaagctcaacctctcctgcggtacctaccacgctggtatgtccttctccacccgcaaggacatccaccaccgcttcgtccgtgacgaaatccaatgcgtcatcgctaccatcgctttcggaatgggcatcaacaaggctgacatccgccaggtgatccactacggcgcccccaaggacatggaatcctactaccaggaaatcggtcgcgccggtcgcgacggtctgcagtcttcctgtcacgtgctgtgggcccccgctgacatcaacctgaaccgccacctgctgaccgaaatccgcaacgagaagttccgcctgtacaagctcaagatgatggctaagatggagaagtacctgcactcctcccgctgtcgccgtcagatcatcctctcccacttcgaggacaagcaagtgcaaaaggctagcctgggtatcatgggcaccgaaaagtgttgtgacaactgccgctcccgcctcgaccactgctactccatggacgacagcgaggacacctcctgggacttcggtcctcaagctttcaagctcttgtccgctgtggacatcctgggcgagaagttcggtatcggtctccccatcctcttcctgcgtggtagcaactcccaacgcctggctgaccagtaccgccgccactccctcttcggtaccggtaaggaccagaccgagtcctggtggaaggctttctctcgccaactgatcaccgaaggtttcctggtggaggtgtcccgctacaacaagttcatgaagatctgcgctctcactaagaagggaaggaactggctgcacaaggctaacactgagtcccaatccctcatcctgcaggctaacgaggagctgtgccctaagaagttcctgctgccttcctccaagaccgtgtcctccggaacaaaggaacactgctacaaccaagtccctgtggagctctccaccgagaagaagtccaacctggagaagctgtacagctacaagccttgcgacaagatcagctccggttccaacatcagcaagaagtccatcatggtgcaatcccctgaaaaggcctactccagctcccaacctgtcatctccgctcaagagcaagagacccagatcgtgctgtacggtaagctggtcgaagcccgccaaaagcacgctaacaagatggacgtccctcccgctatcctcgccaccaacaagatcctcgtggatatggctaagatgcgccccaccaccgtcgagaacgtgaagcgcatcgacggtgtctccgagggtaaggccgctatgctggctcctctgctggaagtgatcaagcacttctgccagaccaactccgtgcagaccgacctgttcagtagtgagaacctgtacttccaaggccaccatcatcatcatcatcaccactaa

SEQ ID NO: 4 - protein, 735 aa, synthetic construct
MNEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKFLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSENLYFQGHHHHHHHH

SEQ ID NO: 5 - protein, 8 aa, synthetic construct
HHHHHHHH